(19) United States
(12) Patent Application Publication
(10) Pub. No.: US 2024/0150440 A1
(43) Pub. Date: May 9, 2024

(54) CONSTRUCTS COMPRISING SINGLE DOMAIN VHH ANTIBODIES AGAINST SARS COV-2

(71) Applicant: Lumen Bioscience, Inc., Seattle, WA (US)

(72) Inventors: James Michael ROBERTS, Seattle, WA (US); Benjamin JESTER, Seattle, WA (US); Mesfin Mulugeta GEWE, Bothell, WA (US); Nhi Yen KHUONG, Kenmore, WA (US)

(21) Appl. No.: 18/268,844

(22) PCT Filed: Dec. 23, 2021
(86) PCT No.: PCT/US21/65138

§ 371 Date: Jun. 21, 2023

Related U.S. Application Data
(60) Provisional application No. 63/129,877, filed on Dec. 23, 2020.

(51) Int. Cl. C07K 16/10; A61K 39/00; C07K 14/705

(52) U.S. Cl. CPC C07K 16/1003; C07K 14/7056; A61K 2039/517; A61K 2039/543; C07K 2317/14; C07K 2317/569; C07K 2317/92

(57) ABSTRACT

The present disclosure relates to single domain antibodies (“VHHs”) against SARS-CoV-2, as well as to polypeptides comprising one or more of such VHHs. The disclosure also relates to nucleic acids encoding such VHHs and polypeptides; to methods of preparing such VHHs and polypeptides; to host cells expressing or capable of expressing such VHHs or polypeptides; to compositions comprising such VHHs, polypeptides, nucleic acids or host cells; and to uses of such VHHs, such polypeptides, such nucleic acids, such host cells or such compositions, in particular for prophylactic, therapeutic or diagnostic purposes.

### CROSS REFERENCE TO RELATED APPLICATIONS

This Application claims priority to U.S. Provisional Patent Application No. 63/129,877 filed Dec. 23, 2020, the entire contents of which are incorporated by reference herein.

### GOVENRMENT FUNDING

This disclosure was made with Government support under grant number MTEC-20-09-COVID19-029. The U.S. Government has certain rights to this disclosure.

### INCORPORATION BY REFERENCE OF THE SEQUENCE LISTING

The contents of the text file submitted electronically herewith are incorporated herein by reference in their entirety: A computer readable format copy of the Sequence Listing (filename: LUBI-033-01WO_SeqList_ST25.txt, date recorded: Dec. 21, 2021, file size ≈238 kilobytes).

### FIELD

The present disclosure relates to single domain antibodies (“VHHs”) against SARS-CoV-2, as well as to polypeptides comprising one or more of such VHHs. The disclosure also relates to nucleic acids encoding such VHHs and polypeptides; to methods of preparing such VHHs and polypeptides; to host cells expressing or capable of expressing such VHHs or polypeptides; to compositions comprising such VHHs, polypeptides, nucleic acids or host cells; and to uses of such VHHs, such polypeptides, such nucleic acids, such host cells or such compositions, in particular for prophylactic, therapeutic or diagnostic purposes.

### BACKGROUND OF THE DISCLOSURE

Coronaviruses (CoV) are known to cause relatively mild upper respiratory tract infections, and account for approximately 30% of the cases of the common cold in humans. However, a recently identified CoV, severe acute respiratory syndrome coronavirus (SARS-CoV-2), causes severe respiratory distress in humans leading to significant mortality in individuals infected. In 2020, SARS-CoV-2 established efficient human to human transmission resulting in a worldwide pandemic. By Dec. 20, 2021, SARS-CoV-2 was responsible for more than 5, 331, 019 confirmed deaths and 271, 963, 258 confirmed cases worldwide (see World Health Organization website, Coronavirus disease (COVID-19) pandemic).

The majority of non-hospitalized subjects infected with SARS-COV-2 experience both GI and respiratory symptoms, and 25% experience only GI symptoms¹. Historically, 25% of MERS cases² and 16-73% of SARS cases in 2002-2003³ presented with significant gastrointestinal distress. SARS-CoV-1 is able to replicate in the GI tract and live virus can be isolated from stool samples⁴. SARS-CoV-2 shares these characteristics. Both use the ACE2 receptor, which is expressed on intestinal epithelial cells at levels nearly 100 times greater than on respiratory epithelial cells⁵; capsid proteins from SARS-CoV-2 have been identified in GI epithelial cells from subjects infected with SARS-COV-2⁶; viral RNA is present in rectal and stool samples in 53% of hospitalized subjects and detected in stool samples in 23% of subjects after respiratory symptoms have resolved and nasopharyngeal swabs test negative6,7. SARS-CoV-2 also clearly infects GI tissues of the best available animal model, shedding live virus in the stool⁸. In populations with weakened immune systems (e.g., the elderly) GI colonization may result in self-infection of the airways. The systemic inflammatory response associated with GI infection may also exacerbate pulmonary disease. Blunting the cytokine storm emanating from the gastrointestinal tract could reduce the frequency or severity of acute respiratory distress syndrome in infected subjects.

The Amoy Gardens SARS super-cluster was traced to faulty engineering that enhanced fecal-oral transmission⁹. Very high amounts of SARS-CoV-2 viral RNA are present in stool samples⁶, even in subjects who never suffer any respiratory symptoms¹. Like norovirus, SARS-CoV-2 outbreaks occur on cruise ships and in nursing homes¹⁰, where the risk of fecal-oral transmission is especially high. Similarly, a US aircraft carrier was recently forced to return to port after a SARS-CoV-2 outbreak, demonstrating the important implications for operational readiness¹⁰. Therapeutics and preventatives to treat this documented route of infection, this reservoir of ongoing viral disease, and this potential means of transmission should not be overlooked. However, no such therapeutic specifically targeting the GI viral reservoir has been developed.

The present disclosure relates to therapeutics derived from ultra-simple antibodies unique to the camelid family that possess antigen binding affinities and specificities that are comparable to the more complex heavy/light chain human antibodies. These therapeutics require only the isolated, small, single polypeptide antigen-binding protein domains, referred to as “VHHs.” VHHs require no complex modifications for full bioactivity (i.e., no supramolecular assembly, no essential disulfide bonds for correct folding, and no glycosylation). VHHs are superior to human antibodies in their ability to bind to epitopes inaccessible to the larger heavy/light chain antibodies. VHHs also tend to be more stable on the shelf and in the proteolytic environment of the gut lumen.

Antibody therapies, including VHHs, are proven therapies for enteric diseases. First successfully demonstrated in human clinical trials in 1988, the efficacy of anti-pathogen antibodies has since been replicated many times using a variety of antibody formats, disease targets, and various in vivo efficacy trial designs with both humans and a wide range of animal models. VHHs given orally have been shown to be effective in treating rotavirus infection in human infants, and in preventing pathogen infections in a variety of animal models including ETEC and rotavirus infection. Oral VHHs targeting TNF-alpha are an effective therapy in inflammatory bowel disease. For example, oral hyperimmune bovine colostrum is effective for preventing and treating C. difficile infection in piglets, and for preventing ETEC infection in humans.

Antibodies are powerful therapeutic tools; broadly reactive as a class, yet individually possessing exquisite specificity. But the enormous risk and complexity associated with their clinical development, and their slow and cumbersome manufacturing processes, have made them a poor choice for rapidly responding to threats like SARS-CoV-2. Accordingly, there remains an urgent need for a therapeutic to eliminate SARS-CoV-2 viral reservoirs to reduce overall viral burden, inhibit disease progression, accelerate viral clearance, and block the fecal-oral transmission route. The present disclosure addresses this urgent need by providing neutralizing VHHs against SARS-CoV-2.

### BRIEF SUMMARY

Provided herein is a VHH antibody that binds SARS-CoV-2, wherein the VHH antibody comprises three complementarity determining regions (CDR1, CDR2, and CDR3) each of which is selected from an amino acid sequence comprising at least about 85% identity to a sequence in Table 1. In embodiments, the CDR1, CDR2, and CDR3 each comprise at least about 90%, 95%, 97%, or 99% identity to a sequence in Table 1. In embodiments, the CDR1, CDR2, and CDR3 correspond to SEQ ID NO: 610, 611, and 612. In embodiments, the VHH antibody comprises at least 85% identity with SEQ ID NO: 365. In embodiments, CDR1, CDR2, and CDR3 correspond to SEQ ID NO: 421, 422, and 423. In embodiments, the VHH antibody comprises at least 85% identity with SEQ ID NO: 302.

In embodiments, provided is a polypeptide comprising a VHH antibody. In embodiments, provided is a polypeptide comprising at least two VHH antibodies. In embodiments, the at least two VHH antibodies comprise SEQ ID NO: 365 or SEQ ID NO: 302. In embodiments, the at least two VHH antibodies are connected to each other via a linker. In embodiments, the linker comprises at least about 85% identity to SEQ ID NO: 390. In embodiments, provided is a polypeptide comprising at least three VHH antibodies. In embodiments, provided is a polypeptide comprising at least four VHH antibodies. In embodiments, the VHH antibodies are different. In embodiments, the VHH antibodies are the same.

Provided is a pharmaceutical composition comprising a VHH antibody and a pharmaceutically acceptable carrier.

Provided is a method of treating a disease or disorder related to SARS-CoV-2, the method comprising: administering a pharmaceutical composition to a subject in need thereof, thereby treating the disease or disorder related to SARS-CoV-2. In embodiments, the subject in need thereof is infected with SARS-CoV-2 as determined by PCR assay. In embodiments, the subject in need thereof has been diagnosed as being infected with SARS-CoV-2. In embodiments, the subject in need thereof is at risk of developing Coronavirus Disease (COVID). In embodiments, the subject in need thereof is at risk of developing severe COVID. In embodiments, the subject in need thereof has one or more comorbidities. In embodiments, the subject in need thereof is treated for the one or more comorbidities. In embodiments, the pharmaceutical composition is delivered encapsulated in a spirulina cell. In embodiments, the spirulina cell is dried. In embodiments, the spirulina cell is of strain A. platensis. In embodiments, the pharmaceutical composition is delivered orally. In embodiments, the pharmaceutical composition is delivered via the airway. In embodiments, the pharmaceutical composition is delivered via inhalation. In embodiments, the pharmaceutical composition is delivered nasally. In embodiments, the SARS-CoV-2 is a variant of concern. In embodiments, the variant of concern is selected from the group consisting of: Alpha, Beta, Gamma, Delta, and Omicron. In embodiments, the SARS-CoV-2 evades an immune response associated with vaccine therapy.

Provided is a method of treating a disease or disorder related to SARS-CoV-2, the method comprising: administering a pharmaceutical composition to a subject in need thereof thereby treating the disease or disorder related to SARS-CoV-2, wherein the disease or disorder is recalcitrant to anti-SARS-CoV-2 vaccine therapy. In embodiments, the subject in need thereof has previously been administered an anti-SARS-CoV-2 monoclonal therapy. In embodiments, the subject in need thereof has received a booster of the anti-SARS-CoV-2 vaccine therapy. In embodiments, the subject in need thereof has not received a booster of the anti-SARS-CoV-2 vaccine therapy. In embodiments, the administering is effective in reducing or eliminating a symptom of the disease or disorder related to the SARS-CoV-2. In embodiments, the symptom is a gastrointestinal symptom. In embodiments, the symptom is a respiratory symptom. In embodiments, the administering is effective in reducing a period of illness associated with the disease or disorder related to the SARS-CoV-2 by at least about 1-3 days.

It is another object of the disclosure to provide compositions comprising a recombinant spirulina, wherein the recombinant spirulina comprises at least one VHH. In embodiments, the VHH is delivered orally to the gastrointestinal tract. In embodiments, the VHH is delivered by other non-parenteral routes such as the airway, inhalation, and/or intranasally. In embodiments, the VHH is delivered parenterally.

In embodiments, the disclosure relates to host or host cell that expresses or can express a VHH of the disclosure and/or a polypeptide of the disclosure; and/or that contains a nucleic acid encoding a VHH of the disclosure and/or a polypeptide of the disclosure.

The disclosure further relates to a product or composition containing or comprising a VHH of the disclosure, a polypeptide of the disclosure; and/or a nucleic acid of the disclosure. Such a product or composition may for example be a pharmaceutical composition or a product or composition for diagnostic use.

The disclosure further relates to methods for preparing or generating the VHHs, polypeptides, nucleic acids, host cells, products and compositions as described herein, which methods are as further described below.

The disclosure further relates to applications and uses of the above VHHs, polypeptides, nucleic acids, host cells, products and compositions described herein, which applications and uses include, but are not limited to, the applications and uses described herein below.

Other embodiments, embodiments, advantages, and applications of the disclosure will become clear from the further description herein below.

### BRIEF DESCRIPTION OF DRAWINGS

FIG. 1 shows antigen binding kinetics of anti-SARS-CoV-2 VHHs of the disclosure. In brief, VHHs were purified from E. coli. Overall protein expression and quality were assessed using SDS-PAGE gel and protein concentrations were measured by absorbance. The antigen, recombinant RBD with the C-terminal biotin acceptor AviTag was expressed and purified from mammalian systems and the resultant protein was biotinylated using E. coli biotin ligase enzyme BirA (Avidity Bio). Biotinylated RBD was captured on streptavidin biosensors (ForteBio) and antigen loading was quenched using 50 μM biocytin. Binding kinetics for anti-SARS-CoV-2 VHHs and SARS-CoV-2 RBD assayed using the BLI system. Binding curves were generated using global fitting with 1:1 interaction model and the kD is indicated in red.

FIG. 2 shows characterization of the VHHs of the disclosure through neutralization, binding, and competition assays.

FIG. 3 shows microneutralization assay results of 141 VHHs screened at 10 μg/mL.

FIG. 4 shows representative results of microneutralization assay with serial dilution of VHHs.

FIG. 5 shows BLI-based epitope binning of SARS VHH-72 and VHHs of the disclosure. BLI-based epitope binning of SARS VHH-72 and anti-SARS-CoV-2 VHHs. Biotinylated RBD was captured on streptavidin biosensors (ForteBio) and antigen loading was quenched by incubating with 50 μM Biocytin. RBD-loaded biosensors were initially incubated with 10 μg/mL of VHH1 until saturation to assess epitope binning. This initial association step assays binding of VHH1. VHH1-saturated biosensors were transitioned to microwells containing 10 μg/mL of VHH2. Association of VHH2 can be measured during the second association step only if VHH1 and VHH2 bind different epitopes.

FIG. 6A -FIG. 6C show competition of selected VHHs of the disclosure against human ACE2-FC dimer, SARS VHH-72 dimer, and VHH R2B-D5 dimer for SARS-CoV-2-RBD binding. High-binding ELISA plates are coated with 1 μg/mL of the specified competing protein. Separately, SARS-CoV-2 RBD with C-terminal AviTag at 0.5 μg/mL was incubated for 1 hour at room temperature with a 5-fold VHH dilution series, starting at 20 μg/mL. After incubation, 100 μL of the VHH-RBD mix was added on to antigen-coated plates. RBD binding to ACE2-FC was detected using mouse anti-AviTag primary antibody (Avidity Bio) at 1:5,000 dilution and HRP conjugated goat anti-mouse secondary antibody at 1:5,000 dilution (BioRad). FIG. 6A shows ELISA-based ACE2 competition for RBD binding where ELISA plates are coated with human ACE2-FC at 1 μg/mL. FIG. 6B shows ELISA based SARS VHH-72-Ank1C2 dimer competition for RBD binding where ELISA plates are coated with SARS VHH-72-Ankl C2 at 1 μg/mL. FIG. 6C shows ELISA based VHH R2B-D5-5HVZ-MBP dimer competition for RBD binding where ELISA plates are coated with R2B-D5-5HVZ-MBP at 1 μg/mL.

FIG. 7 shows protease resistance results.

FIG. 8A shows an image of an expression gel. FIG. 8B shows an image of a gel showing soluble protein. FIG. 8C shows an image of a gel showing the final analysis.

FIG. 9A shows an image of a gel showing protein expression. FIG. 9B shows absorbance. Of note, is the presence of the two free Cys amino acids result in aggregate formation.

FIG. 10A shows an image of a gel showing protein expression. FIG. 10B shows absorbance. Of note, is that mutating the Cys residues to Ser resulted in monodispersed monomer protein.

FIG. 11A shows an image of a gel showing protein expression. FIG. 11B shows absorbance. Docking peptide fused VHH R2B-D5 expressed as MBP-fusion.

FIG. 12 shows Size-Exclusion Chromatography based comparison of the PP758 (2-R2B-D5), PP1625 (1-S3b-C8), and PP1896 (2-R2B-D5 and 1-S3b-C8) constructs. The Cerberbody PP1896 forms a complex when mixed 1:1.5 molar ratio between the dimeric PP758 and the monomeric PP1625, thereby increasing binding avidity.

FIG. 13 shows kinetics binding responses for the Cerberbody forming components, PP758, PP917, and the Cerberbody PP1896.

FIG. 14 shows Size-Exclusion Chromatography based comparison of Cerberbody, PP1897 and complex forming components, PP917, and PP1625.

FIG. 15 shows kinetics binding responses for the Cerberbody forming components, PP758, PP917, and the Cerberbody complex PP1897.

FIG. 16 shows Size-Exclusion Chromatography based comparison of Cerberbody complex PP1898, and complex forming components, PP917, and PP1823.

FIG. 17 shows kinetics binding responses for the Cerberbody forming components, PP758, PP917, and the Cerberbody complex PP1898.

FIG. 18A shows a graphic of an exemplary Hydrabody complex formation. FIG. 18B shows a graphic of an exemplary Cerberbody complex formation.

FIG. 19 shows a gel of protein expression of exemplary Hydrabody forming monomeric dimer constructs PP1892, PP1893, PP1894, and PP1895.

FIG. 20A shows an image of a gel of Ni-NTA purification results of construct PP1892. FIG. 20B shows sizing analysis of Hydrabody forming dimeric construct PP1892.

FIG. 21A shows an image of a gel of Hydrabody 1894. FIG. 21B shows Size-Exclusion Chromatography analysis result for the same.

FIG. 22A shows an image of a gel of Hydrabody 1895. FIG. 22B shows Size-Exclusion Chromatography based analysis result for the same construct.

FIG. 23 shows a comparison of binding between referenced monomer, dimer, and trimer constructs.

FIG. 24 shows results of a binding assay showing a comparison of binding among Hydrabody forming components.

FIG. 25A and FIG. 25B show Cryo-EM structures of PP758 in complex with SARS-CoV-2 Spike. FIG. 25A shows a cryo-EM reconstruction of PP758 in complex with prefusion stabilized SARS-CoV-2 S trimer at 3.9 Å. Two receptor binding domains (RBDs) are visible, colored in shades of green, with the VHH in magenta, and the rest of the spike colored in light purple with glycans in darker purple. FIG. 25B is a close-up view of the VHH bound to the RBD. The VHH model was generated by homology modeling and rigid body docked into the electron density map.

FIG. 26 shows a graphic of epitope binning.

FIG. 27 shows results of an ELISA assay completed evaluating binding of exemplary VHHs against SARS-CoV-2 RBD variants Alpha, Beta, Gamma, and Delta.

FIG. 28 shows IC50 measurement of purified VHHs PP917, PP758, PP1146 and PP1148.

FIG. 29A-FIG. 2C is a comparison of an in-house pseudovirus assay and IITRI live virus assay. FIG. 29A shows % reduction of infectivity of an initial in-house pseudovirus assay. FIG. 29B shows % reduction of infectivity of a pre-live in-house pseudovirus assay. FIG. 29C shows % reduction of infectivity of an IITR pseudovirus assay.

FIG. 30A-FIG. 30AK show percent reduction of viral growth per the IITRI live virus assay. P: purified VHH, P+M: Purified VHH+matrix, C: cell extract, and REM: Remdesivir (positive control).

FIG. 31 shows TRII live virus assay results, position effect corrected and rescaled. Observed (points) rescaled TRII assay data (y-axis) are shown by dilution (x-axis) with model fit (lines) superimposed. Fits with quality parameter estimates (defined as a Dil80 estimate with p<0.1 significance) are shown with solid lines; fits lacking quality parameter estimates are shown as dashed lines. Top and bottom of the assay values are given as dotted, horizontal line.

FIG. 32 shows estimates of dilution yielding 80% inhibition of viral infection (Dil80Dil180) parameter estimates are shown as points with 95% confidence intervals (range, if available) shown. An exact match between live virus assay and pseudovirus assay assessment of potency would be when Dil80Dil80 is 1.0. At values>1.0, the live virus assessment of potency is lower than the pseudovirus assessment. At values<1.0, the live virus assessment of potency is higher than the pseudovirus assessment.

FIG. 33A shows LabChip Results of Non-Reduced (left) and Reduced (right) Proj113R3 Airway Powder: Size and Estimated Purity shown on expected peak. FIG. 33B shows airway powder from Proj113: Absorbance 450 nm vs In (protein ug/ml) with 4-Par Logistic Curve Fit to Estimate EC50 ug/ml and ELISA results for PP917 Standard and the Proj113R3 Airway Powder applying Reduced Model. FIG. 33C shows Airway Powder from Proj137: Absorbance 450 nm vs log (protein ug/ml) with 4-Par Logistic Curve Fit to Estimate EC50 ug/ml and ELISA results for the PP917 Standard and the Proj137R1 Airway Powder applying Reduced Mode.

### OVERVIEW

### Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art.

Following long-standing patent law convention, the terms “a”, “an”, and “the” refer to “one or more” when used in this application, including the claims, unless clearly indicated otherwise.

Any ranges listed herein are intended to be inclusive of endpoints. For example, a range of 2-4 includes 2 and 4 and values between.

The term “about” or “approximately” when immediately preceding a numerical value means a range plus or minus 10% of that value. For example, “about 50” can mean 45 to 55, “about 25,000” can mean 22,500 to 27,500, etc., unless the context of the disclosure indicates otherwise, or is inconsistent with such an interpretation. In the context of a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

When referring to a nucleic acid sequence or protein sequence, the term “identity” is used to denote similarity between two sequences. Unless otherwise indicated, percent identities described herein are determined using the BLAST algorithm available at the world wide web address: blast.ncbi.nlm.nih.gov/Blast.cgi using default parameters.

The term “subject” as used herein refers to a vertebrate or an invertebrate, and includes mammals, birds, fish, reptiles, and amphibians. Subjects include humans and other primates, including non-human primates such as chimpanzees and other apes and monkey species. Subjects include farm animals such as cattle, sheep, pigs, goats, and horses; domestic mammals such as dogs and cats; laboratory animals including rodents such as mice, rats, and guinea pigs; birds, including domestic, wild, and game birds such as chickens, turkeys and other gallinaceous birds, ducks, geese, and the like; and aquatic animals such as fish, shrimp, and crustaceans.

### Anti-SARS-CoV-2 Single Variable Domain on a Heavy Chain (VHH) Antibodies

In embodiments, provided herein are single variable domain on a heavy chain (VHH) antibodies that target SARS-CoV-2 as well as to polypeptides comprising or essentially consisting of one or more of such VHHs. The present disclosure also relates to nucleic acids encoding such VHHs and polypeptides; to methods for preparing such VHHs and polypeptides; to host cells expressing or capable of expressing such VHHs or polypeptides; to compositions comprising such VHHs, polypeptides, nucleic acids or host cells; and to uses of such VHHs, such polypeptides, such nucleic acids, such host cells or such compositions, in particular for prophylactic, therapeutic or diagnostic purposes mentioned below. Advantages and applications of the disclosure will become clear from the further description herein below.

It is a general object of the present disclosure is to provide VHHs that specifically bind to SARS-CoV-2. In particular, it is an object of the present disclosure to provide VHHs that specifically bind to SARS-CoV-2, and to provide proteins or polypeptides comprising the same, that are suitable for therapeutic and/or diagnostic use for the prevention, treatment and/or diagnosis of infection caused by SARS-CoV-2, and/or that can be used in the preparation of a pharmaceutical composition for the prevention and/or treatment of infection caused by SARS-CoV-2.

These objects are achieved by the VHHs, proteins and polypeptides described herein. These VHHs are also referred to herein as “VHHs of the disclosure”; and these proteins and polypeptides are also collectively referred to herein “polypeptides of the disclosure”.

The present disclosure is not particularly limited to or defined by a specific antigenic determinant, epitope, part, domain, subunit or confirmation (where applicable) of SARS-CoV-2 against which the VHHs and polypeptides of the disclosure are directed. In embodiments, the VHHs of the disclosure can bind any region of SARS-CoV-2.

In embodiments, a VHH of the disclosure targets any one of the four structural proteins of SARS-CoV-2 including but not limited to: spike protein (S), envelope protein (E), membrane protein (M) and/or nucleocapsid protein (INI). The spike protein (S protein) is responsible for receptor-recognition, attachment to the cell, infection via the endosomal pathway, and the genomic release driven by fusion of viral and endosomal membranes. Though sequences between the different family members vary, there are conserved regions and motifs within the S protein making it possible to divide the S protein into two subdomains: S1 and S2. While the S2, with its transmembrane domain, is responsible for membrane fusion, the S1 domain recognizes the virus-specific receptor and binds to the target host cell. In embodiments, the VHHs of the disclosure recognize the receptor binding domain (RBD) or spike protein of SARS-CoV-2.

In embodiments, VHHs comprising one or more of the CDR's explicitly listed in Table 1 below are particularly preferred. In embodiments, VHHs comprising two or more of the CDR's explicitly listed in Table 1 to Table 3 are more particularly preferred. In embodiments, VHHs comprising three of the CDR's explicitly listed in any one of Table 1 to Table 3 are most particularly preferred.

Some particularly preferred, but non-limiting combinations of CDR sequences can be seen in Table 1 to Table 3 below, which lists the CDR's and framework sequences that are present in a number of preferred (but non-limiting) VHHs of the disclosure. As will be clear to the skilled person, a combination of CDR1, CDR2 and CDR3 sequences that occur in the same clone (i.e., CDR1, CDR2 and CDR3 sequences which are mentioned on the same line in any one of Table 1 to Table 3) will usually be preferred (although the disclosure in its broadest sense is not limited thereto, and also comprises other suitable combinations of the CDR sequences mentioned in any one of Table 1 to Table 3).

In another embodiment, a VHH that specifically binds to a SARS-CoV-2 antigen or component comprises, or alternatively consists of, a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to any one of the amino acid sequences recited in Table 2 or Table 3. In another embodiment, a VHH that specifically binds to a SARS-CoV-2 antigen or component comprises, or alternatively consists of, a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to any one of the CDRs recited in Table 1. Nucleic acid molecules encoding these antibodies are also encompassed by the disclosure.

In embodiments, the VHHs of the disclosure comprise combinations of CDRs recited in Table 1 or Table 3, where each CDR is replaced by a CDR having at least 80%, preferably at least 90%, more preferably at least 95%, even more preferably at least 99% sequence identity to one or more of the CDRs listed in Table 1.

However, as will be clear to the skilled person, the combinations of CDR sequences recited in Table 1 are generally preferred.

Thus, in the VHHs of the disclosure, at least one of the CDR1, CDR2 and/or CDR3 sequences present is chosen from the group of the CDR1, CDR2 and CDR3 sequences, respectively, listed in Table 1 or Table 3. In embodiments, the VHHs of the disclosure comprise at least one of the CDR1, CDR2, and CDR3 sequences; or from the group of CDR1, CDR2 and CDR3 sequences, respectively, that have at least 80%, preferably at least 90%, more preferably at least 95%, even more preferably at least 99% sequence identity with at least one of the CDR1, CDR2 and CDR3 sequences, respectively, listed in Table 1.

Preferably, in the VHHs of the disclosure, at least two of the CDR1, CDR2 and CDR3 sequences present are chosen from the group of CDR1, CDR2 and CDR3 sequences, respectively, listed in Table 1 or from the group of CDR1, CDR2 and CDR3 sequences, respectively, that have at least 80%, preferably at least 90%, more preferably at least 95%, even more preferably at least 99% sequence identity with at least one of the CDR1, CDR2 and CDR3 sequences, respectively, listed in Table 1.

Most preferably, in the VHHs of the disclosure, all three CDR1, CDR2 and CDR3 sequences present are chosen from the group of the CDR1, CDR2 and CDR3 sequences, respectively, listed in Table 1 or from the group of CDR1, CDR2 and CDR3 sequences, respectively, that have at least 80%, preferably at least 90%, more preferably at least 95%, even more preferably at least 99% sequence identity with at least one of the CDR1, CDR2 and CDR3 sequences, respectively, listed in Table 1.

Even more preferably, in the VHHs of the disclosure, all three CDR1, CDR2 and CDR3 sequences present are suitably chosen from the group of the CDR1, CDR2 and CDR3 sequences, respectively, listed in Table 1.

Also, generally, the combinations of CDR's listed in Table 1 (i.e., those mentioned on the same line in Table 1) are preferred. Thus, it is generally preferred that, when a CDR in a VHH of the disclosure is a CDR sequence mentioned in Table 1 or is chosen from the group of CDR sequences that have at least 80%, preferably at least 90%, more preferably at least 95%, even more preferably at least 99% sequence identity with a CDR sequence listed in Table 1, that at least one and preferably both of the other CDR's are chosen from the CDR sequences that belong to the same combination in Table 1 (i.e. mentioned on the same line in Table 1) or are chosen from the group of CDR sequences that have at least 80%, preferably at least 90%, more preferably at least 95%, even more preferably at least 99% sequence identity with the CDR sequence(s) belonging to the same combination.

Thus, by means of non-limiting examples, a VHH of the disclosure can for example comprise a CDR1 sequence that has more than 80% sequence identity with one of the CDR1 sequences mentioned in Table 1, a CDR2 sequence that has 3, 2 or 1 amino acid difference with one of the CDR2 sequences mentioned in Table 1 (but belonging to a different combination), and a CDR3 sequence.

In the most preferred in the VHHs of the disclosure, the CDR1, CDR2 and CDR3 sequences present are suitably chosen from the one of the combinations of CDR1, CDR2 and CDR3 sequences, respectively, listed in Table 1.

TABLE 1
Exemplary CDR Sequences of single domain VHH antibodies

| | | SEQ | | SEQ | | SEQ |
|---|---|---|---|---|---|---|
| | | ID | | ID | | ID |
| Construct | CDR1 | NO | CDR2 | NO | CDR3 | NO |
| R1E-B2 | GQITAENY | 391 | ITTGGST | 392 | NADRSYFPDY | 393 |
| R1E-D4 | GSIREINA | 394 | IDSGSLD | 395 | AASRSGDYRY | 396 |
| R1I-A11 | GSILQINA | 397 | ITSGGST | 398 | AATSTRGGLDSDY | 399 |
| R1I-C8 | GRQFGAWR | 400 | ITSGTSG | 401 | AATSHTTYHAEY | 402 |
| R1I-E7 | GTEYDISA | 403 | VTSDNST | 404 | TATVKTSQYRY | 405 |
| R1I-G7 | GSSININY | 406 | ITSGGGT | 407 | AASEAWRRFDS | 408 |
| R2A-C5 | GTWGNIQY | 409 | ITPNNGT | 410 | TAVLWYTTKEGPYDY | 411 |
| R2A-F3 | GSITSINW | 412 | ISQTGRG | 413 | AAASRTGTPYNY | 414 |
| R2B-C4 | GSSESWNA | 415 | ITSGTST | 416 | NGYEHTTGYDY | 417 |
| R2B-D4 | GSYASIAS | 418 | LTSGGSR | 419 | TALILHYAGMYDY | 420 |
| R2B-D5 | GYADEINM | 421 | IASGGST | 422 | AAIPRPSLSYAHDYAY | 423 |
| R2B-E6 | GGVTRGNA | 424 | IDNGGSH | 425 | AVFPDHDSEQASIDRVY | 426 |
| R2C-B3 | GTYASYET | 427 | ITGAGSS | 428 | NADSIPPALRAANTGY | 429 |
| R2C-F6 | GRILAANI | 430 | ITTGGSN | 431 | NALGLGSASGSYES | 432 |
| R2C-G6 | GSIYKYSD | 433 | LTGAGTT | 434 | AAGQGFKATRTFDS | 435 |
| R2D-A6 | GSGASIGA | 436 | ITSAGNT | 437 | NALAGANYESQWEEYNY | 438 |
| R2D-D3 | GHIFSINN | 439 | IHSGGGT | 440 | NAEVSTGAGSRGRVSDVY | 441 |
| R2D-G5 | GSAMMINR | 442 | ITWQGST | 443 | NALSADGAYVRRYKDY | 444 |
| R2E-D2 | GHQLSTNA | 445 | IGSGSSS | 446 | TALGGDELDGMTYDY | 447 |
| R2E-F4 | GETFSSNA | 448 | ITSGGND | 449 | NAPATEDTSHYPRNY | 450 |
| R5A-A1 | GAELSYTA | 451 | INSGGSS | 452 | TALLDSTYDY | 453 |
| R5A-A5 | GSAFATNG | 454 | ITSNGSI | 455 | TALLMSYGGGEEYDY | 456 |
| R5A-B3 | GOLDRSNA | 457 | ITSGAST | 458 | AADRYRAIQRTQYS | 459 |
| R5A-B5 | GDDFIENA | 460 | VTLGGHS | 461 | NYPVGIPAVAIDGRRSYGY | 462 |
| R5A-B7 | GAFFSYGA | 463 | ITSGGST | 464 | NAGVVWLPSNAAYNL | 465 |
| R5A-C5 | GDIFSINA | 466 | ITSGGST | 467 | AAQQARGGLDSDY | 468 |
| R5A-D4 | GSAMMINR | 469 | ITWQGST | 470 | NAVAPVHENENY | 471 |
| R5A-D6 | GNDWYINS | 472 | VSRGGSN | 473 | AALLETSSGDYTY | 474 |
| R5A-E5 | GYDFDILR | 475 | ITSAESV | 476 | ATYDYTDDYGYAS | 477 |
| R5A-F2 | GSISSIEA | 478 | MASGGDT | 479 | AAQTYRPDTWGRYTF | 480 |
| R5A-F5 | GYHASILA | 481 | MTWSDMT | 482 | AATYVTRDRPWTH | 483 |
| R5B-C3 | GGISVANV | 484 | LTSEGSR | 485 | AAEPTGNTHEFEY | 486 |
| R5B-C5 | GSPFKTQT | 487 | ITSGSGT | 488 | AAANDGYGVD | 489 |
| R5B-G1 | GSIGQIVL | 490 | ITSPSST | 491 | NTELEGKTQGFTGKTVWYDY | 492 |
| R5C-A1 | GLDVSINF | 493 | ITWQGST | 494 | ATVWAGGTPNDY | 495 |
| R5C-A2 | GDIFSKNA | 496 | IYSDGSS | 497 | AALDYSARYRT | 498 |
| R5C-B1 | GSGFSNNA | 499 | ITSGSVS | 500 | AAVTGSSTGNAGQGYLT | 501 |
| R5C-B2 | GSIFSLNW | 502 | INSGGHA | 503 | AAGPYGAYNFNS | 504 |
| R5C-E1 | YIFSATV | 505 | INSTGSS | 506 | AADGADVVYGNRY | 507 |
| R5C-E3 | SFFSINA | 508 | ISVGDSR | 509 | AAQIYITTYELYDY | 510 |
| R5C-F3 | TIPSINY | 511 | LTSGDNR | 512 | NAKTVGSSYRDSYNY | 513 |
| R5C-F5 | SYFKINS | 514 | IHSGDGT | 515 | AADQAWVRAGTYDY | 516 |
| R5C-F6 | GQTNSILA | 517 | ITSGSNS | 518 | AARNGKLSPKANQRLY | 519 |
| R5C-G2 | GSATSINR | 520 | IDSGGYT | 521 | NAVPVTTDKTPMYQY | 522 |
| R5C-G6 | GSIESINA | 523 | ITENDVD | 524 | ASLSSGYQYVGFRY | 525 |
| R5D-A1 | GSISSYNA | 526 | LTSSGSD | 527 | NAFSYHTYSRYDF | 528 |
| R5D-B2 | GSIWSIAF | 529 | ITTGNDE | 530 | AANTYERTPDGRTFSY | 531 |
| R5D-B4 | GSSTSINI | 532 | ITGGSNG | 533 | AAVATSYTGVEYEY | 534 |
| R5D-B5 | GSRDSIVA | 535 | ITSRWVT | 536 | NAPVYYDTEWYDI | 537 |
| R5D-B6 | GRILAANI | 538 | ITSGGGS | 539 | NALGLGQSSLYRSADPYDS | 540 |
| R5D-D4 | GSDFTINA | 541 | ITSTSST | 542 | AAFAVRDADTAGWTARDY | 543 |
| R5D-E1 | GSSFSINW | 544 | INSTGSD | 545 | AAAITQPMPQGKGAPPGFYY | 546 |
| R5D-E3 | GSIYVINT | 547 | IAKGGSG | 548 | AADRTYGYEYSTYQY | 549 |
| R5D-E4 | GQISSNNG | 550 | ATTGGTT | 551 | TALLMSRAGEFDY | 552 |
| R5D-F3 | GSQLSINY | 553 | ITITGSS | 554 | NTVDRTDNYGYDY | 555 |
| R5D-G1 | GSIFGIND | 556 | ITNGLSA | 557 | AAQVYITTYELYDY | 558 |
| R5D-G6 | GRILAANI | 559 | ITSGGST | 560 | AAKPLGSPDDFDY | 561 |
| R5E-C1 | GSIPYVQA | 562 | IGWGGGT | 563 | AAKFRENFSFYDY | 564 |
| R5E-D1 | GFEFSTSA | 565 | ITSGGSD | 566 | AAYVGTDNYGYDY | 567 |
| R5E-G2 | GTIKSINA | 568 | IGSNGAK | 569 | TALITVAESDDYDY | 570 |
| S1a-C4 | GSRDSINT | 571 | LDNSTTG | 572 | AAVTGTPGGLGFYDY | 573 |
| S1b-E11 | GSYASIAS | 574 | VTSGGST | 575 | NALAGANYESQWEEYNY | 576 |
| S1b-E7 | GGEFSDNG | 577 | ASSDGST | 578 | TALLMSSAGEYDV | 579 |
| S2a-C4 | GFSESINA | 580 | LDGGGSD | 581 | AAQLTSSFRGYDFTPSNYDY | 582 |
| S2b-A7 | GSSDSWNA | 583 | ISSAGSG | 584 | NGYEHTTGYDY | 585 |
| S2b-C10 | GSAFSLNY | 586 | IGSGGSS | 587 | AAQPNGSPDDFDY | 588 |
| S2b-F7 | GSTLSNNA | 589 | IGSSDST | 590 | TALLDSGPGEYNY | 591 |
| S2b-G8 | GSIQSYEA | 592 | ITWGDST | 593 | AADVRQGYSS | 594 |
| S3a-C5 | GSIKNTNA | 595 | ITSGGNS | 596 | AAFGYRYDQDYTYDY | 597 |
| S3a-H1 | GGIYSPYS | 598 | IVGGGGT | 599 | NANYQEYDEYDY | 600 |
| S3a-H2 | GAAFSKNA | 601 | ITSGSGT | 602 | AALAYSGGYDY | 603 |
| S3b-A7 | GSIASINA | 604 | ITSGGST | 605 | AAQPGQSYAV | 606 |
| S3b-A8 | GSIISRVK | 607 | IDSGTAS | 608 | AAENRGYGVD | 609 |
| S3b-C8 | GSIENIER | 610 | IDSGGTT | 611 | AAVWLTNAESTQEY | 612 |
| S3b-E11 | GDIYSQSA | 613 | ITTTGSR | 614 | AAFAVRSSSVSDAYDY | 615 |
| RN-01 | GYIFEGGP | 616 | ITTSGET | 617 | NATDRDETGYDY | 618 |
| RN-02 | GSYFSKNA | 619 | LTNSGSG | 620 | TANISESTGQGYDY | 621 |
| RN-03 | GFEFTYRA | 622 | VVSGGST | 623 | NAGPQAEEDTYDY | 624 |
| RN-05 | GSSFRILA | 625 | ITSNGNT | 626 | NADTLDLDDGATYNY | 627 |
| RN-09 | GLRFAMTE | 628 | ITEGGSG | 629 | AALHYTEGANTESATTFDY | 630 |
| RN-12 | GSSFEGNA | 631 | ITTSTSY | 632 | AARLKDRGPQSEYDY | 633 |
| RN-14 | GENFSGNL | 634 | VTSTGSG | 635 | AAKHQKGEIPYYD | 636 |
| RN-16 | GADFSSNA | 637 | IDSDKSG | 638 | NAYSGTDNYGYDY | 639 |
| RN-17 | GSMYSRAV | 640 | IGRTGDS | 641 | AADNLDVVYGNRY | 642 |
| RN-18 | GYYFQIVA | 643 | ITSTGST | 644 | NAVSTDAQGAAEY | 645 |
| RN-19 | GIDDSIGA | 646 | LSGTGTT | 647 | AAAFPAGGDY | 648 |
| RN-20 | GNYFEINK | 649 | IGSGGST | 650 | NALIYLADRRDGYYSYDY | 651 |
| RN-21 | GLQTSDNA | 652 | LTSGTSR | 653 | AAFPDVGDASSIGFVY | 654 |
| RN-22 | GSISEINL | 655 | MTSGGST | 656 | AADEFTKYGHGRY | 657 |
| RN-23 | GGEKSISA | 658 | ITAASTT | 659 | TAVLYDSYDY | 660 |
| RN-25 | GEDYSYNA | 661 | INSGGGT | 662 | AYLGALDRDGYDA | 663 |
| RN-26 | GSISSINS | 664 | VSSYGSD | 665 | AARTVTGFTYGDY | 666 |
| RN-27 | GPQFSSNA | 667 | ITSGGST | 668 | TAPSVSDADRRTRDY | 669 |
| RN-28 | GSQFSHNL | 670 | ITSFGST | 671 | AALSSEDTSHYPRNY | 672 |
| RN-29 | GYILSENL | 673 | VTSGDYT | 674 | AAGTSDWEVKWSH | 675 |
| RN-30 | GIPASYLN | 676 | AGGTNSN | 677 | AALDGAESYDYVYPV | 678 |
| RN-31 | GSTGSDYA | 679 | ISSYGSD | 680 | AALQASAGAGYYYDY | 681 |

TABLE 2
Exemplary Framework Region 2 (FW2) Sequences of single domain VHH antibodies

| | | SEQ | | SEQ | | SEQ |
|---|---|---|---|---|---|---|
| | | ID | | ID | | ID |
| ID | CDR1 + FW2: | NO: | FW2 + CDR2: | NO: | CDR3: | NO: |
| R1E- | GQITAENYMHW | 1 | KQREWVAAITTGGSTL | 98 | CNADRSYFPDYW | 195 |
| B2 | YR | | YAD | | | |
| R1E- | GSIREINAMHWV | 2 | KGRELVASIDSGSLDYY | 99 | CAASRSGDYRYW | 196 |
| D4 | R | | AD | | | |
| R1I- | GSILQINAIHWFR | 3 | KQREEVAAITSGGSTGY | 100 | CAATSTRGGLDSDYW | 197 |
| A11 | | | AD | | | |
| R1I-C8 | GRQFGAWRMH | 4 | KQREWVAGITSGTSGN | 101 | CAATSHTTYHAEYW | 198 |
| | WYR | | YAD | | | |
| R1I-E7 | GTEYDISAAHWY | 5 | KQREWVAGVTSDNSTF | 102 | CTATVKTSQYRYW | 199 |
| | R | | YAD | | | |
| R1I- | GSSININYMHWF | 6 | KGREGVASITSGGGTYY | 103 | CAASEAWRREDSW | 200 |
| G7 | R | | ED | | | |
| R2A- | GTWGNIQYLSW | 7 | KRREFVAAITPNNGTNY | 104 | CTAVLWYTTKEGPYDYW | 201 |
| C5 | FR | | AD | | | |
| R2A- | GSITSINWMHWF | 8 | KDREGVASISQTGRGSY | 105 | CAAASRTGTPYNYW | 202 |
| F3 | R | | AD | | | |
| R2B- | GSSESWNAIHWF | 9 | KQREFVARITSGTSTYY | 106 | CNGYEHTTGYDYW | 203 |
| C4 | R | | VD | | | |
| R2B- | GSYASIASMHWF | 10 | KQREFVAGLTSGGSRIY | 107 | CTALILHYAGMYDYW | 204 |
| D4 | R | | GD | | | |
| R2B- | GYADEINMVHW | 11 | KGREFVAAIASGGSTFY | 108 | CAAIPRPSLSYAHDYAYW | 205 |
| D5 | YR | | AD | | | |
| R2B- | GGVTRGNAMAW | 12 | KOREIVAAIDNGGSHAY | 109 | CAVFPDHDSEQASIDRVYW | 206 |
| E6 | YR | | GD | | | |
| R2C- | GTYASYETIAWY | 13 | KQREKVARITGAGSSFY | 110 | CNADSIPPALRAANTGYW | 207 |
| B3 | R | | AD | | | |
| R2C- | GRILAANIVHWY | 14 | KEREGVAGITTGGSNYY | 111 | CNALGLGSASGSYESW | 208 |
| F6 | R | | SD | | | |
| R2C- | GSIYKYSDVHWF | 15 | KAREAVALLTGAGTTIY | 112 | CAAGQGFKATRTFDSW | 209 |
| G6 | R | | GD | | | |
| R2D- | GSGASIGAMHWF | 16 | KGREWVASITSAGNTYY | 113 | CNALAGANYESQWEEYNY | 210 |
| A6 | R | | GD | | W | |
| R2D- | GHIFSINNAAWV | 17 | KSREGVATIHSGGGTYY | 114 | CNAEVSTGAGSRGRVSDVY | 211 |
| D3 | R | | SD | | W | |
| R2D- | GSAMMINRTHW | 18 | KEREGVAGITWQGSTY | 115 | CNALSADGAYVRRYKDYW | 212 |
| G5 | FR | | YAD | | | |
| R2E- | GHQLSTNATHW | 19 | KNREFVASIGSGSSSYYA | 116 | CTALGGDELDGMTYDYW | 213 |
| D2 | FR | | D | | | |
| R2E- | GETFSSNAMHWF | 20 | KGREFVAAITSGGNDNY | 117 | CNAPATEDTSHYPRNYW | 214 |
| F4 | R | | SD | | | |
| R5A- | GAELSYTAIHWF | 21 | KQREFVATINSGGSSYY | 118 | CTALLDSTYDYW | 215 |
| A1 | R | | AD | | | |
| R5A- | GSAFATNGLHWF | 22 | KQREFVAGITSNGSINY | 119 | CTALLMSYGGGEEYDYW | 216 |
| A5 | R | | KD | | | |
| R5A- | GQLDRSNAMAW | 23 | KGREWVASITSGASTQY | 120 | CAADRYRAIQRTQYSW | 217 |
| B3 | FR | | AD | | | |
| R5A- | GDDFIENAISWY | 24 | KGREWVAAVTLGGHSN | 121 | CNYPVGIPAVAIDGRRSYGY | 218 |
| B5 | R | | YAD | | W | |
| R5A- | GAFFSYGAIHWF | 25 | KQREEVAAITSGGSTGY | 122 | CNAGVVWLPSNAAYNLW | 219 |
| B7 | R | | AD | | | |
| R5A- | GDIFSINALHWFR | 26 | KQREEVAAITSGGSTGY | 123 | CAAQQARGGLDSDYW | 220 |
| C5 | | | AD | | | |
| R5A- | GSAMMINRTHW | 27 | KEREGVAGITWQGSTY | 124 | CNAVAPVHENENYW | 221 |
| D4 | FR | | YAD | | | |
| R5A- | GNDWYINSAHW | 28 | KEREFVASVSRGGSNNY | 125 | CAALLETSSGDYTYW | 222 |
| D6 | YR | | RD | | | |
| R5A- | GYDFDILRMAWF | 29 | KEREMVAAITSAESVNY | 126 | CATYDYTDDYGYASW | 223 |
| E5 | R | | ED | | | |
| R5A- | GSISSIEAMSWFR | 30 | KQREGVATMASGGDTS | 127 | CAAQTYRPDTWGRYTFW | 224 |
| F2 | | | YAD | | | |
| R5A- | GYHASILAMHW | 31 | KEREWVASMTWSDMT | 128 | CAATYVTRDRPWTHW | 225 |
| F5 | VR | | QYSD | | | |
| R5B- | GGISVANVMHW | 32 | KEREGVAGLTSEGSRYY | 129 | CAAEPTGNTHEFEYW | 226 |
| C3 | FR | | AD | | | |
| R5B- | GSPFKTQTVHWF | 33 | KGREWVASITSGSGTQY | 130 | CAAANDGYGVDW | 227 |
| C5 | R | | MD | | | |
| R5B- | GSIGQIVLVHWF | 34 | KQRELVAGITSPSSTYY | 131 | CNTELEGKTQGFTGKTVW | 228 |
| G1 | R | | AD | | YDYW | |
| R5C- | GLDVSINFMHWY | 35 | KEREGVAGITWQGSTY | 132 | CATVWAGGTPNDYW | 229 |
| A1 | R | | YAD | | | |
| R5C- | GDIFSKNAMHW | 36 | KQREFVAGIYSDGSSLY | 133 | CAALDYSARYRTW | 230 |
| A2 | VR | | AD | | | |
| R5C- | GSGFSNNAMHW | 37 | KQREGVAGITSGSVSFY | 134 | CAAVTGSSTGNAGQGYLTW | 231 |
| B1 | YR | | AD | | | |
| R5C- | GSIFSLNWVHWF | 38 | KSREGVASINSGGHADY | 135 | CAAGPYGAYNFNSW | 232 |
| B2 | R | | SD | | | |
| R5C- | GYIFSATVMHWF | 39 | KQREGVASINSTGSSQY | 136 | CAADGADVVYGNRYW | 233 |
| E1 | R | | AD | | | |
| R5C- | GSFFSINAVHWF | 40 | KEREFVASISVGDSRSYR | 137 | CAAQIYITTYELYDYW | 234 |
| E3 | R | | D | | | |
| R5C- | GTIPSINYTAWYR | 41 | KQRELVASLTSGDNRYY | 138 | CNAKTVGSSYRDSYNYW | 235 |
| F3 | | | TD | | | |
| R5C- | GSYFKINSLAWF | 42 | KAREVVAAIHSGDGTN | 139 | CAADQAWVRAGTYDYW | 236 |
| F5 | R | | YAD | | | |
| R5C- | GQTNSILAMHW | 43 | KOREAVATITSGSNSTY | 140 | CAARNGKLSPKANQRLYW | 237 |
| F6 | YR | | AD | | | |
| R5C- | GSATSINRMAWV | 44 | KEREWVAVIDSGGYTY | 141 | CNAVPVTTDKTPMYQYW | 238 |
| G2 | R | | YAD | | | |
| R5C- | GSIESINAIHWFR | 45 | KAREEVAAITENDVDYY | 142 | CASLSSGYQYVGFRYW | 239 |
| G6 | | | AD | | | |
| R5D- | GSISSYNAMHWF | 46 | KQREWVAGLTSSGSDG | 143 | CNAFSYHTYSRYDFW | 240 |
| A1 | R | | YVD | | | |
| R5D- | GSIWSIAFMSWY | 47 | KGREWVAAITTGNDEA | 144 | CAANTYERTPDGRTFSYW | 241 |
| B2 | R | | YAD | | | |
| R5D- | GSSTSINIMAWY | 48 | KQREGVAGITGGSNGY | 145 | CAAVATSYTGVEYEYW | 242 |
| B4 | R | | YAD | | | |
| R5D- | GSRDSIVAMHWY | 49 | KGREGVAGITSRWVTY | 146 | CNAPVYYDTEWYDIW | 243 |
| B5 | R | | YAD | | | |
| R5D- | GRILAANIVHWY | 50 | KEREGVAGITSGGGSYY | 147 | CNALGLGQSSLYRSADPYDS | 244 |
| B6 | R | | AD | | W | |
| R5D- | GSDFTINAMHWF | 51 | KQREFVATITSTSSTAYR | 148 | CAAFAVRDADTAGWTARD | 245 |
| D4 | R | | D | | YW | |
| R5D- | GSSFSINWMHWF | 52 | KQREGVASINSTGSDYY | 149 | CAAAITQPMPQGKGAPPGF | 246 |
| E1 | R | | AD | | YYW | |
| R5D- | GSIYVINTMHWF | 53 | KORELVALIAKGGSGYY | 150 | CAADRTYGYEYSTYQYW | 247 |
| E3 | R | | AD | | | |
| R5D- | GQISSNNGMHWF | 54 | KRREFVATATTGGTTSY | 151 | CTALLMSRAGEFDYW | 248 |
| E4 | R | | AD | | | |
| R5D- | GSQLSINYVHWF | 55 | KQREFVAAITITGSSYYA | 152 | CNTVDRTDNYGYDYW | 249 |
| F3 | R | | D | | | |
| R5D- | GSIFGINDVHWF | 56 | KEREFVATITNGLSARY | 153 | CAAQVYITTYELYDYW | 250 |
| G1 | R | | KD | | | |
| R5D- | GRILAANIVHWY | 57 | KNREGVAGITSGGSTYY | 154 | CAAKPLGSPDDFDYW | 251 |
| G6 | R | | AD | | | |
| R5E- | GSIPYVQAMSWF | 58 | KGREGVASIGWGGGTY | 155 | CAAKFRENFSFYDYW | 252 |
| C1 | R | | YAD | | | |
| R5E- | GFEFSTSAMHWY | 59 | KQREWVARITSGGSDN | 156 | CAAYVGTDNYGYDYW | 253 |
| D1 | R | | YGD | | | |
| R5E- | GTIKSINAMHWF | 60 | KAREFVASIGSNGAKSY | 157 | CTALITVAESDDYDYW | 254 |
| G2 | R | | SD | | | |
| S1a-C4 | GSRDSINTAHWY | 61 | KAREWVASLDNSTTGS | 158 | CAAVTGTPGGLGFYDYW | 255 |
| | R | | YAD | | | |
| S1b- | GSYASIASMHWF | 62 | KEREFVAGVTSGGSTNY | 159 | CNALAGANYESQWEEYNY | 256 |
| E11 | R | | GD | | W | |
| S1b-E7 | GGEFSDNGLHWF | 63 | KQREFVATASSDGSTQY | 160 | CTALLMSSAGEYDVW | 257 |
| | R | | RD | | | |
| S2a-C4 | GFSESINATAWF | 64 | KQREGVAGLDGGGSDY | 161 | CAAQLTSSFRGYDFTPSNYD | 258 |
| | R | | YSD | | YW | |
| S2b- | GSSDSWNALHWF | 65 | KEREFVAGISSAGSGYY | 162 | CNGYEHTTGYDYW | 259 |
| A7 | R | | AD | | | |
| S2b- | GSAFSLNYMAWF | 66 | KQREGVAIIGSGGSSDY | 163 | CAAQPNGSPDDFDYW | 260 |
| C10 | R | | AD | | | |
| S2b-F7 | GSTLSNNAMHW | 67 | KAREFVAAIGSSDSTDY | 164 | CTALLDSGPGEYNYW | 261 |
| | FR | | KD | | | |
| S2b- | GSIQSYEAMHWF | 68 | KAREWVAVITWGDSTQ | 165 | CAADVRQGYSSW | 262 |
| G8 | R | | YGD | | | |
| S3a-C5 | GSIKNTNAMHWF | 69 | KDREWVAGITSGGNSN | 166 | CAAFGYRYDQDYTYDYW | 263 |
| | R | | YGD | | | |
| S3a- | GGIYSPYSMHWF | 70 | KAREWVAGIVGGGGTT | 167 | CNANYQEYDEYDYW | 264 |
| H1 | R | | YSD | | | |
| S3a- | GAAFSKNALHWF | 71 | KGREWVASITSGSGTQY | 168 | CAALAYSGGYDYW | 265 |
| H2 | R | | MD | | | |
| S3b- | GSIASINAIHWVR | 72 | KQREEVAAITSGGSTGY | 169 | CAAQPGQSYAVW | 266 |
| A7 | | | AD | | | |
| S3b- | GSIISRVKVHWF | 73 | KSREWVAGIDSGTASYY | 170 | CAAENRGYGVDW | 267 |
| A8 | R | | SD | | | |
| S3b- | GSIENIERMHWY | 74 | KQRELVAVIDSGGTTRY | 171 | CAAVWLTNAESTQEYW | 268 |
| C8 | R | | AD | | | |
| S3b- | GDIYSQSAMAW | 75 | KEREFVAAITTTGSRSY | 172 | CAAFAVRSSSVSDAYDYW | 269 |
| E11 | YR | | KD | | | |
| RN-01 | GYIFEGGPTAWF | 76 | KGREFVAAITTSGETSY | 173 | CNATDRDETGYDYW | 270 |
| | R | | AY | | | |
| RN-02 | GSYFSKNAVHW | 77 | KGREWVAALTNSGSGN | 174 | CTANISESTGQGYDYW | 271 |
| | YR | | YKD | | | |
| RN-03 | GFEFTYRAVHWF | 78 | KGREWVAGVVSGGSTY | 175 | CNAGPQAEEDTYDYW | 272 |
| | R | | YAD | | | |
| RN-05 | GSSFRILAMHWY | 79 | KNREWVAVITSNGNTY | 176 | CNADTLDLDDGATYNYW | 273 |
| | R | | YAD | | | |
| RN-09 | GLRFAMTEMAW | 80 | KQREFVALITEGGSGSY | 177 | CAALHYTEGANTESATTFD | 274 |
| | FR | | GD | | YW | |
| RN-12 | GSSFEGNAVHWF | 81 | KNREFVATITTSTSYYY | 178 | CAARLKDRGPQSEYDYW | 275 |
| | R | | AD | | | |
| RN-14 | GENFSGNLVHWF | 82 | KAREGVATVTSTGSGY | 179 | CAAKHQKGEIPYYDW | 276 |
| | R | | YAD | | | |
| RN-16 | GADFSSNAMHW | 83 | KAREFVASIDSDKSGYY | 180 | CNAYSGTDNYGYDYW | 277 |
| | FR | | AD | | | |
| RN-17 | GSMYSRAVMHW | 84 | KEREGVASIGRTGDSYY | 181 | CAADNLDVVYGNRYW | 278 |
| | FR | | AD | | | |
| RN-18 | GYYFQIVAMHW | 85 | KEREGVAGITSTGSTYY | 182 | CNAVSTDAQGAAEYW | 279 |
| | YR | | GD | | | |
| RN-19 | GIDDSIGAMHWF | 86 | KGREFVAALSGTGTTSY | 183 | CAAAFPAGGDYW | 280 |
| | R | | AD | | | |
| RN-20 | GNYFEINKMHW | 87 | KQREFVATIGSGGSTAY | 184 | CNALIYLADRRDGYYSYDY | 281 |
| | FR | | AD | | W | |
| RN-21 | GLQTSDNAMHW | 88 | KNRELVAGLTSGTSRNY | 185 | CAAFPDVGDASSIGFVYW | 282 |
| | FR | | GD | | | |
| RN-22 | GSISEINLLHWFR | 89 | KQREWVATMTSGGSTD | 186 | CAADEFTKYGHGRYW | 283 |
| | | | YAD | | | |
| RN-23 | GGEKSISAAHWY | 90 | KEREFVAAITAASTTSY | 187 | CTAVLYDSYDYW | 284 |
| | R | | AD | | | |
| RN-25 | GEDYSYNAAHW | 91 | KEREWVAAINSGGGTY | 188 | CAYLGALDRDGYDAW | 285 |
| | FR | | YAD | | | |
| RN-26 | GSISSINSMHWYR | 92 | KEREWVAAVSSYGSDY | 189 | CAARTVTGFTYGDYW | 286 |
| | | | YAD | | | |
| RN-27 | GPQFSSNAVSWF | 93 | KEREWVAGITSGGSTDY | 190 | CTAPSVSDADRRTRDYW | 287 |
| | R | | AD | | | |
| RN-28 | GSQFSHNLMSWF | 94 | KQREFVAAITSFGSTGY | 191 | CAALSSEDTSHYPRNYW | 288 |
| | R | | AD | | | |
| RN-29 | GYILSENLTAWF | 95 | KAREVVATVTSGDYTN | 192 | CAAGTSDWEVKWSHW | 289 |
| | R | | YDD | | | |
| RN-30 | GIPASYLNMHWF | 96 | KQREWVAGAGGTNSNY | 193 | CAALDGAESYDYVYPVW | 290 |
| | R | | YQD | | | |
| RN-31 | GSTGSDYAMHW | 97 | KDREWVAGISSYGSDSY | 194 | CAALQASAGAGYYYDYW | 291 |
| | FR | | GD | | | |

The bolded sequences refer to a CDR sequence.

TABLE 3
Exemplary sequences of single domain VHH antibodies

| ID | VHH Sequence: | SEQ ID NO: |
|---|---|---|
| R1E-B2 | QVQLVESGGGLVQPGGSLRLSCAASGQITAENYMHWY | 292 |
| | RQAPGKQREWVAAITTGGSTLYADSVKGRFTISRDNAK | |
| | NTVYLQMNSLKPEDTAVYYCNADRSYFPDYWGQGTQV | |
| | TVSS | |
| R1E-D4 | QVQLVESGGGLVQPGGSLRLSCAASGSIREINAMHWVR | 293 |
| | QAPGKGRELVASIDSGSLDYYADSVKGRFTISRDNAKNT | |
| | VYLQMNSLKPEDTAVYYCAASRSGDYRYWGQGTQVT | |
| | VSS | |
| R1I-A11 | QVQLVESGGGLVQPGGSLRLSCAASGSILQINAIHWFRQ | 294 |
| | APGKQREEVAAITSGGSTGYADSVKGRFTISRDNAKNTV | |
| | YLQMNSLKPEDTAVYYCAATSTRGGLDSDYWGQGTQV | |
| | TVSS | |
| R1I-C8 | QVQLVESGGGLVQPGGSLRLSCAASGRQFGAWRMHWY | 295 |
| | RQAPGKQREWVAGITSGTSGNYADSVKGRFTISRDNAK | |
| | NTVYLQMNSLKPEDTAVYYCAATSHTTYHAEYWGQGT | |
| | QVTVSS | |
| R1I-E7 | QVQLVESGGGLVQPGGSLRLSCAASGTEYDISAAHWYR | 296 |
| | QAPGKQREWVAGVTSDNSTFYADSVKGRFTISRDNAKN | |
| | TVYLQMNSLKPEDTAVYYCTATVKTSQYRYWGQGTQV | |
| | TVSS | |
| R1I-G7 | QVQLVESGGGLVQPGGSLRLSCAASGSSININYMHWFR | 297 |
| | QAPGKGREGVASITSGGGTYYEDSVKGRFTISRDNAKNT | |
| | VYLQMNSLKPEDTAVYYCAASEAWRRFDSWGQGTQVT | |
| | VSS | |
| R2A-C5 | QVQLVESGGGLVQPGGSLRLSCAASGTWGNIQYLSWFR | 298 |
| | QAPGKRREFVAAITPNNGTNYADSVKGRFTISRDNAKN | |
| | TVYLQMNSLKPEDTAVYYCTAVLWYTTKEGPYDYWG | |
| | QGTQVTVSS | |
| R2A-F3 | QVQLVESGGGLVQPGGSLRLSCAASGSITSINWMHWFR | 299 |
| | QAPGKDREGVASISQTGRGSYADSVKGRFTISRDNAKNT | |
| | VYLQMNSLKPEDTAVYYCAAASRTGTPYNYWGQGTQV | |
| | TVSS | |
| R2B-C4 | QVQLVESGGGLVQPGGSLRLSCAASGSSESWNAIHWFR | 300 |
| | QAPGKQREFVARITSGTSTYYVDSVKGRFTISRDNAKNT | |
| | VYLQMNSLKPEDTAVYYCNGYEHTTGYDYWGQGTQV | |
| | TVSS | |
| R2B-D4 | QVQLVESGGGLVQPGGSLRLSCAASGSYASIASMHWFR | 301 |
| | QAPGKQREFVAGLTSGGSRIYGDSVKGRFTISRDNAKNT | |
| | VYLQMNSLKPEDTAVYYCTALILHYAGMYDYWGQGT | |
| | QVTVSS | |
| R2B-D5 | QVQLVESGGGLVQPGGSLRLSCAASGYADEINMVHWY | 302 |
| | RQAPGKGREFVAAIASGGSTFYADSVKGRFTISRDNAKN | |
| | TVYLQMNSLKPEDTAVYYCAAIPRPSLSYAHDYAYWG | |
| | QGTQVTVSS | |
| R2B-E6 | QVQLVESGGGLVQPGGSLRLSCAASGGVTRGNAMAWY | 303 |
| | RQAPGKQREIVAAIDNGGSHAYGDSVKGRFTISRDNAK | |
| | NTVYLQMNSLKPEDTAVYYCAVFPDHDSEQASIDRVY | |
| | WGQGTQVTVSS | |
| R2C-B3 | QVQLVESGGGLVQPGGSLRLSCAASGTYASYETIAWYR | 304 |
| | QAPGKQREKVARITGAGSSFYADSVKGRFTISRDNAKNT | |
| | VYLQMNSLKPEDTAVYYCNADSIPPALRAANTGYWGQ | |
| | GTQVTVSS | |
| R2C-F6 | QVQLVESGGGLVQPGGSLRLSCAASGRILAANIVHWYR | 305 |
| | QAPGKEREGVAGITTGGSNYYSDSVKGRFTISRDNAKNT | |
| | VYLQMNSLKPEDTAVYYCNALGLGSASGSYESWGQGT | |
| | QVTVSS | |
| R2C-G6 | QVQLVESGGGLVQPGGSLRLSCAASGSIYKYSDVHWFR | 306 |
| | QAPGKAREAVALLTGAGTTIYGDSVKGRFTISRDNAKN | |
| | TVYLQMNSLKPEDTAVYYCAAGQGFKATRTFDSWGQG | |
| | TQVTVSS | |
| R2D-A6 | QVQLVESGGGLVQPGGSLRLSCAASGSGASIGAMHWFR | 307 |
| | QAPGKGREWVASITSAGNTYYGDSVKGRFTISRDNAKN | |
| | TVYLQMNSLKPEDTAVYYCNALAGANYESQWEEYNY | |
| | WGQGTQVTVSS | |
| R2D-D3 | QVQLVESGGGLVQPGGSLRLSCAASGHIFSINNAAWVR | 308 |
| | QAPGKSREGVATIHSGGGTYYSDSVKGRFTISRDNAKNT | |
| | VYLQMNSLKPEDTAVYYCNAEVSTGAGSRGRVSDVYW | |
| | GQGTQVTVSS | |
| R2D-G5 | QVQLVESGGGLVQPGGSLRLSCAASGSAMMINRTHWFR | 309 |
| | QAPGKEREGVAGITWQGSTYYADSVKGRFTISRDNAKN | |
| | TVYLQMNSLKPEDTAVYYCNALSADGAYVRRYKDYW | |
| | GQGTQVTVSS | |
| R2E-D2 | QVQLVESGGGLVQPGGSLRLSCAASGHQLSTNATHWFR | 310 |
| | QAPGKNREFVASIGSGSSSYYADSVKGRFTISRDNAKNT | |
| | VYLQMNSLKPEDTAVYYCTALGGDELDGMTYDYWGQ | |
| | GTQVTVSS | |
| R2E-F4 | QVQLVESGGGLVQPGGSLRLSCAASGETFSSNAMHWFR | 311 |
| | QAPGKGREFVAAITSGGNDNYSDSVKGRFTISRDNAKN | |
| | TVYLQMNSLKPEDTAVYYCNAPATEDTSHYPRNYWGQ | |
| | GTQVTVSS | |
| R5A-A1 | QVQLVESGGGLVQPGGSLRLSCAASGAELSYTAIHWFR | 312 |
| | QAPGKQREFVATINSGGSSYYADSVKGRFTISRDNAKNT | |
| | VYLQMNSLKPEDTAVYYCTALLDSTYDYWGQGTQVTV | |
| | SS | |
| R5A-A5 | QVQLVESGGGLVQPGGSLRLSCAASGSAFATNGLHWFR | 313 |
| | QAPGKQREFVAGITSNGSINYKDSVKGRFTISRDNAKNT | |
| | VYLQMNSLKPEDTAVYYCTALLMSYGGGEEYDYWGQ | |
| | GTQVTVSS | |
| R5A-B3 | QVQLVESGGGLVQPGGSLRLSCAASGQLDRSNAMAWF | 314 |
| | RQAPGKGREWVASITSGASTQYADSVKGRFTISRDNAK | |
| | NTVYLQMNSLKPEDTAVYYCAADRYRAIQRTQYSWGQ | |
| | GTQVTVSS | |
| R5A-B5 | QVQLVESGGGLVQPGGSLRLSCAASGDDFIENAISWYR | 315 |
| | QAPGKGREWVAAVTLGGHSNYADSVKGRFTISRDNAK | |
| | NTVYLQMNSLKPEDTAVYYCNYPVGIPAVAIDGRRSYG | |
| | YWGQGTQVTVSS | |
| R5A-B7 | QVQLVESGGGLVQPGGSLRLSCAASGAFFSYGAIHWFR | 316 |
| | QAPGKQREEVAAITSGGSTGYADSVKGRFTISRDNAKNT | |
| | VYLQMNSLKPEDTAVYYCNAGVVWLPSNAAYNLWGQ | |
| | GTQVTVSS | |
| R5A-C5 | QVQLVESGGGLVQPGGSLRLSCAASGDIFSINALHWFRQ | 317 |
| | APGKQREEVAAITSGGSTGYADSVKGRFTISRDNAKNTV | |
| | YLQMNSLKPEDTAVYYCAAQQARGGLDSDYWGQGTQ | |
| | VTVSS | |
| R5A-D4 | QVQLVESGGGLVQPGGSLRLSCAASGSAMMINRTHWFR | 318 |
| | QAPGKEREGVAGITWQGSTYYADSVKGRFTISRDNAKN | |
| | TVYLQMNSLKPEDTAVYYCNAVAPVHENENYWGQGT | |
| | QVTVSS | |
| R5A-D6 | QVQLVESGGGLVQPGGSLRLSCAASGNDWYINSAHWY | 319 |
| | RQAPGKEREFVASVSRGGSNNYRDSVKGRFTISRDNAK | |
| | NTVYLQMNSLKPEDTAVYYCAALLETSSGDYTYWGQG | |
| | TQVTVSS | |
| R5A-E5 | QVQLVESGGGLVQPGGSLRLSCAASGYDFDILRMAWFR | 320 |
| | QAPGKEREMVAAITSAESVNYEDSVKGRFTISRDNAKN | |
| | TVYLQMNSLKPEDTAVYYCATYDYTDDYGYASWGQG | |
| | TQVTVSS | |
| R5A-F2 | QVQLVESGGGLVQPGGSLRLSCAASGSISSIEAMSWFRQ | 321 |
| | APGKQREGVATMASGGDTSYADSVKGRFTISRDNAKNT | |
| | VYLQMNSLKPEDTAVYYCAAQTYRPDTWGRYTFWGQ | |
| | GTQVTVSS | |
| R5A-F5 | QVQLVESGGGLVQPGGSLRLSCAASGYHASILAMHWV | 322 |
| | RQAPGKEREWVASMTWSDMTQYSDSVKGRFTISRDNA | |
| | KNTVYLQMNSLKPEDTAVYYCAATYVTRDRPWTHWG | |
| | QGTQVTVSS | |
| R5B-C3 | QVQLVESGGGLVQPGGSLRLSCAASGGISVANVMHWFR | 323 |
| | QAPGKEREGVAGLTSEGSRYYADSVKGRFTISRDNAKN | |
| | TVYLQMNSLKPEDTAVYYCAAEPTGNTHEFEYWGQGT | |
| | QVTVSS | |
| R5B-C5 | QVQLVESGGGLVQPGGSLRLSCAASGSPFKTQTVHWFR | 324 |
| | QAPGKGREWVASITSGSGTQYMDSVKGRFTISRDNAKN | |
| | TVYLQMNSLKPEDTAVYYCAAANDGYGVDWGQGTQV | |
| | TVSS | |
| R5B-G1 | QVQLVESGGGLVQPGGSLRLSCAASGSIGQIVLVHWFR | 325 |
| | QAPGKQRELVAGITSPSSTYYADSVKGRFTISRDNAKNT | |
| | VYLQMNSLKPEDTAVYYCNTELEGKTQGFTGKTVWYD | |
| | YWGQGTQVTVSS | |
| R5C-A1 | QVQLVESGGGLVQPGGSLRLSCAASGLDVSINFMHWYR | 326 |
| | QAPGKEREGVAGITWQGSTYYADSVKGRFTISRDNAKN | |
| | TVYLQMNSLKPEDTAVYYCATVWAGGTPNDYWGQGT | |
| | QVTVSS | |
| R5C-A2 | QVQLVESGGGLVQPGGSLRLSCAASGDIFSKNAMHWVR | 327 |
| | QAPGKQREFVAGIYSDGSSLYADSVKGRFTISRDNAKNT | |
| | VYLQMNSLKPEDTAVYYCAALDYSARYRTWGQGTQVT | |
| | VSS | |
| R5C-B1 | QVQLVESGGGLVQPGGSLRLSCAASGSGFSNNAMHWY | 328 |
| | RQAPGKQREGVAGITSGSVSFYADSVKGRFTISRDNAKN | |
| | TVYLQMNSLKPEDTAVYYCAAVTGSSTGNAGQGYLTW | |
| | GQGTQVTVSS | |
| R5C-B2 | QVQLVESGGGLVQPGGSLRLSCAASGSIFSLNWVHWFR | 329 |
| | QAPGKSREGVASINSGGHADYSDSVKGRFTISRDNAKNT | |
| | VYLQMNSLKPEDTAVYYCAAGPYGAYNFNSWGQGTQ | |
| | VTVSS | |
| R5C-E1 | QVQLVESGGGLVQPGGSLRLSCAASGYIFSATVMHWFR | 330 |
| | QAPGKQREGVASINSTGSSQYADSVKGRFTISRDNAKNT | |
| | VYLQMNSLKPEDTAVYYCAADGADVVYGNRYWGQGT | |
| | QVTVSS | |
| R5C-E3 | QVQLVESGGGLVQPGGSLRLSCAASGSFFSINAVHWFR | 331 |
| | QAPGKEREFVASISVGDSRSYRDSVKGRFTISRDNAKNT | |
| | VYLQMNSLKPEDTAVYYCAAQIYITTYELYDYWGQGT | |
| | QVTVSS | |
| R5C-F3 | QVQLVESGGGLVQPGGSLRLSCAASGTIPSINYTAWYRQ | 332 |
| | APGKQRELVASLTSGDNRYYTDSVKGRFTISRDNAKNT | |
| | VYLQMNSLKPEDTAVYYCNAKTVGSSYRDSYNYWGQ | |
| | GTQVTVSS | |
| R5C-F5 | QVQLVESGGGLVQPGGSLRLSCAASGSYFKINSLAWFR | 333 |
| | QAPGKAREVVAAIHSGDGTNYADSVKGRFTISRDNAKN | |
| | TVYLQMNSLKPEDTAVYYCAADQAWVRAGTYDYWGQ | |
| | GTQVTVSS | |
| R5C-F6 | QVQLVESGGGLVQPGGSLRLSCAASGQTNSILAMHWYR | 334 |
| | QAPGKQREAVATITSGSNSTYADSVKGRFTISRDNAKNT | |
| | VYLQMNSLKPEDTAVYYCAARNGKLSPKANQRLYWG | |
| | QGTQVTVSS | |
| R5C-G2 | QVQLVESGGGLVQPGGSLRLSCAASGSATSINRMAWVR | 335 |
| | QAPGKEREWVAVIDSGGYTYYADSVKGRFTISRDNAKN | |
| | TVYLQMNSLKPEDTAVYYCNAVPVTTDKTPMYQYWG | |
| | QGTQVTVSS | |
| R5C-G6 | QVQLVESGGGLVQPGGSLRLSCAASGSIESINAIHWFRQ | 336 |
| | APGKAREEVAAITENDVDYYADSVKGRFTISRDNAKNT | |
| | VYLQMNSLKPEDTAVYYCASLSSGYQYVGFRYWGQGT | |
| | QVTVSS | |
| R5D-A1 | QVQLVESGGGLVQPGGSLRLSCAASGSISSYNAMHWFR | 337 |
| | QAPGKQREWVAGLTSSGSDGYVDSVKGRFTISRDNAKN | |
| | TVYLQMNSLKPEDTAVYYCNAFSYHTYSRYDFWGQGT | |
| | QVTVSS | |
| R5D-B2 | QVQLVESGGGLVQPGGSLRLSCAASGSIWSIAFMSWYR | 338 |
| | QAPGKGREWVAAITTGNDEAYADSVKGRFTISRDNAKN | |
| | TVYLQMNSLKPEDTAVYYCAANTYERTPDGRTFSYWG | |
| | QGTQVTVSS | |
| R5D-B4 | QVQLVESGGGLVQPGGSLRLSCAASGSSTSINIMAWYR | 339 |
| | QAPGKQREGVAGITGGSNGYYADSVKGRFTISRDNAKN | |
| | TVYLQMNSLKPEDTAVYYCAAVATSYTGVEYEYWGQG | |
| | TQVTVSS | |
| R5D-B5 | QVQLVESGGGLVQPGGSLRLSCAASGSRDSIVAMHWYR | 340 |
| | QAPGKGREGVAGITSRWVTYYADSVKGRFTISRDNAKN | |
| | TVYLQMNSLKPEDTAVYYCNAPVYYDTEWYDIWGQGT | |
| | QVTVSS | |
| R5D-B6 | QVQLVESGGGLVQPGGSLRLSCAASGRILAANIVHWYR | 341 |
| | QAPGKEREGVAGITSGGGSYYADSVKGRFTISRDNAKN | |
| | TVYLQMNSLKPEDTAVYYCNALGLGQSSLYRSADPYDS | |
| | WGQGTQVTVSS | |
| R5D-D4 | QVQLVESGGGLVQPGGSLRLSCAASGSDFTINAMHWFR | 342 |
| | QAPGKQREFVATITSTSSTAYRDSVKGRFTISRDNAKNT | |
| | VYLQMNSLKPEDTAVYYCAAFAVRDADTAGWTARDY | |
| | WGQGTQVTVSS | |
| R5D-E1 | QVQLVESGGGLVQPGGSLRLSCAASGSSFSINWMHWFR | 343 |
| | QAPGKQREGVASINSTGSDYYADSVKGRFTISRDNAKN | |
| | TVYLQMNSLKPEDTAVYYCAAAITQPMPQGKGAPPGFY | |
| | YWGQGTQVTVSS | |
| R5D-E3 | QVQLVESGGGLVQPGGSLRLSCAASGSIYVINTMHWFR | 344 |
| | QAPGKQRELVALIAKGGSGYYADSVKGRFTISRDNAKN | |
| | TVYLQMNSLKPEDTAVYYCAADRTYGYEYSTYQYWG | |
| | QGTQVTVSS | |
| R5D-E4 | QVQLVESGGGLVQPGGSLRLSCAASGQISSNNGMHWFR | 345 |
| | QAPGKRREFVATATTGGTTSYADSVKGRFTISRDNAKN | |
| | TVYLQMNSLKPEDTAVYYCTALLMSRAGEFDYWGQGT | |
| | QVTVSS | |
| R5D-F3 | QVQLVESGGGLVQPGGSLRLSCAASGSQLSINYVHWFR | 346 |
| | QAPGKQREFVAAITITGSSYYADSVKGRFTISRDNAKNT | |
| | VYLQMNSLKPEDTAVYYCNTVDRTDNYGYDYWGQGT | |
| | QVTVSS | |
| R5D-G1 | QVQLVESGGGLVQPGGSLRLSCAASGSIFGINDVHWFRQ | 347 |
| | APGKEREFVATITNGLSARYKDSVKGRFTISRDNAKNTV | |
| | YLQMNSLKPEDTAVYYCAAQVYITTYELYDYWGQGTQ | |
| | VTVSS | |
| R5D-G6 | QVQLVESGGGLVQPGGSLRLSCAASGRILAANIVHWYR | 348 |
| | QAPGKNREGVAGITSGGSTYYADSVKGRFTISRDNAKN | |
| | TVYLQMNSLKPEDTAVYYCAAKPLGSPDDFDYWGQGT | |
| | QVTVSS | |
| R5E-C1 | QVQLVESGGGLVQPGGSLRLSCAASGSIPYVQAMSWFR | 349 |
| | QAPGKGREGVASIGWGGGTYYADSVKGRFTISRDNAKN | |
| | TVYLQMNSLKPEDTAVYYCAAKFRENFSFYDYWGQGT | |
| | QVTVSS | |
| R5E-D1 | QVQLVESGGGLVQPGGSLRLSCAASGFEFSTSAMHWYR | 350 |
| | QAPGKQREWVARITSGGSDNYGDSVKGRFTISRDNAKN | |
| | TVYLQMNSLKPEDTAVYYCAAYVGTDNYGYDYWGQG | |
| | TQVTVSS | |
| R5E-G2 | QVQLVESGGGLVQPGGSLRLSCAASGTIKSINAMHWFR | 351 |
| | QAPGKAREFVASIGSNGAKSYSDSVKGRFTISRDNAKNT | |
| | VYLQMNSLKPEDTAVYYCTALITVAESDDYDYWGQGT | |
| | QVTVSS | |
| S1a-C4 | QVQLVESGGGLVQPGGSLRLSCAASGSRDSINTAHWYR | 352 |
| | QAPGKAREWVASLDNSTTGSYADSVKGRFTISRDNAKN | |
| | TVYLQMNSLKPEDTAVYYCAAVTGTPGGLGFYDYWGQ | |
| | GTQVTVSS | |
| S1b-E11 | QVQLVESGGGLVQPGGSLRLSCAASGSYASIASMHWFR | 353 |
| | QAPGKEREFVAGVTSGGSTNYGDSVKGRFTISRDNAKN | |
| | TVYLQMNSLKPEDTAVYYCNALAGANYESQWEEYNY | |
| | WGQGTQVTVSS | |
| S1b-E7 | QVQLVESGGGLVQPGGSLRLSCAASGGEFSDNGLHWFR | 354 |
| | QAPGKQREFVATASSDGSTQYRDSVKGRFTISRDNAKN | |
| | TVYLQMNSLKPEDTAVYYCTALLMSSAGEYDVWGQGT | |
| | QVTVSS | |
| S2a-C4 | QVQLVESGGGLVQPGGSLRLSCAASGFSESINATAWFRQ | 355 |
| | APGKQREGVAGLDGGGSDYYSDSVKGRFTISRDNAKNT | |
| | VYLQMNSLKPEDTAVYYCAAQLTSSFRGYDFTPSNYDY | |
| | WGQGTQVTVSS | |
| S2b-A7 | QVQLVESGGGLVQPGGSLRLSCAASGSSDSWNALHWFR | 356 |
| | QAPGKEREFVAGISSAGSGYYADSVKGRFTISRDNAKNT | |
| | VYLQMNSLKPEDTAVYYCNGYEHTTGYDYWGQGTQV | |
| | TVSS | |
| S2b-C10 | QVQLVESGGGLVQPGGSLRLSCAASGSAFSLNYMAWFR | 357 |
| | QAPGKQREGVAIIGSGGSSDYADSVKGRFTISRDNAKNT | |
| | VYLQMNSLKPEDTAVYYCAAQPNGSPDDFDYWGQGTQ | |
| | VTVSS | |
| S2b-F7 | QVQLVESGGGLVQPGGSLRLSCAASGSTLSNNAMHWFR | 358 |
| | QAPGKAREFVAAIGSSDSTDYKDSVKGRFTISRDNAKNT | |
| | VYLQMNSLKPEDTAVYYCTALLDSGPGEYNYWGQGTQ | |
| | VTVSS | |
| S2b-G8 | QVQLVESGGGLVQPGGSLRLSCAASGSIQSYEAMHWFR | 359 |
| | QAPGKAREWVAVITWGDSTQYGDSVKGRFTISRDNAK | |
| | NTVYLQMNSLKPEDTAVYYCAADVRQGYSSWGQGTQ | |
| | VTVSS | |
| S3a-C5 | QVQLVESGGGLVQPGGSLRLSCAASGSIKNTNAMHWFR | 360 |
| | QAPGKDREWVAGITSGGNSNYGDSVKGRFTISRDNAKN | |
| | TVYLQMNSLKPEDTAVYYCAAFGYRYDQDYTYDYWG | |
| | QGTQVTVSS | |
| S3a-H1 | QVQLVESGGGLVQPGGSLRLSCAASGGIYSPYSMHWFR | 361 |
| | QAPGKAREWVAGIVGGGGTTYSDSVKGRFTISRDNAKN | |
| | TVYLQMNSLKPEDTAVYYCNANYQEYDEYDYWGQGT | |
| | QVTVSS | |
| S3a-H2 | QVQLVESGGGLVQPGGSLRLSCAASGAAFSKNALHWFR | 362 |
| | QAPGKGREWVASITSGSGTQYMDSVKGRFTISRDNAKN | |
| | TVYLQMNSLKPEDTAVYYCAALAYSGGYDYWGQGTQ | |
| | VTVSS | |
| S3b-A7 | QVQLVESGGGLVQPGGSLRLSCAASGSIASINAIHWVRQ | 363 |
| | APGKQREEVAAITSGGSTGYADSVKGRFTISRDNAKNTV | |
| | YLQMNSLKPEDTAVYYCAAQPGQSYAVWGQGTQVTVS | |
| | S | |
| S3b-A8 | QVQLVESGGGLVQPGGSLRLSCAASGSIISRVKVHWFRQ | 364 |
| | APGKSREWVAGIDSGTASYYSDSVKGRFTISRDNAKNT | |
| | VYLQMNSLKPEDTAVYYCAAENRGYGVDWGQGTQVT | |
| | VSS | |
| S3b-C8 | QVQLVESGGGLVQPGGSLRLSCAASGSIENIERMHWYR | 365 |
| | QAPGKQRELVAVIDSGGTTRYADSVKGRFTISRDNAKN | |
| | TVYLQMNSLKPEDTAVYYCAAVWLTNAESTQEYWGQ | |
| | GTQVTVSS | |
| S3b-E11 | QVQLVESGGGLVQPGGSLRLSCAASGDIYSQSAMAWYR | 366 |
| | QAPGKEREFVAAITTTGSRSYKDSVKGRFTISRDNAKNT | |
| | VYLQMNSLKPEDTAVYYCAAFAVRSSSVSDAYDYWGQ | |
| | GTQVTVSS | |
| RN-01 | QVQLVESGGGLVQPGGSLRLSCAASGYIFEGGPTAWFR | 367 |
| | QAPGKGREFVAAITTSGETSYAYSVKGRFTISRDNAKNT | |
| | VYLQMNSLKPEDTAVYYCNATDRDETGYDYWGQGTQ | |
| | VTVSS | |
| RN-02 | QVQLVESGGGLVQPGGSLRLSCAASGSYFSKNAVHWY | 368 |
| | RQAPGKGREWVAALTNSGSGNYKDSVKGRFTISRDNA | |
| | KNTVYLQMNSLKPEDTAVYYCTANISESTGQGYDYWG | |
| | QGTQVTVSS | |
| RN-03 | QVQLVESGGGLVQPGGSLRLSCAASGFEFTYRAVHWFR | 369 |
| | QAPGKGREWVAGVVSGGSTYYADSVKGRFTISRDNAK | |
| | NTVYLQMNSLKPEDTAVYYCNAGPQAEEDTYDYWGQ | |
| | GTQVTVSS | |
| RN-05 | QVQLVESGGGLVQPGGSLRLSCAASGSSFRILAMHWYR | 370 |
| | QAPGKNREWVAVITSNGNTYYADSVKGRFTISRDNAKN | |
| | TVYLQMNSLKPEDTAVYYCNADTLDLDDGATYNYWG | |
| | QGTQVTVSS | |
| RN-09 | QVQLVESGGGLVQPGGSLRLSCAASGLRFAMTEMAWF | 371 |
| | RQAPGKQREFVALITEGGSGSYGDSVKGRFTISRDNAKN | |
| | TVYLQMNSLKPEDTAVYYCAALHYTEGANTESATTFDY | |
| | WGQGTQVTVSS | |
| RN-12 | QVQLVESGGGLVQPGGSLRLSCAASGSSFEGNAVHWFR | 372 |
| | QAPGKNREFVATITTSTSYYYADSVKGRFTISRDNAKNT | |
| | VYLQMNSLKPEDTAVYYCAARLKDRGPQSEYDYWGQ | |
| | GTQVTVSS | |
| RN-14 | QVQLVESGGGLVQPGGSLRLSCAASGENFSGNLVHWFR | 373 |
| | QAPGKAREGVATVTSTGSGYYADSVKGRFTISRDNAKN | |
| | TVYLQMNSLKPEDTAVYYCAAKHQKGEIPYYDWGQGT | |
| | QVTVSS | |
| RN-16 | QVQLVESGGGLVQPGGSLRLSCAASGADFSSNAMHWF | 374 |
| | RQAPGKAREFVASIDSDKSGYYADSVKGRFTISRDNAK | |
| | NTVYLQMNSLKPEDTAVYYCNAYSGTDNYGYDYWGQ | |
| | GTQVTVSS | |
| RN-17 | QVQLVESGGGLVQPGGSLRLSCAASGSMYSRAVMHWF | 375 |
| | RQAPGKEREGVASIGRTGDSYYADSVKGRFTISRDNAK | |
| | NTVYLQMNSLKPEDTAVYYCAADNLDVVYGNRYWGQ | |
| | GTQVTVSS | |
| RN-18 | QVQLVESGGGLVQPGGSLRLSCAASGYYFQIVAMHWY | 376 |
| | RQAPGKEREGVAGITSTGSTYYGDSVKGRFTISRDNAKN | |
| | TVYLQMNSLKPEDTAVYYCNAVSTDAQGAAEYWGQG | |
| | TQVTVSS | |
| RN-19 | QVQLVESGGGLVQPGGSLRLSCAASGIDDSIGAMHWFR | 377 |
| | QAPGKGREFVAALSGTGTTSYADSVKGRFTISRDNAKN | |
| | TVYLQMNSLKPEDTAVYYCAAAFPAGGDYWGQGTQV | |
| | TVSS | |
| RN-20 | QVQLVESGGGLVQPGGSLRLSCAASGNYFEINKMHWFR | 378 |
| | QAPGKQREFVATIGSGGSTAYADSVKGRFTISRDNAKNT | |
| | VYLQMNSLKPEDTAVYYCNALIYLADRRDGYYSYDYW | |
| | GQGTQVTVSS | |
| RN-21 | QVQLVESGGGLVQPGGSLRLSCAASGLQTSDNAMHWF | 379 |
| | RQAPGKNRELVAGLTSGTSRNYGDSVKGRFTISRDNAK | |
| | NTVYLQMNSLKPEDTAVYYCAAFPDVGDASSIGFVYW | |
| | GQGTQVTVSS | |
| RN-22 | QVQLVESGGGLVQPGGSLRLSCAASGSISEINLLHWFRQ | 380 |
| | APGKQREWVATMTSGGSTDYADSVKGRFTISRDNAKN | |
| | TVYLQMNSLKPEDTAVYYCAADEFTKYGHGRYWGQG | |
| | TQVTVSS | |
| RN-23 | QVQLVESGGGLVQPGGSLRLSCAASGGEKSISAAHWYR | 381 |
| | QAPGKEREFVAAITAASTTSYADSVKGRFTISRDNAKNT | |
| | VYLQMNSLKPEDTAVYYCTAVLYDSYDYWGQGTQVT | |
| | VSS | |
| RN-25 | QVQLVESGGGLVQPGGSLRLSCAASGEDYSYNAAHWF | 382 |
| | RQAPGKEREWVAAINSGGGTYYADSVKGRFTISRDNAK | |
| | NTVYLQMNSLKPEDTAVYYCAYLGALDRDGYDAWGQ | |
| | GTQVTVSS | |
| RN-26 | QVQLVESGGGLVQPGGSLRLSCAASGSISSINSMHWYRQ | 383 |
| | APGKEREWVAAVSSYGSDYYADSVKGRFTISRDNAKNT | |
| | VYLQMNSLKPEDTAVYYCAARTVTGFTYGDYWGQGT | |
| | QVTVSS | |
| RN-27 | QVQLVESGGGLVQPGGSLRLSCAASGPQFSSNAVSWFR | 384 |
| | QAPGKEREWVAGITSGGSTDYADSVKGRFTISRDNAKN | |
| | TVYLQMNSLKPEDTAVYYCTAPSVSDADRRTRDYWGQ | |
| | GTQVTVSS | |
| RN-28 | QVQLVESGGGLVQPGGSLRLSCAASGSQFSHNLMSWFR | 385 |
| | QAPGKQREFVAAITSFGSTGYADSVKGRFTISRDNAKNT | |
| | VYLQMNSLKPEDTAVYYCAALSSEDTSHYPRNYWGQG | |
| | TQVTVSS | |
| RN-29 | QVQLVESGGGLVQPGGSLRLSCAASGYILSENLTAWFR | 386 |
| | QAPGKAREVVATVTSGDYTNYDDSVKGRFTISRDNAKN | |
| | TVYLQMNSLKPEDTAVYYCAAGTSDWEVKWSHWGQG | |
| | TQVTVSS | |
| RN-30 | QVQLVESGGGLVQPGGSLRLSCAASGIPASYLNMHWFR | 387 |
| | QAPGKQREWVAGAGGINSNYYQDSVKGRFTISRDNAK | |
| | NTVYLQMNSLKPEDTAVYYCAALDGAESYDYVYPVW | |
| | GQGTQVTVSS | |
| RN-31 | QVQLVESGGGLVQPGGSLRLSCAASGSTGSDYAMHWF | 388 |
| | RQAPGKDREWVAGISSYGSDSYGDSVKGRFTISRDNAK | |
| | NTVYLQMNSLKPEDTAVYYCAALQASAGAGYYYDYW | |
| | GQGTQVTVSS | |

In embodiments, the VHHs of the disclosure include one or more amino acid substitutions, deletions, or additions. As indicated, changes are preferably of a minor nature, such as conservative amino acid substitutions that do not significantly affect the folding or activity of the VHHs of the disclosure.

The present disclosure provides VHHs that comprise, or alternatively consist of, variants (including derivatives) of the VHH CDRs described herein that specifically bind to a SARS-CoV-2 antigen or component. In embodiments, the present disclosure provides VHHs that comprise, or alternatively consist of, variants (including derivatives) of the VHH CDRs described herein that specifically bind to the spike protein of SARS-CoV-2. Standard techniques known to those of skill in the art can be used to introduce mutations in the nucleotide sequence encoding a VHH of the disclosure, including, for example, site-directed mutagenesis and PCR-mediated mutagenesis which result in amino acid substitutions. Preferably, the variants (including derivatives) encode less than 10 amino acid substitutions, less than 5 amino acid substitutions, less than 4 amino acid substitutions, less than 3 amino acid substitutions, or less than 2 amino acid substitutions relative to the reference VHH domain. In a preferred embodiment, the variants have conservative amino acid substitutions at one or more predicted non-essential amino acid residues. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., the ability to bind a SARS-CoV-2 antigen or component). Following mutagenesis, the encoded protein may routinely be expressed and the functional and/or biological activity of the encoded protein, (e.g., ability to specifically bind to a SARS-CoV-2 antigen or component) can be determined using techniques described herein or by routinely modifying techniques known in the art.

In embodiments, the disclosure relates to a polypeptide that comprises at least one VHH against SARS-CoV-2 as defined herein. In embodiments, the disclosure also relates to a polypeptide that comprises at least two or at least three anti-SARS-CoV-2 binding moieties such constructs enabling multimeric interactions are herein referred to as a Cerberbody and/or a Hydrabody complex. Complexes provided herein can be formed using any means. In embodiments, any number of higher-order complexes are formed. Higher order complexes can be formed that comprise 5HVZ. A 5HVZ can be a smAKAP AKB domain bound RIa dimerization/docking (D/D) complex. In embodiments, a composition provided herein comprises one or more of a 5HVZ.

### Cerberbody

In embodiments, a Cerberbody construct can be formed when a Spirulina strain (either two independent strains or a single strain from different ORFs) express a 5HVZ mediated homodimer molecule and smAKAP linked monomer VHH. These independent complexes form a further complex structure that will have three antigen binding sites. The presence of increased antigen binding reagents (VHHs) results in increased binding affinity, mainly due to avidity-based interaction. The three binding VHHs can be derived from the same VHH forming a homo-trimer. The design also enables mixing diverse VHHs with desirable binding and virus neutralization property. Such a molecule complex will be able to bind multiple epitopes and mutant variants of a virus such as novel SARS-Cov-2 variants that may not be captured by conventional immune surveillance or conventional therapies. We have demonstrated that the Cerberbody complexes show increased apparent binding when compared to dimeric or monomeric forms.

### Hydrabody

In embodiments, provided are also Hydrabody complexes. In embodiments, a Hydrabody comprises at least four antigen binding VHHs. Hydrabody complexes can be formed when a Spirulina strain (either two independent strains or a single strain from different ORFs) express a 5HVZ mediated homodimer molecule and smAKAP linked dimeric VHHs. The smAKAP mediated dimers contain VHHs on both the N- and C-terminal of smAKAP. The VHHs fused to the smAKAP can form homo-dimeric (the same VHH) or hetero-dimeric (two different VHHs on either end). Such a configuration can enable inclusion of multiple, therapeutically effective VHHs in a single complex.

In embodiments, a Cerberbody or a Hydrabody can comprise a sequence from Table 1, Table 2, or Table 3. In embodiments, a Cerberbody or Hydrabody comprise at least 2, 3, 4, 5, 6, 7, or 8 sequences selected from Tables 1-3. A Cerberbody or Hydrabody can comprise multiples of the same sequence or different sequences, for example CDR sequences in Table 1. In embodiments, a Cerberbody or Hydrabody can comprise from 1-4 duplications or combinations of any one of SEQ ID NO: 391-681 or SEQ ID NO: 292-388.

In addition to increased avidity-based apparent binding, the higher order complexes of Cerberbody and Hydrabody enable the formation of super potent and cross-reactive therapeutic complex that can bind and neutralize diverse virus variants. For example, in embodiments, a Hydrabody is administered to a subject in need thereof to neutralize two or more SARS-COV-2 variants. A Hydrabody that contains a dimer of dimers of two VHHs that bind and neutralize single variants can bind better than each separately and neutralize both variants. Such a design combines multiple components easily where a diverse epitope can be engaged resulting in decreased virus escape.

In embodiments, a composition provided herein, for example a Cerberbody and/or a Hydrabody can bind from about 1, 2, 3, 4, 5, 6, 7, or up to about 8 different SARS-CoV-2 variants.

Also provided are nucleic acids that encode any of the VHH of the disclosure and/or a polypeptide of the disclosure such as a Cerberbody and/or a Hydrabody. Such a nucleic acid will also be referred to below as a “nucleic acid of the disclosure” and may for example be in the form of a genetic construct. In embodiments, a multimeric construct, such as a Cerberbody or a Hydrabody, is more effective at neutralizing SARS-CoV-2 as compared to a monomeric or dimeric construct. In embodiments, the effectiveness is at least about 1-fold, 10-fold, 20-fold, 30-fold, 40-fold, 50-fold, 60-fold, 70-fold, 80-fold, 90-fold, 100-fold, 1000-fold, 5000-fold, 10,000-fold, 15,000-fold, 100,000-fold, 200,000-fold, 300,000-fold, 500,000-fold, 800,000-fold, or 1,000,000-fold more effective as compared to a non-multimeric construct. In embodiments, a multimeric construct, such as a Cerberbody or a Hydrabody, is more effective at reducing a symptom or disease of SARS-CoV-2 in a subject in need thereof as compared to a monomeric or dimeric construct. In embodiments, the effectiveness at reducing the symptom or disease is at least about 1-fold, 10-fold, 20-fold, 30-fold, 40-fold, 50-fold, 60-fold, 70-fold, 80-fold, 90-fold, 100-fold, 1000-fold, 5000-fold, 10,000-fold, 15,000-fold, 100,000-fold, 200,000-fold, 300,000-fold, 500,000-fold, 800,000-fold, or 1,000,000-fold more effective as compared to a non-multimeric construct. In embodiments, a multimeric construct, such as a Cerberbody or a Hydrabody, is more effective at neutralizing a variant of concern of SARS-CoV-2 that has evaded a vaccine therapy as compared to a monomeric or dimeric construct. In embodiments, the effectiveness is at least about 1-fold, 10-fold, 20-fold, 30-fold, 40-fold, 50-fold, 60-fold, 70-fold, 80-fold, 90-fold, 100-fold, 1000-fold, 5000-fold, 10,000-fold, 15,000-fold, 100,000-fold, 200,000-fold, 300,000-fold, 500,000-fold, 800,000-fold, or 1,000,000-fold more effective as compared to the vaccine therapy.

In embodiments, provided are also linkers that can be utilized in the compositions and methods provided herein. Any linker can be utilized in the compositions of the disclosure for example to join one or more VHHs of the disclosure. In embodiments, a linker can also be utilized to join a Cerberbody and/or Hydrabody. In embodiments, a linker comprises smAKAP peptide. In embodiments, the smAKAP peptide can be modified. A modification can be done for example to increase soluble protein expression. In embodiments, one or more residues in a linker can be mutated. In embodiments, two Cys residues in an exemplary linker, e.g., smAKAP peptide, are mutated to serine. In embodiments, a cleavable linker can also be utilized in compositions provided herein.

In embodiments, a linker comprises at least about or at most about: 80%, 82%, 84%, 86%, 88%, 90%, 92%, 94%, 96%, 98%, 100% identity to SEQ ID NO: 389 or SEQ ID NO: 390.

In embodiments, a linker can also be of any length. In embodiments, a linker is from about: 1-3, 2-6, 1-8, 3-10, 5-20, 3-15, 10-30, 15-35, 20-35, or 25-35 residues in length. In embodiments, a linker is about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length.

### SARS-CoV-2

In embodiments, compositions and methods provided herein are used to reduce or treat a SARS-CoV-2 related disease or condition. In embodiments the SARS-CoV-2 related disease or condition comprises COVID. In embodiments the SARS-CoV-2 related disease or condition comprises COVID-19.

In embodiments, a strain of SARS-CoV-2 is a variant of concern (VOI) or a variant of interest (VOI) as determined by the WHO. In embodiments, a strain of SARS-CoV-2 is not a variant of concern (VOI) as determined by the WHO. In embodiments, a strain of SARS-CoV-2 is not a variant of interest (VOI) as determined by the WHO. A SARS-CoV-2 variant that meets the definition of a VOI and, through a comparative assessment, has been demonstrated to be associated with one or more of the following changes at a degree of global public health significance: (a) increase in transmissibility or detrimental change in COVID-19 epidemiology; (b) increase in virulence or change in clinical disease presentation; and/or (c) decrease in effectiveness of public health and social measures or available diagnostics, vaccines, therapeutics. In embodiments, a VOI or a VOC is selected from the group consisting of Alpha, Beta, Gamma, Delta, and Omicron. In embodiments, a strain of SARS-CoV-2 is one or more of: alpha, beta, gamma, delta, epsilon, zeta, eta, theta, iota, kappa, lambda, mu, nu, xi, omicron, pi, rho, sigma, tau, upsilon, phi, chi, psi, or omega.

In embodiments, a SARS-CoV-2 is a variant as determined by the WHO. A variant may be identified as comprising genetic changes that are predicted or known to affect virus characteristics such as transmissibility, disease severity, immune escape, diagnostic or therapeutic escape; and/or identified to cause significant community transmission or multiple COVID-19 clusters, in multiple countries with increasing relative prevalence alongside increasing number of cases over time, or other apparent epidemiological impacts to suggest an emerging risk to global public health. In embodiments, a SARS-CoV-2 variant is lambda or Mu.

It is now widely accepted that SARS-CoV-2 colonizes the GI tract, and very high amounts of viral RNA are commonly found in stool samples. This viral reservoir exacerbates the pandemic in several ways. First, GI infection directly causes significant disease symptoms, with a majority of cases presenting with both GI and respiratory symptoms, and 25% presenting with only GI symptoms¹. In addition to contributing directly to disease progression and possibly liver damage¹¹, GI colonization may result in subsequent self-infection of the airways, particularly in vulnerable populations with weakened immune systems. Further, a systemic inflammatory response (cytokine storm) emanating from a GI infection may exacerbate pulmonary inflammation and contribute to acute respiratory distress syndrome (ARDS). Second, subjects with ongoing GI infection may test negative in nasopharyngeal swabs, yet silently carry and potentially transmit the virus for weeks.

In addition to colonizing the GI tract, SARS-CoV-2 can also colonize the respiratory tract. In embodiments, SARS-CoV-2, infects the cells along the airways by binding the ACE-2 receptor. Respiratory infection can lead to inflammation resulting in any one of: cough, chest tightness, and/or pain while breathing. In embodiments, airway inflammation can result in pneumonia. In embodiments, respiratory tract infection of SARS-CoV-2 can result in the need for lung transplant.

Respiratory diseases like influenza spread on a “one-to-few” basis through respiratory droplets and fomites. By contrast, diseases like norovirus that spread through the fecal-oral route are much more difficult to control in confined settings (nursing homes, cruise ships, naval vessels) because a single individual can spread the virus to many others—“one-to-many” transmission. Norovirus, is so contagious in confined settings that when researchers conduct human challenge clinical trials, extreme precautions must be taken to avoid infection of clinical staff. Fecal-oral transmission of SARS-CoV-2 may therefore explain some of the most dramatic local infection clusters. This is supported by the well-documented fecal-oral transmission of the SARS virus in 2003 (the Amoy Gardens event)^{9,12}, and the fact that many outbreak events apparently share “one-to-many” similarities to norovirus. The first major rich-world outbreak was a cruise ship (Diamond Princess) and the first major cluster of Covid-19 deaths in the U.S. occurred in a nursing home in Kirkland, WA. The foregoing is supported by the following observations.

The human host cell receptor used by SARS-CoV-2 for cell entry (ACE2) is “highly expressed in the lung AT2 cells, but also in upper and stratified epithelial cells and absorptive enterocytes from ileum and colon . . . ”¹³. Infective virus is found in stool from infected humans⁶ and animals⁸. Many subjects test positive in rectal swabs after symptoms resolve and oral swabs are negative⁷. Some individuals who test positive in rectal swabs are completely asymptomatic for Covid-19⁷. Positive test results in rectal samples occur even when oral swabs show negative results¹⁴. Modeling of outbreak dynamics China indicate that, “due to their greater numbers, undocumented infections were the source for 79% of documented cases”¹⁵ leading to speculation that GI-driven transmission may be responsible. Covid-19's epidemiology may in part explain why SARS-CoV-2 has spread more rapidly than prior coronavirus outbreaks.¹⁶

The compositions of the disclosure are safe to be used prophylactically and unlike a vaccine, work immediately upon administration and do not require a competent host immune system. The approach also side-steps the risks of vaccine development, which can sometimes exacerbate the disease through the phenomenon of antibody-dependent enhancement. In embodiments, a composition of the disclosure is effective at reducing or eliminating COVID when a SARS-CoV-2 infection evades vaccine therapy.

Compositions of the disclosure may be delivered through non-parenteral routes of administration, has no known toxicity risks, and is shelf stable for >1 year at 42° C. Therefore, neither cold-chain distribution nor trained medical personnel for administration are required.

In embodiments, an infection with SARS-CoV-2 is determined utilizing an in vitro assay. Suitable in vitro assays comprise RT-PCR, ELISA, RT-LAMP, transcription-mediated amplification (TMA), rolling cycle amplification (RCA), CRISPR, microarray, or any combination thereof. In embodiments, serological testing may also be utilized to monitor progress of disease. In embodiments, serological testing comprises an analysis of blood serum, plasma, saliva, sputum, and other biological fluids for the presence of immunoglobulin M (IgM) and immunoglobulin G (IgG) antibodies. Serological testing can provide an assessment of both short-term (days to weeks) and long-term (years or permanence) trajectories of antibody response, as well as antibody abundance and diversity.

### Spirulina

Compositions of the present disclosure include recombinant spirulina in a non-living form, wherein the spirulina is engineered to contain at least one VHH. Using spirulina as both a manufacturing system and delivery vehicle enables faster development without compromising safety. These non-living spirulina containing at least one VHH are then administered to a subject to elicit a response in the subject. In embodiments, non-living recombinant spirulina comprising at least one VHH is prepared by drying the live culture of the recombinant spirulina. Methods of drying include heat drying, e.g., drying in an oven; air-drying, spray drying, lyophilizing, freeze-drying, thin-film drying, spray granulating, or spray-freeze drying. Accordingly, in embodiments, compositions of the present disclosure comprise a dried biomass of a recombinant spirulina comprising at least one VHH as described herein.

Neutralizing VHH genes are stably engineered into spirulina, which manufactures and stores the antibodies in its cytoplasm. For oral administration, no trained medical personnel are required: the entire biomass is simply dried, whereby the desiccated (inviable) spirulina cell becomes an edible organic mini-capsule containing the VHH drug ‘payload’. VHHs are stable within the dried biomass for at least 12 months without refrigeration (>42° C.), facilitating stockpiling and eliminating cold-chain distribution requirements.

There are several advantages to delivering VHHs encapsulated in spirulina. One of these advantages is the increased resistance of the encapsulated VHH to proteolysis. Pharmacokinetic studies show that the dry spirulina biomass retains the bio-encapsulated VHH and protects it from gastric digestion, and that the VHHs are rapidly released from the biomass following exit from the stomach. Following release, the VHH antibodies are bioactive for hours in the GI tract. Ingestion of different capsule or tablet formulations can modulate the site of VHH release from the spirulina biomass, thus allowing for esophageal, small intestine or colon delivery depending on the specific pathogen target. After release, the mode of action is to bind directly to pathogen-specific proteins present in the gut lumen that are required for infectivity, thus blocking interaction with the host and allowing them to pass harmlessly out of the body. Development is easier and faster than vaccine development and carries the added advantages of working immediately and equally well in subjects who may be immunocompromised (e.g., due to stress or age).

For example, encapsulation in spirulina protects the VHH from the enzymes and conditions of the digestive tract, thereby allowing the therapeutic to be delivered to the portion of the digestive tract that digests the spirulina cells and releases the therapeutic. In embodiments, the compositions of the present disclosure survive (e.g., remain substantially intact) at a pH of about 1.3 to about 8.0. In embodiments, the compositions of the present disclosure survive in the oral cavity. In embodiments, the compositions of the present disclosure survive in the stomach. In embodiments, the compositions survive in the small intestine. In embodiments, the compositions survive in a simulated stomach environment. In embodiments, the simulated stomach environment has an acidic pH and contains pepsin. In embodiments, the simulated stomach environment has a pH of about 3.0 and about 2000 U/mL of pepsin. In embodiments, the compositions can survive in the gastrointestinal conditions or simulated stomach environment for about 5 minutes to about 1 day. In embodiments, the compositions survive in the gastrointestinal conditions or simulated stomach environment for about 5 minutes, about 10 minutes, about 20 minutes, about 30 minutes, about 1 hour, about 2 hours, about 3 hours, about 4 hours, about 5 hours, about 6 hours, about 7 hours, about 8 hours, about 9 hours, about 10 hours, about 12 hours, about 24 hours. In embodiments, the compositions survive in the gastrointestinal conditions or simulated stomach environment overnight.

To facilitate delivery to the airway, the VHH may be extracted from the spirulina by lysing the spirulina, partially purifying the soluble proteins including the VHH, and drying the partially purified protein by an appropriate drying method including any drying method provided herein. For example, the harvested spirulina slurry may be lysed using high pressure homogenization, and the soluble protein separated from insoluble materials by filtration, then diafiltered and concentrated by tangential flow ultrafiltration. The partially purified extract may be formulated with appropriate stabilizers and mucoadhesives, including carbohydrates such as trehalose, hyaluronic acid, or chitosan; amino acids such as lysine or glycine; and polymers such as polyvinyl alcohol or polyvinyl pyrrolidone. In embodiments, the VHH will comprise a purity from about: 4%, 6%, 8%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, or up to about 70% of solids.

Another advantage of the compositions of the present disclosure is their stability in storage. In embodiments, the compositions of the present disclosure are stable at elevated temperatures (e.g., greater than room temperature). In embodiments, the compositions of the present disclosure are stable at 42° C. In embodiments, the compositions of the present disclosure are stable at 42° C. for about one day to 5 years. In embodiments, the compositions of the present disclosure are stable at 42° C. for about one day, two days, three days, four days, five days, six days, seven days, one week, two weeks, three weeks, four weeks, one month, two months, three months, four months, five months, six months, or one year. In embodiments, the compositions of the present disclosure are stable at 42° C. for one month or three months. In embodiments, the compositions of the present disclosure are stable at room temperature (e.g., about 20° to about 29° C.). In embodiments, the compositions of the present disclosure are stable at 27° C. In embodiments, the compositions of the present disclosure are stable at 27° C. for about one day to 5 years. In embodiments, the compositions of the present disclosure are stable at 27° C. for about one day, two days, three days, four days, five days, six days, seven days, one week, two weeks, three weeks, four weeks, one month, two months, three months, four months, five months, six months, or one year. In embodiments, the compositions of the present disclosure are stable at 27° C. for one month or three months.

As used herein “spirulina” is synonymous with “Arthrospira.” Compositions of the present disclosure can comprise any one of the following species of spirulina: A. amethystine, A. ardissonei, A. argentina, A. balkrishnanii, A. baryana, A. boryana, A. braunii, A. breviarticulata, A. brevis, A. curta, A. desikacharyiensis, A. funiformis, A. fusiformis, A. ghannae, A. gigantean, A. gomontiana, A. gomontiana var. crassa, A. indica, A. jenneri var. platensis, A. jenneri Stizenberger, A. jenneri f. purpurea, A. joshii, A. khannae, A. laxa, A. laxissima, A. laxissima, A. leopoliensis, A. major, A. margaritae, A. massartii, A. massartii var. indica, A. maxima, A. meneghiniana, A. miniata var. constricta, A. miniata, A. miniata f. acutissima, A. neapolitana, A. nordstedtii, A. oceanica, A. okensis, A. pellucida, A. platensis, A. platensis var. non-constricta, A. platensis f. granulate, A. platensis fminor, A. platensis var. tenuis, A. santannae, A. setchellii, A. skujae, A. spirulinoides f. tenuis, A. spirulinoides, A. subsalsa, A. subtilissima, A. tenuis, A. tenuissima, and A. versicolor. In embodiments, spirulina is A. platensis.

### Pharmaceutical Compositions and Dosing

As used herein, the terms “oral composition” or “orally delivered composition” comprise compositions administered or delivered to the gastrointestinal tract (e.g., orally, compositions administered to the stomach via a feeding tube, etc.). Any appropriate area of the gastrointestinal tract may be targeted by the compositions of the present disclosure.

In embodiments, the compositions of the present disclosure are administered via the airway. In embodiments, the compositions of the present disclosure are administered by inhalation. In embodiments, the compositions of the present disclosure are administered intranasally. In embodiments, the compositions of the present disclosure are administered by a nebulizer, an inhaler, or a mist. In embodiments, the compositions of the present disclosure are lyophilized and delivered as a powder or a powder resuspended in a liquid.

In embodiments, the compositions of the present disclosure are formulated for administration via the airway. In embodiments, the compositions of the present disclosure are formulated for administration by inhalation. In embodiments, the compositions of the present disclosure are formulated for intranasal administration. In embodiments, the compositions of the present disclosure are formulated for administration by a nebulizer, an inhaler, a dry powder inhalation device, or a mist.

In embodiments, compositions of the present disclosure can comprise one or more pharmaceutically acceptable excipients. Pharmaceutically acceptable carriers include but are not limited to saline, buffered saline, dextrose, water, glycerol, sterile isotonic aqueous buffer, and combinations thereof. In embodiments, a pharmaceutically acceptable excipient is sodium bicarbonate.

### Uses of Compositions

In embodiments, compositions of the present disclosure can be used to reduce the severity of a disease or disorder in a subject in need thereof. In embodiments, compositions can be used to prevent a disease or disorder in a subject. In embodiments, compositions can be used to prevent initiation of a disease or disorder in a subject. In embodiments, compositions can be used to reduce the severity of a disease or disorder in a subject caused by a coronavirus e.g., SARS-CoV-2. In embodiments, compositions can be used to prevent or delay recurrence of a disease in a subject such as COVID-19.

### Methods of Treatment

In embodiments, the present disclosure provides a method of treating COVID-19, ARDS, inflammation, organ or tissue damage, or inflammatory lung damage or symptoms, and/or related conditions comprising administering compositions comprising a VHH of the present disclosure to a subject in need.

In embodiments, the present disclosure provides a method of treating COVID-19, ARDS, inflammation, organ or tissue damage, inflammatory lung damage symptoms, and/or a COVID-19 related GI symptom in a subject in need thereof comprising administering a composition comprising a VHH of the present disclosure. In embodiments, the present disclosure provides a method of preventing COVID-19, ARDS, inflammation, organ or tissue damage, inflammatory lung damage symptoms, and/or a COVID-19 related GI symptom in a subject in need thereof comprising administering a composition comprising a VHH of the present disclosure to the subject. In embodiments, the present disclosure provides a method of slowing the progression of COVID-19, ARDS, inflammation, organ or tissue damage, inflammatory lung damage symptoms, and/or a COVID-19 related GI symptom in a subject in need thereof comprising administering compositions comprising a VHH of the present disclosure to the subject. In embodiments, the present disclosure provides a method of ameliorating the symptoms of COVID-19, ARDS, inflammation, organ or tissue damage, inflammatory lung damage symptoms, and/or a COVID-19 related GI symptom and/or respiratory symptom in a subject in need thereof comprising administering a composition comprising a VHH of the present disclosure to the subject.

In embodiments, the present disclosure provides a method of decreasing inflammation in a subject in need thereof comprising administering compositions comprising a VHH of the disclosure to the subject. In embodiments, the present disclosure provides a method of decreasing fibrosis in a subject in need thereof comprising administering compositions comprising a VHH of the disclosure to the subject. In embodiments, the present disclosure provides a method of decreasing expression of markers of inflammation or fibrosis in the organ or tissue in a subject in need thereof comprising administering compositions comprising a VHH of the disclosure to the subject. In embodiments, the present disclosure provides a method of decreasing expression of one or more pro-inflammatory cytokines in a subject in need thereof comprising administering compositions comprising a VHH of the disclosure to the subject.

In embodiments, the one or more pro-inflammatory cytokines is implicated in the inflammatory response to lung injury. In embodiments, the one or more pro-inflammatory cytokines include, but are not limited to, IL-17A, TNFα, NF-κβ, NLRP3, IL-18, IL-1, IL-1R, TNFαR, and IL-18R.

In embodiments, the present disclosure provides a method of increasing the expression of one or more anti-inflammatory cytokines in a subject in need thereof comprising administering compositions comprising a VI-11-1 of the disclosure to the subject. In embodiments, the one or more anti-inflammatory cytokines include, but are not limited to the receptors that bind to one or more pro-inflammatory cytokines, interleukin-1 receptor antagonist, IL-4, IL-6, IL-10, IL-11, and IL-13. In embodiments, the present disclosure provides a method of inducing the proliferation of protective T-cells in a subject in need thereof comprising administering compositions comprising a VHH of the disclosure to the subject.

The treatment, prevention, delay, or amelioration of inflammatory lung damage and/or the symptoms thereof may be measured by any means known in the art. For example, evaluation may include measurement of oxygen levels in the subject, exhaled nitric oxide test, measurement of respiratory rate, spirometry, and/or inflammatory lung damage is treated, prevented, delayed, or ameliorated for about 1 week, about 1 month, about 2 months, about 3 months, about 4 months, about 5 months, about 6 months, about 8 months, about 1 year, about 2 years, about 5 years, and/or about 10 years compared with the blood pressure in an untreated SARS-CoV-2 infected subject. In embodiments, inflammatory lung damage is prevented, delayed, or ameliorated by about 1%, about 5%, about 10%, about 20%, about 30%, about 40%, about 50%, about 60%, about 70%, about 80%, or about 90% compared with blood pressure in an untreated SARS-CoV-2 infected subject. In embodiments, this prevention, delay, or amelioration of inflammatory lung damage is observed at the time points disclosed herein.

In embodiments, administration of the compositions disclosed herein improve inflammatory lung damage in a subject compared to an untreated inflammatory lung damage subject. In embodiments, inflammatory lung damage is improved for about 1 week, about 1 month, about 2 months, about 3 months, about 4 months, about 5 months, about 6 months, about 8 months, about 1 year, about 2 years, about 5 years, and/or about 10 years compared with cardiac function of an untreated SARS-CoV-2 infected subject. In embodiments, inflammatory lung damage is improved by about 1%, about 5%, about 10%, about 20%, about 30%, about 40%, about 50%, about 60%, about 70%, about 80%, or about 90% compared with cardiac function of an untreated SARS-CoV-2 infected subject. In embodiments, this improvement in inflammatory lung damage is observed at the time points disclosed herein.

The treatment, prevention, delay, or amelioration of COVID-19, ARDS, inflammation, organ, or tissue damage and/or the symptoms thereof may be measured by any means known in the art. In embodiments, COVID-19, ARDS, inflammation, organ, or tissue damage and/or symptoms thereof are treated, prevented, delayed, or ameliorated for about 1 week, about 1 month, about 2 months, about 3 months, about 4 months, about 5 months, about 6 months, about 8 months, about 1 year, about 2 years, about 5 years, and/or about 10 years compared with the blood pressure in an untreated SARS-CoV-2 infected subject. In embodiments, COVID-19, ARDS, inflammation, organ, or tissue damage and/or the symptoms thereof are prevented, delayed, or ameliorated by about 1%, about 5%, about 10%, about 20%, about 30%, about 40%, about 50%, about 60%, about 70%, about 80%, or about 90% compared with blood pressure in an untreated SARS-CoV-2 infected subject. In embodiments, this prevention, delay, or amelioration of COVID-19, ARDS, inflammation, organ, or tissue damage and/or the symptoms thereof is observed at the time points disclosed herein. In embodiments, administration of a composition of the disclosure is effective in reducing a period of illness associated with the disease or disorder related to SARS-CoV-2 by at least about 1-3 days, 3-5 days, 4-6 days, a week, two weeks, or up to about three weeks. In embodiments, administration of a composition of the disclosure is effective in reducing a period of illness associated with the disease or disorder related to SARS-CoV-2 by at least about 1 day, 2 days, 4 days, 6 days, 8 days, 10 days, 12 days, 14 days, 16 days, 18 days, 20 days, 22 days, 24 days, 26 days, 28 days, or 30 days.

In embodiments, administration of the compositions disclosed herein improve COVID-19, ARDS, inflammation, organ, or tissue damage and/or the symptoms thereof in a subject compared to an untreated SARS-CoV-2 infected subject. In embodiments, COVID-19, ARDS, inflammation, organ, or tissue damage and/or the symptoms thereof are improved for about 1 week, about 1 month, about 2 months, about 3 months, about 4 months, about 5 months, about 6 months, about 8 months, about 1 year, about 2 years, about 5 years, and/or about 10 years compared with cardiac function of an untreated SARS-CoV-2 infected subject. In embodiments, COVID-19, ARDS, inflammation, organ, or tissue damage and/or the symptoms thereof are improved by about 1%, about 5%, about 10%, about 20%, about 30%, about 40%, about 50%, about 60%, about 70%, about 80%, or about 90% compared with cardiac function of an untreated SARS-CoV-2 infected subject. In embodiments, this improvement in COVID-19, ARDS, inflammation, organ, or tissue damage and/or the symptoms thereof is observed at the time points disclosed herein.

### Subject Populations

Any appropriate subject may be administered the compositions of the present disclosure. In embodiments, the subject is at risk of developing COVID-19, ARDS, inflammation, organ, or tissue damage and/or the symptoms thereof In embodiments, the subject is experiencing COVID-19, ARDS, inflammation, organ, or tissue damage and/or the symptoms thereof In embodiments, the subject is at risk of developing inflammatory lung damage. In embodiments, the subject is experiencing inflammatory lung damage. In embodiments, the subject is infected with, or presumed to be infected with, SARS-CoV-2. In embodiments, the subject has COVID-19 and/or is experiencing symptoms associated with COVID-19. In embodiments, the subject is hospitalized. In embodiments, the subject has a mild case of COVID-19, and/or is experiencing mild symptoms associated with COVID-19. In embodiments, the subject has a moderate case of COVID-19, and/or is experiencing moderate symptoms associated with COVID-19. In embodiments, the subject has a severe case of COVID-19, and/or is experiencing severe symptoms associated with COVID-19 (e.g., rapid clinical deterioration, ARDS, and/or death). In embodiments, the subject requires assistance breathing. In embodiments, the subject is receiving oxygen. In embodiments, the subject is receiving oxygen by face mask or nasal cannula with prongs. In embodiments, the subject requires a ventilator to breathe. In embodiments, the subject exhibits low oxygen saturation levels. In embodiments, the subject exhibits an increased respiratory rate (e.g., greater than 24 breaths/minute). In embodiments, the subject exhibits an accompanying fever (e.g., temperature greater than 101° F.). In embodiments, the subject is at risk of progressing to more severe COVID-19 or symptoms, and the composition of the present disclosure is administered before symptoms worsen.

In embodiments, a subject has been vaccinated against SARS-CoV-2. In embodiments, a subject has not been vaccinated against SARS-CoV-2. Suitable vaccines comprise mRNA vaccines, vector vaccines, protein subunit vaccines, and combinations thereof. In embodiments, a subject has received at least about 1, 2, 3, or 4 administrations of the vaccine. In embodiments, a subject has received a booster of the vaccine, such as a third or fourth administration.

In embodiments, a subject has previously been treated with an anti-SARS-CoV-2 therapy or COVID-19 therapy. Suitable therapies comprise: monoclonal antibodies (e.g., bamlanivimab, evusheld, sotrovimab, Regen-Cov, and etesevimab), Remdesivir, mechanical ventilation, oxygen therapy, and combinations thereof. In embodiments, a subject in need thereof is recalcitrant to an anti-SARS-CoV-2 therapy or COVID-19 therapy for example those suitable therapies provided herein.

In embodiments the subject is between the ages of 18 and 100. In embodiments, the subject is between the ages of 18 and 85. In embodiments, the subject is between the ages of 50 and 100. In embodiments, the subject is older than 65 years old. In embodiments, the subject is 60, 61, 62, 63, 64, 65, 66, 67, 68, 69, 70 ,71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 years old or older. In embodiments, the subject who is older than 50 years old is considered high risk of developing severe disease (e.g., COVID-19 or ARDS). In embodiments, the subject who is older than 65 years old is considered high risk of developing severe disease (e.g., COVID-19 or ARDS).

In embodiments, a subject who possess one or more comorbidities is considered high risk for developing severe disease (e.g., COVID-19 or ARDS). In embodiments, the comorbidity includes, but is not limited to, obesity, hypertension, diabetes, an autoimmune disorder (e.g., rheumatoid arthritis), heart disease, heart failure, atherosclerosis, cancer (e.g., lung cancer), a history of smoking or exposure to other lung-damaging agents), liver disease, alcoholism, other pulmonary infection, and chronic kidney disease. In embodiments, the subject at risk presents with elevated markers of cardiac injury or dysfunction (e.g., hsTnI, NT-proBNP). In embodiments, race is a factor in a subject being considered at high risk of developing severe disease (e.g., COVID-19 or ARDS). In embodiments, socioeconomic status is a factor in a subject being considered at high risk of developing severe disease (e.g., COVID-19 or ARDS).

In embodiments, while being administered with a composition of the present disclosure, the subject continues to receive standard of care for any comorbidities.

### Combination Therapies

The compositions disclosed herein may be administered with various therapies used to treat, prevent, delay, or ameliorate inflammatory lung damage, COVID-19, and/or ARDS. In embodiments, the composition is administered concomitantly with standard of care medications. The one or more therapeutic agents may be any compound, molecule, or substance that exerts therapeutic effect to a subject in need thereof.

In embodiments, the compositions disclosed herein are administered with therapeutic agents including, but not limited to, antiviral agents, anti-malarial agents, agents that protect epithelial cells, defibrotide, convalescent plasma, chloroquine, hydroxychloroquine, remdesivir, desferal, favipiravir, corticosteroids, clevudine, anti-inflammatory agents, anti-oxidant agents, dapaglifiozin, IFX-1, ruxolitinib, baricitinib, interferon beta la, azithromycin, tocilizumab, acalabrutinib, umifenovir, ciclesonide, sarilumab, anti-interleukin agents, and telmisartan.

The one or more therapeutic agents may be “co-administered”, i.e., administered together in a coordinated fashion to a subject, either as separate compositions or admixed in a single composition. By “co-administered”, the one or more therapeutic agents may also be administered simultaneously with the present compositions, or be administered separately, including at different times and with different frequencies. The one or more therapeutic agents may be administered by any known route, such as orally, intravenously, intramuscularly, nasally, via aerosol, subcutaneously, intra-vaginally, intra-rectally, and the like; and the therapeutic agent may also be administered by any conventional route. In embodiments, the composition is administered subcutaneously.

When two or more therapeutic agents are used in combination, the dosage of each therapeutic agent is commonly identical to the dosage of the agent when used independently. However, when a therapeutic agent interferes with the metabolism of others, the dosage of each therapeutic agent is properly adjusted. Alternatively, where the two or more therapeutic agents show synergistic effects, the dose of one or more may be reduced. Each therapeutic agent may be administered simultaneously or separately in an appropriate time interval.

### Methods of Making Compositions

Provided are methods of making compositions described herein. Methods of making compositions comprise introducing into a spirulina a nucleic acid sequence encoding the at least one VHH. In embodiments, the methods of making compositions comprise introducing into a spirulina a polypeptide. In embodiments, the spirulina is A. platensis.

Any appropriate means for transforming spirulina may be used in the present disclosure. Exemplary methods for transforming spirulina to express a heterologous protein are described in U.S. Pat. No. 10,131,870, which is incorporated by reference herein in its entirety.

In embodiments, methods of making a composition include introducing an expression vector having a nucleic acid sequence encoding the at least one VHH into a spirulina cell. In embodiments, the vector is not integrated into the spirulina genome. In embodiments, the vector is a high copy or a high expression vector. In embodiments the nucleic acid sequence encoding the at least one VHH is under the control of a strong promoter. In embodiments the nucleic acid sequence encoding the at least one VHH is under the control of a constitutive promoter. In embodiments the nucleic acid sequence encoding the at least one VHH is under the control of an inducible promoter.

In embodiments, the vector or a portion thereof (e.g., the VHH coding portion) is integrated into the spirulina genome. In embodiments, methods of making a composition include introducing a vector (e.g., via homologous recombination) having homology arms and a nucleic acid sequence encoding the at least one VHH into a spirulina cell.

In embodiments, a vector having homology arms and a nucleic acid sequence encoding the at least one VHH can be introduced into spirulina using electroporation. The electroporation is preferably carried out in the presence of an appropriate osmotic stabilizer.

Prior to introduction of the vector into spirulina, the spirulina may be cultured in any suitable media for growth of cyanobacteria such as SOT medium. SOT medium includes NaHCO₃ 1.68 g, K₂HPO₄ 50 mg, NaNO₃ 250 mg, K₂50₄ 100 mg, NaCl 100 mg, MgSO₄.7H₂O, 20 mg, CaCl₂.2H₂O 4 mg, FeSO₄.7H₂O 1 mg, Na₂EDTA.2H₂O 8 mg, A5 solution 0.1 mL, and distilled water 99.9 mL. As solution includes H₃BO₃ 286 mg, MnSO4.5H₂O 217 mg, ZnSO₄, 7H₂O 22.2 mg, CuSO_{4.5}H₂O 7.9 mg, Na₂MoO_{4.}2H₂O 2.1 mg, and distilled water 100 mL. Cultivation may occur with shaking (e.g., 100-300 rpm) at a temperature higher than room temperature (e.g., 25-37° C.) and under continuous illumination (e.g., 20-2,000, 50-500, or 100-200 μmol photon m⁻²s⁻¹). The growing cells may be harvested when the optical density at 750 nm reaches a predetermined threshold (e.g., OD₇₅₀ of 0.3-2.0, 0.5-1.0, or 0.6-0.8). A volume of the harvested cells may be concentrated by centrifugation then resuspended in a solution of pH balancer and salt. The pH balancer may be any suitable buffer that maintains viability of spirulina while keeping pH of the media between 6 and 9 pH, between 6.5 and 8.5 pH, or between 7 and 8 pH. Suitable pH balancers include HEPES, HEPES-NaOH, sodium or potassium phosphate buffer, and TES. The salt solution may be NaCl at a concentration of between 50 mM and 500 mM, between 100 mM and 400 mM, or between 200 mM and 300 mM. In an embodiment between 1-50 mL of 1-100 mM pH balance may be used to neutralize the pH.

Cells collected by centrifugation may be washed with an osmotic stabilizer and optionally a salt solution (e.g., 1-50 mL of 0.1-100 mM NaCl). Any amount of the culture may be concentrated by centrifugation. In an embodiment between 5-500 mL of the culture may be centrifuged. The osmotic stabilizer may be any type of osmotic balancer that stabilizes cell integrity of spirulina during electroporation. In an embodiment, the osmotic stabilizer may be a sugar (e.g., w/v 0.1-25%) such as glucose or sucrose. In an embodiment the osmotic stabilizer may be a simple polyol (e.g., v/v 1-25%) including glycerine, glycerin, or glycerol. In an embodiment the osmotic stabilizer may be a polyether including (e.g., w/v 0.1-20%) polyethylene glycol (PEG), poly(oxyethylene), or poly (ethylene oxide) (PEO). The PEG or PEO may have any molecular weight from 200 to 10,000, from 1000 to 6000, or from 2000 to 4000. In an embodiment the pH balancer or buffer may be used instead of or in addition to the osmotic stabilizer.

A vector having homology arms and a nucleic acid sequence encoding the at least one exogenous polypeptide can be introduced into spirulina cells (e.g., A. platensis.) that are cultured and washed with an osmotic stabilizer as described above. Electroporation can be used to introduce the vector.

Electroporation may be performed in a 0.1-, 0.2- or 0.4-cm electroporation cuvette at between 0.6 and 10 kV/cm, between 2.5 and 6.5 kV/cm, or between 4.0 and 5.0 kV/cm; between 1 and 100 μF, between 30 and 70 μF, or between 45 and 55 g; and between 10 and 500 ma between 50 and 250 mΩ or between 90 and 110 mΩ. In embodiments, electroporation may be performed at 4.5 kV/cm, 50 μf, and 100 mΩ.

Following electroporation, the cells may be grown in the presence of one or more antibiotics selected based on resistance conferred through successful transformation with the plasmid. Post-electroporation culturing may be performed at reduced illumination levels (e.g., 5-500, 10-100, or 30-60 μmol photon m⁻²s⁻¹). The culturing may also be performed with shaking (e.g., 100-300 rpm). The level of antibiotics in the media may be between 5 and 100 μg/mL. Post-electroporation culturing may be continued for 1-5 days or longer. Successful transformants identified by antibiotic resistance may be selected over a time course of 1 week to 1 month on plates or in 5-100 mL of SOT medium supplemented with 0.1-2.0 μg of appropriate antibiotics.

A vector used in the methods may be a plasmid, bacteriophage, or a viral vector into which a nucleic acid sequence encoding the at least one VHH may be inserted or cloned. A vector may comprise one or more specific sequences that allow recombination into a particular, desired site of the spirulina' s chromosome. These specific sequences may be homologous to sequences present in the wild-type spirulina. A vector system can comprise a single vector or plasmid, two or more vectors or plasmids, some of which increase the efficiency of targeted mutagenesis, or a transposition. The choice of the vector will typically depend on the compatibility of the vector with the spirulina cell into which the vector is to be introduced. The vector can include a reporter gene, such as a green fluorescent protein (GFP), which can be either fused in frame to one or more of the encoded VHHs, or expressed separately. The vector can also include a positive selection marker such as an antibiotic resistance gene that can be used for selection of suitable transformants. The vector can also include a negative selection marker such as the type II thioesterase (tesA) gene or the Bacillus subtilis structural gene (sacB). Use of a reporter or marker allows for identification of those cells that have been successfully transformed with the vector.

In embodiments, the vector includes one or two homology arms that are homologous to DNA sequences of the spirulina genome that are adjacent to the targeted locus. The sequence of the homology arms can be partially or fully complementary to the regions of spirulina genome adjacent to the targeted locus.

The homology arms can be of any length that allows for site-specific homologous recombination. A homology arm may be any length between about 2000 bp and 500 bp. For example, a homology arm may be about 2000 bp, about 1500 bp, about 1000 bp, or about 500 bp. In embodiments having two homology arms, the homology arms may be the same or different length. Thus, each of the two homology arms may be any length between about 2000 bp and 500 bp. For example, each of the two homology arms may be about 2000 bp, about 1500 bp, about 1000 bp, or about 500 bp.

A portion of the vector adjacent to one homology arm or flanked by two homology arms modifies the targeted locus in the spirulina genome by homologous recombination. The modification may change a length of the targeted locus including a deletion of nucleotides or addition of nucleotides. The addition or deletion may be of any length. The modification may also change a sequence of the nucleotides in the targeted locus without changing the length. The targeted locus may be any portion of the spirulina genome including coding regions, non-coding regions, and regulatory sequences.

### NUMBERED EMBODIMENTS

Notwithstanding the appended claims, the following numbered embodiments also form part of the instant disclosure.
1. A VHH antibody that binds SARS-CoV-2, wherein the VHH antibody comprises three complementarity determining regions (CDR1, CDR2, and CDR3) each of which is selected from an amino acid sequence comprising at least about 85% identity to a sequence in Table 1.
2. The VHH antibody of embodiment 1, wherein the CDR1, CDR2, and CDR3 each comprise at least about 90%, 95%, 97%, or 99% identity to a sequence in Table 1.
3. The VHH antibody of any one of embodiments 1-2, wherein CDR1, CDR2, and CDR3 correspond to SEQ ID NO: 610, 611, and 612.
4. The VHH antibody of embodiment 3, wherein the VHH antibody comprises at least 85% identity with SEQ ID NO: 365.
5. The VHH antibody of any one of embodiments 1-2, wherein CDR1, CDR2, and CDR3 correspond to SEQ ID NO: 421, 422, and 423.
6. The VHH antibody of embodiment 5, wherein the VHH antibody comprises at least 85% identity with SEQ ID NO: 302.
7. A polypeptide comprising the VHH antibody of any one of embodiments 1-6.
8. A polypeptide comprising at least two VHH antibodies of any one of embodiments 1-6.
9. The polypeptide of embodiment 8, wherein the at least two VHH antibodies comprise SEQ ID NO: 365 or SEQ ID NO: 302.
10. The polypeptide of embodiments 8-9, wherein the at least two VHH antibodies are connected to each other via a linker.
11. The polypeptide of embodiment 10, wherein the linker comprises at least about 85% identity to SEQ ID NO: 390.
12. A polypeptide comprising at least three VHH antibodies of any one of embodiments 1-6.
13. A polypeptide comprising at least four VHH antibodies of any one of embodiments 1-6.
14. The polypeptide of embodiment 12 or 13, wherein the VHH antibodies are different.
15. The polypeptide of embodiment 12 or 13, wherein the VHH antibodies are the same.
16. A pharmaceutical composition comprising the VHH antibody of any one of embodiments 1-6 and a pharmaceutically acceptable carrier.
17. A pharmaceutical composition comprising the polypeptide of any one of embodiments 7-15 and a pharmaceutically acceptable carrier.
18. A method of treating a disease or disorder related to SARS-CoV-2, the method comprising: administering the pharmaceutical composition of embodiment 16 to a subject in need thereof, thereby treating the disease or disorder related to SARS-CoV-2.
19. A method of treating a disease or disorder related to SARS-CoV-2, the method comprising: administering the pharmaceutical composition of embodiment 17 to a subject in need thereof, thereby treating the disease or disorder related to SARS-CoV-2.
20. The method of embodiment 18 or 19, wherein the subject in need thereof is infected with SARS-CoV-2 as determined by PCR assay.
21. The method of any one of embodiments 18-20, wherein the subject in need thereof has been diagnosed as being infected with SARS-CoV-2.
22. The method of any one of embodiments 18-21, wherein the subject in need thereof is at risk of developing Coronavirus Disease (COVID).
23. The method of embodiment 22, wherein the subject in need thereof is at risk of developing severe COVID.
24. The method of any one of embodiments 18-23, wherein the subject in need thereof has one or more comorbidities.
25. The method of embodiment 24, wherein the subject in need thereof is treated for the one or more comorbidities.
26. The method of any one of embodiments 18-25, wherein the pharmaceutical composition is delivered encapsulated in a spirulina cell.
27. The method of embodiment 26, wherein the spirulina cell is dried.
28. The method of embodiment 26 or 27, wherein the spirulina cell is of strain A. platensis.
29. The method of any one of embodiments 18-28, wherein the pharmaceutical composition is delivered orally.
30. The method of any one of embodiments 18-28, wherein the pharmaceutical composition is delivered via the airway.
31. The method of embodiment 30, wherein, the pharmaceutical composition is delivered via inhalation.
32. The method of embodiment 30 or 31, wherein the pharmaceutical composition is delivered nasally.
33. The method of any one of embodiments 18-32, wherein the SARS-CoV-2 is a variant of concern.
34. The method of embodiment 33, wherein the variant of concern is selected from the group consisting of: alpha, beta, gamma, delta, and omicron.
35. The method of any one of embodiments 18-34, wherein the SARS-CoV-2 evades an immune response associated with vaccine therapy.
36. A method of treating a disease or disorder related to SARS-CoV-2, the method comprising: administering the pharmaceutical composition of embodiment 16 or 17 to a subject in need thereof thereby treating the disease or disorder related to SARS-CoV-2, wherein the disease or disorder is recalcitrant to anti-SARS-CoV-2 vaccine therapy.

37. The method of embodiment 36, wherein the subject in need thereof has previously been administered an anti-SARS-CoV-2 monoclonal therapy.
38. The method of embodiments 36-37, wherein the subject in need thereof has received a booster of the anti-SARS-CoV-2 vaccine therapy.
39. The method of embodiments 36-37, wherein the subject in need thereof has not received a booster of the anti-SARS-CoV-2 vaccine therapy.
40. The method of any one of embodiments 36-39, wherein the administering is effective in reducing or eliminating a symptom of the disease or disorder related to the SARS-CoV-2.
41. The method of embodiment 40, wherein the symptom is a gastrointestinal symptom.
42. The method of embodiment 40, wherein the symptom is a respiratory symptom.
43. The method of any one of embodiments 36-42, wherein the administering is effective in reducing a period of illness associated with the disease or disorder related to the SARS-CoV-2 by at least about 1-3 days.

### EXAMPLES

### Example 1—Binding Characterization of Anti-SARS-CoV-2 VHHs

The VHHs of the disclosure were assessed for affinity for the RBD or intact spike protein as measured by biolayer interferometry (BLI). The VHHs of the disclosure were expressed with scaffolds to promote solubility and bioactivity. Specifically, the VHHs were designed as monomers (MBP-VHH-6His) and dimers (MBP-5HVZ-VHH-6His) for expression and purification from E. coli, and subsequent in vitro characterization and expression in spirulina. An additional set of VHHs were built into alternative multimeric scaffolds using the proline-rich linker.

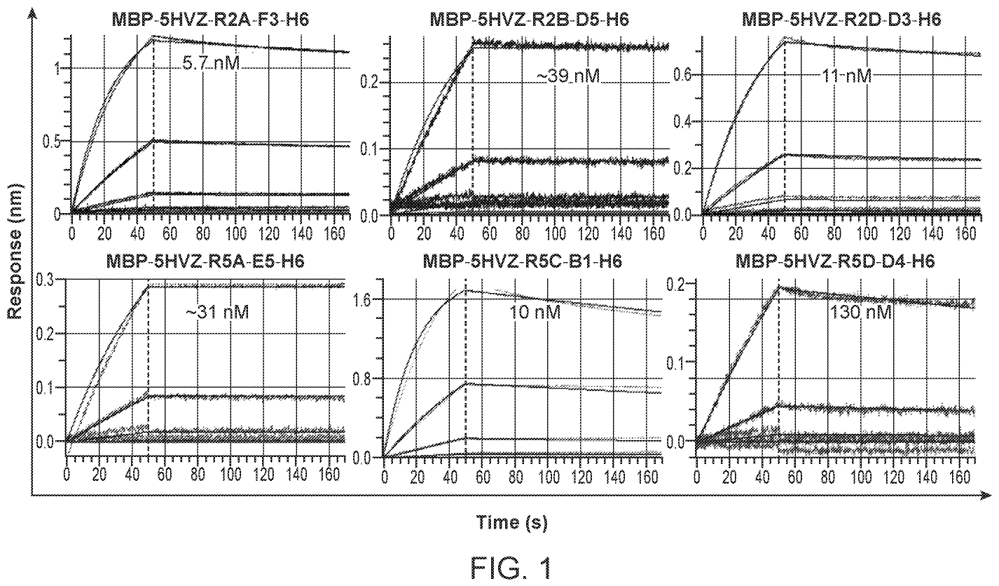

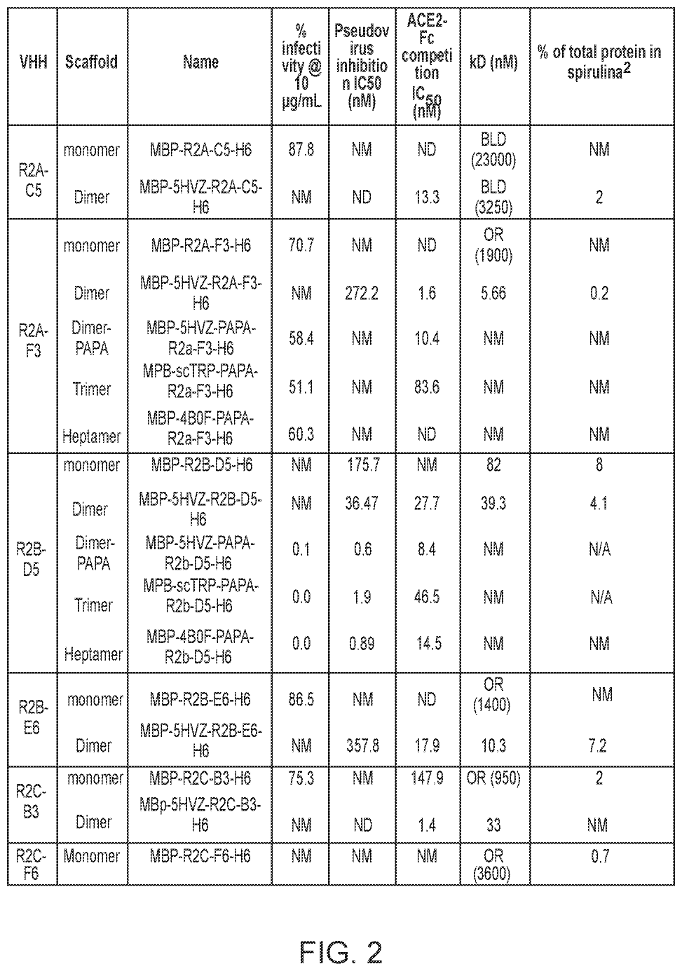

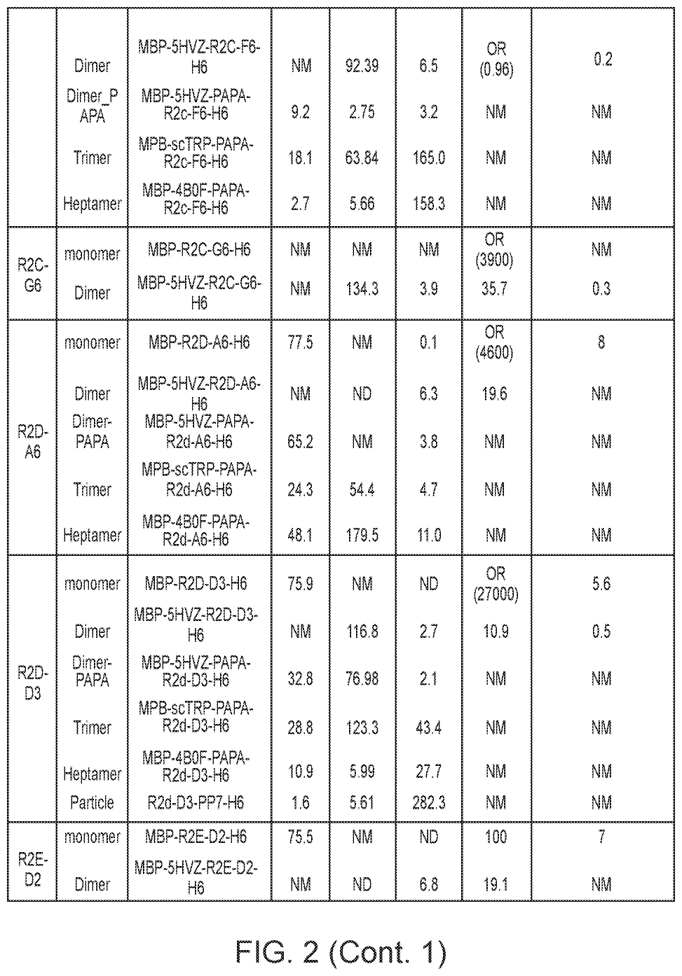

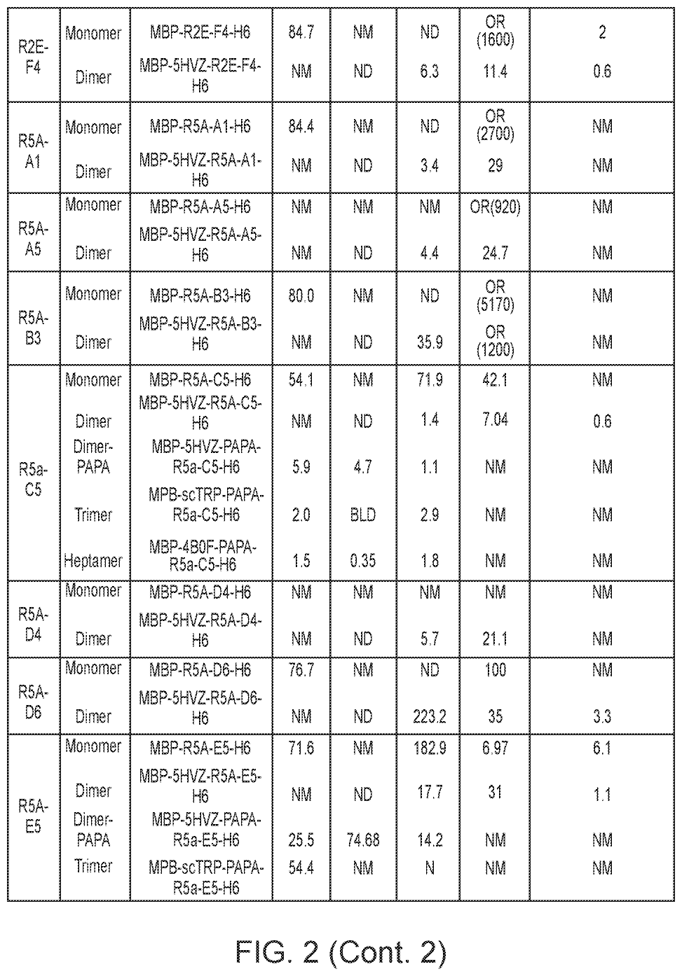

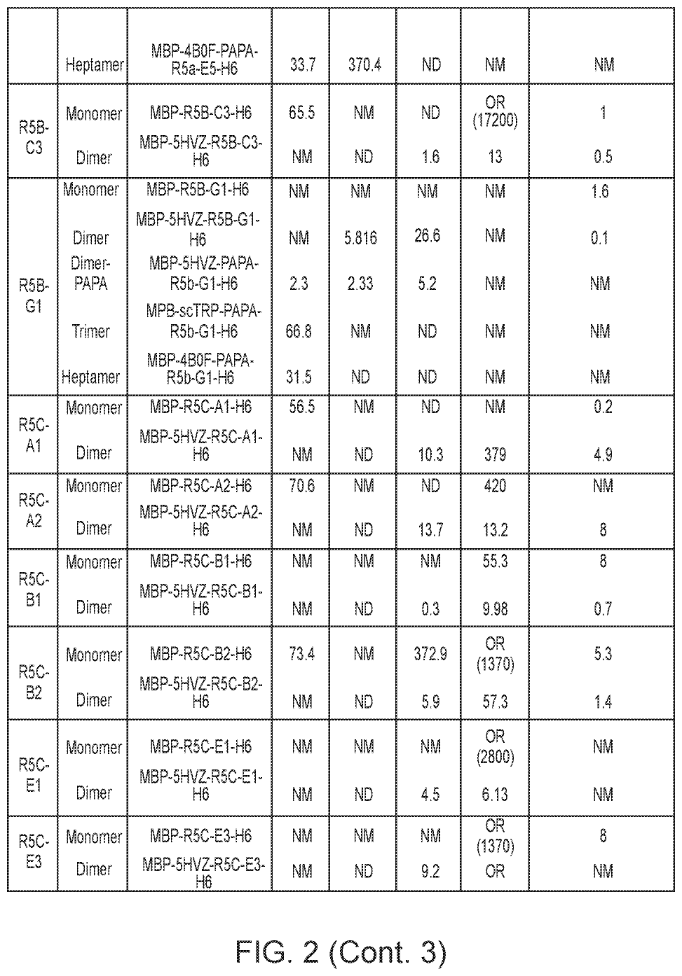

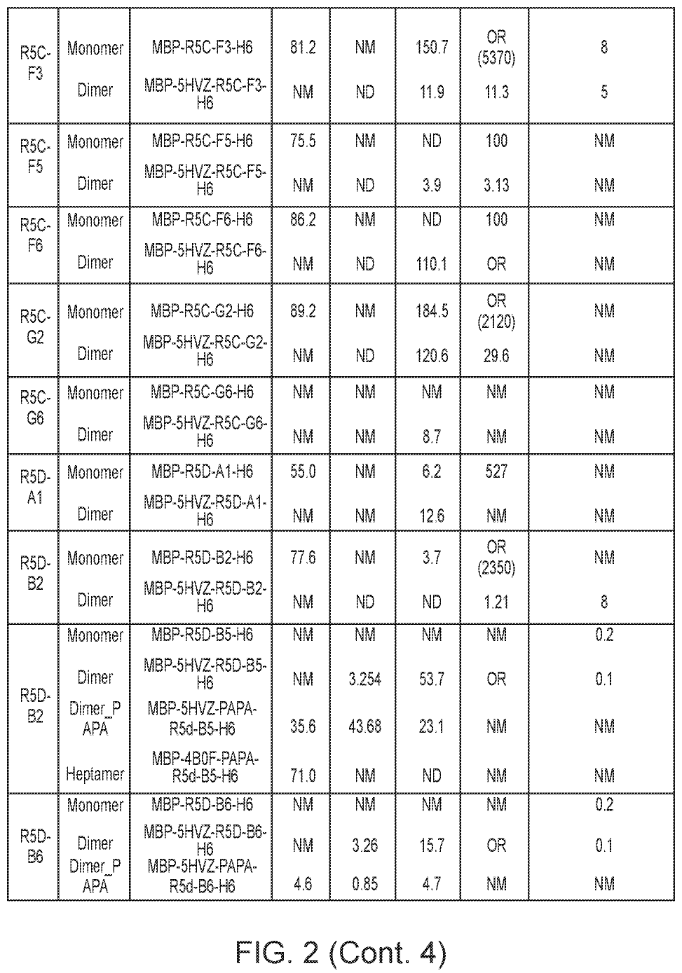

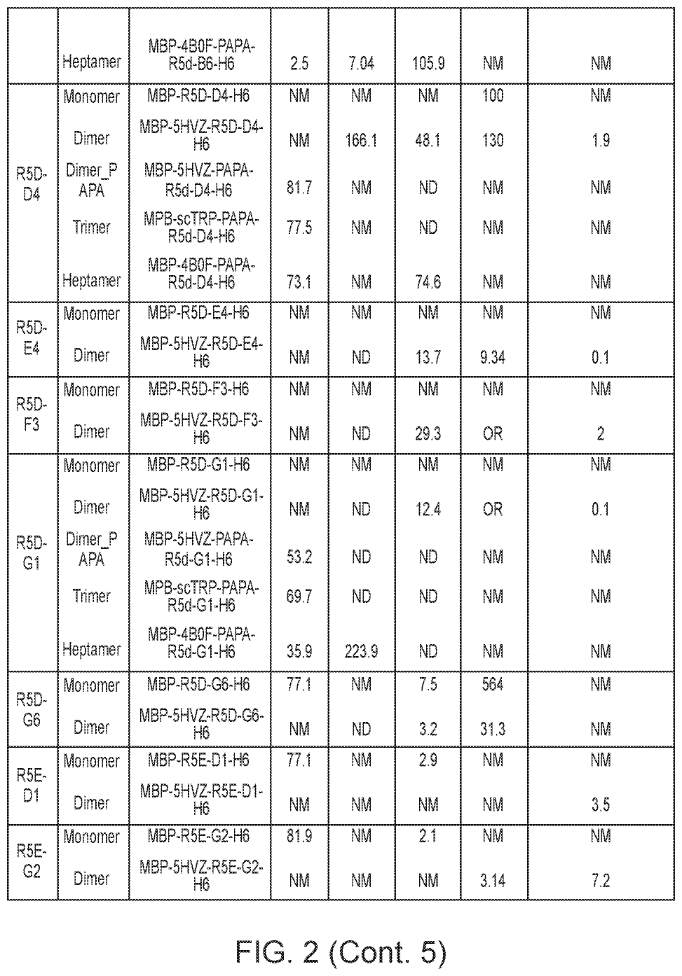

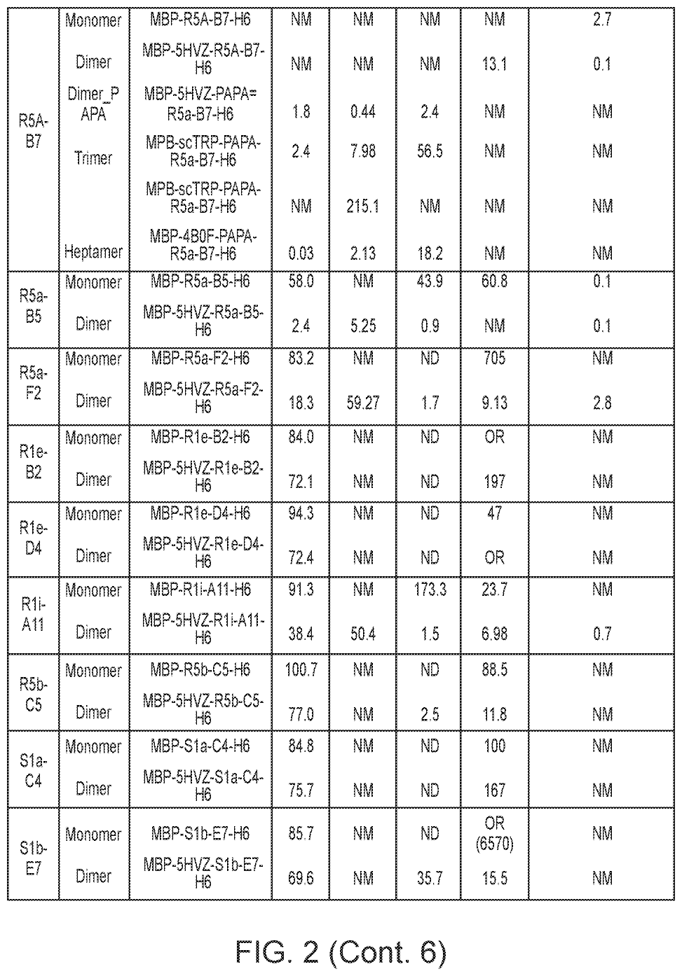

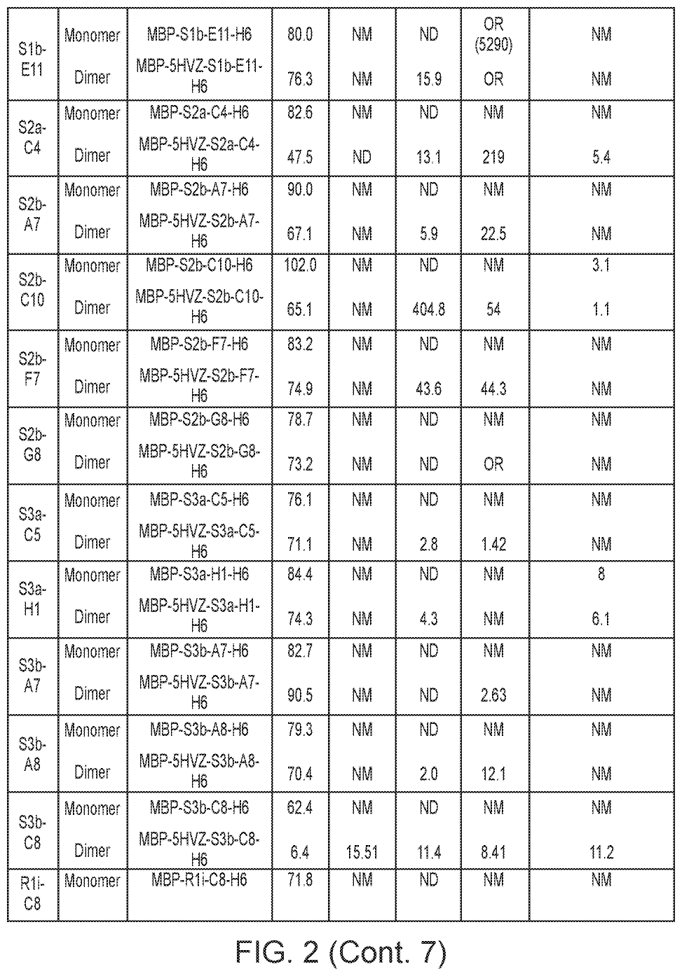

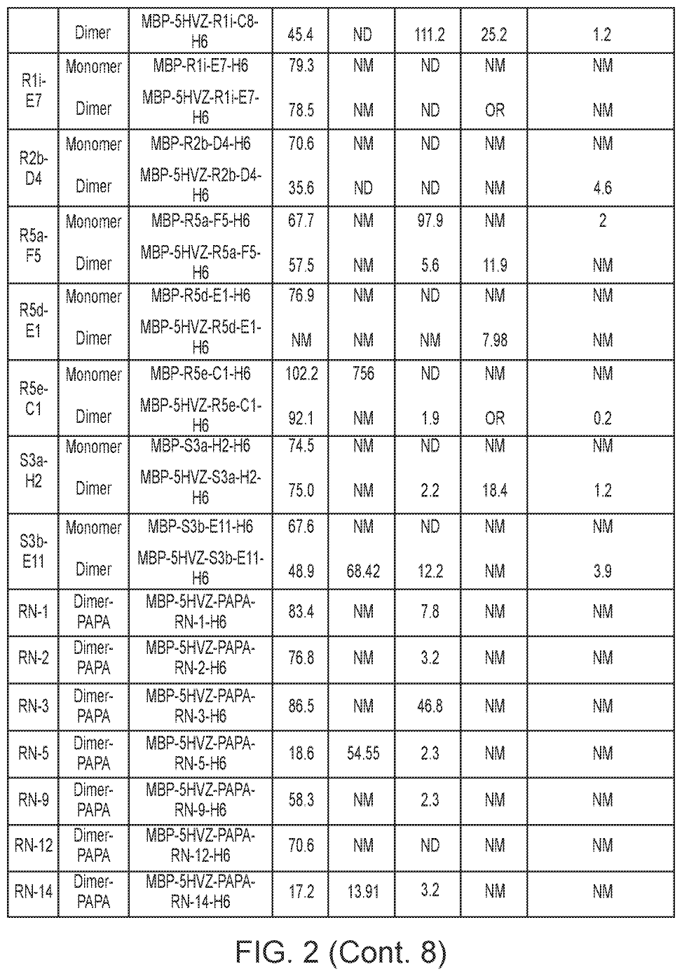

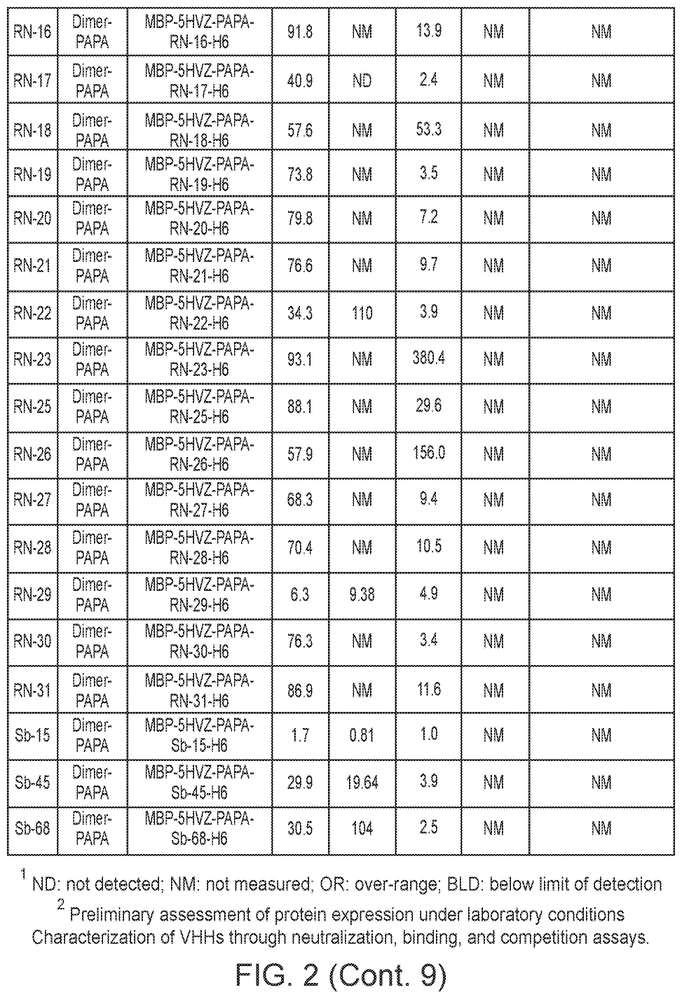

To measure VHH binding affinity to RBD, binding kinetics were assessed by BLI. VHH binding activity was determined by a quantitative label-free assay using a biolayer interferometry instrument, a ForteBio Octet, which can measure the kinetic parameters kₒₙ and k_{off} to determine the dissociation constant K_{d}. Rates of association and dissociation were calculated by incubating antigen-loaded biosensors with various concentrations of VHHs (monomers at 363.6 nM, 36.4 nM, and 3.64 nM and dimers at 163.9 nM, 16.4 nM, and 1.64 nM) (FIG. 1). The binding kinetics confirm increased binding affinity by dimeric VHHs, mainly due to decreased off-rate. To further assess the effect of multimerization on binding, selected VHHs were designed as dimers, trimers, heptamers, and higher order multimers using a viral particle scaffold. RBD binding to these multimerized VHHs was assessed using BLI (FIG. 2).

### Example 2—Viral Neutralization Characterization of Anti-SARS-CoV-2 VHHs

The VHHs of the disclosure were assessed for neutralizing activity in an infectivity assay using SARS-CoV-2 pseudotyped lentivirus. A key criterion is neutralizing activity, since it is expected that some VHHs will bind to the target antigen without interfering with its function. Pseudoviruses expressing SARS-CoV-2 spike protein are produced by co-transfection into 293T cells of plasmids encoding a lentivirus backbone containing a luciferase reporter and the spike protein. Tested VHHs were mixed with the harvested pseudovirions, added to ACE2 expressing cells, and luciferase quantified at 3 days.

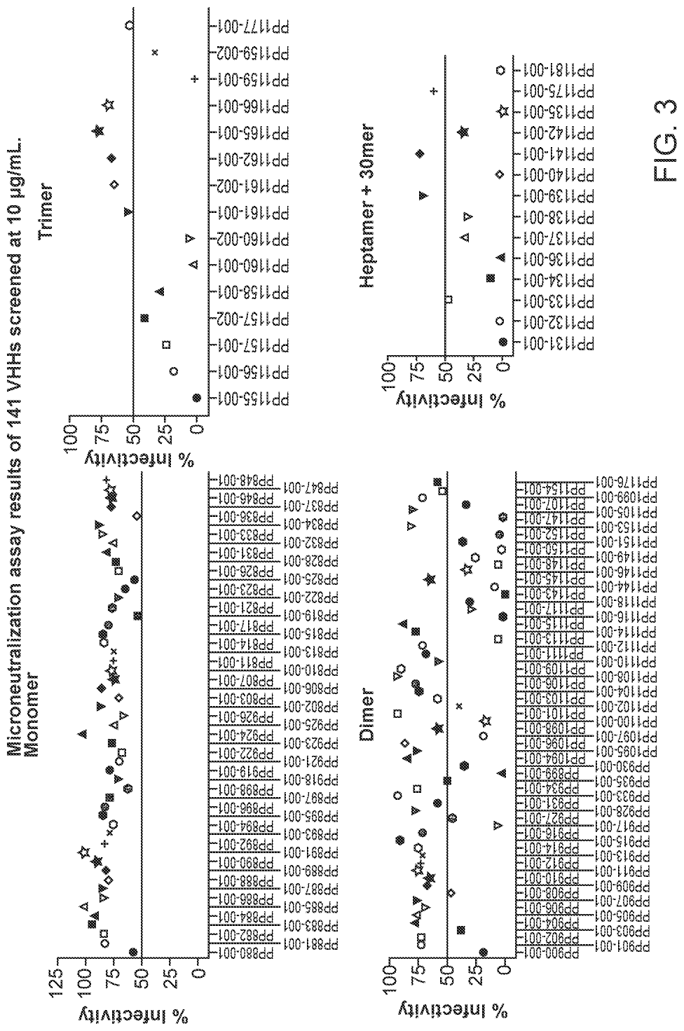

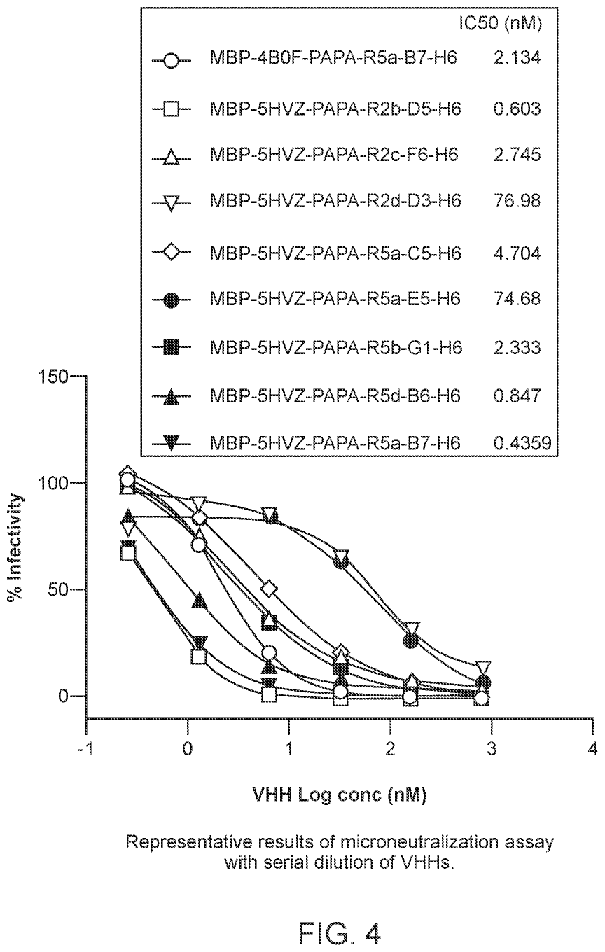

VHHs were screened at a single concentration of 10 μg/mL, equivalent to 150-180 nM (FIG. 3) VHHs exhibiting more than 50% inhibition of infectivity were selected for further analysis. As shown in FIG. 3, among the 141 VHHs screened, none of the monomers (as MBP fusion proteins) exhibited more than 50% inhibition of the infectivity. Twenty-five dimers, six trimers, ten heptamers and one 30-mer showed more than 50% inhibition of the infectivity (FIG. 3). Neutralization assays were then performed on this set of 42 VHHs over a serial dilution curve ranging from 50 μg/mL to 0.016 μg/mL (FIG. 4). IC₅₀ values were calculated and are shown in FIG. 2.

### Example 3—Epitope Binning of Anti-SARS-CoV-2 VHHs

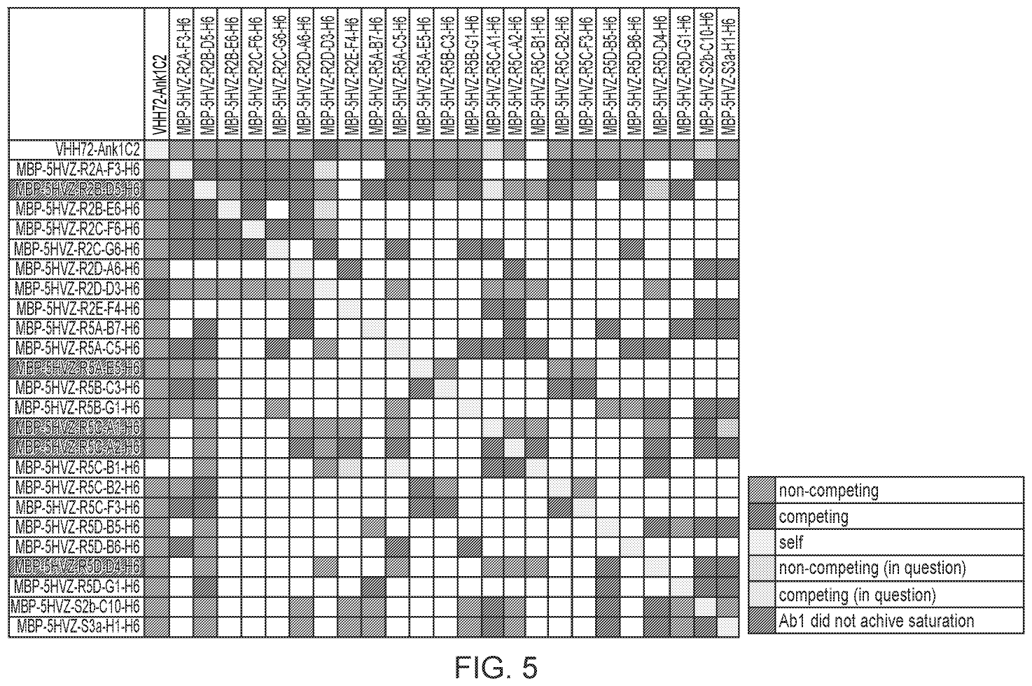

VHHs that exhibited measurable affinity binding and bioactivity in pseudovirus neutralization were examined for competitive binding. This epitope binning analysis determines compatible VHHs for a therapeutic cocktail. BLI methods are used in epitope binning. In this experiment, VHHs were compared for successive binding in an N-by-N format (FIG. 5). Initial epitope binning experiments were conducted using dimeric forms of selected VHHs and SARS VHH-72, a neutralizing antibody developed by the McLellan lab (University of Texas at Austin), to exploit the slow off-rate due to high avidity interactions. The preliminary data showed one VHH that directly competes with SARS VHH-72 (FIG. 4). The dimeric anti-SARS-CoV-2 VHH, R2D-D3, competed with SARS VHH-72 dimer. Still, no competition was observed against other VHHs tested in this initial binning experiment, suggesting the presence of a shared epitope region between SARS VHH-72 and R2D-D3. On the other hand, dimeric VHHs (VHH-5HVZ-MBP) R2A-F3, R2B-D5, R2B-E6, R2C-F6, and R2C-G6 compete for RBD binding and appear to recognize an overlapping epitope (FIG. 4). These data define at least two neutralizing epitopes in the RBD that can be independently targeted by two different classes of VHHs.

### Example 4—ELISA-Based Competition of Anti-SARS-CoV-2 VHHs

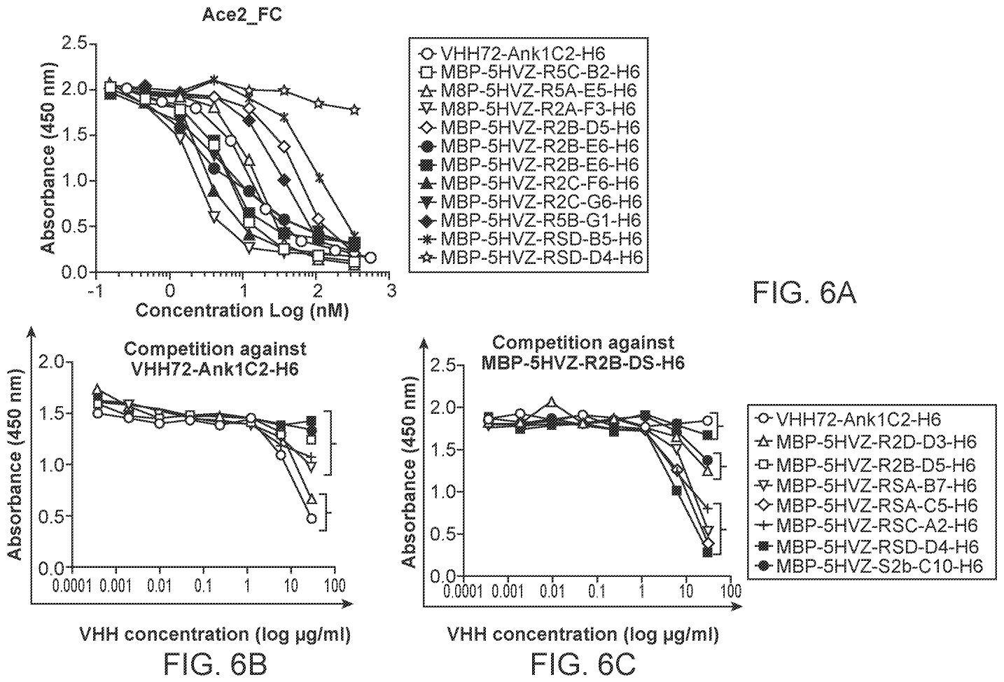

Orthogonal to BLI-based binning experiment, ELISA-based competition experiments against SARS VHH-72 and VHH R2B-D5 were used to assess the epitope space on SARS-CoV-2 RBD recognized by VHHs. In agreement with epitope binning experiments, the data showed that dimeric R2D-D3 competed with SARS VHH-72 while the other VHHs tested showed minimal or no competition against SARS VHH-72. In contrast, the competition experiment with R2B-D5 showed three possible groups where SARS VHH-72 and R5D-D4 did not compete for RBD binding at all, R2D-D3 S2B-C10 exhibited minimal competition while R5C-A2, R5A-B7, R5A-05, and R2B-D5 showed good competition (FIG. 6).

### Example 5—ACE2-Competition of Anti-SARS-CoV-2 VHHs

To complement pseudovirus-based virus neutralization, the VHHs of the disclosure were assayed in human ACE2 competition assay. In this assay, high binding ELISA plates are coated with human ACE2-FC and SARS-CoV-2 RBD incubated with a 5-fold VHH dilution series before application to the ACE2-FC coated plates. RBD binding to ACE2-FC was detected using mouse anti-AviTag antibody (Avidity Bio). In general, almost all monomeric VHHs showed little or no ACE2 competition while higher-order forms of some VHHs (dimeric, trimeric, or heptameric forms) competed for ACE2 strongly (FIG. 6).

### Example 6—Anti-SARS-CoV-2 VHHs Were Assessed for Resistance to Cleavage by Intestinal Proteases

The VHHs of the disclosure were assessed for resistance to intestine proteases using an ELISA based assay developed at Lumen. Desirable VHH pharmacokinetics within the GI tract are strongly dependent on the degree of resistance to GI proteases, and monomer VHHs differ dramatically their ability to remain functionally active following incubations with GI proteases (chyme). To rapidly screen VHHs for susceptibility to GI proteases VHH binding activity was evaluated after treatment with an intestinal protease. Following protease incubation, protease was neutralized with inhibitors and VHH binding activity was measured by capture ELISA using the target antigen. VHHs that functionally survived the protease incubations were able to bind the target antigen, which was detected with-an anti-VHH or anti-VHH conjugate antibody. An EC₅₀ binding curve was calculated using a 4-parameter logistic curve fit of the results and the value was compared to an undigested control to calculate the percent activity remaining.

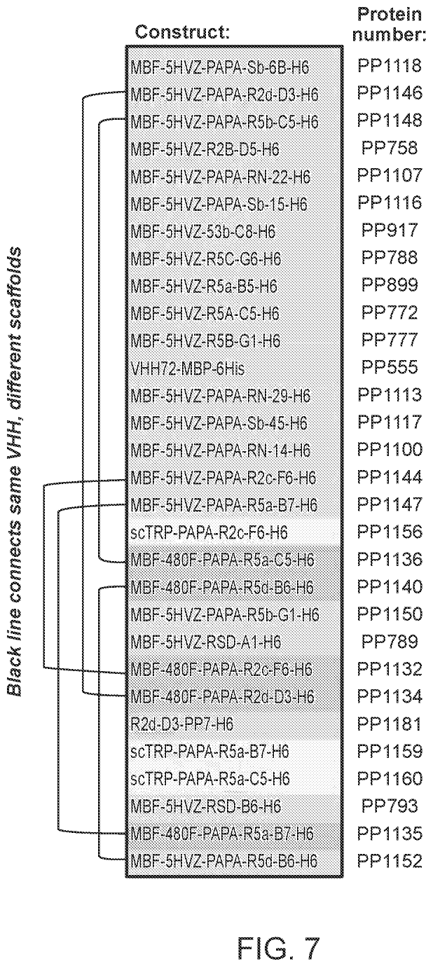

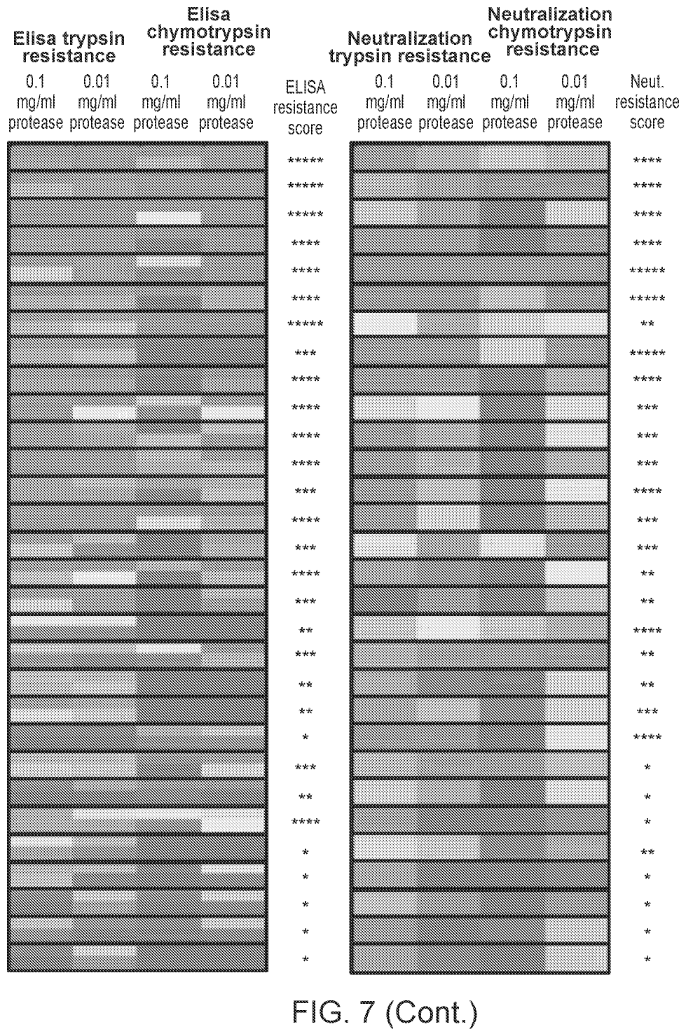

The VHHs of the invention were incubated with trypsin, chymotrypsin, and elastase. Protease resistance results agreed well between the ELISA and neutralization assays. Protease resistance was observed with trypsin, chymotrypsin, and elastase (FIG. 7).

### Example 7—Protein Design to Improve Anti-RBD VHH Binding

Exploiting the hetero-trimeric interaction between the PKA-R dimerization motif and the region of smAKAP that binds the dimer, high order complexes were generated that enable multimeric interactions of diverse molecules. Receptor Binding Domain (RBD)-interacting VHHs were designed in either N- and/or C-terminus of the smAKAP peptide. The new higher order forming (Cerberbody or Hydrabody) substrates were designed with or without the solubility enhancer scaffold, MBP. To increase soluble protein expression, two Cys residues in the smAKAP peptide were mutated to serine.

### Mutated positions—C16S and C24S

Wild type
(SEQ ID NO: 389)
TVILEYAHRLSQDILCDALQQWAC
Mutant
(SEQ ID NO: 390)
TVILEYAHRLSQDILSDALQQWAS

Cerberbody constructs are formed when a spirulina strain (either two independent strains or a single strains from different ORFs) express a 5HVZ mediated homodimer molecule and smAKAP linked monomer VHH. These independent complexes form a further complex structure that will have a valence of three binding moieties. The increased valance will result in increased apparent binding affinity, mainly due to avidity-based interaction. The three binding moieties can be derived from the same VHH, forming a homo-trimer. The design also enables mixing diverse VHHs with desirable binding and virus neutralization property. Such a molecule complex will be able to bind mutant variants of a virus.

As shown here, the Cerberbody complexes show increased apparent binding when compared to dimeric or monomeric forms. Building on the Cerberbody complexes, components were designed that are used to form Hydrabody which consists of four binding moieties. Hydrabody complexes are formed when spirulina strain (either two independent strains or a single strain from different ORFs) express a 5HVZ mediated homodimer molecule and smAKAP linked dimeric VHHs. The smAKAP containing dimers contain VHHs on both the N- and C-terminal of smAKAP. The VHHs fused to the smAKAP can form homo-dimeric (the same VHH) or hetero-dimeric (two different VHHs on either end). Such a configuration enables inclusion of multiple, therapeutically effective VHHs in a single complex.

In addition to increased avidity-based apparent binding, the higher order complexes of Cerberbody and Hydrabody enable the formation of super potent and cross-reactive therapeutic complex that can bind and neutralize diverse virus variants. For example, PP758 neutralizes SARS-COV-2 variants alpha, beta, and gamma but not delta while PP917 neutralizes alpha and delta SARS-CoV-2 variants. A Hydrabody that contains a dimer of dimers of these two VHHs will be able to bind better than each separately and neutralize both variants. Such a design combines multiple components easily where diverse epitope can be engaged resulting in decreased virus escape mutants.

Dimerization of the VHH sequences was shown to increase binding avidity of the VHHs to the receptor binding domain (RBD) of the SARS-CoV-2 spike protein. The presence of a 5HVZ dimerization motif in a polypeptide acts as a docking platform which mediates protein-protein interactions between three α-helical chains, placing the monomers antiparallel to one another. Asymmetric disulfide bond formation between the promoters of the construct further strengthens the dimerization. The cAMP dependent protein kinase (PKA) regulatory subunit (R) PKA-R dimerizes through the 5HVZ motif. (See Burgers et al. (2016) FEB S J. 283(11):2132-2148, the contents of which are incorporated by reference herein). This dimerization motif mediates the cellular interaction between cAMP dependent kinase and the A-kinase anchoring protein. The sAKAP polypeptide is a small membrane anchored peptide that bids the PKA-R dimer and localizes the enzyme to the substrate. The smAKAP used herein can comprise two mutated Cys residues (SEQ ID NO: 390) to improve protein folding and expression.

Table 4 shows anti-RBD VHHs that exhibit acceptable antigen binding as dimers and virus neutralization for complex formation. VHH S3B-C8 is the VHH of the dimeric PP917 construct; R2B-D5 is the VHH in the dimeric PP758 construct. In addition, monomeric forms were designed as fusion proteins with the wild type docking peptide, smAKAP (SEQ ID NO: 389) and cysteine-to-Serine mutant forms of the smAKAP docking peptide (SEQ ID NO: 390). These constructs were also designed with and without maltose binding protein (MBP) as a solubility enhancer scaffold.

| Lane | Sample | Description | MW |
|---|---|---|---|
| 1 | Ladder | | |
| 2 | PP1625- | S3b-C8_smAKAP_C16S_8H | 17 |
| | 001 | | |
| 3 | PP1864- | R2B-D5_smAKAP_C16S_H8 * | 17 |
| | 001 | | |
| 4 | PP1823- | H8_MBP_smAKAP-C16S_R2B-D5 | 61 |
| | 001 | | |
| 5 | PP1824- | R2B-D5_smAKAP_H8 * | 17 |
| | 001 | | |
| 6 | PP1624- | S3b-C8_smAKAP_8H ** | 17 |
| | 001 | | |

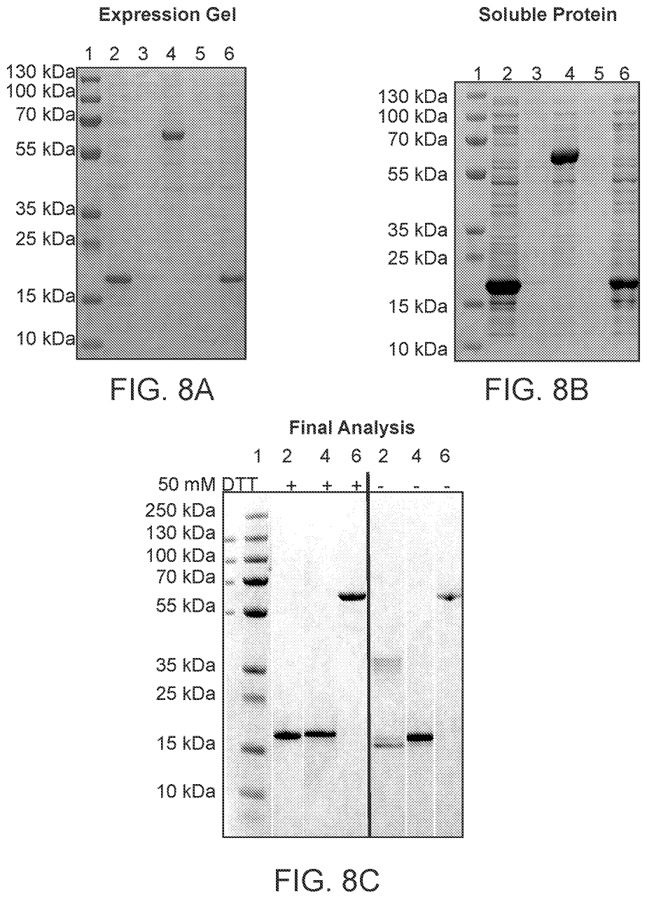

These constructs were produced for expression in E. coli. As shown in FIG. 8A-Figure C, the VHH R2B-D5 (indicated with * in Table 4) containing constructs (VHH in PP758 construct) did not express in an E. coli inducible expression system. Further, the constructs containing the wild type (e.g., cysteine containing) docking peptide (indicated with ** in Table 4) show higher order oligomerization through the free cysteine residues (see Lane 6 in the “non-reduced portion of FIG. 8C).

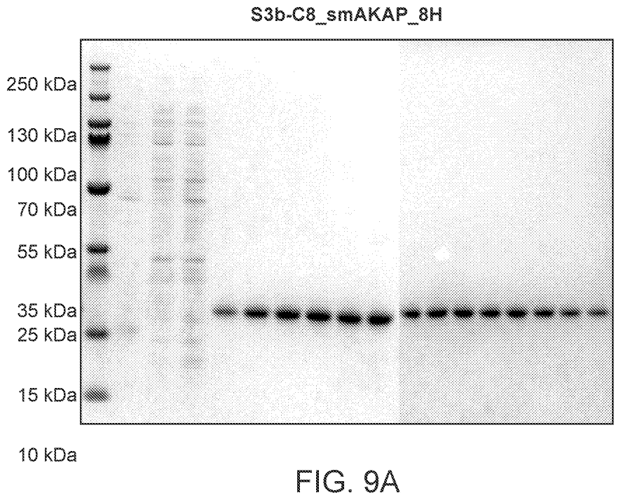

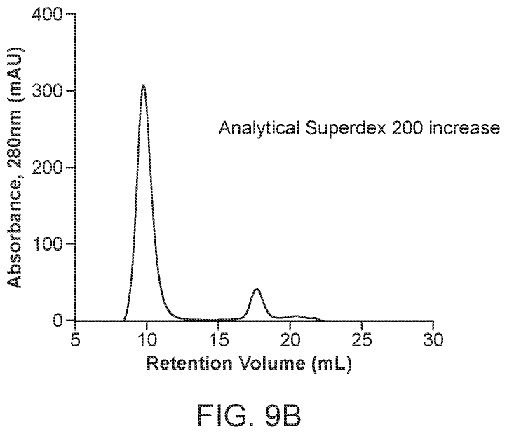

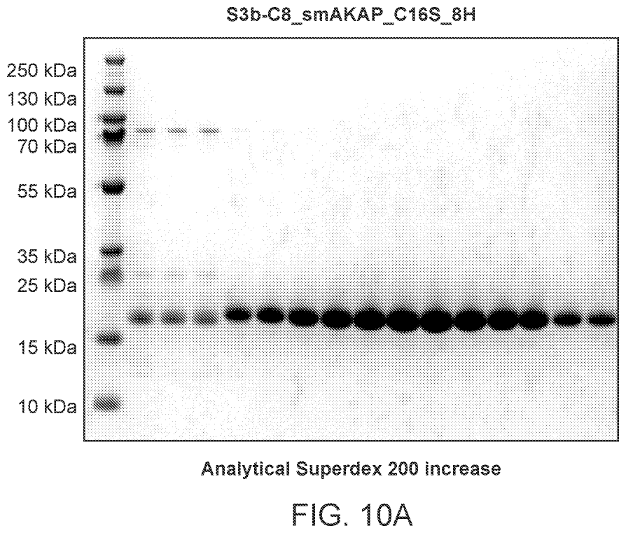

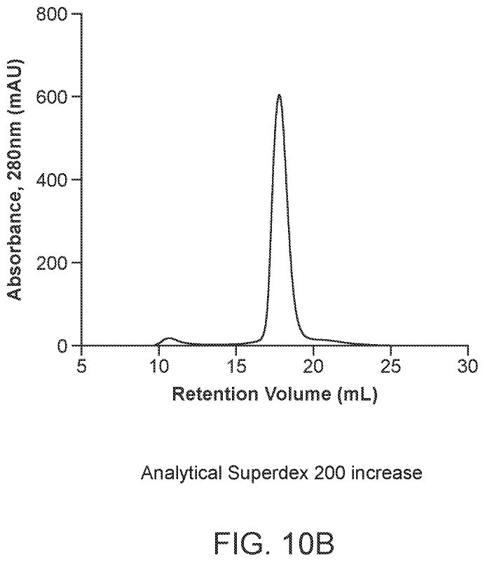

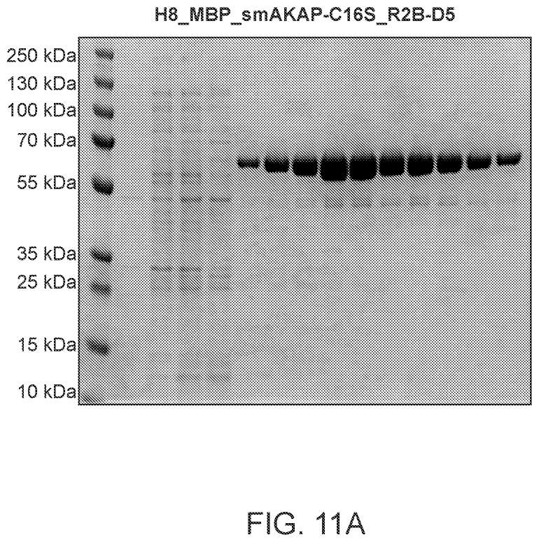

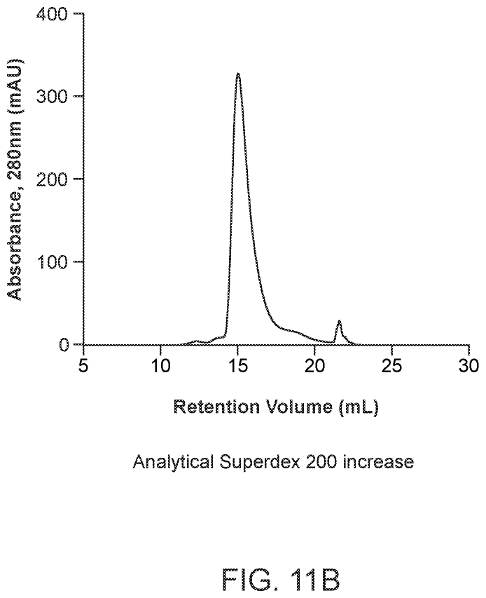

The constructs were purified on a nickel charged affinity resin (Ni-NTA) and the subsequent elutions were then subjected to SEC purification and run on an agarose gel. The samples were also subjected to high resolution gel filtration to study aggregate formation. FIG. 9A-FIG. 9B shows the expression of the S3b-C8_smAKAP_8H construct which comprises the wild type smAKAP protein. The presence of the two free cysteine residues results in aggregate formation. In contrast, mutating the cysteine residues in the smAKAP protein resulted in monodispersed monomer protein. See FIG. 10A-FIG. 10B. Finally, the H8_MBP_smAKAP-C16S_R2B-D5 construct which comprises the smAKAP peptide fused to the R2B-D5 VHH and expressed as a MBP-fusion also resulted in monodispersed monomer protein See FIG. 11A-FIG. 11B.

### Example 8—Comparison of Binding of VHH Complexes to Receptor Binding Domain of SARS-CoV-2 Spike Protein

VHH sequences can associate to form a “Cerberbody” through the association of a dimeric VHH construct to another through a smAKAP protein. Several different configurations of VHH sequences (see Table 5) were compared to determine which showed increased binding avidity to the receptor binding domain of the SARS-CoV-2 spike protein.

TABLE 5
Configurations of VHH sequences in exemplary Cerberbodies

| Sample ID | Description | VHH1 | VHH2 |
|---|---|---|---|
| PP758 | 2-R2B-D5 | R2B-D5 | |
| PP917 | 2-S3b-C8 | S3b-C8 | |
| PP1625 | 1-S3b-C8 | | S3b-C8 |
| PP1823 | | R2B-D5 | R2B-D5 |
| PP1896 | 2-R2B-D5 and 1-S3b-C8 | R2B-D5 | S3b-C8 |
| PP1897 | 3-S3b-C8 | S3b-C8 | S3b-C8 |
| PP1898 | 1-R2B-D5 and 2-S3b-C8 | S3b-C8 | R2B-D5 |

### Hypotheses:

Compared to the dimeric PP758, the Cerberbody complex PP1896 will exhibit comparable binding and neutralization
Compared to the monomeric PP917, the Cerberbody complex PP1896 will exhibit increased binding and virus neutralization potency
Compared to the monomeric PP917, the Cerberbody complex PP1897 will exhibit increased binding due to avidity-based interactions
Compared to the monomeric PP917, the Cerberbody complex PB1898 will exhibit increased binding due to avidity-based interactions

### Study and Results:

The constructs were purified on a nickel charged affinity resin (Ni-NTA) and the subsequent elutions were then subjected to SEC purification and run on an agarose gel. The samples were also subjected to high resolution gel filtration to study aggregate formation.

Increased binding was observed when the VHH valency was increased from monomer to trimer. This increase in binding is mediated by avid interactions. Additionally, the effect of antigen density on binding kinetics was studied. The data shows that when antigen density is increased, a concomitant increase in binding was observed. This effect may be due to a decreased rate of dissociation.

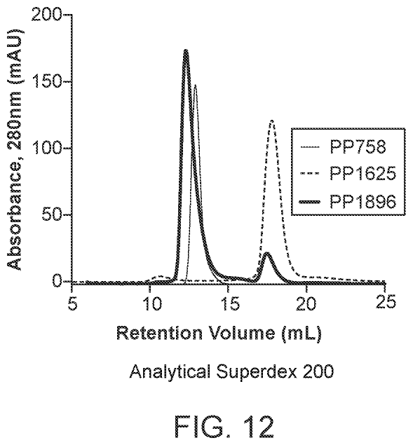

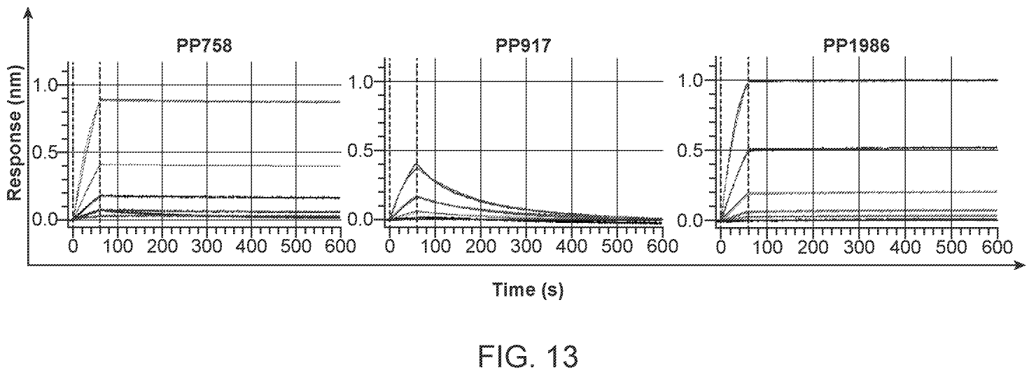

FIG. 12 shows the monomeric v dimeric v aggregate complex formation of PP758, PP1625, and PP1896. The Cerberbody PP1896 form a complex when mixed 1:1.5 molar ratio between the dimeric P758 and the monomeric PP1625. FIG. 13 and Table 6 show that the Cerberbody complex demonstrates about 100-fold increased binding avidity compared to PP758.

TABLE 6

| | | | | | | | Fold |
|---|---|---|---|---|---|---|---|
| Sample | | | | | | | improvement |
| ID | Description | VHH1 | VHH2 | KD (nM) | ka (1/Ms) | kdis (1/s) | (KD) |
| PP758 | 2-R2B-D5 | R2B-D5 | | 0.76 | 8.202E04 | 6.226E−05 | |
| PP917 | 2-S3b-C8 | S3b-C8 | | 10.80 | 1.012E05 | 1.404E−03 | |
| PP1896 | 2-R2B-D5 | R2B-D5 | S3b-C8 | <0.001 | 2.482E05 | <1.0E−07 | ~100 X compared |
| | and 1-S3b-C8 | | | | | | to PP758 |

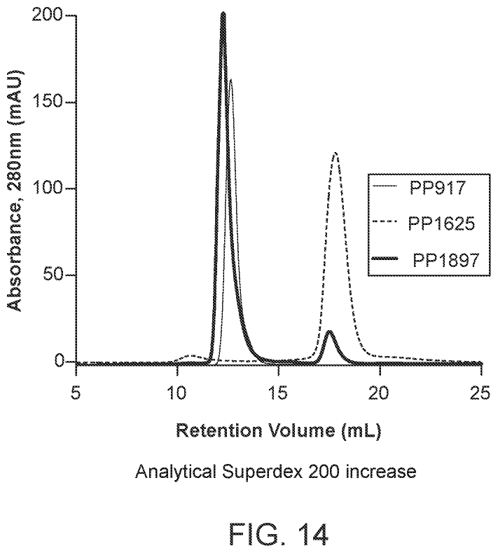

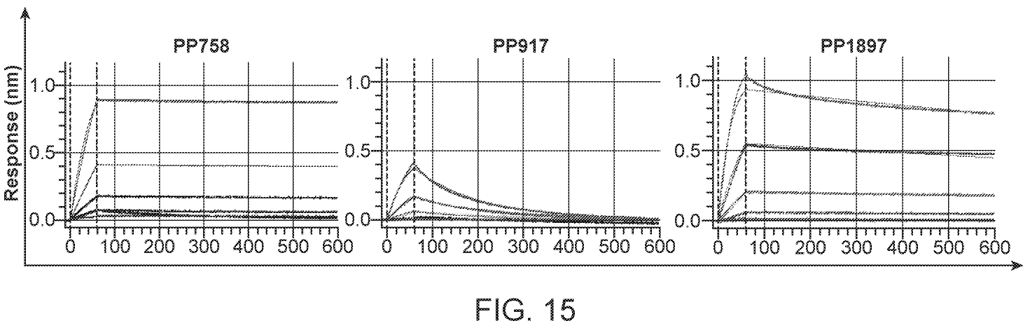

The Cerberbody PP1897 was compared to the dimeric PP917 and the monomeric PP1625. FIG. 14 shows the Cerberbody PP1897 form a complex when mixed 1:15 molar ratio between the dimeric pP917 and the monomeric PP1625. FIG. 15 and Table 7 show that the Cerberbody complex demonstrates about 15-fold greater binding avidity compared to PP917.

TABLE 7

| | | | | | | | Fold |
|---|---|---|---|---|---|---|---|
| Sample | | | | | | | improvement |
| ID | Description | VHH1 | VHH2 | KD (nM) | ka (1/Ms) | kdis (1/s) | (KD) |
| PP758 | 2-R2B-D5 | R2B-D5 | | 0.76 | 8.202E04 | 6.226E−05 | |
| PP917 | 2-S3b-C8 | S3b-C8 | | 10.80 | 1.012E05 | 1.404E−03 | |
| PP1897 | 3-S3b-C8 | S3b-C8 | S3b-C8 | 0.73 | 5.212E05 | 3.818E−04 | ~15 X compared |
| | | | | | | | to PP917 |

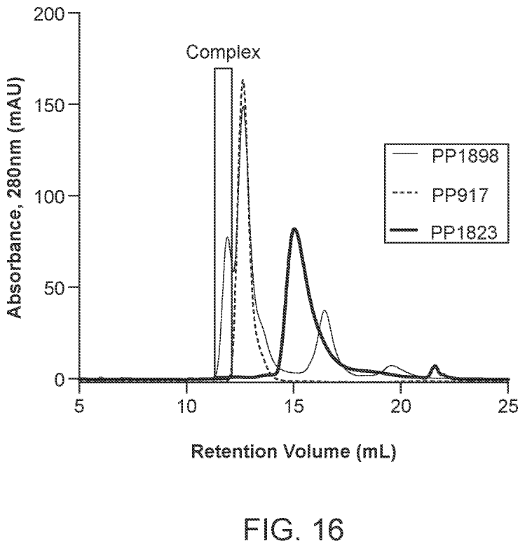

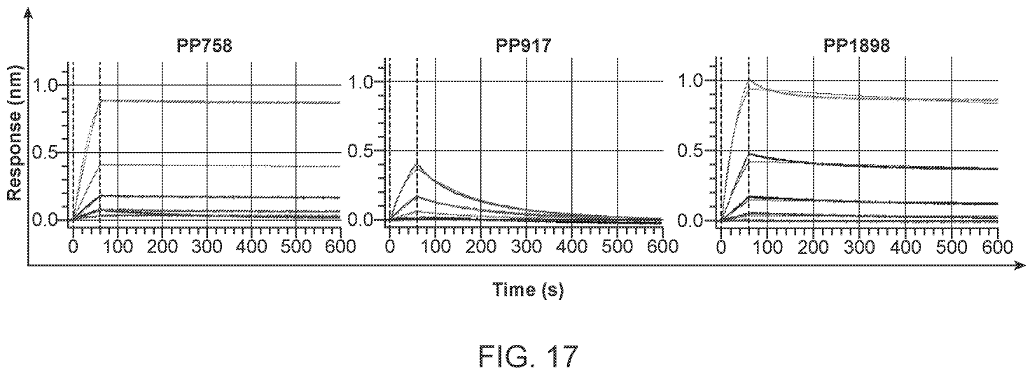

FIG. 16 shows that the Cerberbody PP1897 form a minor complex when mixed 1:1.5 molar ratio between the dimeric PP917 and the monomeric PP1823. Most of the dimer remained uncomplexed, and SDS-PAGE analysis of the MBP fused monomer PP1898 show degradation. FIG. 17 and Table 8 show that PP1898 demonstrates about 30-fold binding avidity compared to PP917.

TABLE 8

| | | | | | | | Fold |
|---|---|---|---|---|---|---|---|
| Sample | | | | | | | improvement |
| ID | Description | VHH1 | VHH2 | KD (nM) | ka (1/Ms) | kdis (1/s) | (KD) |
| PP758 | 2-R2B-D5 | R2B-D5 | | 0.76 | 8.202E04 | 6.226E−05 | |
| PP917 | 2-S3b-C8 | S3b-C8 | | 10.80 | 1.012E05 | 1.404E−03 | |
| PP1898 | 1-R2B-D5 | S3b-C8 | R2B-D5 | 0.40 | 5.405E05 | 2.181E−04 | ~30 X compared |
| | and 2-S3b-C8 | | | | | | to PP917 |

### Example 9—Hydrabody Design, Protein Purification, and Binding

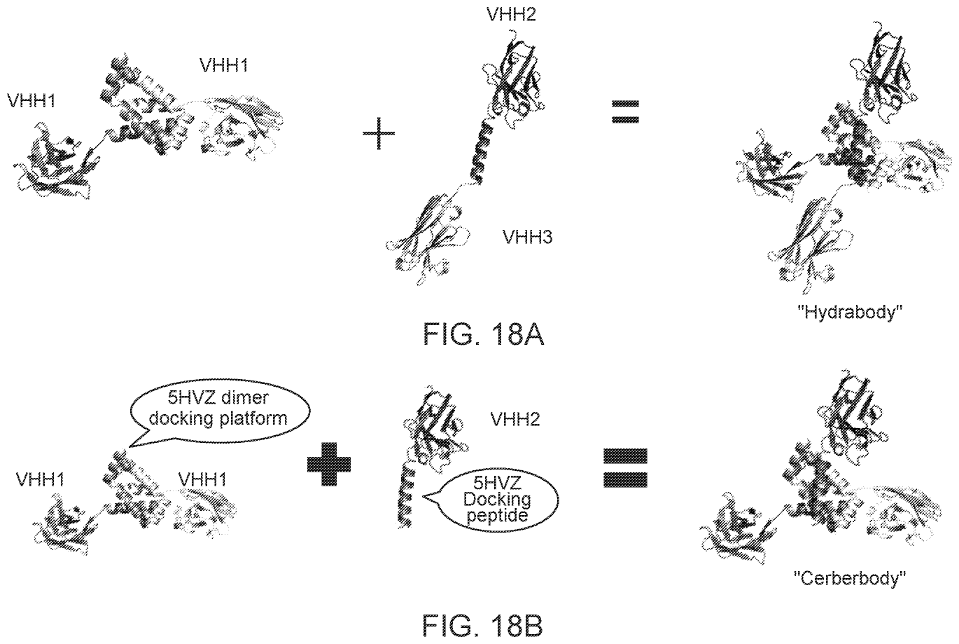

In contrast with the Cerberbody complexes described above, which comprise three VHH sequences, constructs comprising four VHH sequences (“Hydrabodies”) were constructed and characterized. An exemplary Hydrabody is shown in FIG. 18A. Table 9 describes various constructs used in this study.

TABLE 9

| Protein | | Dimeric | | | | |
|---|---|---|---|---|---|---|
| product | Constructs | VHH | VHH1 | VHH2 | VHH3 | Complex |
| PP1892 | R2B-D5_smAKAP- | PP758 | R2B-D5 | R2B-D5 | S3B-C8 | 3xR2B-D5 and 1xS3B-C8 |
| | C16S_S3B-C8_H6 | PP917 | S3B-C8 | R2B-D5 | S3B-C8 | 3xS3B-C8 and 1xR2B-D5 |
| PP1893 | R2B-D5_smAKAP- | PP758 | R2B-D5 | R2B-D5 | R2B-D5 | 4xR2B-D5 |
| | C16S_R2B-D5_H6 | PP917 | S3B-C8 | R2B-D5 | R2B-D5 | 2xR2B-D5 and 2xS3B-C8 |
| PP1894 | S3B-C8_smAKAP- | PP758 | R2B-D5 | S3B-C8 | R2B-D5 | 3xR2B-D5 and 1xS3B-C8 |
| | C16S_R2B-D5_H6 | PP917 | S3B-C8 | S3B-C8 | R2B-D5 | 3xS3B-C8 and 1xR2B-D5 |
| PP1895 | S3B-C8_smAKAP- | PP758 | R2B-D5 | S3B-C8 | S3B-C8 | 2xR2B-D5 and 2xS3B-C8 |
| | C16S_S3B-C8_H6 | PP917 | S3B-C8 | S3B-C8 | S3B-C8 | 4xS3B-C8 |

In Table 9, the protein product column describes the Hydrabody component that contains the smAKAP docking peptide. The construct column describes the VHHs included and their orientation in the polypeptide chain. The third column describes an 5HVZ-containing protein construct. Columns 4 through 6 describe the different VHHs in the reaction mixture: VHH1 is from the 5HVZ motif dimerized construct (e.g., column 3; FIG. 18A), VHH2 is the N-terminal VHH in the smAKAP-containing dimer, and VHH3 is the C-terminal VHH in the smAKAP-containing dimer.

### Hypotheses:

The Hydrabody complex formed between the 5HVZ-containing dimers and the smAKAP-containing dimers will be compared with the dimers themselves (FIG. 18A).
PP1892 will demonstrate increased binding avidity and higher neutralization potency
PP1893 will demonstrate increased binding avidity and higher neutralization potency.
PP1984 will demonstrate increased binding avidity and higher neutralization potency
PP1895 will demonstrate increased binding avidity and higher neutralization potency.

### Study Design and Results:

The constructs were purified on a nickel charged affinity resin (Ni-NTA) and the subsequent elutions were then subjected to SEC purification and run on an agarose gel. The samples were also subjected to high resolution gel filtration to study aggregate formation.

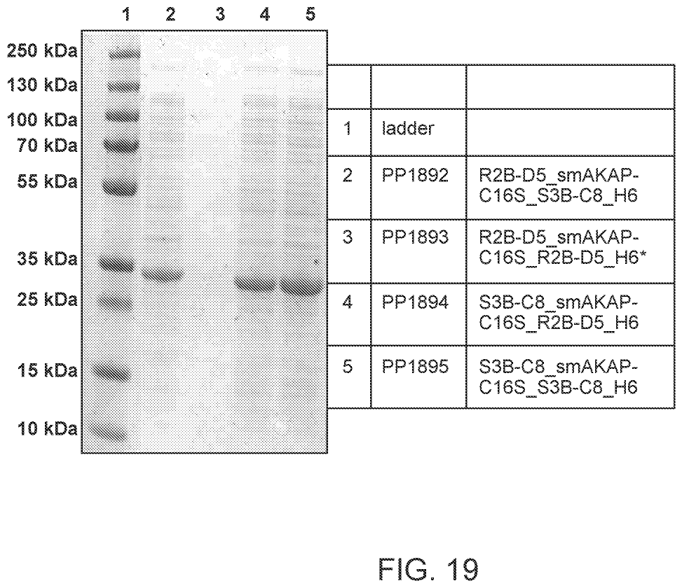

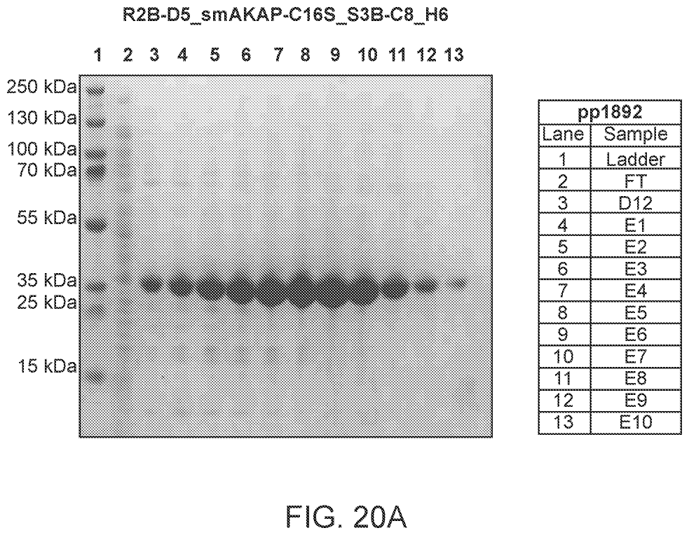

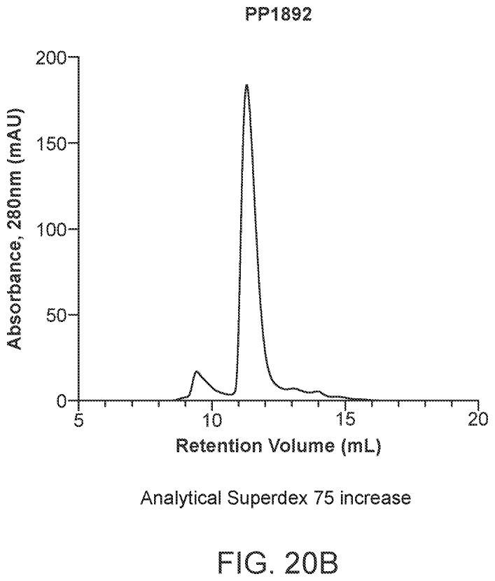

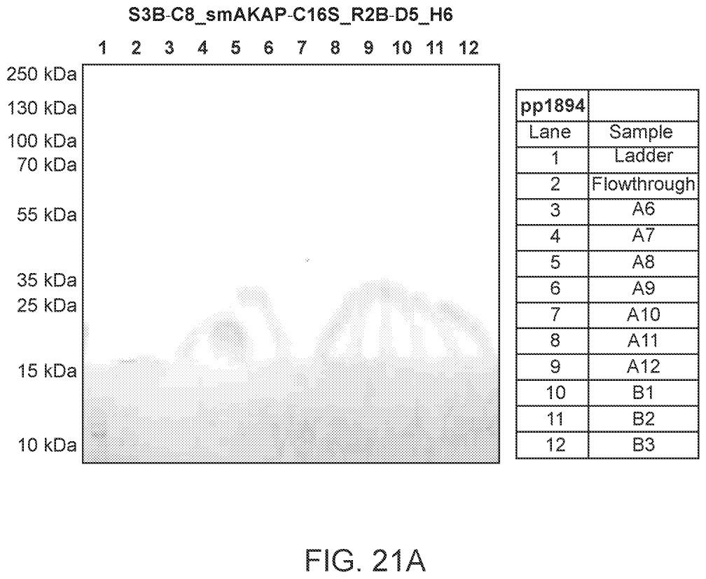

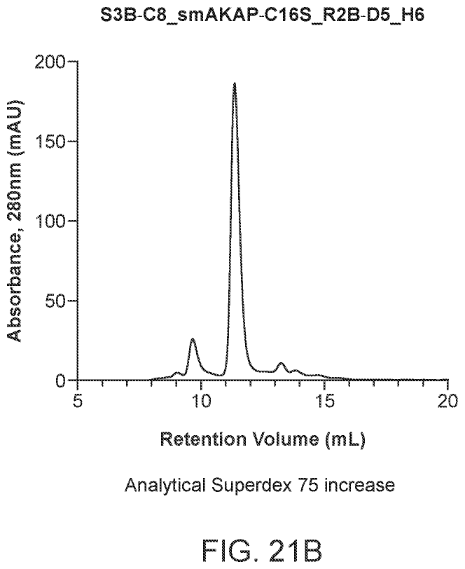

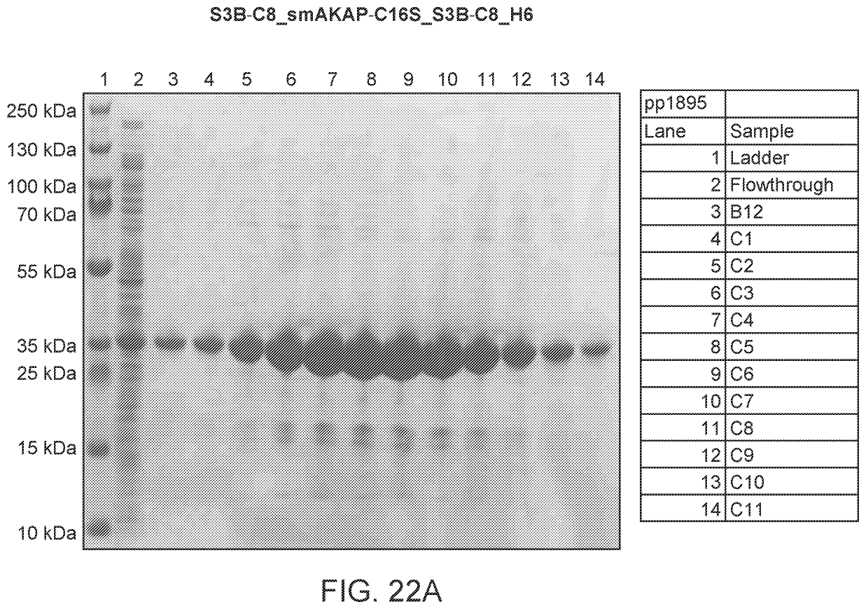

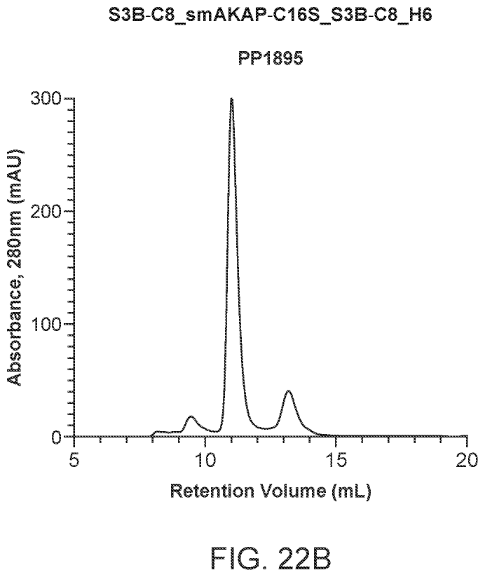

FIG. 19 shows that the dimeric R2B-D5-VHH did not express, likely due to the intrinsic toxicity of the protein. FIG. 20A- FIG. 10B show the Ni-NTA purification and dimeric nature of R2B-D5_smAKAP-C16S_S3B-C8_H8. Lanes 2-13 show the IMAC wash and elution fractions following imidazole-based elution. FIG. 21A- FIG. 21B show the Ni-NTA purification and dimeric nature of S3B-C8_smAKAP-C16S_R2B-D5_H6. Lanes 2-12 show the IMAC wash and elution fractions following imidazole-based elution. FIG. 22A-FIG. 22B show the Ni-NTA purification and dimeric nature of S3B-C8_smAKAP-C16S_S3B-C8_H6. Lanes 2-14 show the IMAC wash and elution fractions following imidazole-based elution.

### Binding Assay

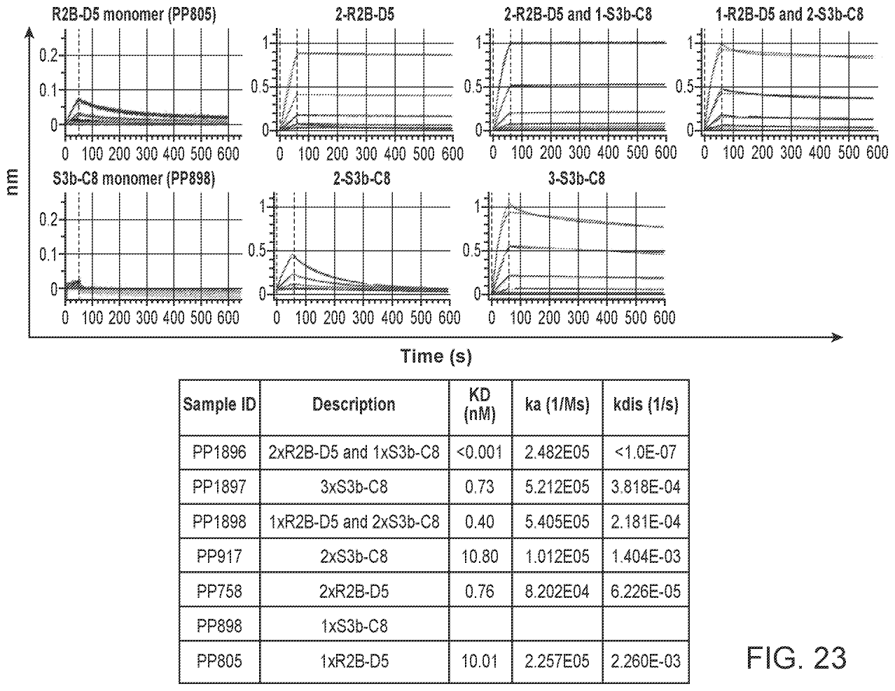

The Cerberbodies show increased apparent binding when compared to dimeric or monomeric forms, see FIG. 23, and Tables 10 and 11 below. When compared to 1×VHH, 2×VHH and 3×VHH constructs show increased binding. The increased apparent binding with increased valency of VHH indicate an avidity-based interaction.

TABLE 10
RBD at 1 ug/mL

| | KD | | |
|---|---|---|---|
| Sample ID | (nM) | ka (1/Ms) | kdis (1/s) |
| PP758 (Dimer) | 0.78 | 1.40E05 | 1.102E−04 |
| PP917 (Dimer) | 10.8 | 1.095E06 | 1.185E−02 |
| Cerberbody | 0.061 | 2.840E05 | 1.728E−05 |

TABLE 11
RBD at 10 ug/mL

| | KD | | |
|---|---|---|---|
| Sample ID | (nM) | ka (1/Ms | kdis (1/s) |
| PP758 | 0.52 | 4.656E04 | 2.435E−05 |
| PP917 | 13.87 | 1.012E05 | 1.404E−03 |
| Cerberbody | <0.001 | 9.296E04 | <1.0E−07 |

By increasing the valance of the VHHs, we hypothesize that the Hydrabodies will show better binding. The other advantage of the Cerberbody and Hydrabody complexes is the ability to form a complex using VHHs that can bind and neutralize diverse variants. For example, PP758 neutralizes SARS-COV-2 variants alpha, beta, and gamma but not delta while PP917 neutralizes alpha and delta variants. A Hydrabody that contains these two VHHs will be able to bind better than each and neutralize both variants. The design of these constructs combine multiple components easily where diverse epitope can be engaged resulting in decreased escape mutants.

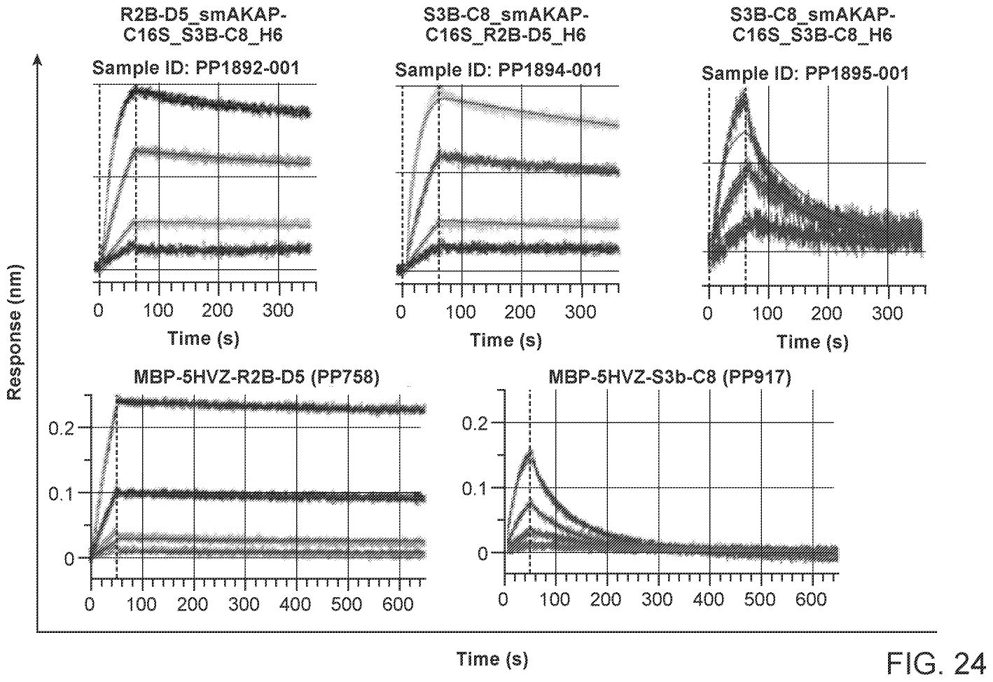

To evaluate binding of Hydrabody complexes, a binding assay was completed and showed that the 5HVZ-containing two chain dimers and the smAKAP-containing single-chain dimer VHHs exhibit comparable binding (KD comparison between PP1895 and PP917, 5×better binding). The single-chain heterodimer (PP1892) exhibits comparable binding to the high-affinity homodimer PP758, 2×better binding, and about 30×better binding when compared to the weak binding homodimer PP917, see Table 12 and FIG. 24.

TABLE 12
Hydrabody forming components binding comparison

| Sample ID | KD (nM) | ka (1/Ms) | kdis (1/s) |
|---|---|---|---|
| R2B-DS_smAKAP-C16S_S3B-C8_H6 | 0.33 | 1.226E06 | 4.115E−04 |
| (PP1892) | | | |
| S3B-C8_smAKAP-C16S_R2B-D5_H6 | 0.44 | 1.343E06 | 5.995E−04 |
| (PP1894) | | | |
| S3B-C8_smAKAP-C16S_S3B-C8_H6 | 2.06 | 3.693E06 | 7.612E−03 |
| (PP1895) | | | |
| MBP-5HVZ-R2B-D5_(PP758) | 0.78 | 4.656E+04 | 2.435E−05 |
| MBP-5HVZ-S3b-C8_(PP917) | 10.8 | 1.012E+05 | 1.404E−03 |

### Example 10—Production of Spirulina Extract Powder for Intranasal Delivery

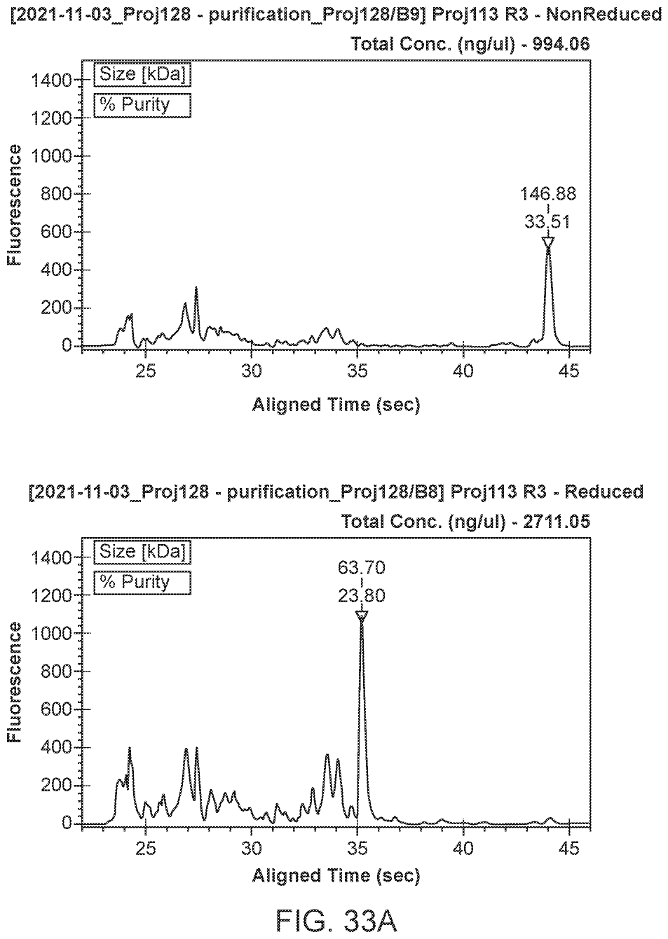

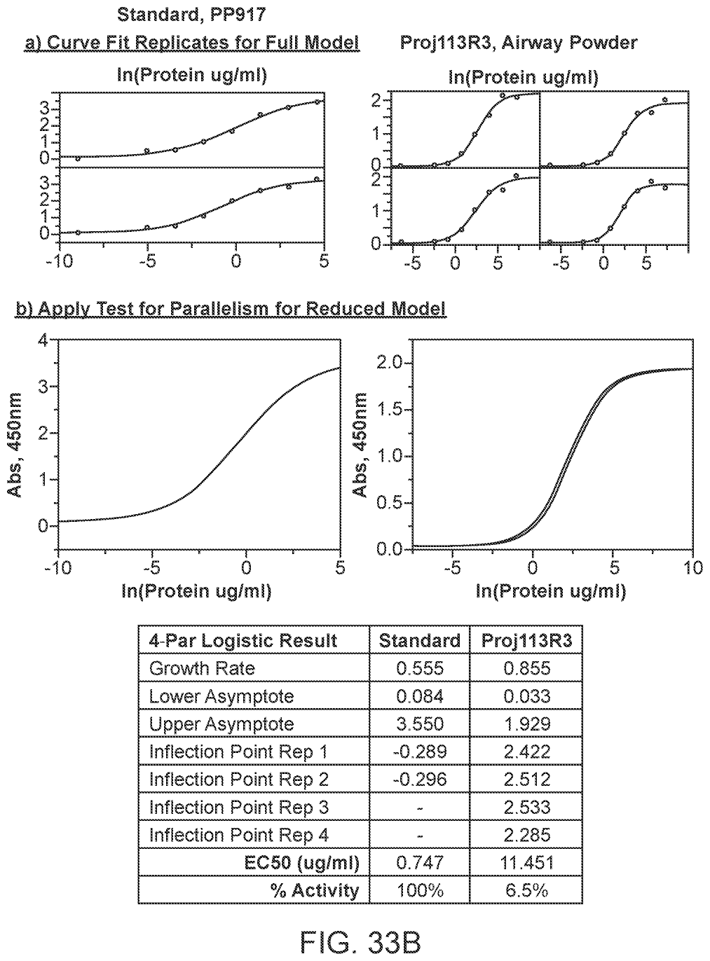

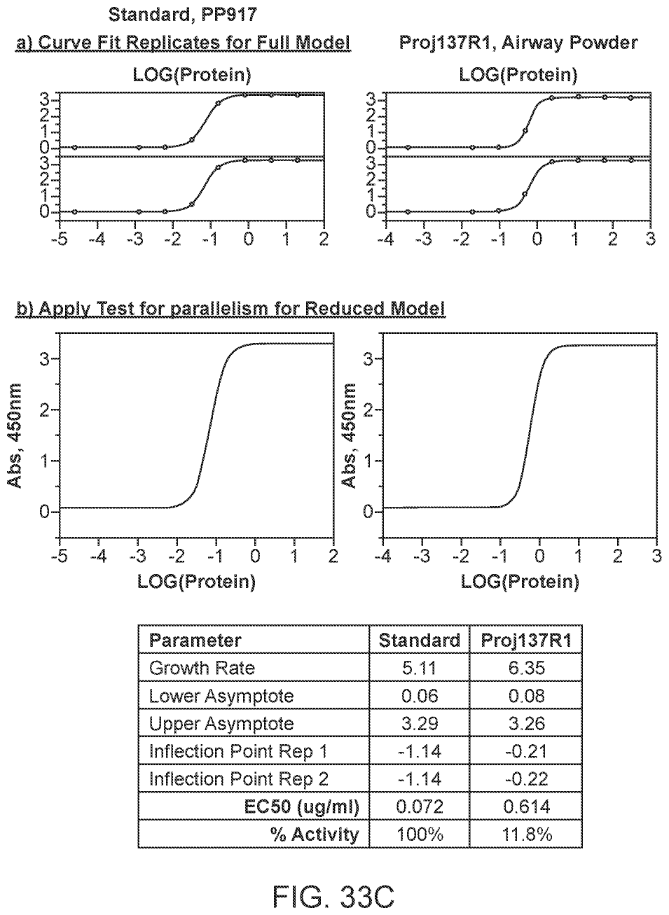

Frozen SP1741 biomass was added to 1 mM ammonium acetate pH 9.3 yield a final concentration of between 10 and 20 g/L ash free dry weight. The suspension was then hand homogenized with a hand immersion blender before high pressure homogenization through a Microfluidics LM-20 high pressure homogenizer set to 14,000 psi, collecting the lysate on ice. Phenylmethylsulfonyl fluoride was added to the lysate to a final concentration of 2 mM and mixed. The mixture was then heated to 30° C. and 2 mM magnesium chloride was added, followed by benzonase nuclease HC added to a concentration of 1 microgram per 10 grams of biomass. The mixture was allowed to react at 30 ° C. for 30 minutes, then chilled to 20° C., pH adjusted to pH 9.18 with ammonium hydroxide, and then filtered through a 1000 kDa tangential flow membrane. The permeate was then concentrated and diafiltered using a 30 kDa tangential flow membrane using 1 mM ammonium acetate pH 9. The concentrate was split into several samples and formulated with trehalose (ranging from 50:1 to 300:1 trehalose to protein molar ratios) and lysine (ranging from 75:1 to 200:1 lysine to protein molar ratio). The resulting aqueous formulations were then spray dried using an outlet temperature between about 60° C. The resulting powder was analyzed by capillary electrophoresis under reducing and non-reducing conditions (FIG. 33A), and by ELISA using purified PP917 protein (FIG. 33B).

In another example, Frozen SP1741 biomass was added to 1 mM ammonium acetate pH 8.3 yield a final concentration of 15 g/L ash free dry weight. The pH was adjusted with ammonium hydroxide to 9.0. The suspension was then hand homogenized with a hand immersion blender before high pressure homogenization through a Microfluidics LM-20 high pressure homogenizer set to 20,000 psi, collecting the lysate on ice. Phenyl methyl sulfonyl fluoride was added to the lysate to a final concentration of 2 mM and mixed. The mixture was then heated to 37° C. and 2 mM magnesium chloride was added, the pH was adjusted to pH 8.22 with ammonium hydroxide, and benzonase nuclease added to a concentration of 1 microgram per 10 grams of biomass. The mixture was allowed to react at 37° C. for 30 minutes, then chilled to 20° C. and filtered through a 1000 kDa tangential flow membrane. The permeate was then concentrated and diafiltered using a 30 kDa tangential flow membrane using 1 mM ammonium acetate pH 7.95. After concentration the pH was adjusted to 7.4 with monobasic sodium phosphate and NaCl added to a final concentration of 130 mM. The resulting aqueous formulations were then spray dried using an outlet temperature between about 60° C. The resulting powder was analyzed by ELISA using purified PP917 protein (FIG. 33C).

### Example 11—Structural Analysis of Complex Binding

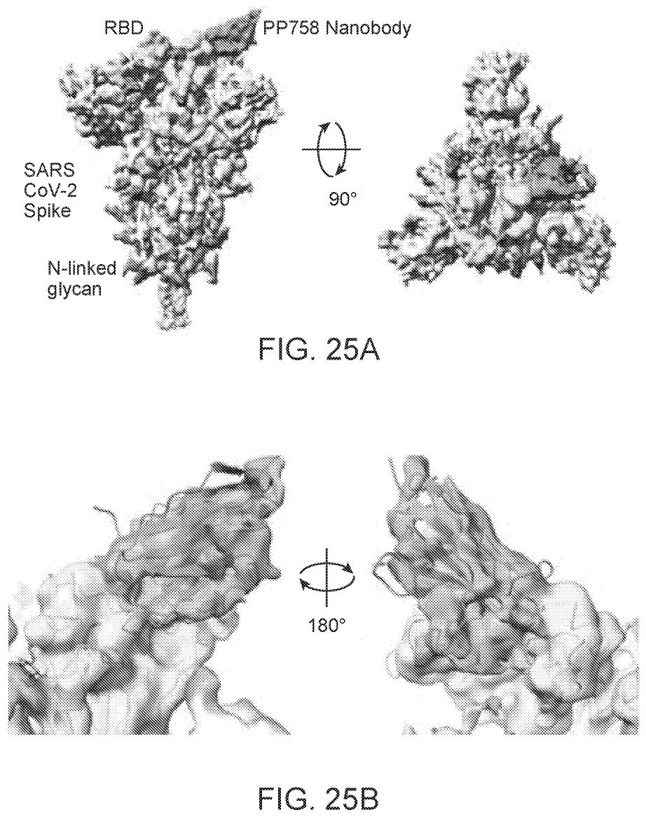

To evaluate binding of an exemplary nanobody, the complex formed between PP758 and SARS-CoV-2 was evaluated using Cryo-EM. Structural analysis helps determine the binding epitope and mode of virus neutralization. The binding epitope of PP758 was elucidated using single-particle cryo-EM reconstructions in complex with SARS-CoV-2 S2P-prefusion-stabilized trimer by imaging with a Titan Krios. In the distribution of particles of the complex, two predominant populations were observed: one particle class with three PP758 VHHs bound per spike in a 1-RBD-up conformation and a second particle class with two partially occupied PP758 VHH bound per spike in a 3-RBD-down conformation with missing density for one of the RBDs (FIG. 25A). Nonuniform refinement for this particle class revealed density for the VHH and majority of the binding interface near the receptor binding motif (RBM), however resolution was limited by molecular motion. The rigid body docked model (FIG. 25A and FIG. 25B) show that PP758 recognizes a region on the ACE2 binding site of RBD. The positioning of the VHH would cause clashes with ACE2 in the spike-ACE2 complex, suggesting that blockage of receptor binding likely accounts for neutralization.

### Example 12—Epitope Binning

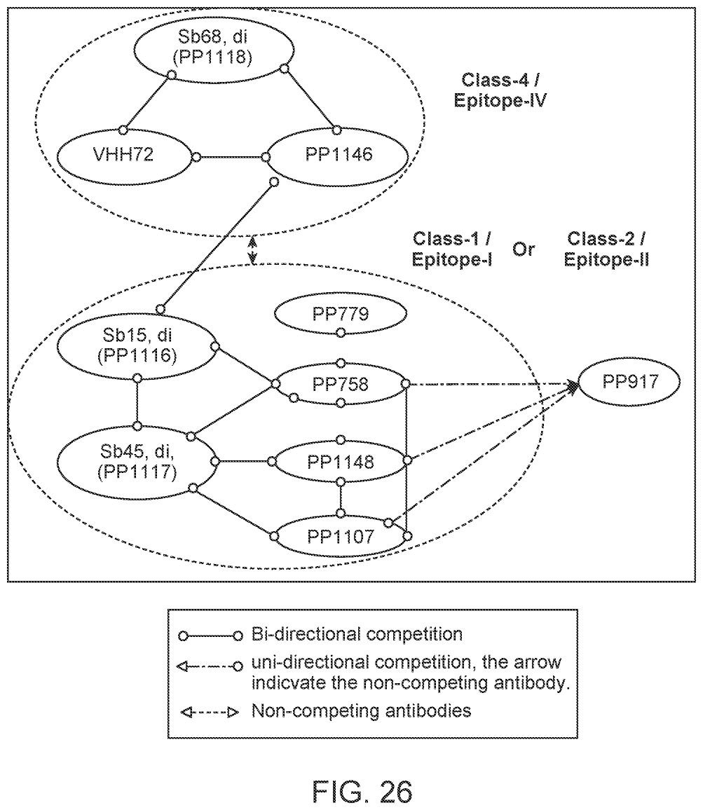

Antibody therapeutics that recognize multiple neutralizing epitopes are potent neutralizers when used as a cocktail. Notable advantages of delivering two or more antibodies against a target pathogen include the decreased likelihood that mutants will emerge which escape neutralization, and the potential for greater-than-additive therapeutic efficacy of non-competing antibodies (Einav and Bloom, 2020). A potential disadvantage is antagonism, for example by antibody interactions with overlapping epitopes. To determine possible VHHs that can be used in cocktail therapeutics, we assessed the epitope classes recognized by exemplary disclosed VHHs, see FIG. 26. The epitope classes are also compared with previously described VHHs, including VHH 72, Sb15, Sb45, and Sb68. We used BLI to evaluate binning competition to immobilized RBD in an epitope binning experiment. In general, most RBD binding and ACE2 competing VHHs cluster with Sb45 and compete with one another. This cluster (Cluster 1) is centered around PP758 and includes PP1148 and PP779. A unidirectional competition between Cluster 1 and PP917 was observed, where the unidirectional competition is only observed when PP917 is the secondary antibody in the competition. This unidirectional competition can be a function of the binding kinetics of PP917, where the VHH dimer exhibits a fast rate of association followed by a faster rate of dissociation.

Structural analysis done on RBD binding VHHs elucidate that Sb45-like VHHs bind to the class 2 neutralizing epitope on RBD. Cluster 1 VHHs, including PP758 and PP1148 share binding epitope with Sb45 and hence, we hypothesize these VHHs bind to the class 2 neutralizing epitopes. Antibodies that bind to this epitope class directly compete ACE2 binding and neutralize by sterilization. Structural analysis of human antibodies and VHHs using spike trimer complex structures indicate, the antibodies can bind RBD both in its “up” and “down” conformations. The epitope interacting paratope of these human antibodies heavy chain and VHHs exhibit long CDR3. Structural and sequence analysis of PP758 confirm binding to RBD in both “up” and “down” confirmation and the presence of long CDR3 at 14 amino acids. Given the observed unidirectional competition between PP917 and Cluster 2 VHHs, PP917 and some of the VHHs from cluster 1 can be binding to class-1 or class-2 epitope classes on RBD.

The binding experiment also delineated a second group of VHHs that competes with VHH-72 and Sb68 (Cluster 2). PP1146 clusters in this group. In addition to VHH-72 and Sb68, PP1146 also competes with Sb15. The bi-directional competition between PP1146 and Sb15 links the two clusters of VHHs, Cluster 1 and Cluster 2. VHHs in these cluster, including VHH-72 and Sb45 bind to RBD on the class 4 neutralizing epitope. These classes of antibodies do not directly inhibit ACE2 binding to RBD but cause steric hindrance that result in virus neutralization. Binning analysis groups PP1146 in the same epitope class as VHH-72 and Sb45, and so, we hypothesize that the mode of virus neutralization by PP1146 is not by direct competition with ACE2 for RBD binding. Taken together, VHHs from the two epitope classes are possible candidates for a cocktail mix to improve therapeutic potency and breadth of neutralization.

### Example 13—Binding of Exemplary VHHs to SARS-CoV-2 RBD Variants by ELISA

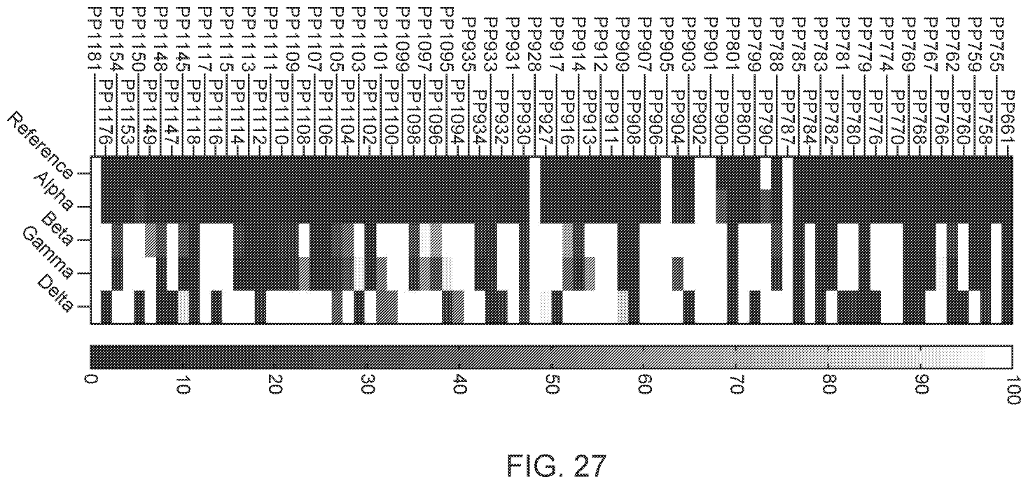

To assess the cross-reactive nature of exemplary VHHs, binding of VHHs to RBD derived from spike protein of 5 SARS-CoV-2 variants was completed. In brief, high binding ELISA plates were coated with 1 μg/mL. VHHs were incubated in 5-fold dilution concentration series starting at 25 μg/mL, see FIG. 27 and Table 13.

TABLE 13

| | ELISA based binding EC50 (nM) | | | | |
|---|---|---|---|---|---|
| | SARS- | SARS- | SARS- | SARS- | SARS- |
| | COV-2 | CoV-2 | CoV-2 | CoV-2 | CoV- |
| | Wuhan | Alpha | Beta | Gamma | 2 Delta |
| Protein | Reference | Variant | Variant | Variant | Variant |
| Product | RBD | RBD | RBD | RBD | RBD |
| PP755 | 3.41 | 4.41 | 544.50 | 778.70 | NB |
| PP758 | 0.29 | 0.41 | 1.20 | 0.92 | 16.47 |
| PP759 | 1.78 | 2.77 | 4.12 | 3.11 | NB |
| PP760 | 0.28 | 0.30 | NB | 117.20 | 0.29 |
| PP762 | 0.28 | 0.30 | 22.90 | 7.64 | 5.12 |
| PP766 | 0.87 | 2.19 | 146.80 | 95.82 | 364.20 |
| PP767 | 0.36 | 0.42 | 0.69 | 0.54 | NB |
| PP768 | 0.38 | 0.41 | 1.83 | 0.76 | 8.40 |
| PP769 | 0.40 | 0.44 | 6.80 | 3.56 | 0.45 |
| PP770 | 0.54 | 0.83 | 261.20 | 126.90 | NB |
| PP774 | 1.64 | 1.94 | NB | NB | 181.10 |
| PP776 | 0.39 | 0.35 | 269.70 | 186.60 | 0.36 |
| PP779 | 0.37 | 0.37 | 1.10 | 0.81 | 1.66 |
| PP780 | 1.55 | 0.46 | NB | NB | 9.62 |
| PP781 | 0.36 | 0.31 | NB | NB | 1.32 |
| PP782 | 0.73 | 1.93 | 2.46 | 1.61 | NB |
| PP783 | 0.32 | 0.27 | 1.38 | 0.68 | 0.42 |
| PP784 | 0.26 | 0.26 | 255.00 | 165.10 | NB |
| PP785 | 1.27 | 2.03 | 8.55 | 4.21 | 3.76 |
| PP787 | 260.80 | NB | NB | NB | NB |
| PP788 | 1.56 | 5.88 | 27.58 | 16.78 | NB |
| PP790 | NB | 32.24 | NB | NB | NB |
| PP799 | 0.54 | 0.56 | 579.80 | 147.20 | 0.61 |
| PP800 | 1.06 | 1.19 | NB | 927.50 | 257.70 |
| PP801 | 0.33 | 0.30 | 3.13 | 2.25 | 6.52 |
| PP900 | 0.47 | 34.00 | 708.60 | 339.70 | NB |
| PP901 | NB | NB | NB | NB | NB |
| PP902 | NB | 123.20 | NB | 450.70 | NB |
| PP903 | 0.27 | 0.28 | NB | NB | 0.26 |
| PP904 | 1.06 | 12.98 | 138.70 | 34.65 | NB |
| PP905 | NB | 447.20 | NB | NB | NB |
| PP906 | 2.00 | 1.97 | 987.80 | 378.40 | 204.80 |
| PP907 | 3.00 | 3.70 | 262.90 | 120.40 | NB |
| PP908 | 1.59 | 1.74 | 7.42 | 3.75 | 2.15 |
| PP909 | 0.62 | 0.44 | 8.10 | 5.37 | 81.40 |
| PP911 | 2.36 | 2.19 | 545.00 | 316.80 | NB |
| PP912 | 2.33 | 1.96 | 365.00 | 176.80 | 621.70 |
| PP913 | 1.53 | 1.33 | 113.50 | 61.08 | NB |
| PP914 | 3.24 | 2.94 | 19.36 | 10.48 | NB |
| PP915 | 1.05 | 0.52 | NB | NB | 37.80 |
| PP916 | 1.19 | 2.04 | 66.29 | 42.89 | NB |
| PP917 | 0.98 | 0.77 | NB | NB | 8.47 |
| PP927 | 1.43 | 2.22 | NB | NB | 94.20 |
| PP928 | NB | NB | NB | NB | NB |
| PP930 | 3.18 | 3.90 | 13.02 | 10.21 | 21.12 |
| PP931 | 1.93 | 2.07 | 134.30 | 110.40 | 126.70 |
| PP932 | 0.37 | 0.41 | NB | NB | 15.35 |
| PP933 | 4.90 | 9.74 | 10.15 | 9.73 | 8.87 |
| PP934 | 1.07 | 1.56 | 13.50 | 4.78 | NB |
| PP935 | 0.47 | 0.47 | 343.40 | 166.60 | 151.10 |
| PP1094 | 0.96 | 0.89 | 315.00 | 160.20 | 49.90 |
| PP1095 | 0.78 | 1.18 | 192.90 | 90.92 | 16.94 |
| PP1096 | 1.47 | 1.92 | 63.16 | 28.36 | NB |
| PP1097 | 0.25 | 0.30 | 95.43 | 58.61 | 244.00 |
| PP1098 | 0.83 | 0.97 | 24.81 | 11.23 | NB |
| PP1099 | 4.24 | 4.95 | 193.50 | 160.40 | NB |
| PP1100 | 0.31 | 0.35 | NB | NB | 46.52 |
| PP1101 | 1.56 | 1.77 | 114.60 | 51.53 | 42.57 |
| PP1102 | 0.97 | 1.55 | 2.00 | 1.26 | NB |
| PP1103 | 2.40 | 1.45 | 226.60 | 92.75 | 2.69 |
| PP1104 | 1.21 | 1.09 | 44.92 | 29.52 | 294.70 |
| PP1105 | 1.24 | 1.41 | 26.57 | 15.42 | 36.61 |
| PP1106 | 2.52 | 2.05 | 3.17 | 7.84 | NB |
| PP1107 | 0.27 | 0.28 | 0.25 | 0.18 | NB |
| PP1108 | 2.61 | 1.89 | 121.10 | 57.12 | NB |
| PP1109 | 1.06 | 1.12 | 17.06 | 11.02 | 115.00 |
| PP1110 | 3.50 | 4.98 | 17.00 | 8.98 | 407.00 |
| PP1111 | 0.33 | 0.31 | 1.57 | 1.60 | 231.20 |
| PP1112 | 1.36 | 1.76 | 4.77 | 3.01 | 15.09 |
| PP1113 | 0.34 | 0.72 | 5.64 | 3.37 | NB |
| PP1114 | 1.14 | 1.64 | 18.92 | 6.12 | NB |
| PP1115 | 4.90 | 6.74 | 857.00 | 162.00 | NB |
| PP1145 | 1.33 | 1.94 | 30.88 | 5.49 | 88.95 |
| PP1147 | 0.36 | 0.33 | NB | NB | 0.49 |
| PP1148 | 0.30 | 0.33 | 30.48 | 9.06 | 0.22 |
| PP1149 | 2.50 | 3.01 | 57.98 | 133.50 | NB |
| PP1150 | 0.64 | 20.13 | NB | NB | 0.86 |
| PP1153 | 1.23 | 0.91 | NB | NB | 222.70 |
| PP1154 | 2.42 | 2.37 | 24.72 | 15.85 | 221.20 |
| PP1176 | 0.29 | 0.27 | NB | NB | 2.67 |
| PP1181 | NB | NB | NB | NB | NB |

NB = not binding, EC50 > 1000 nM

### Example 14—Live Virus Assay

To test if exemplary VHHs identified through ELISA and pseudovirus assay can actively neutralize live Covid virus, the top 4 candidates, PP917, PP758, PP1146 and PP1148 were tested against 2019 Novel Coronavirus, Isolate USA-WA1/2020 (SARS-CoV-2) with African green monkey kidney (Vero E6) cells. The test was conducted by IIT Research Institute in Chicago.

Combinations of VHHs PP917, PP758, PP1146 and PP1148 were prepared in three forms in total of 31 samples (shown in Table 14). Samples #1-10 are purified proteins. Samples #11-20 are purified proteins mixed with 5 mg/mL of cell extract from wild type cells SP003 (matrix). Samples #21-30 are cell extract from the corresponding strains SP1741, SP1641, SP1861 and SP1862.

TABLE 14
List of samples for Live virus assay

1. 917 @ IC₈₀ (purified protein)
2. 758 @ IC₈₀ (purified protein)
3. 1148 @ IC₈₀ (purified protein)
4. 1146 @ IC₈₀ (purified protein)
5. Isotype control (purified protein)
6. 917 and 758 each @ IC₈₀ (purified protein)
7. 917 and 1146 each @ IC₈₀ (purified protein)
8. 917 and 1148 each @ IC₈₀ (purified protein)
9. 1148 and 1146 each @ IC₈₀ (purified protein)
10. 917, 1146, 1148 each @ IC₈₀ (purified protein)
11. 917 @ IC₈₀ (purified protein + matrix)
12. 758 @ IC₈₀ (purified protein + matrix)
13. 1148 @ IC₈₀ (purified protein + matrix)
14. 1146 @ IC₈₀ (purified protein + matrix)
15. Isotype control (purified protein + matrix)
16. 917 and 758 each @ IC₈₀ (purified protein + matrix)
17. 917 and 1146 each @ IC₈₀ (purified protein + matrix)
18. 917 and 1148 each @ IC₈₀ (purified protein + matrix)
19. 1148 and 1146 each @ IC₈₀ (purified protein + matrix)
20. 917, 1146, 1148 each @ IC₈₀ (purified protein + matrix)
21. 917 @ IC₈₀ (cell extract)
22. 758 @ IC₈₀ (cell extract)
23. 1148 @ IC₈₀ (cell extract)
24. 1146 @ IC₈₀ (cell extract)
25. Isotype control (cell extract) (SP1182 extract)
26. 917 and 758 each @ IC₈₀ (cell extract)
27. 917 and 1146 each @ IC₈₀ (cell extract)
28. 917 and 1148 each @ IC₈₀ (cell extract)
29. 1148 and 1146 each @ IC₈₀ (cell extract)
30. 917, 1146, 1148 each @ IC₈₀ (cell extract)
31. Matrix only (SP3 extract)
32. 917 (purified protein), 10x of sample #1.
33. 917 and 758 (purified protein), 10x of sample #6.
34. 917 (purified protein + matrix), 10x of sample #11
35. 917 and 758 (purified protein + matrix), 10x of sample
#16

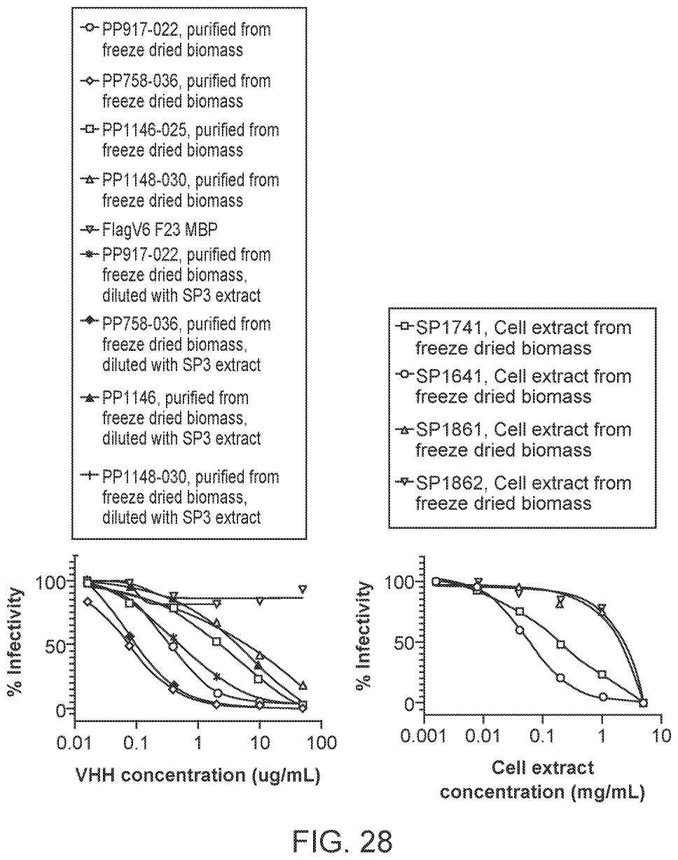

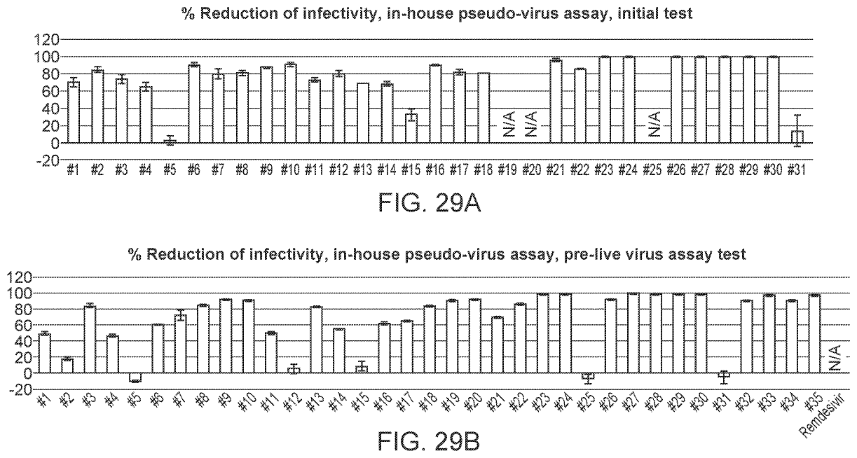

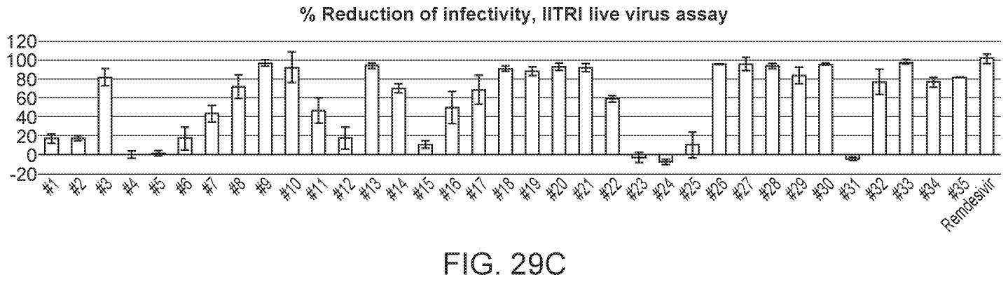

To prepare for the live virus assay, neutralizing potency of VHHs purified from freeze dried biomass was tested with pseudovirus assay. The results are shown in FIG. 28. IC80 of each single VHH was estimated based on the measured neutralization curve and IC50. 31 samples were prepared with IC80 of each VHH and tested with pseudovirus assay. The % reduction of infectivity of each sample are shown in FIG. 29A. All the VHH samples reduced 65-99.9% of the virus infectivity, which is within the expected range. As expected, the non-relevant isotype control PP322 (sample #5) and matrix (sample#31) didn't neutralize the virus. However, when PP322 was mixed with the matrix, ˜30% of the virus infectivity was inhibited. It is not clear why this sample inhibit the infectivity. The experiment will be repeated with another set of samples. Another relevant control, cell extract from SP1182 which express PP322 will be included in the next round of experiment.

The set of 31 samples were prepared in a larger volume and divided into 2 aliquots. The aliquots were stored at −80 C for 24 hours and one set of aliquots were tested with pseudovirus assay to assess the effect of freeze-thaw on the neutralizing potency of the VHHs. The results (FIG. 29B) showed that PP917 lost ˜30% of the potency in all three forms (sample #1, 11, and 21) after the process. Note that purified PP758 lost the majority of the potency (sample #2 and #12), while the cell extract from the corresponding strain (sample #22) was fine. Since PP917 is the top candidate, sample #1,6,11,16 which contain PP917 and PP758 were prepared with 10 times more VHHs (sample #32,33,34,35). These new samples reduced 78-99.7% of the virus infectivity (FIG. 29B). Note that the non-relevant isotype control PP322 (sample #5, 15, 25) and matrix (sample#31) didn't neutralize the virus. Total of 35 samples were sent to IIRI for live virus assay.

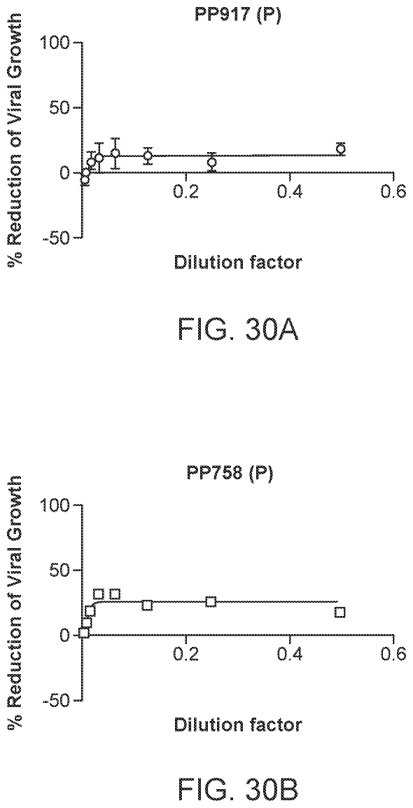

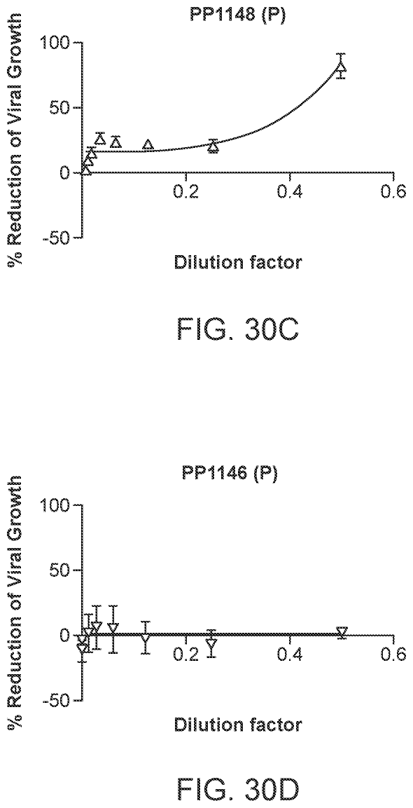

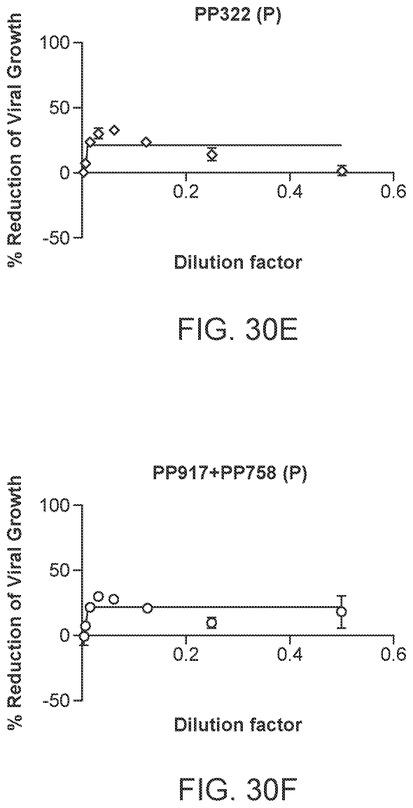

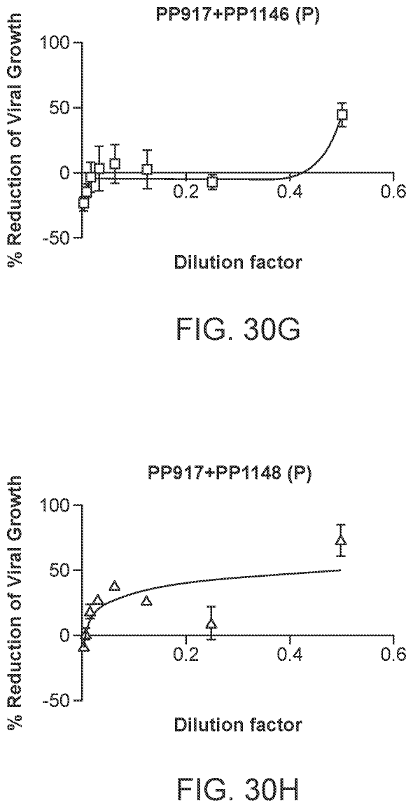

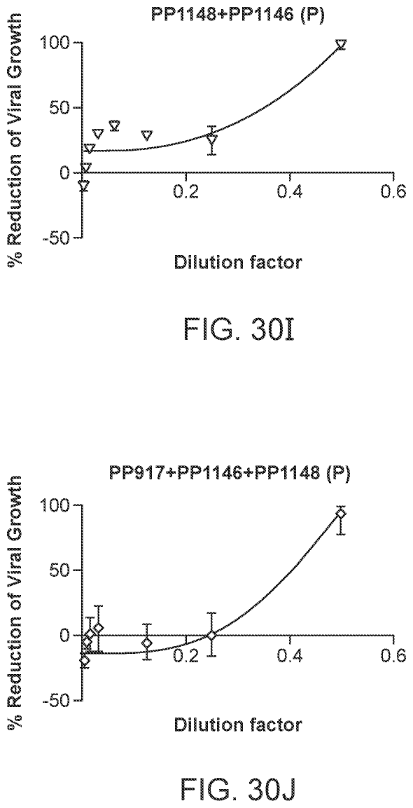

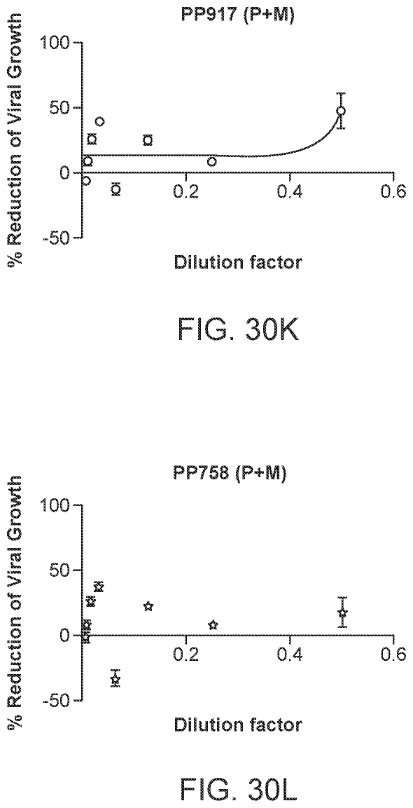

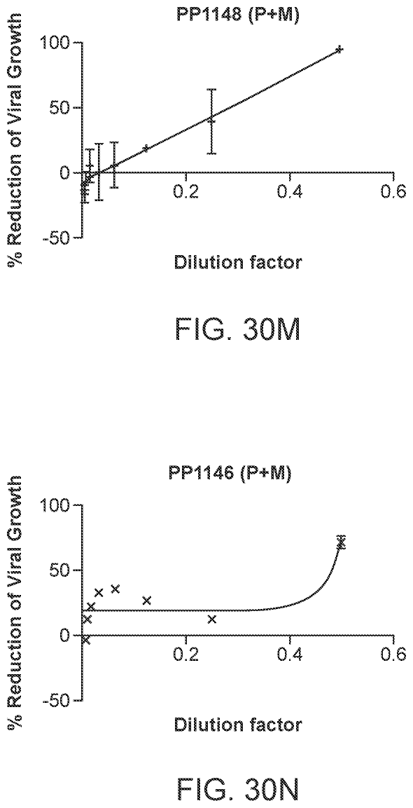

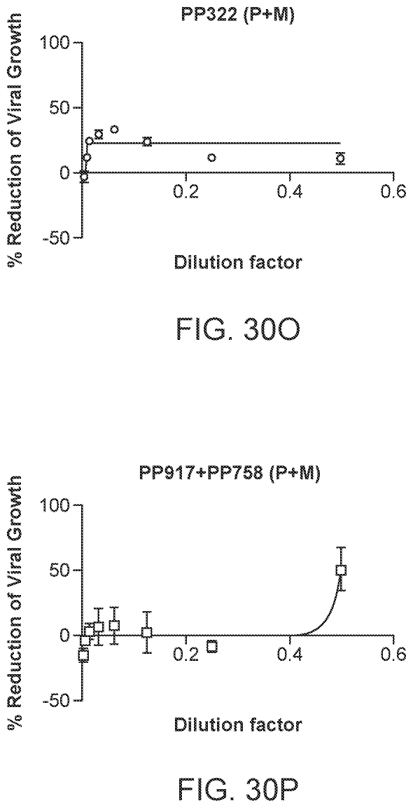

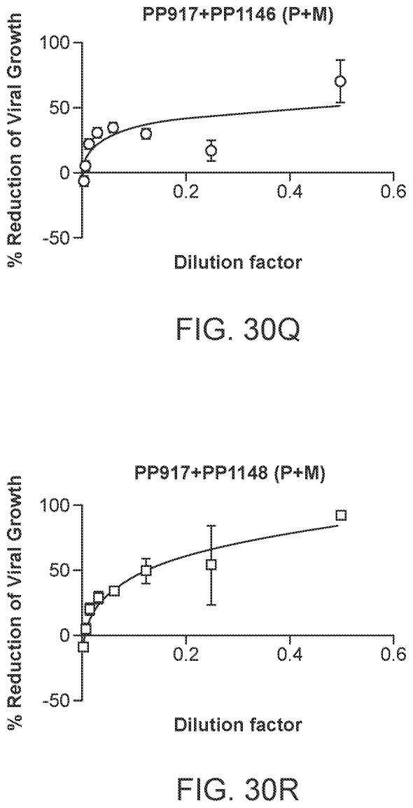

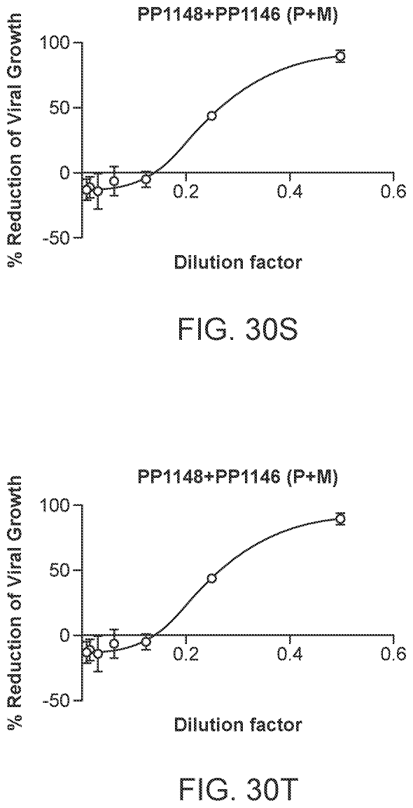

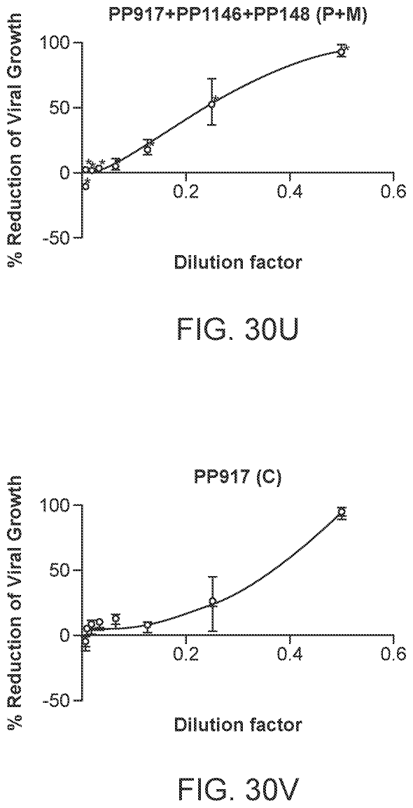

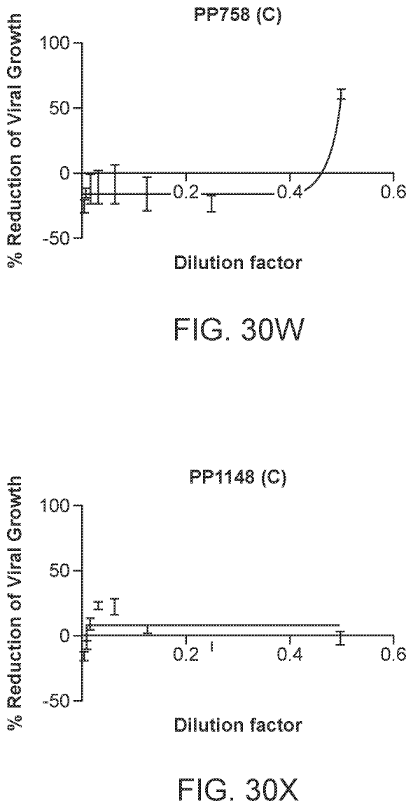

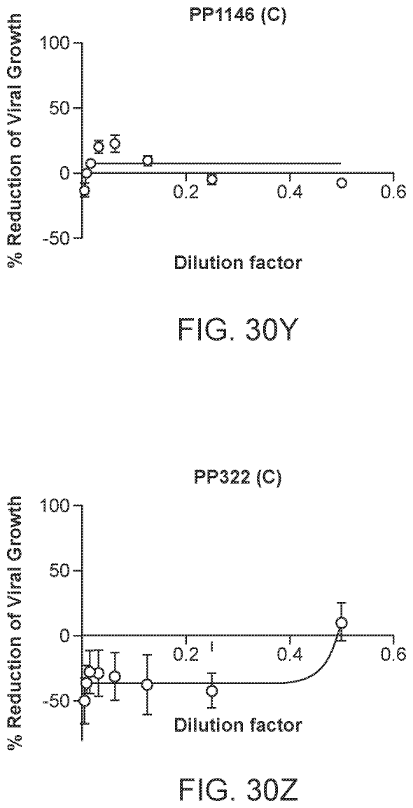

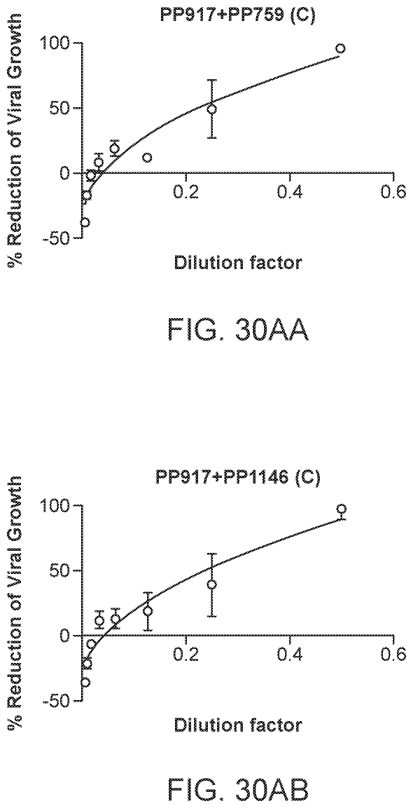

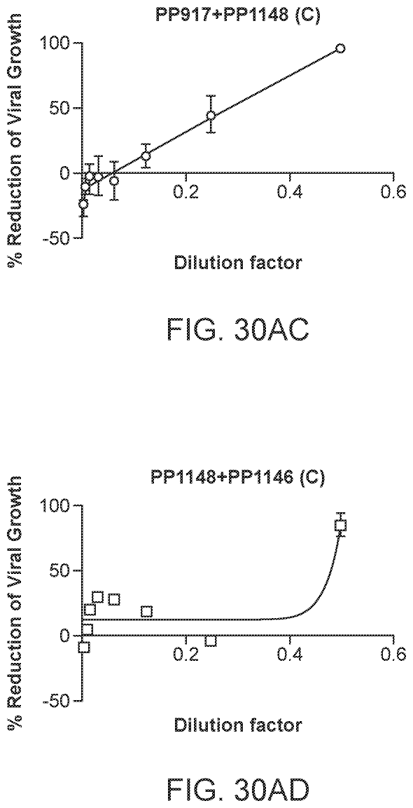

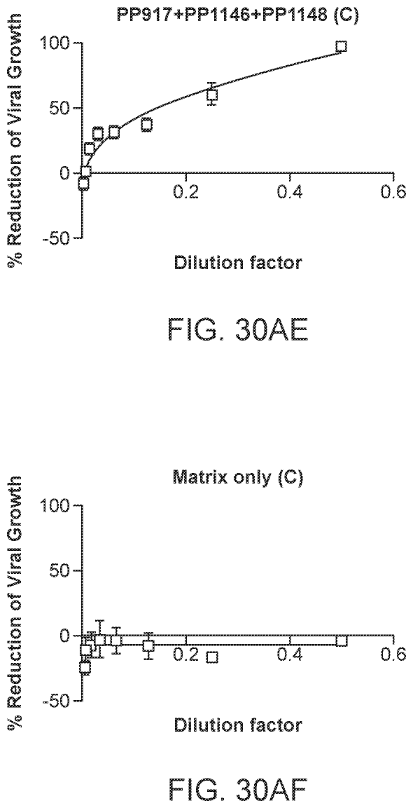

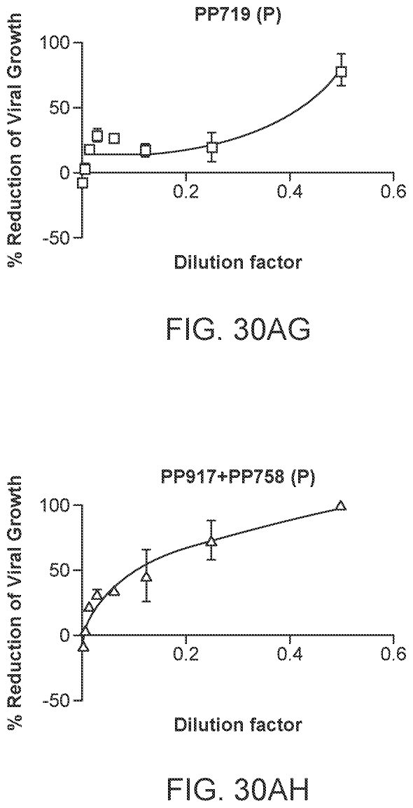

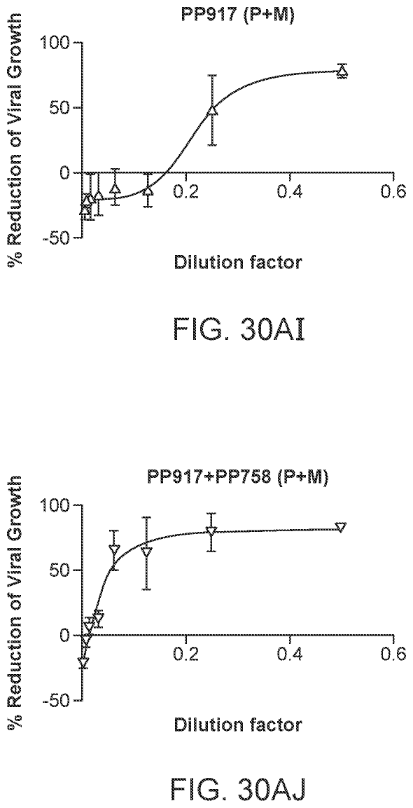

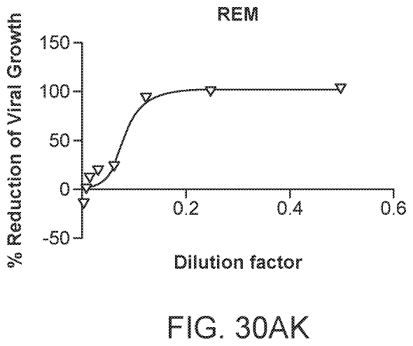

For the live virus titer assay, all samples were serially diluted 2-fold for a total of 8 dilutions, mixed with 200 TCID 50 of virus, then transferred into 3 replicate wells/dilution to corresponding wells in 96-well plate which contains a monolayer of Vero E6 cells for titration. The 96-well plate was incubated in a humidified chamber at 37° C.±2° C. in 5±2% CO2. At 48 hours ±4 hrs. post inoculation, wells were scored for virus replication by immunostaining with an antibody specific for the SARS-CoV-2 nucleoprotein. Results were recorded in FIG. 30A-FIG. 30X. As expected, the non-relevant isotype control PP322 (sample #5, 15 and 25) and matrix (sample#31) didn't neutralize the virus. The positive control, Remdesivir, completely inhibits the infectivity at the first three dilutions. Some of Lumen VHH samples (sample #18, 33,34,35) show similar or stronger effect compared to Remdesivir.

The concentration of the VHHs at dilution factor 0.5 is the same with Lumen pseudovirus assay. The % reduction of infectivity at this dilution was plotted and compared with the results from pseudovirus assay (FIGS. 29B and 29C). Most samples have similar potency in both assays with some exemptions. Purified PP917 (sample #1), PP1146 (sample #4) and PP917+PP758 (sample #6) and cell extract of PP1148 (sample #23) and PP1148 (sample #24) are significantly less potent in the live virus assay. The samples were stored at −80 for extended period of time before live virus assay, which possibly caused the reduced potency of those samples. Nonetheless most of the results from live virus assay conducted by IITRI is consistent with Lumen pseudovirus assay. This suggests the pseudovirus assay is an effective approach to screen and predict the Covid VHH candidates.

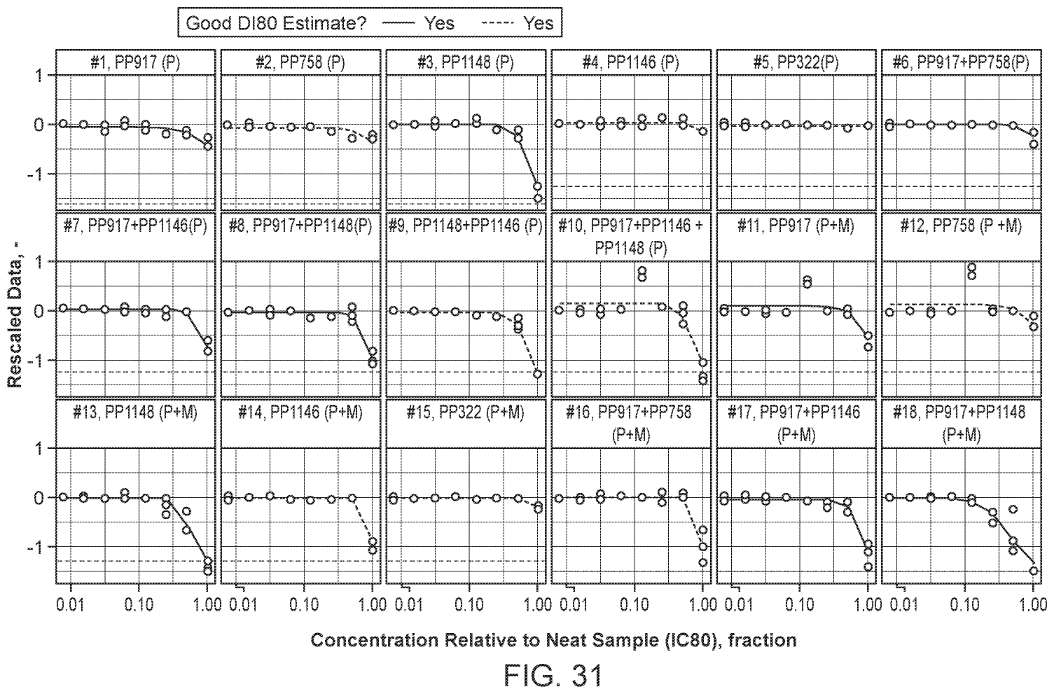

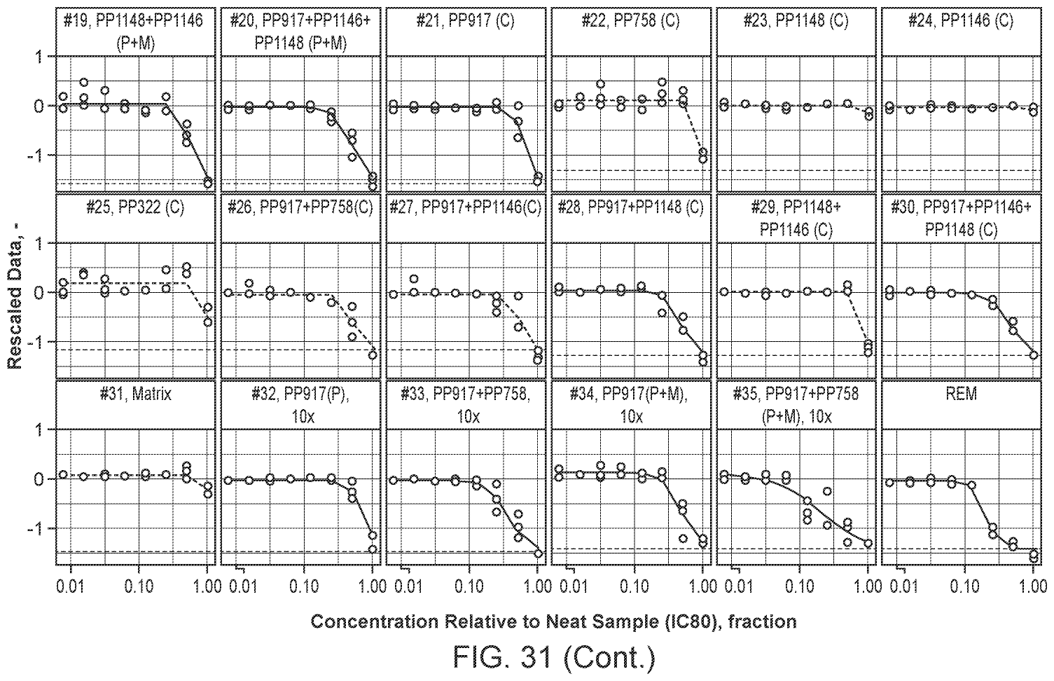

A more thorough analysis was conducted to confirm the correlation between the live virus assay and pseudovirus assay. After edge-effects correction was applied to the IITRI live virus assay data, the rescaled data were fit with a four-parameter model describing the inhibition of viral infection by VHH dilution (FIG. 31). The response model estimated the dilution that that provided a 50% reduction in infectivity in the assay, Dil₅₀.

$${Y}_{i} = Top - ( Top - Bottom ) \cdot \frac{{Dilution}^{H}}{{Dil}_{50}^{H} + {Dilution}^{H}}$$

All of the samples were provided at the IC80 concentration from the pseudovirus assay. As such, it is useful to reparametrize the model to instead estimate the dilution that provides 80% reduction in infectivity in the assay, Dil₈₀.

$${Dil}_{50} = {( \frac{{Dil}_{50}^{H}}{4} )}^{1 / H}$$

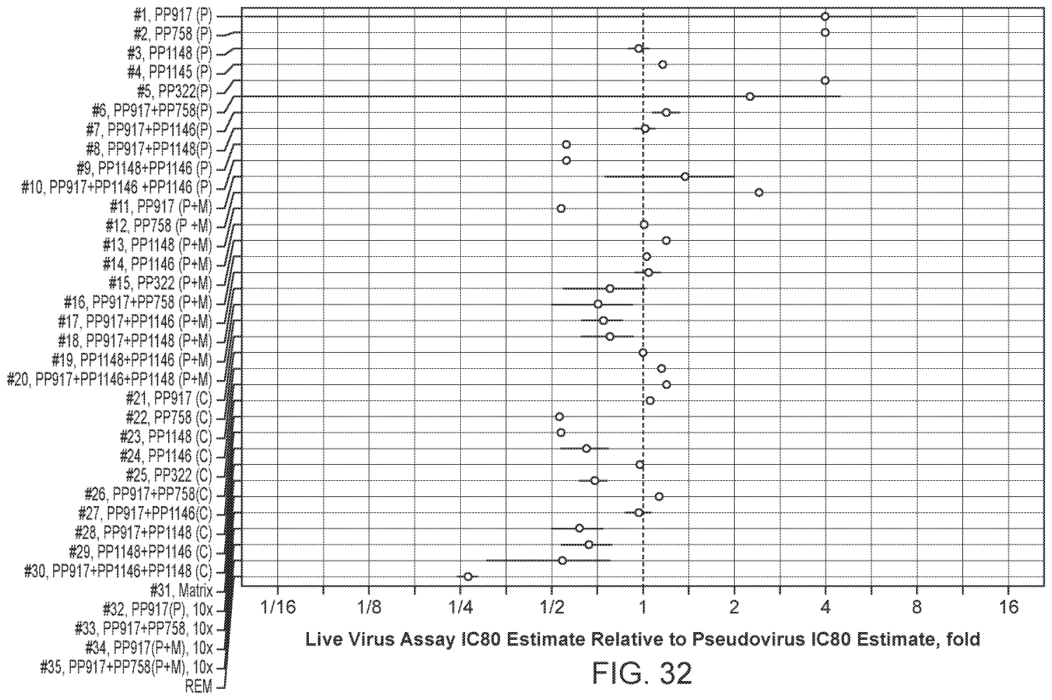

Thus, if a sample estimate of Dil₈₀ is equal to 1.0, then the live virus assessment of potency is the same as the pseudovirus assessment. At values>1.0, the live virus assessment of potency is lower than the pseudovirus assessment. At values<1.0, the live virus assessment of potency is higher than the pseudovirus assessment. The analysis results are shown in FIG. 32.

In conclusion, the live virus and pseudovirus estimates of potency (IC80) are in alignment. Initial purified protein (P; samples 1-10) showed the largest difference, where the potency according to the live virus assay was 1-fold to 4-fold lower than in the pseudovirus assay. Initial purified protein plus matrix (P+M; samples 11-20) showed fair agreement, where the potency according to the live virus assay was 0.5-fold to 3-fold lower than in the pseudovirus assay. Cell-extract (C; samples 21-30) and follow-up purified protein (10×; samples 32-35) showed greater potency in the live virus assay, where the potency according to the live virus assay was 1-fold to 2-fold greater than in the pseudovirus assay. In conclusion, lysates from the biomass of Covid candidate strains effectively neutralize SARS-CoV-2 virus (USA-WA1/2020).

### Example 15: Functional Assays

Expression of VHH antibodies in spirulina was evaluated. In brief, recombinant protein expression in spirulina was measured by capillary electrophoresis immunoassay (CEIA) using a Jess system (ProteinSimple). The Jess system was run as recommended by the manufacturer. Briefly, to determine the expression of heterologous protein as a percentage of total mass, dried biomass samples were diluted to a concentration of 0.1-1 mg/mL using water and a 5×master mix prepared from an EZ Standard Pack 1 (Bio-Techne). Similarly, to determine the proportion of heterlogous protein in total soluble Spirulina extract, total soluble protein was extracted from dried spirulina biomass using bead beating extracting method. Total protein is determined using Pierce BCA Protein Assay Kit (Thermo Fisher). Jess samples were prepared as described above at concentrations of 0.1-1 mg/mL. Purified protein controls used to generate standard curves were typically loaded at a range of concentrations from 0.25-8 μg/mL. A 12-230 kDa Jess/Wes Separation Module (ProteinSimple) was used for separation. A mouse anti-His-Tag antibody (GenScript) was diluted 1:100 and used as the primary antibody. An anti-mouse NIR fluorescence-conjugated secondary antibody (ProteinSimple) was primarily used for detection. Data analysis was performed using the Protein Simple Compass software and Microsoft Excel. Expression for exemplary VHHs is shown in Table 15.

TABLE 15
Spirulina Expression

| | Spirulina Expression | | | |
|---|---|---|---|---|
| | % of Total Protein | | % of Total Mass | |
| | Experiment1 | Experiment2 | Experiment1 | Experiment2 |
| PP661 | 1.4 | | 0.5 | |
| PP755 | 2 | 3 | 0.9 | 1.4 |
| PP757 | 0.03 | 0.03 | 0.01 | 0.01 |
| PP758 | 1.2 | 2.9 | | |
| PP759 | 10.8 | | 4 | |
| PP760 | 0.2 | | 0.1 | |
| PP762 | 0.1 | 0.1 | 0.03 | 0.04 |
| PP764 | 0.4 | | | |
| PP765 | 0.1 | 0.1 | 0.03 | 0.04 |
| PP767 | 0.1 | 0.05 | 0.03 | 0.02 |
| PP768 | 5.2 | 5.2 | 2 | 2 |
| PP769 | 0.3 | 0.4 | 0.1 | 0.1 |
| PP770 | 0.4 | 0.5 | 0.1 | 0.2 |
| PP771 | 0.1 | | | |
| PP772 | 0.4 | | | |
| PP773 | 0.1 | 0.1 | 0.02 | 0.03 |
| PP774 | 2.7 | 7.3 | 0.9 | 2.4 |
| PP775 | 1.1 | | | |
| PP776 | 0.1 | 0.1 | 0.03 | 0.04 |
| PP777 | 0.1 | | | |
| PP778 | 3.9 | 5.5 | 1.3 | 1.9 |
| PP779 | 8.4 | 21.7 | 2.5 | 6.5 |
| PP780 | 0.6 | 3.6 | 0.2 | 1 |
| PP781 | 0.7 | 1.1 | 0.2 | 0.3 |
| PP782 | 0.7 | 0.9 | 0.2 | 0.3 |
| PP784 | 2.6 | 5.2 | 0.9 | 1.7 |
| PP785 | 0.5 | 0.1 | 0.5 | 0.1 |
| PP787 | 3 | | | |
| PP788 | 0.1 | 0.1 | 0.02 | 0.03 |
| PP789 | 0.1 | 0.1 | 0.05 | 0.05 |
| PP790 | 5.6 | 5.4 | 1.6 | 1.5 |
| PP791 | 0.9 | 0.6 | 0.3 | 0.2 |
| PP793 | 0.01 | | | |
| PP796 | 0.04 | 0.03 | 0.01 | 0.01 |
| PP797 | 4 | 3.1 | 1.2 | 0.9 |
| PP798 | 0.1 | | | |
| PP799 | 0.5 | 0.5 | 0.1 | 0.1 |
| PP800 | 15.7 | 11.7 | 5.4 | 4 |
| PP801 | 9.9 | 10.4 | 3.8 | 4 |
| PP899 | 0.02 | 0.05 | 0.01 | 0.03 |
| PP900 | 8.3 | 4.6 | 5.2 | 2.9 |
| PP901 | 6.6 | 5.5 | 2.4 | 2 |
| PP902 | 2.6 | 3.4 | 1 | 1.3 |
| PP903 | 0.2 | 0.2 | 0.1 | 0.1 |
| PP904 | 3.3 | 4.9 | 0.8 | 1.2 |
| PP905 | 4.6 | 1.14 | 2 | |
| PP906 | 0.28 | 0.79 | 0.09 | 0.12 |
| PP907 | 4.51 | 4.55 | 1.85 | 1.87 |
| PP908 | 8.4 | 3.78 | 4 | 1.8 |
| PP909 | 4.18 | 4.6 | 1.25 | 2 |
| PP910 | 0.28 | 0.25 | | 0.24 |
| PP911 | 0.04 | 0.06 | 0.03 | 0.05 |
| PP912 | 6.01 | 2.92 | 2.81 | 1.37 |
| PP913 | 4.26 | 1.98 | 2.86 | 1.33 |
| PP914 | 3.69 | 12.26 | 1.46 | 4.85 |
| PP915 | 0.96 | 0.52 | 0.4 | 0.21 |
| PP916 | 3.53 | 5.12 | 1.17 | 1.7 |
| PP917 | 6.4 | 9.4 | | |
| PP927 | 0.45 | 0.94 | 0.22 | 0.47 |
| PP928 | 3.73 | 4.6 | 2 | 2 |
| PP929 | 0.03 | 8.73 | 0.01 | 2 |
| PP930 | 3.91 | 5.87 | 1.27 | 1.91 |
| PP931 | 4.95 | 4.95 | 2 | 2 |
| PP932 | 0.69 | 0.84 | 0.3 | 0.36 |
| PP933 | 0.21 | 0.04 | 0.06 | 0.01 |
| PP934 | 1.94 | 1.92 | 0.49 | 0.48 |
| PP935 | 12.85 | 15.16 | 8 | 9.44 |
| PP1094 | 3.06 | 3.06 | 2 | 2 |
| PP1095 | 0.05 | 0.07 | 0.02 | 0.03 |
| PP1096 | 5.17 | 5.17 | 2 | 2 |
| PP1097 | 29.85 | 29.85 | 8 | 8 |
| PP1098 | 5.5 | 5.5 | 2 | 2 |
| PP1099 | 0.71 | 1.54 | 0.22 | 0.48 |
| PP1100 | 0.07 | 0.14 | 0.02 | 0.04 |
| PP1101 | 6.68 | 6.68 | 2 | 2 |
| PP1102 | 2.88 | 7.97 | 0.72 | 2 |
| PP1104 | 7.11 | 7.11 | 2 | 2 |
| PP1105 | 6.16 | 6.16 | 2 | 2 |
| PP1106 | 0.12 | 0.33 | 0.05 | 0.14 |
| PP1107 | 22.77 | 22.77 | 8 | 8 |
| PP1108 | 5.78 | 5.78 | 2 | 2 |
| PP1109 | 4.12 | 7.48 | 1.1 | 2 |
| PP1110 | 5.24 | | 2 | |
| PP1111 | 6.47 | | 2 | |
| PP1112 | 5.69 | | 2 | |
| PP1113 | 2.95 | | 1.4 | |
| PP1114 | 0.33 | | 0.12 | |
| PP1115 | 4.15 | | 2 | |
| PP1116 | 16.33 | | 8 | |
| PP1117 | 3.32 | | 1.24 | |
| PP1118 | 4.18 | | 1.59 | |
| PP1144 | 0.03 | | 0.01 | |
| PP1145 | 14.61 | | 8 | |
| PP1146 | 1 | 1 | | |
| PP1147 | 0.05 | | 0.03 | |
| PP1148 | 0.7 | 1 | | |
| PP1149 | 0.7 | | 0.24 | |
| PP1150 | 0.22 | | 0.07 | |
| PP1151 | 0.06 | | 0.02 | |
| PP1152 | 0.02 | | 0.01 | |
| PP1153 | 2.48 | | 1.11 | |
| PP1176 | 0.01 | | 0.01 | |

### Infectivity Analysis

Virus neutralization potency by VHHs at single concentration was assessed following the neutralization protocol which the following modifications. Plates were seeded with 293-ACE2 cell line (Jessie Bloom lab, FHCRC) at a density of 1.25×10⁴ cells per well in 100 μL volume. 12-16 hours after seeding, VHHs were tested at 10 μg/mL final concentration. Virus titer and controls were prepared as describe above. Virus infectivity was assessed by measuring luminescence as above. Luminescence RLUs from virus only wells were normalized as 100% infectivity and RLUs from cells only were normalized as 0% infectivity. Pseudotyped virus infectivity were calculated as a proportion of the 100% and 0% infectivity, results are shown in Table 16.

TABLE 16

| | | | infectivity |
|---|---|---|---|
| | | | % under |
| | | | 10 ug/mL |
| | | | VHH |
| PP# | Description | Form | treatment |
| PP802 | MBP-R2A-C5-H6 | Monomer | 87.79 |
| PP803 | MBP-R2A-F3-H6 | Monomer | 70.66 |
| PP806 | MBP-R2B-E6-H6 | Monomer | 86.46 |
| PP807 | MBP-R2C-B3-H6 | Monomer | 75.26 |
| PP810 | MBP-R2D-A6-H6 | Monomer | 77.48 |
| PP811 | MBP-R2D-D3-H6 | Monomer | 75.92 |
| PP813 | MBP-R2E−D2-H6 | Monomer | 75.51 |
| PP814 | MBP-R2E−F4-H6 | Monomer | 84.69 |
| PP815 | MBP-R5A-A1-H6 | Monomer | 84.41 |
| PP817 | MBP-R5A-B3-H6 | Monomer | 80.01 |
| PP819 | MBP-R5A-C5-H6 | Monomer | 54.15 |
| PP821 | MBP-R5A-D6-H6 | Monomer | 76.7 |
| PP822 | MBP-R5A-E5-H6 | Monomer | 71.62 |
| PP823 | MBP-R5B-C3-H6 | Monomer | 65.49 |
| PP825 | MBP-R5C-A1-H6 | Monomer | 56.52 |
| PP826 | MBP-R5C-A2-H6 | Monomer | 70.56 |
| PP828 | MBP-R5C-B2-H6 | Monomer | 73.37 |
| PP831 | MBP-R5C-F3-H6 | Monomer | 81.23 |
| PP832 | MBP-R5C-F5-H6 | Monomer | 75.46 |
| PP833 | MBP-R5C-F6-H6 | Monomer | 86.16 |
| PP834 | MBP-R5C-G2-H6 | Monomer | 89.22 |
| PP836 | MBP-R5D-A1-H6 | Monomer | 54.98 |
| PP837 | MBP-R5D-B2-H6 | Monomer | 77.62 |
| PP846 | MBP-R5D-G6-H6 | Monomer | 77.07 |
| PP847 | MBP-R5E−D1-H6 | Monomer | 77.14 |
| PP848 | MBP-R5E−G2-H6 | Monomer | 81.92 |
| PP880 | MBP-R5a-B5-H6 | Monomer | 57.96 |
| PP881 | MBP-R5a-F2-H6 | Monomer | 83.22 |
| PP882 | MBP-R1e-B2-H6 | Monomer | 84.01 |
| PP883 | MBP-R1e-D4-H6 | Monomer | 94.32 |
| PP884 | MBP-R1i-A11-H6 | Monomer | 91.32 |
| PP885 | MBP-R5b-C5-H6 | Monomer | 100.74 |
| PP886 | MBP-S1a-C4-H6 | Monomer | 84.79 |
| PP887 | MBP-S1b-E7-H6 | Monomer | 85.74 |
| PP888 | MBP-S1b-E11-H6 | Monomer | 79.99 |
| PP889 | MBP-S2a-C4-H6 | Monomer | 82.55 |
| PP890 | MBP-S2b-A7-H6 | Monomer | 89.97 |
| PP891 | MBP-S2b-C10-H6 | Monomer | 102.03 |
| PP892 | MBP-S2b-F7-H6 | Monomer | 83.18 |
| PP893 | MBP-S2b-G8-H6 | Monomer | 78.7 |
| PP894 | MBP-S3a-C5-H6 | Monomer | 76.11 |
| PP895 | MBP-S3a-H1-H6 | Monomer | 84.42 |
| PP896 | MBP-S3b-A7-H6 | Monomer | 82.75 |
| PP897 | MBP-S3b-A8-H6 | Monomer | 79.28 |
| PP898 | MBP-S3b-C8-H6 | Monomer | 62.37 |
| PP918 | MBP-R1i-C8-H6 | Monomer | 71.83 |
| PP919 | MBP-R1i-E7-H6 | Monomer | 79.31 |
| PP921 | MBP-R2b-D4-H6 | Monomer | 70.58 |
| PP922 | MBP-R5a-F5-H6 | Monomer | 67.74 |
| PP923 | MBP-R5d-E1-H6 | Monomer | 76.93 |
| PP924 | MBP-R5e-C1-H6 | Monomer | 102.23 |
| PP925 | MBP-S3a-H2-H6 | Monomer | 74.53 |
| PP926 | MBP-S3b-E11-H6 | Monomer | 67.58 |
| PP899 | MBP-5HVZ-R5a-B5-H6 | Dimer | 2.43 |
| PP900 | MBP-5HVZ-R5a-F2-H6 | Dimer | 18.27 |
| PP901 | MBP-5HVZ-R1e-B2-H6 | Dimer | 72.07 |
| PP902 | MBP-5HVZ-R1e-D4-H6 | Dimer | 72.4 |
| PP903 | MBP-5HVZ-R1i-A11-H6 | Dimer | 38.41 |
| PP904 | MBP-5HVZ-R5b-C5-H6 | Dimer | 77.02 |
| PP904 | MBP-5HVZ-R5b-C5-H6 | Dimer | 92.03 |
| PP905 | MBP-5HVZ-S1a-C4-H6 | Dimer | 75.66 |
| PP906 | MBP-5HVZ-S1b-E7-H6 | Dimer | 69.61 |
| PP907 | MBP-5HVZ-S1b-E11-H6 | Dimer | 76.34 |
| PP908 | MBP-5HVZ-S2a-C4-H6 | Dimer | 47.47 |
| PP909 | MBP-5HVZ-S2b-A7-H6 | Dimer | 67.07 |
| PP909 | MBP-5HVZ-S2b-A7-H6 | Dimer | 54.05 |
| PP910 | MBP-5HVZ-S2b-C10-H6 | Dimer | 65.07 |
| PP911 | MBP-5HVZ-S2b-F7-H6 | Dimer | 74.92 |
| PP912 | MBP-5HVZ-S2b-G8-H6 | Dimer | 73.24 |
| PP913 | MBP-5HVZ-S3a-C5-H6 | Dimer | 71.09 |
| PP913 | MBP-5HVZ-S3a-C5-H6 | Dimer | 62.68 |
| PP914 | MBP-5HVZ-S3a-H1-H6 | Dimer | 74.35 |
| PP914 | MBP-5HVZ-S3a-H1-H6 | Dimer | 77.51 |
| PP915 | MBP-5HVZ-S3b-A7-H6 | Dimer | 90.5 |
| PP916 | MBP-5HVZ-S3b-A8-H6 | Dimer | 70.45 |
| PP916 | MBP-5HVZ-S3b-A8-H6 | Dimer | 55.41 |
| PP917 | MBP-5HVZ-S3b-C8-H6 | Dimer | 6.45 |
| PP927 | MBP-5HVZ-R1i-C8-H6 | Dimer | 45.38 |
| PP928 | MBP-5HVZ-R1i-E7-H6 | Dimer | 78.55 |
| PP930 | MBP-5HVZ-R2b-D4-H6 | Dimer | 35.57 |
| PP931 | MBP-5HVZ-R5a-F5-H6 | Dimer | 57.5 |
| PP933 | MBP-5HVZ-R5e-C1-H6 | Dimer | 92.08 |
| PP933 | MBP-5HVZ-R5e-C1-H6 | Dimer | 73.99 |
| PP934 | MBP-5HVZ-S3a-H2-H6 | Dimer | 75.01 |
| PP934 | MBP-5HVZ-S3a-H2-H6 | Dimer | 84.12 |
| PP935 | MBP-5HVZ-S3b-E11-H6 | Dimer | 48.92 |
| PP1094 | MBP-5HVZ-PAPA-RN-1-H6 | Dimer | 83.42 |
| PP1095 | MBP-5HVZ-PAPA-RN-2-H6 | Dimer | 76.8 |
| PP1096 | MBP-5HVZ-PAPA-RN-3-H6 | Dimer | 86.48 |
| PP1097 | MBP-5HVZ-PAPA-RN-5-H6 | Dimer | 18.59 |
| PP1098 | MBP-5HVZ-PAPA-RN-9-H6 | Dimer | 58.26 |
| PP1099 | MBP-5HVZ-PAPA-RN-12-H6 | Dimer | 70.58 |
| PP1100 | MBP-5HVZ-PAPA-RN-14-H6 | Dimer | 17.22 |
| PP1101 | MBP-5HVZ-PAPA-RN-16-H6 | Dimer | 91.78 |
| PP1102 | MBP-5HVZ-PAPA-RN-17-H6 | Dimer | 40.91 |
| PP1103 | MBP-5HVZ-PAPA-RN-18-H6 | Dimer | 57.57 |
| PP1104 | MBP-5HVZ-PAPA-RN-19-H6 | Dimer | 73.81 |
| PP1105 | MBP-5HVZ-PAPA-RN-20-H6 | Dimer | 79.82 |
| PP1106 | MBP-5HVZ-PAPA-RN-21-H6 | Dimer | 76.58 |
| PP1107 | MBP-5HVZ-PAPA-RN-22-H6 | Dimer | 34.33 |
| PP1108 | MBP-5HVZ-PAPA-RN-23-H6 | Dimer | 93.09 |
| PP1109 | MBP-5HVZ-PAPA-RN-25-H6 | Dimer | 88.05 |
| PP1110 | MBP-5HVZ-PAPA-RN-26-H6 | Dimer | 57.89 |
| PP1111 | MBP-5HVZ-PAPA-RN-27-H6 | Dimer | 68.3 |
| PP1112 | MBP-5HVZ-PAPA-RN-28-H6 | Dimer | 70.44 |
| PP1113 | MBP-5HVZ-PAPA-RN-29-H6 | Dimer | 6.25 |
| PP1114 | MBP-5HVZ-PAPA-RN-30-H6 | Dimer | 76.26 |
| PP1115 | MBP-5HVZ-PAPA-RN-31-H6 | Dimer | 86.87 |
| PP1116 | MBP-5HVZ-PAPA-Sb-15-H6 | Dimer | 1.69 |
| PP1117 | MBP-5HVZ-PAPA-Sb-45-H6 | Dimer | 29.89 |
| PP1118 | MBP-5HVZ-PAPA-Sb-68-H6 | Dimer | 30.51 |
| PP1143 | MBP-5HVZ-PAPA-R2b-D5-H6 | Dimer | 0.09 |
| PP1144 | MBP-5HVZ-PAPA-R2c-F6-H6 | Dimer | 9.18 |
| PP1145 | MBP-5HVZ-PAPA-R2d-A6-H6 | Dimer | 65.24 |
| PP1146 | MBP-5HVZ-PAPA-R2d-D3-H6 | Dimer | 32.75 |
| PP1147 | MBP-5HVZ-PAPA-R5a-B7-H6 | Dimer | 1.79 |
| PP1148 | MBP-5HVZ-PAPA-R5a-C5-H6 | Dimer | 5.95 |
| PP1149 | MBP-5HVZ-PAPA-R5a-E5-H6 | Dimer | 25.51 |
| PP1150 | MBP-5HVZ-PAPA-R5b-G1-H6 | Dimer | 2.31 |
| PP1151 | MBP-5HVZ-PAPA-R5d-B5-H6 | Dimer | 35.62 |
| PP1152 | MBP-5HVZ-PAPA-R5d-B6-H6 | Dimer | 4.64 |
| PP1153 | MBP-5HVZ-PAPA-R5d-D4-H6 | Dimer | 81.66 |
| PP1154 | MBP-5HVZ-PAPA-R5d-G1-H6 | Dimer | 53.2 |
| PP1176 | MBP-5HVZ-PAPA-R2a-F3-H6 | Dimer | 58.44 |
| PP1131 | MBP-4B0F-PAPA-R2b-D5-H6 | Heptamer | -0.01 |
| PP1132 | MBP-4B0F-PAPA-R2c-F6-H6 | Heptamer | 2.65 |
| PP1133 | MBP-4B0F-PAPA-R2d-A6-H6 | Heptamer | 48.14 |
| PP1134 | MBP-4B0F-PAPA-R2d-D3-H6 | Heptamer | 10.86 |
| PP1135 | MBP-4B0F-PAPA-R5a-B7-H6 | Heptamer | -0.03 |
| PP1136 | MBP-4B0F-PAPA-R5a-C5-H6 | Heptamer | 1.53 |
| PP1137 | MBP-4B0F-PAPA-R5a-E5-H6 | Heptamer | 33.69 |
| PP1138 | MBP-4B0F-PAPA-R5b-G1-H6 | Heptamer | 31.54 |
| PP1139 | MBP-4B0F-PAPA-R5d-B5-H6 | Heptamer | 70.97 |
| PP1140 | MBP-4B0F-PAPA-R5d-B6-H6 | Heptamer | 2.47 |
| PP1141 | MBP-4B0F-PAPA-R5d-D4-H6 | Heptamer | 73.11 |
| PP1142 | MBP-4B0F-PAPA-R5d-G1-H6 | Heptamer | 35.91 |
| PP1175 | MBP-4B0F-PAPA-R2a-F3-H6 | Heptamer | 60.29 |
| PP1155 | MPB-scTRP-PAPA-R2b-D5-H6 | Trimer | 0.01 |
| PP1156 | MPB-scTRP-PAPA-R2c-F6-H6 | Trimer | 18.14 |
| PP1157 | MPB-scTRP-PAPA-R2d-A6-H6 | Trimer | 32.92 |
| PP1158 | MPB-scTRP-PAPA-R2d-D3-H6 | Trimer | 28.8 |
| PP1159 | MPB-scTRP-PAPA-R5a-B7-H6 | Trimer | 17.72 |
| PP1160 | MPB-scTRP-PAPA-R5a-C5-H6 | Trimer | 4.22 |
| PP1161 | MPB-scTRP-PAPA-R5a-E5-H6 | Trimer | 59.86 |
| PP1162 | MPB-scTRP-PAPA-R5b-G1-H6 | Trimer | 66.78 |
| PP1165 | MPB-scTRP-PAPA-R5d-D4-H6 | Trimer | 77.51 |
| PP1166 | MPB-scTRP-PAPA-R5d-G1-H6 | Trimer | 69.73 |
| PP1177 | MPB-scTRP-PAPA-R2a-F3-H6 | Trimer | 51.09 |
| PP1181 | R2d-D3-PP7-H6 | Particle | 1.57 |

### Pseudovirus Neutralization

Constructs were evaluated for virus neutralization by way of a pseudovirus neutralization assay. Neutralization assays determine the ability of a VHH antibody to inhibit virus infection of cultured cells with SARS-CoV-2 variants Wuhan, Delta, or Gamma. If neutralizing antibodies are present, their levels can be measured by determining the threshold at which they are able to prevent viral replication in the infected cell cultures.

The SARS-CoV-2 pseudotyped lentiviral particle generation, tittering and neutralization assays were performed following the protocol outline in Crawford K. H. D. et al., 2020 with minor modifications. In brief, poly-L-lysine coated clear bottom 96-well black plates (Thermo Scientific, 12-566-70) were seeded with 293-ACE2 cell line (Jessie Bloom lab, FHCRC) at a density of 1.25×10⁴ cells per well in 100 μL volume. 12-16 hours after seeding, 5-fold antibody dilutions, starting at 100 μg/mL, were prepared. Control, virus only and cell only were prepared by as previously reported. 60 μL of titered virus was added and mixed with antibody dilutions and virus only wells. The mix was incubated at 37° C. for 1 hour. Gently, 100 μL of growth media was removed from each well and 100 μL of the mixture were added to corresponding well on 293-ACE2 cells seeded plates. Polybrene (Sigma Aldrich, P4707) was added to each well at a final concentration of 5 μg/mL. Plates were incubated at 37° C. for 48-60-hour post infection. Virus neutralization was assessed by measuring luminescence. While incubation, Bright-Glo Luciferase reagents (Promega, E2610) were thawed, equilibrated at room temperature, and prepared following the manufacturer's recommendation. 100 μL of growth media was removed from each well and 30 μL per well of luciferase reagent added. Plates were incubated for 2 min at room temperature in the dark and luminescence was measured using M2 plate reader (Molecular Devices). Luminescence RLUs from virus only wells were normalized as 100% infectivity and RLUs from cells only were normalized as 0% infectivity. Infectivity and IC50 were calculated using Four Parameter Logistic Regression on GraphPad Prism (GraphPad Software), see Table 17.

TABLE 17

| | Pseudovirus neutralization IC50 (nM) | | | |
|---|---|---|---|---|
| | | SARS | SARS- | |
| | SARS-COV-2 | CoV-2 | CoV-2 | |
| | Wuhan | Delta | Gamma | |
| | Reference | Variant | Variant | |
| PP# | Pseudovirus | Pseudovirus | Pseudovirus | Notes |
| PP756 | 210 | | | |
| PP758 | 0.39 | NN | 3.73 | |
| PP759 | 138.1 | | | |
| PP760 | | 0.8 | NN | |
| PP762 | | 39.28 | 70.91 | |
| PP764 | ~25471 | | | |
| PP767 | | | 40.71 | |
| PP768 | | NN | NN | |
| PP769 | | 35.18 | NN | |
| PP772 | 42.73 | | | |
| PP776 | | 1.62 | NN | |
| PP777 | 0.7873 | | | |
| PP779 | | 85.46 | 1.821 | |
| PP780 | NN | 4.44E−17 | NN | |
| PP781 | NN | | NN | |
| PP782 | NN | NN | NN | |
| PP783 | | NN | NN | |
| PP785 | NN | 64.27 | 781.4 | |
| PP787 | | | NN | |
| PP799 | NN | NN | NN | |
| PP801 | | NN | NN | |
| PP899 | 2.29 | | | |
| PP900 | 29.63 | | | |
| PP903 | 56.67 | NN | NN | |
| PP908 | 1618 | NN | 63.13 | |
| PP909 | | NN | NN | |
| PP913 | NN | | | |
| PP915 | | NN | NN | |
| PP916 | | | NN | |
| PP917 | 6.59 | 0.81 | NN | |
| PP927 | NN | NN | NN | |
| PP930 | NN | NN | NN | |
| PP932 | | NN | NN | |
| PP933 | | NN | NN | |
| PP934 | | | 46310 | |
| PP935 | 34.21 | | | |
| PP1094 | | NN | NN | |
| PP1095 | | NN | NN | |
| PP1097 | 27.27 | NN | NN | |
| PP1098 | | | NN | |
| PP1099 | NN | | | |
| PP1100 | 6.953 | NN | NN | |
| PP1101 | | NN | NN | |
| PP1102 | ~5113 | | 21.5 | |
| PP1103 | | 2.49 | NN | |
| PP1105 | | NN | NN | |
| PP1106 | | | 63.41 | |
| PP1107 | 32.86 | NN | 0.6491 | |
| PP1112 | | NN | NN | |
| PP1113 | 7.09 | | | |
| PP1131 | 0.93 | | | |
| PP1132 | 4.14 | | | |
| PP1133 | 186.29 | | | |
| PP1134 | 3.97 | | | |
| PP1135 | 0.5335 | | | |
| PP1136 | 0.05 | | | |
| PP1137 | 77.315 | | | |
| PP1138 | NN | | | |
| PP1140 | 2.07 | | | |
| PP1142 | 107.485 | | | |
| PP1143 | 0.3015 | | 3.132 | |
| PP1144 | 11.87 | | | |
| PP1145 | 146.2 | NN | NN | |
| PP1146 | 36.17 | 4.17 | 6.617 | |
| PP1147 | 12.3 | 1.13 | NN | |
| PP1148 | 15.67 | 0.53 | NN | |
| PP1149 | 37.34 | | | |
| PP1150 | 2.72 | 0.43 | NN | |
| PP1151 | 15.2965 | | | |
| PP1152 | 3.87 | | | |
| PP1153 | | | 62.91 | |
| PP1154 | 271.6 | | | |
| PP1155 | 0.6337 | | | |
| PP1156 | 36.8 | | | |
| PP1157 | 18.95 | | | |
| PP1157 | 19.75 | | | |
| PP1158 | 27.41 | | | |
| PP1159 | 31.9 | | | |
| PP1160 | 2.41 | | | |
| PP1176 | | NN | NN | |
| PP1181 | 6.95 | | | |
| PP1816 | 0.4334 | | | Cerberbody |
| PP1896 | | 34.57 | | Cerberbody |
| PP1897 | | 0.09 | | Cerberbody |
| PP1898 | | 0.46 | | Cerberbody |

(NN = not neutralizing)

### Protease Resistance

Constructs were evaluated for protease resistance. A general strategy to rapidly screen VHHs for susceptibility to GI proteases is to evaluate VHH binding activity after treatment with an intestinal protease. Following protease incubation, protease is neutralized with inhibitors and VHH binding is activity is measured by capture ELISA using the target antigen. VHHs that functionally survive the protease incubations are able to bind the target antigen, which can then be detected with an anti-VHH or anti-VHH conjugate antibody. An EC50 binding curve is calculated using a 4-parameter logistic curve fit of the results and the value is compared to an undigested control to calculate the percent activity remaining. The VHHs of the disclosure were incubated with trypsin, chymotrypsin, and elastase. Protease resistance results agreed well between the ELISA and neutralization assays. Protease resistance was observed with trypsin, chymotrypsin, and elastase. Binding activity after a 1 hour digestion with exemplary proteases is shown in Table 18.

TABLE 18

| | | ELISA binding activity after 1 h digestion with the indicated protease | | | | | |
|---|---|---|---|---|---|---|---|
| Protein | | 0.1 mg/mL | 0.01 mg/mL | 0.1 mg/mL | 0.01 mg/mL | 5 μg/mL | 0.5 μg/mL |
| Product | VHH | trypsin | trypsin | chymotrypsin | chymotrypsin | elastase | elastase |
| PP917 | S3b-C8 | 84% | 92% | 111% | 110% | 118% | 103% |
| PP1113 | RN-29 | 100% | 114% | 6% | 93% | 29% | 68% |
| PP758 | R2B-D5 | 110% | 107% | 1% | 57% | 118% | 72% |
| PP1146 | R2d-D3 | 0% | 0% | 48% | 66% | 13% | 90% |
| PP1148 | R5a-C5 | 0% | 0% | 19% | 71% | 47% | 86% |
| PP1107 | RN-22 | 0% | 7% | 13% | 132% | 34% | 103% |
| PP1144 | R2c-F6 | 0% | 43% | 3% | 6% | 61% | 61% |
| PP1150 | R5b-G1 | not tested | not tested | 0% | 0% | 16% | 56% |
| PP1147 | R5a-B7 | not tested | not tested | 9% | 21% | 31% | 64% |
| PP1100 | RN-14 | not tested | not tested | 7% | 112% | 5% | 47% |
| PP899 | R5a-B5 | not tested | not tested | 0% | 17% | 0% | 63% |

### REFERENCES

1. Han et al., Am J Gastroenterol 2020.
2. Assiri et al., Lancet Infect Dis 2013.
3. WHO, WER 78 (43), 2003: 373-375.
4. Leung et al., Gastroenterol 2003.
5. Yeo et al., Lancet Gastroent Hepatol 2020.
6. Xiao et al., Gastroenterol 2020.
7. Xu et al., Nat Med 2020.
8. Kim et al., Cell Host Microbe 2020.
9. Gormley et al., PLOS ONE 2017.
10. Larter, Navy Times 2020.
11. Agarwal et al., J Clin Exp Hepatol 2020.
12. Li et al., Indoor Air 2005.
13. Gu et al., Gastroenterol 2020.
14. Zhang et al., Emerg Microbs Infect 2020.
15. Li et al., Science 2020.
16. Rothan & Byrareddy. J Autoimmun 2020.
17. Wrapp et al. bioRxiv 2020.
18. Burgers, P. P. et al. Structure of smAKAP and its regulation by PKA-mediated phosphorylation. Febs J 283, 2132-2148 (2016).

### INCORPORATION BY REFERENCE

This patent application incorporates by reference in their entireties for all purposes the following patent publications and applications: U.S. Ser. No. 10,131,870, U.S. Ser. No. 62/672,891 filed May 17, 2018, and PCT/US2019/032998 filed May 17, 2019.

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

1-43. (canceled)

44. A recombinant Spirulina, comprising: (a) a 5HVZ domain; and (b) an smAKAP domain.

45. The recombinant Spirulina of claim 44, wherein the 5HVZ domain and the smAKAP domain are each linked to one or more VHH antibodies.

46. The recombinant Spirulina of claim 45, wherein the 5HVZ domain is linked to two VHH antibodies; and the smAKAP domain is linked to one VHH antibody.

47. The recombinant Spirulina of claim 45, wherein the 5HVZ domain is linked to two VHH antibodies; and the smAKAP domain is linked to two VHH antibodies.

48. The recombinant Spirulina of claim 46, wherein the 5HVZ domain comprises: a first VHH on the N-terminus; and a second VHH on the C-terminus.

49. The recombinant Spirulina of claim 47, wherein the smAKAP domain comprises a third VHH on the N-terminus and a fourth VHH on the C-terminus.

50. The recombinant Spirulina of claim 44, wherein the recombinant Spirulina comprises a maltose binding protein (MBP).

51. The recombinant Spirulina of claim 44, wherein the smAKAP peptide comprises a mutation as compared to the WT smAKAP peptide of SEQ ID NO: 389.

52. An engineered polypeptide, comprising from N- to C-terminus, a first VHH antibody sequence; an smAKAP domain; and a second VHH antibody sequence.

53. The engineered polypeptide of claim 52, wherein the smAKAP domain comprises mutations selected from the group consisting of: C16S of SEQ ID NO: 389, C24S of SEQ ID NO: 389, and both C16S of SEQ ID NO: 389 and C24S of SEQ ID NO: 389.

54. The engineered polypeptide of claim 52, wherein the smAKAP domain comprises SEQ ID NO: 390.

55. A recombinant Spirulina expressing the engineered polypeptide of claim 52.

56. A nucleic acid comprising a sequence encoding for the engineered polypeptide of claim 52.

57. A composition comprising the recombinant Spirulina of claim 44.


SEQUENCE LISTING (681 sequences)

SEQ ID NO: 1 - protein, 13 aa, Artificial Sequence
  synthetic construct
GQITAENYMHWYR

SEQ ID NO: 2 - protein, 13 aa, Artificial Sequence
  synthetic construct
GSIREINAMHWVR

SEQ ID NO: 3 - protein, 13 aa, Artificial Sequence
  synthetic construct
GSILQINAIHWFR

SEQ ID NO: 4 - protein, 13 aa, Artificial Sequence
  synthetic construct
GRQFGAWRMHWYR

SEQ ID NO: 5 - protein, 13 aa, Artificial Sequence
  synthetic construct
GTEYDISAAHWYR

SEQ ID NO: 6 - protein, 13 aa, Artificial Sequence
  synthetic construct
GSSININYMHWFR

SEQ ID NO: 7 - protein, 13 aa, Artificial Sequence
  synthetic construct
GTWGNIQYLSWFR

SEQ ID NO: 8 - protein, 13 aa, Artificial Sequence
  synthetic construct
GSITSINWMHWFR

SEQ ID NO: 9 - protein, 13 aa, Artificial Sequence
  synthetic construct
GSSESWNAIHWFR

SEQ ID NO: 10 - protein, 13 aa, Artificial Sequence
  synthetic construct
GSYASIASMHWFR

SEQ ID NO: 11 - protein, 13 aa, Artificial Sequence
  synthetic construct
GYADEINMVHWYR

SEQ ID NO: 12 - protein, 13 aa, Artificial Sequence
  synthetic construct
GGVTRGNAMAWYR

SEQ ID NO: 13 - protein, 13 aa, Artificial Sequence
  synthetic construct
GTYASYETIAWYR

SEQ ID NO: 14 - protein, 13 aa, Artificial Sequence
  synthetic construct
GRILAANIVHWYR

SEQ ID NO: 15 - protein, 13 aa, Artificial Sequence
  synthetic construct
GSIYKYSDVHWFR

SEQ ID NO: 16 - protein, 13 aa, Artificial Sequence
  synthetic construct
GSGASIGAMHWFR

SEQ ID NO: 17 - protein, 13 aa, Artificial Sequence
  synthetic construct
GHIFSINNAAWVR

SEQ ID NO: 18 - protein, 13 aa, Artificial Sequence
  synthetic construct
GSAMMINRTHWFR

SEQ ID NO: 19 - protein, 13 aa, Artificial Sequence
  synthetic construct
GHQLSTNATHWFR

SEQ ID NO: 20 - protein, 13 aa, Artificial Sequence
  synthetic construct
GETFSSNAMHWFR

SEQ ID NO: 21 - protein, 13 aa, Artificial Sequence
  synthetic construct
GAELSYTAIHWFR

SEQ ID NO: 22 - protein, 13 aa, Artificial Sequence
  synthetic construct
GSAFATNGLHWFR

SEQ ID NO: 23 - protein, 13 aa, Artificial Sequence
  synthetic construct
GQLDRSNAMAWFR

SEQ ID NO: 24 - protein, 13 aa, Artificial Sequence
  synthetic construct
GDDFIENAISWYR

SEQ ID NO: 25 - protein, 13 aa, Artificial Sequence
  synthetic construct
GAFFSYGAIHWFR

SEQ ID NO: 26 - protein, 13 aa, Artificial Sequence
  synthetic construct
GDIFSINALHWFR

SEQ ID NO: 27 - protein, 13 aa, Artificial Sequence
  synthetic construct
GSAMMINRTHWFR

SEQ ID NO: 28 - protein, 13 aa, Artificial Sequence
  synthetic construct
GNDWYINSAHWYR

SEQ ID NO: 29 - protein, 13 aa, Artificial Sequence
  synthetic construct
GYDFDILRMAWFR

SEQ ID NO: 30 - protein, 13 aa, Artificial Sequence
  synthetic construct
GSISSIEAMSWFR

SEQ ID NO: 31 - protein, 13 aa, Artificial Sequence
  synthetic construct
GYHASILAMHWVR

SEQ ID NO: 32 - protein, 13 aa, Artificial Sequence
  synthetic construct
GGISVANVMHWFR

SEQ ID NO: 33 - protein, 13 aa, Artificial Sequence
  synthetic construct
GSPFKTQTVHWFR

SEQ ID NO: 34 - protein, 13 aa, Artificial Sequence
  synthetic construct
GSIGQIVLVHWFR

SEQ ID NO: 35 - protein, 13 aa, Artificial Sequence
  synthetic construct
GLDVSINFMHWYR

SEQ ID NO: 36 - protein, 13 aa, Artificial Sequence
  synthetic construct
GDIFSKNAMHWVR

SEQ ID NO: 37 - protein, 13 aa, Artificial Sequence
  synthetic construct
GSGFSNNAMHWYR

SEQ ID NO: 38 - protein, 13 aa, Artificial Sequence
  synthetic construct
GSIFSLNWVHWFR

SEQ ID NO: 39 - protein, 13 aa, Artificial Sequence
  synthetic construct
GTIFSQNAVHWFR

SEQ ID NO: 40 - protein, 13 aa, Artificial Sequence
  synthetic construct
GSFFSINAVHWFR

SEQ ID NO: 41 - protein, 13 aa, Artificial Sequence
  synthetic construct
GTIPSINYTAWYR

SEQ ID NO: 42 - protein, 13 aa, Artificial Sequence
  synthetic construct
GGIFSINSMAWYR

SEQ ID NO: 43 - protein, 13 aa, Artificial Sequence
  synthetic construct
GQTNSILAMHWYR

SEQ ID NO: 44 - protein, 13 aa, Artificial Sequence
  synthetic construct
GSATSINRMAWVR

SEQ ID NO: 45 - protein, 13 aa, Artificial Sequence
  synthetic construct
GSIESINAIHWFR

SEQ ID NO: 46 - protein, 13 aa, Artificial Sequence
  synthetic construct
GSISSYNAMHWFR

SEQ ID NO: 47 - protein, 13 aa, Artificial Sequence
  synthetic construct
GSIWSIAFMSWYR

SEQ ID NO: 48 - protein, 13 aa, Artificial Sequence
  synthetic construct
GSSTSINIMAWYR

SEQ ID NO: 49 - protein, 13 aa, Artificial Sequence
  synthetic construct
GSRDSIVAMHWYR

SEQ ID NO: 50 - protein, 13 aa, Artificial Sequence
  synthetic construct
GRILAANIVHWYR

SEQ ID NO: 51 - protein, 13 aa, Artificial Sequence
  synthetic construct
GSDFTINAMHWFR

SEQ ID NO: 52 - protein, 13 aa, Artificial Sequence
  synthetic construct
GSSFSINWMHWFR

SEQ ID NO: 53 - protein, 13 aa, Artificial Sequence
  synthetic construct
GSIYVINTMHWFR

SEQ ID NO: 54 - protein, 13 aa, Artificial Sequence
  synthetic construct
GQISSNNGMHWFR

SEQ ID NO: 55 - protein, 13 aa, Artificial Sequence
  synthetic construct
GSQLSINYVHWFR

SEQ ID NO: 56 - protein, 13 aa, Artificial Sequence
  synthetic construct
GSIFGINDVHWFR

SEQ ID NO: 57 - protein, 13 aa, Artificial Sequence
  synthetic construct
GRILAANIVHWYR

SEQ ID NO: 58 - protein, 13 aa, Artificial Sequence
  synthetic construct
GSIPYVQAMSWFR

SEQ ID NO: 59 - protein, 13 aa, Artificial Sequence
  synthetic construct
GFEFSTSAMHWYR

SEQ ID NO: 60 - protein, 13 aa, Artificial Sequence
  synthetic construct
GTIKSINAMHWFR

SEQ ID NO: 61 - protein, 13 aa, Artificial Sequence
  synthetic construct
GSRDSINTAHWYR

SEQ ID NO: 62 - protein, 13 aa, Artificial Sequence
  synthetic construct
GSYASIASMHWFR

SEQ ID NO: 63 - protein, 13 aa, Artificial Sequence
  synthetic construct
GGEFSDNGLHWFR

SEQ ID NO: 64 - protein, 13 aa, Artificial Sequence
  synthetic construct
GFSESINATAWFR

SEQ ID NO: 65 - protein, 13 aa, Artificial Sequence
  synthetic construct
GSSDSWNALHWFR

SEQ ID NO: 66 - protein, 13 aa, Artificial Sequence
  synthetic construct
GSAFSLNYMAWFR

SEQ ID NO: 67 - protein, 13 aa, Artificial Sequence
  synthetic construct
GSTLSNNAMHWFR

SEQ ID NO: 68 - protein, 13 aa, Artificial Sequence
  synthetic construct
GSIQSYEAMHWFR

SEQ ID NO: 69 - protein, 13 aa, Artificial Sequence
  synthetic construct
GSIKNTNAMHWFR

SEQ ID NO: 70 - protein, 13 aa, Artificial Sequence
  synthetic construct
GGIYSPYSMHWFR

SEQ ID NO: 71 - protein, 13 aa, Artificial Sequence
  synthetic construct
GAAFSKNALHWFR

SEQ ID NO: 72 - protein, 13 aa, Artificial Sequence
  synthetic construct
GSIASINAIHWVR

SEQ ID NO: 73 - protein, 13 aa, Artificial Sequence
  synthetic construct
GSIISRVKVHWFR

SEQ ID NO: 74 - protein, 13 aa, Artificial Sequence
  synthetic construct
GSIENIERMHWYR

SEQ ID NO: 75 - protein, 13 aa, Artificial Sequence
  synthetic construct
GDIYSQSAMAWYR

SEQ ID NO: 76 - protein, 13 aa, Artificial Sequence
  synthetic construct
GYIFEGGPTAWFR

SEQ ID NO: 77 - protein, 13 aa, Artificial Sequence
  synthetic construct
GSYFSKNAVHWYR

SEQ ID NO: 78 - protein, 13 aa, Artificial Sequence
  synthetic construct
GFEFTYRAVHWFR

SEQ ID NO: 79 - protein, 13 aa, Artificial Sequence
  synthetic construct
GSSFRILAMHWYR

SEQ ID NO: 80 - protein, 13 aa, Artificial Sequence
  synthetic construct
GLRFAMTEMAWFR

SEQ ID NO: 81 - protein, 13 aa, Artificial Sequence
  synthetic construct
GSSFEGNAVHWFR

SEQ ID NO: 82 - protein, 13 aa, Artificial Sequence
  synthetic construct
GENFSGNLVHWFR

SEQ ID NO: 83 - protein, 13 aa, Artificial Sequence
  synthetic construct
GADFSSNAMHWFR

SEQ ID NO: 84 - protein, 13 aa, Artificial Sequence
  synthetic construct
GSMYSRAVMHWFR

SEQ ID NO: 85 - protein, 13 aa, Artificial Sequence
  synthetic construct
GYYFQIVAMHWYR

SEQ ID NO: 86 - protein, 13 aa, Artificial Sequence
  synthetic construct
GIDDSIGAMHWFR

SEQ ID NO: 87 - protein, 13 aa, Artificial Sequence
  synthetic construct
GNYFEINKMHWFR

SEQ ID NO: 88 - protein, 13 aa, Artificial Sequence
  synthetic construct
GLQTSDNAMHWFR

SEQ ID NO: 89 - protein, 13 aa, Artificial Sequence
  synthetic construct
GSISEINLLHWFR

SEQ ID NO: 90 - protein, 13 aa, Artificial Sequence
  synthetic construct
GGEKSISAAHWYR

SEQ ID NO: 91 - protein, 13 aa, Artificial Sequence
  synthetic construct
GEDYSYNAAHWFR

SEQ ID NO: 92 - protein, 13 aa, Artificial Sequence
  synthetic construct
GSISSINSMHWYR

SEQ ID NO: 93 - protein, 13 aa, Artificial Sequence
  synthetic construct
GPQFSSNAVSWFR

SEQ ID NO: 94 - protein, 13 aa, Artificial Sequence
  synthetic construct
GSQFSHNLMSWFR

SEQ ID NO: 95 - protein, 13 aa, Artificial Sequence
  synthetic construct
GYILSENLTAWFR

SEQ ID NO: 96 - protein, 13 aa, Artificial Sequence
  synthetic construct
GIPASYLNMHWFR

SEQ ID NO: 97 - protein, 13 aa, Artificial Sequence
  synthetic construct
GSTGSDYAMHWFR

SEQ ID NO: 98 - protein, 19 aa, Artificial Sequence
  synthetic construct
KQREWVAAITTGGSTLYAD

SEQ ID NO: 99 - protein, 19 aa, Artificial Sequence
  synthetic construct
KGRELVASIDSGSLDYYAD

SEQ ID NO: 100 - protein, 19 aa, Artificial Sequence
  synthetic construct
KQREEVAAITSGGSTGYAD

SEQ ID NO: 101 - protein, 19 aa, Artificial Sequence
  synthetic construct
KQREWVAGITSGTSGNYAD

SEQ ID NO: 102 - protein, 19 aa, Artificial Sequence
  synthetic construct
KQREWVAGVTSDNSTFYAD

SEQ ID NO: 103 - protein, 19 aa, Artificial Sequence
  synthetic construct
KGREGVASITSGGGTYYED

SEQ ID NO: 104 - protein, 19 aa, Artificial Sequence
  synthetic construct
KRREFVAAITPNNGTNYAD

SEQ ID NO: 105 - protein, 19 aa, Artificial Sequence
  synthetic construct
KDREGVASISQTGRGSYAD

SEQ ID NO: 106 - protein, 19 aa, Artificial Sequence
  synthetic construct
KQREFVARITSGTSTYYVD

SEQ ID NO: 107 - protein, 19 aa, Artificial Sequence
  synthetic construct
KQREFVAGLTSGGSRIYGD

SEQ ID NO: 108 - protein, 19 aa, Artificial Sequence
  synthetic construct
KGREFVAAIASGGSTFYAD

SEQ ID NO: 109 - protein, 19 aa, Artificial Sequence
  synthetic construct
KQREIVAAIDNGGSHAYGD

SEQ ID NO: 110 - protein, 19 aa, Artificial Sequence
  synthetic construct
KQREKVARITGAGSSFYAD

SEQ ID NO: 111 - protein, 19 aa, Artificial Sequence
  synthetic construct
KEREGVAGITTGGSNYYSD

SEQ ID NO: 112 - protein, 19 aa, Artificial Sequence
  synthetic construct
KAREAVALLTGAGTTIYGD

SEQ ID NO: 113 - protein, 19 aa, Artificial Sequence
  synthetic construct
KGREWVASITSAGNTYYGD

SEQ ID NO: 114 - protein, 19 aa, Artificial Sequence
  synthetic construct
KSREGVATIHSGGGTYYSD

SEQ ID NO: 115 - protein, 19 aa, Artificial Sequence
  synthetic construct
KEREGVAGITWQGSTYYAD

SEQ ID NO: 116 - protein, 19 aa, Artificial Sequence
  synthetic construct
KNREFVASIGSGSSSYYAD

SEQ ID NO: 117 - protein, 19 aa, Artificial Sequence
  synthetic construct
KGREFVAAITSGGNDNYSD

SEQ ID NO: 118 - protein, 19 aa, Artificial Sequence
  synthetic construct
KQREFVATINSGGSSYYAD

SEQ ID NO: 119 - protein, 19 aa, Artificial Sequence
  synthetic construct
KQREFVAGITSNGSINYKD

SEQ ID NO: 120 - protein, 19 aa, Artificial Sequence
  synthetic construct
KGREWVASITSGASTQYAD

SEQ ID NO: 121 - protein, 19 aa, Artificial Sequence
  synthetic construct
KGREWVAAVTLGGHSNYAD

SEQ ID NO: 122 - protein, 19 aa, Artificial Sequence
  synthetic construct
KQREEVAAITSGGSTGYAD

SEQ ID NO: 123 - protein, 19 aa, Artificial Sequence
  synthetic construct
KQREEVAAITSGGSTGYAD

SEQ ID NO: 124 - protein, 19 aa, Artificial Sequence
  synthetic construct
KEREGVAGITWQGSTYYAD

SEQ ID NO: 125 - protein, 19 aa, Artificial Sequence
  synthetic construct
KEREFVASVSRGGSNNYRD

SEQ ID NO: 126 - protein, 19 aa, Artificial Sequence
  synthetic construct
KEREMVAAITSAESVNYED

SEQ ID NO: 127 - protein, 19 aa, Artificial Sequence
  synthetic construct
KQREGVATMASGGDTSYAD

SEQ ID NO: 128 - protein, 19 aa, Artificial Sequence
  synthetic construct
KEREWVASMTWSDMTQYSD

SEQ ID NO: 129 - protein, 19 aa, Artificial Sequence
  synthetic construct
KEREGVAGLTSEGSRYYAD

SEQ ID NO: 130 - protein, 19 aa, Artificial Sequence
  synthetic construct
KGREWVASITSGSGTQYMD

SEQ ID NO: 131 - protein, 19 aa, Artificial Sequence
  synthetic construct
KQRELVAGITSPSSTYYAD

SEQ ID NO: 132 - protein, 19 aa, Artificial Sequence
  synthetic construct
KEREGVAGITWQGSTYYAD

SEQ ID NO: 133 - protein, 19 aa, Artificial Sequence
  synthetic construct
KQREFVAGIYSDGSSLYAD

SEQ ID NO: 134 - protein, 19 aa, Artificial Sequence
  synthetic construct
KQREGVAGITSGSVSFYAD

SEQ ID NO: 135 - protein, 19 aa, Artificial Sequence
  synthetic construct
KSREGVASINSGGHADYSD

SEQ ID NO: 136 - protein, 19 aa, Artificial Sequence
  synthetic construct
KEREAVAIITNASQTLYRD

SEQ ID NO: 137 - protein, 19 aa, Artificial Sequence
  synthetic construct
KEREFVASISVGDSRSYRD

SEQ ID NO: 138 - protein, 19 aa, Artificial Sequence
  synthetic construct
KQRELVASLTSGDNRYYTD

SEQ ID NO: 139 - protein, 19 aa, Artificial Sequence
  synthetic construct
KERELVAAIYSGGNDNYSD

SEQ ID NO: 140 - protein, 19 aa, Artificial Sequence
  synthetic construct
KQREAVATITSGSNSTYAD

SEQ ID NO: 141 - protein, 19 aa, Artificial Sequence
  synthetic construct
KEREWVAVIDSGGYTYYAD

SEQ ID NO: 142 - protein, 19 aa, Artificial Sequence
  synthetic construct
KAREEVAAITENDVDYYAD

SEQ ID NO: 143 - protein, 19 aa, Artificial Sequence
  synthetic construct
KQREWVAGLTSSGSDGYVD

SEQ ID NO: 144 - protein, 19 aa, Artificial Sequence
  synthetic construct
KGREWVAAITTGNDEAYAD

SEQ ID NO: 145 - protein, 19 aa, Artificial Sequence
  synthetic construct
KQREGVAGITGGSNGYYAD

SEQ ID NO: 146 - protein, 19 aa, Artificial Sequence
  synthetic construct
KGREGVAGITSRWVTYYAD

SEQ ID NO: 147 - protein, 19 aa, Artificial Sequence
  synthetic construct
KEREGVAGITSGGGSYYAD

SEQ ID NO: 148 - protein, 19 aa, Artificial Sequence
  synthetic construct
KQREFVATITSTSSTAYRD

SEQ ID NO: 149 - protein, 19 aa, Artificial Sequence
  synthetic construct
KQREGVASINSTGSDYYAD

SEQ ID NO: 150 - protein, 19 aa, Artificial Sequence
  synthetic construct
KQRELVALIAKGGSGYYAD

SEQ ID NO: 151 - protein, 19 aa, Artificial Sequence
  synthetic construct
KRREFVATATTGGTTSYAD

SEQ ID NO: 152 - protein, 19 aa, Artificial Sequence
  synthetic construct
KQREFVAAITITGSSYYAD

SEQ ID NO: 153 - protein, 19 aa, Artificial Sequence
  synthetic construct
KEREFVATITNGLSARYKD

SEQ ID NO: 154 - protein, 19 aa, Artificial Sequence
  synthetic construct
KNREGVAGITSGGSTYYAD

SEQ ID NO: 155 - protein, 19 aa, Artificial Sequence
  synthetic construct
KGREGVASIGWGGGTYYAD

SEQ ID NO: 156 - protein, 19 aa, Artificial Sequence
  synthetic construct
KQREWVARITSGGSDNYGD

SEQ ID NO: 157 - protein, 19 aa, Artificial Sequence
  synthetic construct
KAREFVASIGSNGAKSYSD

SEQ ID NO: 158 - protein, 19 aa, Artificial Sequence
  synthetic construct
KAREWVASLDNSTTGSYAD

SEQ ID NO: 159 - protein, 19 aa, Artificial Sequence
  synthetic construct
KEREFVAGVTSGGSTNYGD

SEQ ID NO: 160 - protein, 19 aa, Artificial Sequence
  synthetic construct
KQREFVATASSDGSTQYRD

SEQ ID NO: 161 - protein, 19 aa, Artificial Sequence
  synthetic construct
KQREGVAGLDGGGSDYYSD

SEQ ID NO: 162 - protein, 19 aa, Artificial Sequence
  synthetic construct
KEREFVAGISSAGSGYYAD

SEQ ID NO: 163 - protein, 19 aa, Artificial Sequence
  synthetic construct
KQREGVAIIGSGGSSDYAD

SEQ ID NO: 164 - protein, 19 aa, Artificial Sequence
  synthetic construct
KAREFVAAIGSSDSTDYKD

SEQ ID NO: 165 - protein, 19 aa, Artificial Sequence
  synthetic construct
KAREWVAVITWGDSTQYGD

SEQ ID NO: 166 - protein, 19 aa, Artificial Sequence
  synthetic construct
KDREWVAGITSGGNSNYGD

SEQ ID NO: 167 - protein, 19 aa, Artificial Sequence
  synthetic construct
KAREWVAGIVGGGGTTYSD

SEQ ID NO: 168 - protein, 19 aa, Artificial Sequence
  synthetic construct
KGREWVASITSGSGTQYMD

SEQ ID NO: 169 - protein, 19 aa, Artificial Sequence
  synthetic construct
KQREEVAAITSGGSTGYAD

SEQ ID NO: 170 - protein, 19 aa, Artificial Sequence
  synthetic construct
KSREWVAGIDSGTASYYSD

SEQ ID NO: 171 - protein, 19 aa, Artificial Sequence
  synthetic construct
KQRELVAVIDSGGTTRYAD

SEQ ID NO: 172 - protein, 19 aa, Artificial Sequence
  synthetic construct
KEREFVAAITTTGSRSYKD

SEQ ID NO: 173 - protein, 19 aa, Artificial Sequence
  synthetic construct
KGREFVAAITTSGETSYAY

SEQ ID NO: 174 - protein, 19 aa, Artificial Sequence
  synthetic construct
KGREWVAALTNSGSGNYKD

SEQ ID NO: 175 - protein, 19 aa, Artificial Sequence
  synthetic construct
KGREWVAGVVSGGSTYYAD

SEQ ID NO: 176 - protein, 19 aa, Artificial Sequence
  synthetic construct
KNREWVAVITSNGNTYYAD

SEQ ID NO: 177 - protein, 19 aa, Artificial Sequence
  synthetic construct
KQREFVALITEGGSGSYGD

SEQ ID NO: 178 - protein, 19 aa, Artificial Sequence
  synthetic construct
KNREFVATITTSTSYYYAD

SEQ ID NO: 179 - protein, 19 aa, Artificial Sequence
  synthetic construct
KAREGVATVTSTGSGYYAD

SEQ ID NO: 180 - protein, 19 aa, Artificial Sequence
  synthetic construct
KAREFVASIDSDKSGYYAD

SEQ ID NO: 181 - protein, 19 aa, Artificial Sequence
  synthetic construct
KEREGVASIGRTGDSYYAD

SEQ ID NO: 182 - protein, 19 aa, Artificial Sequence
  synthetic construct
KEREGVAGITSTGSTYYGD

SEQ ID NO: 183 - protein, 19 aa, Artificial Sequence
  synthetic construct
KGREFVAALSGTGTTSYAD

SEQ ID NO: 184 - protein, 19 aa, Artificial Sequence
  synthetic construct
KQREFVATIGSGGSTAYAD

SEQ ID NO: 185 - protein, 19 aa, Artificial Sequence
  synthetic construct
KNRELVAGLTSGTSRNYGD

SEQ ID NO: 186 - protein, 19 aa, Artificial Sequence
  synthetic construct
KQREWVATMTSGGSTDYAD

SEQ ID NO: 187 - protein, 19 aa, Artificial Sequence
  synthetic construct
KEREFVAAITAASTTSYAD

SEQ ID NO: 188 - protein, 19 aa, Artificial Sequence
  synthetic construct
KEREWVAAINSGGGTYYAD

SEQ ID NO: 189 - protein, 19 aa, Artificial Sequence
  synthetic construct
KEREWVAAVSSYGSDYYAD

SEQ ID NO: 190 - protein, 19 aa, Artificial Sequence
  synthetic construct
KEREWVAGITSGGSTDYAD

SEQ ID NO: 191 - protein, 19 aa, Artificial Sequence
  synthetic construct
KQREFVAAITSFGSTGYAD

SEQ ID NO: 192 - protein, 19 aa, Artificial Sequence
  synthetic construct
KAREVVATVTSGDYTNYDD

SEQ ID NO: 193 - protein, 19 aa, Artificial Sequence
  synthetic construct
KQREWVAGAGGTNSNYYQD

SEQ ID NO: 194 - protein, 19 aa, Artificial Sequence
  synthetic construct
KDREWVAGISSYGSDSYGD

SEQ ID NO: 195 - protein, 12 aa, Artificial Sequence
  synthetic construct
CNADRSYFPDYW

SEQ ID NO: 196 - protein, 12 aa, Artificial Sequence
  synthetic construct
CAASRSGDYRYW

SEQ ID NO: 197 - protein, 15 aa, Artificial Sequence
  synthetic construct
CAATSTRGGLDSDYW

SEQ ID NO: 198 - protein, 14 aa, Artificial Sequence
  synthetic construct
CAATSHTTYHAEYW

SEQ ID NO: 199 - protein, 13 aa, Artificial Sequence
  synthetic construct
CTATVKTSQYRYW

SEQ ID NO: 200 - protein, 13 aa, Artificial Sequence
  synthetic construct
CAASEAWRRFDSW

SEQ ID NO: 201 - protein, 17 aa, Artificial Sequence
  synthetic construct
CTAVLWYTTKEGPYDYW

SEQ ID NO: 202 - protein, 14 aa, Artificial Sequence
  synthetic construct
CAAASRTGTPYNYW

SEQ ID NO: 203 - protein, 13 aa, Artificial Sequence
  synthetic construct
CNGYEHTTGYDYW

SEQ ID NO: 204 - protein, 15 aa, Artificial Sequence
  synthetic construct
CTALILHYAGMYDYW

SEQ ID NO: 205 - protein, 18 aa, Artificial Sequence
  synthetic construct
CAAIPRPSLSYAHDYAYW

SEQ ID NO: 206 - protein, 19 aa, Artificial Sequence
  synthetic construct
CAVFPDHDSEQASIDRVYW

SEQ ID NO: 207 - protein, 18 aa, Artificial Sequence
  synthetic construct
CNADSIPPALRAANTGYW

SEQ ID NO: 208 - protein, 16 aa, Artificial Sequence
  synthetic construct
CNALGLGSASGSYESW

SEQ ID NO: 209 - protein, 16 aa, Artificial Sequence
  synthetic construct
CAAGQGFKATRTFDSW

SEQ ID NO: 210 - protein, 19 aa, Artificial Sequence
  synthetic construct
CNALAGANYESQWEEYNYW

SEQ ID NO: 211 - protein, 20 aa, Artificial Sequence
  synthetic construct
CNAEVSTGAGSRGRVSDVYW

SEQ ID NO: 212 - protein, 18 aa, Artificial Sequence
  synthetic construct
CNALSADGAYVRRYKDYW

SEQ ID NO: 213 - protein, 17 aa, Artificial Sequence
  synthetic construct
CTALGGDELDGMTYDYW

SEQ ID NO: 214 - protein, 17 aa, Artificial Sequence
  synthetic construct
CNAPATEDTSHYPRNYW

SEQ ID NO: 215 - protein, 12 aa, Artificial Sequence
  synthetic construct
CTALLDSTYDYW

SEQ ID NO: 216 - protein, 17 aa, Artificial Sequence
  synthetic construct
CTALLMSYGGGEEYDYW

SEQ ID NO: 217 - protein, 16 aa, Artificial Sequence
  synthetic construct
CAADRYRAIQRTQYSW

SEQ ID NO: 218 - protein, 21 aa, Artificial Sequence
  synthetic construct
CNYPVGIPAVAIDGRRSYGYW

SEQ ID NO: 219 - protein, 17 aa, Artificial Sequence
  synthetic construct
CNAGVVWLPSNAAYNLW

SEQ ID NO: 220 - protein, 15 aa, Artificial Sequence
  synthetic construct
CAAQQARGGLDSDYW

SEQ ID NO: 221 - protein, 14 aa, Artificial Sequence
  synthetic construct
CNAVAPVHENENYW

SEQ ID NO: 222 - protein, 15 aa, Artificial Sequence
  synthetic construct
CAALLETSSGDYTYW

SEQ ID NO: 223 - protein, 15 aa, Artificial Sequence
  synthetic construct
CATYDYTDDYGYASW

SEQ ID NO: 224 - protein, 17 aa, Artificial Sequence
  synthetic construct
CAAQTYRPDTWGRYTFW

SEQ ID NO: 225 - protein, 15 aa, Artificial Sequence
  synthetic construct
CAATYVTRDRPWTHW

SEQ ID NO: 226 - protein, 15 aa, Artificial Sequence
  synthetic construct
CAAEPTGNTHEFEYW

SEQ ID NO: 227 - protein, 12 aa, Artificial Sequence
  synthetic construct
CAAANDGYGVDW

SEQ ID NO: 228 - protein, 22 aa, Artificial Sequence
  synthetic construct
CNTELEGKTQGFTGKTVWYDYW

SEQ ID NO: 229 - protein, 14 aa, Artificial Sequence
  synthetic construct
CATVWAGGTPNDYW

SEQ ID NO: 230 - protein, 13 aa, Artificial Sequence
  synthetic construct
CAALDYSARYRTW

SEQ ID NO: 231 - protein, 19 aa, Artificial Sequence
  synthetic construct
CAAVTGSSTGNAGQGYLTW

SEQ ID NO: 232 - protein, 14 aa, Artificial Sequence
  synthetic construct
CAAGPYGAYNFNSW

SEQ ID NO: 233 - protein, 15 aa, Artificial Sequence
  synthetic construct
CAADGADVVYGNRYW

SEQ ID NO: 234 - protein, 16 aa, Artificial Sequence
  synthetic construct
CAAQIYITTYELYDYW

SEQ ID NO: 235 - protein, 17 aa, Artificial Sequence
  synthetic construct
CNAKTVGSSYRDSYNYW

SEQ ID NO: 236 - protein, 16 aa, Artificial Sequence
  synthetic construct
CAADQAWVRAGTYDYW

SEQ ID NO: 237 - protein, 18 aa, Artificial Sequence
  synthetic construct
CAARNGKLSPKANQRLYW

SEQ ID NO: 238 - protein, 17 aa, Artificial Sequence
  synthetic construct
CNAVPVTTDKTPMYQYW

SEQ ID NO: 239 - protein, 16 aa, Artificial Sequence
  synthetic construct
CASLSSGYQYVGFRYW

SEQ ID NO: 240 - protein, 15 aa, Artificial Sequence
  synthetic construct
CNAFSYHTYSRYDFW

SEQ ID NO: 241 - protein, 18 aa, Artificial Sequence
  synthetic construct
CAANTYERTPDGRTFSYW

SEQ ID NO: 242 - protein, 16 aa, Artificial Sequence
  synthetic construct
CAAVATSYTGVEYEYW

SEQ ID NO: 243 - protein, 15 aa, Artificial Sequence
  synthetic construct
CNAPVYYDTEWYDIW

SEQ ID NO: 244 - protein, 21 aa, Artificial Sequence
  synthetic construct
CNALGLGQSSLYRSADPYDSW

SEQ ID NO: 245 - protein, 20 aa, Artificial Sequence
  synthetic construct
CAAFAVRDADTAGWTARDYW

SEQ ID NO: 246 - protein, 22 aa, Artificial Sequence
  synthetic construct
CAAAITQPMPQGKGAPPGFYYW

SEQ ID NO: 247 - protein, 17 aa, Artificial Sequence
  synthetic construct
CAADRTYGYEYSTYQYW

SEQ ID NO: 248 - protein, 15 aa, Artificial Sequence
  synthetic construct
CTALLMSRAGEFDYW

SEQ ID NO: 249 - protein, 15 aa, Artificial Sequence
  synthetic construct
CNTVDRTDNYGYDYW

SEQ ID NO: 250 - protein, 16 aa, Artificial Sequence
  synthetic construct
CAAQVYITTYELYDYW

SEQ ID NO: 251 - protein, 15 aa, Artificial Sequence
  synthetic construct
CAAKPLGSPDDFDYW

SEQ ID NO: 252 - protein, 15 aa, Artificial Sequence
  synthetic construct
CAAKFRENFSFYDYW

SEQ ID NO: 253 - protein, 15 aa, Artificial Sequence
  synthetic construct
CAAYVGTDNYGYDYW

SEQ ID NO: 254 - protein, 16 aa, Artificial Sequence
  synthetic construct
CTALITVAESDDYDYW

SEQ ID NO: 255 - protein, 17 aa, Artificial Sequence
  synthetic construct
CAAVTGTPGGLGFYDYW

SEQ ID NO: 256 - protein, 19 aa, Artificial Sequence
  synthetic construct
CNALAGANYESQWEEYNYW

SEQ ID NO: 257 - protein, 15 aa, Artificial Sequence
  synthetic construct
CTALLMSSAGEYDVW

SEQ ID NO: 258 - protein, 22 aa, Artificial Sequence
  synthetic construct
CAAQLTSSFRGYDFTPSNYDYW

SEQ ID NO: 259 - protein, 13 aa, Artificial Sequence
  synthetic construct
CNGYEHTTGYDYW

SEQ ID NO: 260 - protein, 15 aa, Artificial Sequence
  synthetic construct
CAAQPNGSPDDFDYW

SEQ ID NO: 261 - protein, 15 aa, Artificial Sequence
  synthetic construct
CTALLDSGPGEYNYW

SEQ ID NO: 262 - protein, 12 aa, Artificial Sequence
  synthetic construct
CAADVRQGYSSW

SEQ ID NO: 263 - protein, 17 aa, Artificial Sequence
  synthetic construct
CAAFGYRYDQDYTYDYW

SEQ ID NO: 264 - protein, 14 aa, Artificial Sequence
  synthetic construct
CNANYQEYDEYDYW

SEQ ID NO: 265 - protein, 13 aa, Artificial Sequence
  synthetic construct
CAALAYSGGYDYW

SEQ ID NO: 266 - protein, 12 aa, Artificial Sequence
  synthetic construct
CAAQPGQSYAVW

SEQ ID NO: 267 - protein, 12 aa, Artificial Sequence
  synthetic construct
CAAENRGYGVDW

SEQ ID NO: 268 - protein, 16 aa, Artificial Sequence
  synthetic construct
CAAVWLTNAESTQEYW

SEQ ID NO: 269 - protein, 18 aa, Artificial Sequence
  synthetic construct
CAAFAVRSSSVSDAYDYW

SEQ ID NO: 270 - protein, 14 aa, Artificial Sequence
  synthetic construct
CNATDRDETGYDYW

SEQ ID NO: 271 - protein, 16 aa, Artificial Sequence
  synthetic construct
CTANISESTGQGYDYW

SEQ ID NO: 272 - protein, 15 aa, Artificial Sequence
  synthetic construct
CNAGPQAEEDTYDYW

SEQ ID NO: 273 - protein, 17 aa, Artificial Sequence
  synthetic construct
CNADTLDLDDGATYNYW

SEQ ID NO: 274 - protein, 21 aa, Artificial Sequence
  synthetic construct
CAALHYTEGANTESATTFDYW

SEQ ID NO: 275 - protein, 17 aa, Artificial Sequence
  synthetic construct
CAARLKDRGPQSEYDYW

SEQ ID NO: 276 - protein, 15 aa, Artificial Sequence
  synthetic construct
CAAKHQKGEIPYYDW

SEQ ID NO: 277 - protein, 15 aa, Artificial Sequence
  synthetic construct
CNAYSGTDNYGYDYW

SEQ ID NO: 278 - protein, 15 aa, Artificial Sequence
  synthetic construct
CAADNLDVVYGNRYW

SEQ ID NO: 279 - protein, 15 aa, Artificial Sequence
  synthetic construct
CNAVSTDAQGAAEYW

SEQ ID NO: 280 - protein, 12 aa, Artificial Sequence
  synthetic construct
CAAAFPAGGDYW

SEQ ID NO: 281 - protein, 20 aa, Artificial Sequence
  synthetic construct
CNALIYLADRRDGYYSYDYW

SEQ ID NO: 282 - protein, 18 aa, Artificial Sequence
  synthetic construct
CAAFPDVGDASSIGFVYW

SEQ ID NO: 283 - protein, 15 aa, Artificial Sequence
  synthetic construct
CAADEFTKYGHGRYW

SEQ ID NO: 284 - protein, 12 aa, Artificial Sequence
  synthetic construct
CTAVLYDSYDYW

SEQ ID NO: 285 - protein, 15 aa, Artificial Sequence
  synthetic construct
CAYLGALDRDGYDAW

SEQ ID NO: 286 - protein, 15 aa, Artificial Sequence
  synthetic construct
CAARTVTGFTYGDYW

SEQ ID NO: 287 - protein, 17 aa, Artificial Sequence
  synthetic construct
CTAPSVSDADRRTRDYW

SEQ ID NO: 288 - protein, 17 aa, Artificial Sequence
  synthetic construct
CAALSSEDTSHYPRNYW

SEQ ID NO: 289 - protein, 15 aa, Artificial Sequence
  synthetic construct
CAAGTSDWEVKWSHW

SEQ ID NO: 290 - protein, 17 aa, Artificial Sequence
  synthetic construct
CAALDGAESYDYVYPVW

SEQ ID NO: 291 - protein, 17 aa, Artificial Sequence
  synthetic construct
CAALQASAGAGYYYDYW

SEQ ID NO: 292 - protein, 116 aa, Artificial Sequence
  synthetic construct
QVQLVESGGGLVQPGGSLRLSCAASGQITAENYMHWYRQAPGKQREWVAAITTGGSTLYADSVKGRFTISRDNAKNTVYLQMNSLKPEDTAVYYCNADRSYFPDYWGQGTQVTVSS

SEQ ID NO: 293 - protein, 116 aa, Artificial Sequence
  synthetic construct
QVQLVESGGGLVQPGGSLRLSCAASGSIREINAMHWVRQAPGKGRELVASIDSGSLDYYADSVKGRFTISRDNAKNTVYLQMNSLKPEDTAVYYCAASRSGDYRYWGQGTQVTVSS

SEQ ID NO: 294 - protein, 119 aa, Artificial Sequence
  synthetic construct
QVQLVESGGGLVQPGGSLRLSCAASGSILQINAIHWFRQAPGKQREEVAAITSGGSTGYADSVKGRFTISRDNAKNTVYLQMNSLKPEDTAVYYCAATSTRGGLDSDYWGQGTQVTVSS

SEQ ID NO: 295 - protein, 118 aa, Artificial Sequence
  synthetic construct
QVQLVESGGGLVQPGGSLRLSCAASGRQFGAWRMHWYRQAPGKQREWVAGITSGTSGNYADSVKGRFTISRDNAKNTVYLQMNSLKPEDTAVYYCAATSHTTYHAEYWGQGTQVTVSS

SEQ ID NO: 296 - protein, 117 aa, Artificial Sequence
  synthetic construct
QVQLVESGGGLVQPGGSLRLSCAASGTEYDISAAHWYRQAPGKQREWVAGVTSDNSTFYADSVKGRFTISRDNAKNTVYLQMNSLKPEDTAVYYCTATVKTSQYRYWGQGTQVTVSS

SEQ ID NO: 297 - protein, 117 aa, Artificial Sequence
  synthetic construct
QVQLVESGGGLVQPGGSLRLSCAASGSSININYMHWFRQAPGKGREGVASITSGGGTYYEDSVKGRFTISRDNAKNTVYLQMNSLKPEDTAVYYCAASEAWRRFDSWGQGTQVTVSS

SEQ ID NO: 298 - protein, 121 aa, Artificial Sequence
  synthetic construct
QVQLVESGGGLVQPGGSLRLSCAASGTWGNIQYLSWFRQAPGKRREFVAAITPNNGTNYADSVKGRFTISRDNAKNTVYLQMNSLKPEDTAVYYCTAVLWYTTKEGPYDYWGQGTQVTVSS

SEQ ID NO: 299 - protein, 118 aa, Artificial Sequence
  synthetic construct
QVQLVESGGGLVQPGGSLRLSCAASGSITSINWMHWFRQAPGKDREGVASISQTGRGSYADSVKGRFTISRDNAKNTVYLQMNSLKPEDTAVYYCAAASRTGTPYNYWGQGTQVTVSS

SEQ ID NO: 300 - protein, 117 aa, Artificial Sequence
  synthetic construct
QVQLVESGGGLVQPGGSLRLSCAASGSSESWNAIHWFRQAPGKQREFVARITSGTSTYYVDSVKGRFTISRDNAKNTVYLQMNSLKPEDTAVYYCNGYEHTTGYDYWGQGTQVTVSS

SEQ ID NO: 301 - protein, 119 aa, Artificial Sequence
  synthetic construct
QVQLVESGGGLVQPGGSLRLSCAASGSYASIASMHWFRQAPGKQREFVAGLTSGGSRIYGDSVKGRFTISRDNAKNTVYLQMNSLKPEDTAVYYCTALILHYAGMYDYWGQGTQVTVSS

SEQ ID NO: 302 - protein, 122 aa, Artificial Sequence
  synthetic construct
QVQLVESGGGLVQPGGSLRLSCAASGYADEINMVHWYRQAPGKGREFVAAIASGGSTFYADSVKGRFTISRDNAKNTVYLQMNSLKPEDTAVYYCAAIPRPSLSYAHDYAYWGQGTQVTVSS

SEQ ID NO: 303 - protein, 123 aa, Artificial Sequence
  synthetic construct
QVQLVESGGGLVQPGGSLRLSCAASGGVTRGNAMAWYRQAPGKQREIVAAIDNGGSHAYGDSVKGRFTISRDNAKNTVYLQMNSLKPEDTAVYYCAVFPDHDSEQASIDRVYWGQGTQVTVSS

SEQ ID NO: 304 - protein, 122 aa, Artificial Sequence
  synthetic construct
QVQLVESGGGLVQPGGSLRLSCAASGTYASYETIAWYRQAPGKQREKVARITGAGSSFYADSVKGRFTISRDNAKNTVYLQMNSLKPEDTAVYYCNADSIPPALRAANTGYWGQGTQVTVSS

SEQ ID NO: 305 - protein, 120 aa, Artificial Sequence
  synthetic construct
QVQLVESGGGLVQPGGSLRLSCAASGRILAANIVHWYRQAPGKEREGVAGITTGGSNYYSDSVKGRFTISRDNAKNTVYLQMNSLKPEDTAVYYCNALGLGSASGSYESWGQGTQVTVSS

SEQ ID NO: 306 - protein, 120 aa, Artificial Sequence
  synthetic construct
QVQLVESGGGLVQPGGSLRLSCAASGSIYKYSDVHWFRQAPGKAREAVALLTGAGTTIYGDSVKGRFTISRDNAKNTVYLQMNSLKPEDTAVYYCAAGQGFKATRTFDSWGQGTQVTVSS

SEQ ID NO: 307 - protein, 123 aa, Artificial Sequence
  synthetic construct
QVQLVESGGGLVQPGGSLRLSCAASGSGASIGAMHWFRQAPGKGREWVASITSAGNTYYGDSVKGRFTISRDNAKNTVYLQMNSLKPEDTAVYYCNALAGANYESQWEEYNYWGQGTQVTVSS

SEQ ID NO: 308 - protein, 124 aa, Artificial Sequence
  synthetic construct
QVQLVESGGGLVQPGGSLRLSCAASGHIFSINNAAWVRQAPGKSREGVATIHSGGGTYYSDSVKGRFTISRDNAKNTVYLQMNSLKPEDTAVYYCNAEVSTGAGSRGRVSDVYWGQGTQVTVSS

SEQ ID NO: 309 - protein, 122 aa, Artificial Sequence
  synthetic construct
QVQLVESGGGLVQPGGSLRLSCAASGSAMMINRTHWFRQAPGKEREGVAGITWQGSTYYADSVKGRFTISRDNAKNTVYLQMNSLKPEDTAVYYCNALSADGAYVRRYKDYWGQGTQVTVSS

SEQ ID NO: 310 - protein, 121 aa, Artificial Sequence
  synthetic construct
QVQLVESGGGLVQPGGSLRLSCAASGHQLSTNATHWFRQAPGKNREFVASIGSGSSSYYADSVKGRFTISRDNAKNTVYLQMNSLKPEDTAVYYCTALGGDELDGMTYDYWGQGTQVTVSS

SEQ ID NO: 311 - protein, 121 aa, Artificial Sequence
  synthetic construct
QVQLVESGGGLVQPGGSLRLSCAASGETFSSNAMHWFRQAPGKGREFVAAITSGGNDNYSDSVKGRFTISRDNAKNTVYLQMNSLKPEDTAVYYCNAPATEDTSHYPRNYWGQGTQVTVSS

SEQ ID NO: 312 - protein, 116 aa, Artificial Sequence
  synthetic construct
QVQLVESGGGLVQPGGSLRLSCAASGAELSYTAIHWFRQAPGKQREFVATINSGGSSYYADSVKGRFTISRDNAKNTVYLQMNSLKPEDTAVYYCTALLDSTYDYWGQGTQVTVSS

SEQ ID NO: 313 - protein, 121 aa, Artificial Sequence
  synthetic construct
QVQLVESGGGLVQPGGSLRLSCAASGSAFATNGLHWFRQAPGKQREFVAGITSNGSINYKDSVKGRFTISRDNAKNTVYLQMNSLKPEDTAVYYCTALLMSYGGGEEYDYWGQGTQVTVSS

SEQ ID NO: 314 - protein, 120 aa, Artificial Sequence
  synthetic construct
QVQLVESGGGLVQPGGSLRLSCAASGQLDRSNAMAWFRQAPGKGREWVASITSGASTQYADSVKGRFTISRDNAKNTVYLQMNSLKPEDTAVYYCAADRYRAIQRTQYSWGQGTQVTVSS

SEQ ID NO: 315 - protein, 125 aa, Artificial Sequence
  synthetic construct
QVQLVESGGGLVQPGGSLRLSCAASGDDFIENAISWYRQAPGKGREWVAAVTLGGHSNYADSVKGRFTISRDNAKNTVYLQMNSLKPEDTAVYYCNYPVGIPAVAIDGRRSYGYWGQGTQVTVSS

SEQ ID NO: 316 - protein, 121 aa, Artificial Sequence
  synthetic construct
QVQLVESGGGLVQPGGSLRLSCAASGAFFSYGAIHWFRQAPGKQREEVAAITSGGSTGYADSVKGRFTISRDNAKNTVYLQMNSLKPEDTAVYYCNAGVVWLPSNAAYNLWGQGTQVTVSS

SEQ ID NO: 317 - protein, 119 aa, Artificial Sequence
  synthetic construct
QVQLVESGGGLVQPGGSLRLSCAASGDIFSINALHWFRQAPGKQREEVAAITSGGSTGYADSVKGRFTISRDNAKNTVYLQMNSLKPEDTAVYYCAAQQARGGLDSDYWGQGTQVTVSS

SEQ ID NO: 318 - protein, 118 aa, Artificial Sequence
  synthetic construct
QVQLVESGGGLVQPGGSLRLSCAASGSAMMINRTHWFRQAPGKEREGVAGITWQGSTYYADSVKGRFTISRDNAKNTVYLQMNSLKPEDTAVYYCNAVAPVHENENYWGQGTQVTVSS

SEQ ID NO: 319 - protein, 119 aa, Artificial Sequence
  synthetic construct
QVQLVESGGGLVQPGGSLRLSCAASGNDWYINSAHWYRQAPGKEREFVASVSRGGSNNYRDSVKGRFTISRDNAKNTVYLQMNSLKPEDTAVYYCAALLETSSGDYTYWGQGTQVTVSS

SEQ ID NO: 320 - protein, 119 aa, Artificial Sequence
  synthetic construct
QVQLVESGGGLVQPGGSLRLSCAASGYDFDILRMAWFRQAPGKEREMVAAITSAESVNYEDSVKGRFTISRDNAKNTVYLQMNSLKPEDTAVYYCATYDYTDDYGYASWGQGTQVTVSS

SEQ ID NO: 321 - protein, 121 aa, Artificial Sequence
  synthetic construct
QVQLVESGGGLVQPGGSLRLSCAASGSISSIEAMSWFRQAPGKQREGVATMASGGDTSYADSVKGRFTISRDNAKNTVYLQMNSLKPEDTAVYYCAAQTYRPDTWGRYTFWGQGTQVTVSS

SEQ ID NO: 322 - protein, 119 aa, Artificial Sequence
  synthetic construct
QVQLVESGGGLVQPGGSLRLSCAASGYHASILAMHWVRQAPGKEREWVASMTWSDMTQYSDSVKGRFTISRDNAKNTVYLQMNSLKPEDTAVYYCAATYVTRDRPWTHWGQGTQVTVSS

SEQ ID NO: 323 - protein, 119 aa, Artificial Sequence
  synthetic construct
QVQLVESGGGLVQPGGSLRLSCAASGGISVANVMHWFRQAPGKEREGVAGLTSEGSRYYADSVKGRFTISRDNAKNTVYLQMNSLKPEDTAVYYCAAEPTGNTHEFEYWGQGTQVTVSS

SEQ ID NO: 324 - protein, 116 aa, Artificial Sequence
  synthetic construct
QVQLVESGGGLVQPGGSLRLSCAASGSPFKTQTVHWFRQAPGKGREWVASITSGSGTQYMDSVKGRFTISRDNAKNTVYLQMNSLKPEDTAVYYCAAANDGYGVDWGQGTQVTVSS

SEQ ID NO: 325 - protein, 126 aa, Artificial Sequence
  synthetic construct
QVQLVESGGGLVQPGGSLRLSCAASGSIGQIVLVHWFRQAPGKQRELVAGITSPSSTYYADSVKGRFTISRDNAKNTVYLQMNSLKPEDTAVYYCNTELEGKTQGFTGKTVWYDYWGQGTQVTVSS

SEQ ID NO: 326 - protein, 118 aa, Artificial Sequence
  synthetic construct
QVQLVESGGGLVQPGGSLRLSCAASGLDVSINFMHWYRQAPGKEREGVAGITWQGSTYYADSVKGRFTISRDNAKNTVYLQMNSLKPEDTAVYYCATVWAGGTPNDYWGQGTQVTVSS

SEQ ID NO: 327 - protein, 117 aa, Artificial Sequence
  synthetic construct
QVQLVESGGGLVQPGGSLRLSCAASGDIFSKNAMHWVRQAPGKQREFVAGIYSDGSSLYADSVKGRFTISRDNAKNTVYLQMNSLKPEDTAVYYCAALDYSARYRTWGQGTQVTVSS

SEQ ID NO: 328 - protein, 123 aa, Artificial Sequence
  synthetic construct
QVQLVESGGGLVQPGGSLRLSCAASGSGFSNNAMHWYRQAPGKQREGVAGITSGSVSFYADSVKGRFTISRDNAKNTVYLQMNSLKPEDTAVYYCAAVTGSSTGNAGQGYLTWGQGTQVTVSS

SEQ ID NO: 329 - protein, 118 aa, Artificial Sequence
  synthetic construct
QVQLVESGGGLVQPGGSLRLSCAASGSIFSLNWVHWFRQAPGKSREGVASINSGGHADYSDSVKGRFTISRDNAKNTVYLQMNSLKPEDTAVYYCAAGPYGAYNFNSWGQGTQVTVSS

SEQ ID NO: 330 - protein, 119 aa, Artificial Sequence
  synthetic construct
QVQLVESGGGLVQPGGSLRLSCAASGTIFSQNAVHWFRQAPGKEREAVAIITNASQTLYRDSVKGRFTISRDNAKNTVYLQMNSLKPEDTAVYYCAADGADVVYGNRYWGQGTQVTVSS

SEQ ID NO: 331 - protein, 120 aa, Artificial Sequence
  synthetic construct
QVQLVESGGGLVQPGGSLRLSCAASGSFFSINAVHWFRQAPGKEREFVASISVGDSRSYRDSVKGRFTISRDNAKNTVYLQMNSLKPEDTAVYYCAAQIYITTYELYDYWGQGTQVTVSS

SEQ ID NO: 332 - protein, 121 aa, Artificial Sequence
  synthetic construct
QVQLVESGGGLVQPGGSLRLSCAASGTIPSINYTAWYRQAPGKQRELVASLTSGDNRYYTDSVKGRFTISRDNAKNTVYLQMNSLKPEDTAVYYCNAKTVGSSYRDSYNYWGQGTQVTVSS

SEQ ID NO: 333 - protein, 120 aa, Artificial Sequence
  synthetic construct
QVQLVESGGGLVQPGGSLRLSCAASGGIFSINSMAWYRQAPGKERELVAAIYSGGNDNYSDSVKGRFTISRDNAKNTVYLQMNSLKPEDTAVYYCAADQAWVRAGTYDYWGQGTQVTVSS

SEQ ID NO: 334 - protein, 122 aa, Artificial Sequence
  synthetic construct
QVQLVESGGGLVQPGGSLRLSCAASGQTNSILAMHWYRQAPGKQREAVATITSGSNSTYADSVKGRFTISRDNAKNTVYLQMNSLKPEDTAVYYCAARNGKLSPKANQRLYWGQGTQVTVSS

SEQ ID NO: 335 - protein, 121 aa, Artificial Sequence
  synthetic construct
QVQLVESGGGLVQPGGSLRLSCAASGSATSINRMAWVRQAPGKEREWVAVIDSGGYTYYADSVKGRFTISRDNAKNTVYLQMNSLKPEDTAVYYCNAVPVTTDKTPMYQYWGQGTQVTVSS

SEQ ID NO: 336 - protein, 120 aa, Artificial Sequence
  synthetic construct
QVQLVESGGGLVQPGGSLRLSCAASGSIESINAIHWFRQAPGKAREEVAAITENDVDYYADSVKGRFTISRDNAKNTVYLQMNSLKPEDTAVYYCASLSSGYQYVGFRYWGQGTQVTVSS

SEQ ID NO: 337 - protein, 119 aa, Artificial Sequence
  synthetic construct
QVQLVESGGGLVQPGGSLRLSCAASGSISSYNAMHWFRQAPGKQREWVAGLTSSGSDGYVDSVKGRFTISRDNAKNTVYLQMNSLKPEDTAVYYCNAFSYHTYSRYDFWGQGTQVTVSS

SEQ ID NO: 338 - protein, 122 aa, Artificial Sequence
  synthetic construct
QVQLVESGGGLVQPGGSLRLSCAASGSIWSIAFMSWYRQAPGKGREWVAAITTGNDEAYADSVKGRFTISRDNAKNTVYLQMNSLKPEDTAVYYCAANTYERTPDGRTFSYWGQGTQVTVSS

SEQ ID NO: 339 - protein, 120 aa, Artificial Sequence
  synthetic construct
QVQLVESGGGLVQPGGSLRLSCAASGSSTSINIMAWYRQAPGKQREGVAGITGGSNGYYADSVKGRFTISRDNAKNTVYLQMNSLKPEDTAVYYCAAVATSYTGVEYEYWGQGTQVTVSS

SEQ ID NO: 340 - protein, 119 aa, Artificial Sequence
  synthetic construct
QVQLVESGGGLVQPGGSLRLSCAASGSRDSIVAMHWYRQAPGKGREGVAGITSRWVTYYADSVKGRFTISRDNAKNTVYLQMNSLKPEDTAVYYCNAPVYYDTEWYDIWGQGTQVTVSS

SEQ ID NO: 341 - protein, 125 aa, Artificial Sequence
  synthetic construct
QVQLVESGGGLVQPGGSLRLSCAASGRILAANIVHWYRQAPGKEREGVAGITSGGGSYYADSVKGRFTISRDNAKNTVYLQMNSLKPEDTAVYYCNALGLGQSSLYRSADPYDSWGQGTQVTVSS

SEQ ID NO: 342 - protein, 124 aa, Artificial Sequence
  synthetic construct
QVQLVESGGGLVQPGGSLRLSCAASGSDFTINAMHWFRQAPGKQREFVATITSTSSTAYRDSVKGRFTISRDNAKNTVYLQMNSLKPEDTAVYYCAAFAVRDADTAGWTARDYWGQGTQVTVSS

SEQ ID NO: 343 - protein, 126 aa, Artificial Sequence
  synthetic construct
QVQLVESGGGLVQPGGSLRLSCAASGSSFSINWMHWFRQAPGKQREGVASINSTGSDYYADSVKGRFTISRDNAKNTVYLQMNSLKPEDTAVYYCAAAITQPMPQGKGAPPGFYYWGQGTQVTVSS

SEQ ID NO: 344 - protein, 121 aa, Artificial Sequence
  synthetic construct
QVQLVESGGGLVQPGGSLRLSCAASGSIYVINTMHWFRQAPGKQRELVALIAKGGSGYYADSVKGRFTISRDNAKNTVYLQMNSLKPEDTAVYYCAADRTYGYEYSTYQYWGQGTQVTVSS

SEQ ID NO: 345 - protein, 119 aa, Artificial Sequence
  synthetic construct
QVQLVESGGGLVQPGGSLRLSCAASGQISSNNGMHWFRQAPGKRREFVATATTGGTTSYADSVKGRFTISRDNAKNTVYLQMNSLKPEDTAVYYCTALLMSRAGEFDYWGQGTQVTVSS

SEQ ID NO: 346 - protein, 119 aa, Artificial Sequence
  synthetic construct
QVQLVESGGGLVQPGGSLRLSCAASGSQLSINYVHWFRQAPGKQREFVAAITITGSSYYADSVKGRFTISRDNAKNTVYLQMNSLKPEDTAVYYCNTVDRTDNYGYDYWGQGTQVTVSS

SEQ ID NO: 347 - protein, 120 aa, Artificial Sequence
  synthetic construct
QVQLVESGGGLVQPGGSLRLSCAASGSIFGINDVHWFRQAPGKEREFVATITNGLSARYKDSVKGRFTISRDNAKNTVYLQMNSLKPEDTAVYYCAAQVYITTYELYDYWGQGTQVTVSS

SEQ ID NO: 348 - protein, 119 aa, Artificial Sequence
  synthetic construct
QVQLVESGGGLVQPGGSLRLSCAASGRILAANIVHWYRQAPGKNREGVAGITSGGSTYYADSVKGRFTISRDNAKNTVYLQMNSLKPEDTAVYYCAAKPLGSPDDFDYWGQGTQVTVSS

SEQ ID NO: 349 - protein, 119 aa, Artificial Sequence
  synthetic construct
QVQLVESGGGLVQPGGSLRLSCAASGSIPYVQAMSWFRQAPGKGREGVASIGWGGGTYYADSVKGRFTISRDNAKNTVYLQMNSLKPEDTAVYYCAAKFRENFSFYDYWGQGTQVTVSS

SEQ ID NO: 350 - protein, 119 aa, Artificial Sequence
  synthetic construct
QVQLVESGGGLVQPGGSLRLSCAASGFEFSTSAMHWYRQAPGKQREWVARITSGGSDNYGDSVKGRFTISRDNAKNTVYLQMNSLKPEDTAVYYCAAYVGTDNYGYDYWGQGTQVTVSS

SEQ ID NO: 351 - protein, 120 aa, Artificial Sequence
  synthetic construct
QVQLVESGGGLVQPGGSLRLSCAASGTIKSINAMHWFRQAPGKAREFVASIGSNGAKSYSDSVKGRFTISRDNAKNTVYLQMNSLKPEDTAVYYCTALITVAESDDYDYWGQGTQVTVSS

SEQ ID NO: 352 - protein, 121 aa, Artificial Sequence
  synthetic construct
QVQLVESGGGLVQPGGSLRLSCAASGSRDSINTAHWYRQAPGKAREWVASLDNSTTGSYADSVKGRFTISRDNAKNTVYLQMNSLKPEDTAVYYCAAVTGTPGGLGFYDYWGQGTQVTVSS

SEQ ID NO: 353 - protein, 123 aa, Artificial Sequence
  synthetic construct
QVQLVESGGGLVQPGGSLRLSCAASGSYASIASMHWFRQAPGKEREFVAGVTSGGSTNYGDSVKGRFTISRDNAKNTVYLQMNSLKPEDTAVYYCNALAGANYESQWEEYNYWGQGTQVTVSS

SEQ ID NO: 354 - protein, 119 aa, Artificial Sequence
  synthetic construct
QVQLVESGGGLVQPGGSLRLSCAASGGEFSDNGLHWFRQAPGKQREFVATASSDGSTQYRDSVKGRFTISRDNAKNTVYLQMNSLKPEDTAVYYCTALLMSSAGEYDVWGQGTQVTVSS

SEQ ID NO: 355 - protein, 126 aa, Artificial Sequence
  synthetic construct
QVQLVESGGGLVQPGGSLRLSCAASGFSESINATAWFRQAPGKQREGVAGLDGGGSDYYSDSVKGRFTISRDNAKNTVYLQMNSLKPEDTAVYYCAAQLTSSFRGYDFTPSNYDYWGQGTQVTVSS

SEQ ID NO: 356 - protein, 117 aa, Artificial Sequence
  synthetic construct
QVQLVESGGGLVQPGGSLRLSCAASGSSDSWNALHWFRQAPGKEREFVAGISSAGSGYYADSVKGRFTISRDNAKNTVYLQMNSLKPEDTAVYYCNGYEHTTGYDYWGQGTQVTVSS

SEQ ID NO: 357 - protein, 119 aa, Artificial Sequence
  synthetic construct
QVQLVESGGGLVQPGGSLRLSCAASGSAFSLNYMAWFRQAPGKQREGVAIIGSGGSSDYADSVKGRFTISRDNAKNTVYLQMNSLKPEDTAVYYCAAQPNGSPDDFDYWGQGTQVTVSS

SEQ ID NO: 358 - protein, 119 aa, Artificial Sequence
  synthetic construct
QVQLVESGGGLVQPGGSLRLSCAASGSTLSNNAMHWFRQAPGKAREFVAAIGSSDSTDYKDSVKGRFTISRDNAKNTVYLQMNSLKPEDTAVYYCTALLDSGPGEYNYWGQGTQVTVSS

SEQ ID NO: 359 - protein, 116 aa, Artificial Sequence
  synthetic construct
QVQLVESGGGLVQPGGSLRLSCAASGSIQSYEAMHWFRQAPGKAREWVAVITWGDSTQYGDSVKGRFTISRDNAKNTVYLQMNSLKPEDTAVYYCAADVRQGYSSWGQGTQVTVSS

SEQ ID NO: 360 - protein, 121 aa, Artificial Sequence
  synthetic construct
QVQLVESGGGLVQPGGSLRLSCAASGSIKNTNAMHWFRQAPGKDREWVAGITSGGNSNYGDSVKGRFTISRDNAKNTVYLQMNSLKPEDTAVYYCAAFGYRYDQDYTYDYWGQGTQVTVSS

SEQ ID NO: 361 - protein, 118 aa, Artificial Sequence
  synthetic construct
QVQLVESGGGLVQPGGSLRLSCAASGGIYSPYSMHWFRQAPGKAREWVAGIVGGGGTTYSDSVKGRFTISRDNAKNTVYLQMNSLKPEDTAVYYCNANYQEYDEYDYWGQGTQVTVSS

SEQ ID NO: 362 - protein, 117 aa, Artificial Sequence
  synthetic construct
QVQLVESGGGLVQPGGSLRLSCAASGAAFSKNALHWFRQAPGKGREWVASITSGSGTQYMDSVKGRFTISRDNAKNTVYLQMNSLKPEDTAVYYCAALAYSGGYDYWGQGTQVTVSS

SEQ ID NO: 363 - protein, 116 aa, Artificial Sequence
  synthetic construct
QVQLVESGGGLVQPGGSLRLSCAASGSIASINAIHWVRQAPGKQREEVAAITSGGSTGYADSVKGRFTISRDNAKNTVYLQMNSLKPEDTAVYYCAAQPGQSYAVWGQGTQVTVSS

SEQ ID NO: 364 - protein, 116 aa, Artificial Sequence
  synthetic construct
QVQLVESGGGLVQPGGSLRLSCAASGSIISRVKVHWFRQAPGKSREWVAGIDSGTASYYSDSVKGRFTISRDNAKNTVYLQMNSLKPEDTAVYYCAAENRGYGVDWGQGTQVTVSS

SEQ ID NO: 365 - protein, 120 aa, Artificial Sequence
  synthetic construct
QVQLVESGGGLVQPGGSLRLSCAASGSIENIERMHWYRQAPGKQRELVAVIDSGGTTRYADSVKGRFTISRDNAKNTVYLQMNSLKPEDTAVYYCAAVWLTNAESTQEYWGQGTQVTVSS

SEQ ID NO: 366 - protein, 122 aa, Artificial Sequence
  synthetic construct
QVQLVESGGGLVQPGGSLRLSCAASGDIYSQSAMAWYRQAPGKEREFVAAITTTGSRSYKDSVKGRFTISRDNAKNTVYLQMNSLKPEDTAVYYCAAFAVRSSSVSDAYDYWGQGTQVTVSS

SEQ ID NO: 367 - protein, 118 aa, Artificial Sequence
  synthetic construct
QVQLVESGGGLVQPGGSLRLSCAASGYIFEGGPTAWFRQAPGKGREFVAAITTSGETSYAYSVKGRFTISRDNAKNTVYLQMNSLKPEDTAVYYCNATDRDETGYDYWGQGTQVTVSS

SEQ ID NO: 368 - protein, 120 aa, Artificial Sequence
  synthetic construct
QVQLVESGGGLVQPGGSLRLSCAASGSYFSKNAVHWYRQAPGKGREWVAALTNSGSGNYKDSVKGRFTISRDNAKNTVYLQMNSLKPEDTAVYYCTANISESTGQGYDYWGQGTQVTVSS

SEQ ID NO: 369 - protein, 119 aa, Artificial Sequence
  synthetic construct
QVQLVESGGGLVQPGGSLRLSCAASGFEFTYRAVHWFRQAPGKGREWVAGVVSGGSTYYADSVKGRFTISRDNAKNTVYLQMNSLKPEDTAVYYCNAGPQAEEDTYDYWGQGTQVTVSS

SEQ ID NO: 370 - protein, 121 aa, Artificial Sequence
  synthetic construct
QVQLVESGGGLVQPGGSLRLSCAASGSSFRILAMHWYRQAPGKNREWVAVITSNGNTYYADSVKGRFTISRDNAKNTVYLQMNSLKPEDTAVYYCNADTLDLDDGATYNYWGQGTQVTVSS

SEQ ID NO: 371 - protein, 125 aa, Artificial Sequence
  synthetic construct
QVQLVESGGGLVQPGGSLRLSCAASGLRFAMTEMAWFRQAPGKQREFVALITEGGSGSYGDSVKGRFTISRDNAKNTVYLQMNSLKPEDTAVYYCAALHYTEGANTESATTFDYWGQGTQVTVSS

SEQ ID NO: 372 - protein, 121 aa, Artificial Sequence
  synthetic construct
QVQLVESGGGLVQPGGSLRLSCAASGSSFEGNAVHWFRQAPGKNREFVATITTSTSYYYADSVKGRFTISRDNAKNTVYLQMNSLKPEDTAVYYCAARLKDRGPQSEYDYWGQGTQVTVSS

SEQ ID NO: 373 - protein, 119 aa, Artificial Sequence
  synthetic construct
QVQLVESGGGLVQPGGSLRLSCAASGENFSGNLVHWFRQAPGKAREGVATVTSTGSGYYADSVKGRFTISRDNAKNTVYLQMNSLKPEDTAVYYCAAKHQKGEIPYYDWGQGTQVTVSS

SEQ ID NO: 374 - protein, 119 aa, Artificial Sequence
  synthetic construct
QVQLVESGGGLVQPGGSLRLSCAASGADFSSNAMHWFRQAPGKAREFVASIDSDKSGYYADSVKGRFTISRDNAKNTVYLQMNSLKPEDTAVYYCNAYSGTDNYGYDYWGQGTQVTVSS

SEQ ID NO: 375 - protein, 119 aa, Artificial Sequence
  synthetic construct
QVQLVESGGGLVQPGGSLRLSCAASGSMYSRAVMHWFRQAPGKEREGVASIGRTGDSYYADSVKGRFTISRDNAKNTVYLQMNSLKPEDTAVYYCAADNLDVVYGNRYWGQGTQVTVSS

SEQ ID NO: 376 - protein, 119 aa, Artificial Sequence
  synthetic construct
QVQLVESGGGLVQPGGSLRLSCAASGYYFQIVAMHWYRQAPGKEREGVAGITSTGSTYYGDSVKGRFTISRDNAKNTVYLQMNSLKPEDTAVYYCNAVSTDAQGAAEYWGQGTQVTVSS

SEQ ID NO: 377 - protein, 116 aa, Artificial Sequence
  synthetic construct
QVQLVESGGGLVQPGGSLRLSCAASGIDDSIGAMHWFRQAPGKGREFVAALSGTGTTSYADSVKGRFTISRDNAKNTVYLQMNSLKPEDTAVYYCAAAFPAGGDYWGQGTQVTVSS

SEQ ID NO: 378 - protein, 124 aa, Artificial Sequence
  synthetic construct
QVQLVESGGGLVQPGGSLRLSCAASGNYFEINKMHWFRQAPGKQREFVATIGSGGSTAYADSVKGRFTISRDNAKNTVYLQMNSLKPEDTAVYYCNALIYLADRRDGYYSYDYWGQGTQVTVSS

SEQ ID NO: 379 - protein, 122 aa, Artificial Sequence
  synthetic construct
QVQLVESGGGLVQPGGSLRLSCAASGLQTSDNAMHWFRQAPGKNRELVAGLTSGTSRNYGDSVKGRFTISRDNAKNTVYLQMNSLKPEDTAVYYCAAFPDVGDASSIGFVYWGQGTQVTVSS

SEQ ID NO: 380 - protein, 119 aa, Artificial Sequence
  synthetic construct
QVQLVESGGGLVQPGGSLRLSCAASGSISEINLLHWFRQAPGKQREWVATMTSGGSTDYADSVKGRFTISRDNAKNTVYLQMNSLKPEDTAVYYCAADEFTKYGHGRYWGQGTQVTVSS

SEQ ID NO: 381 - protein, 116 aa, Artificial Sequence
  synthetic construct
QVQLVESGGGLVQPGGSLRLSCAASGGEKSISAAHWYRQAPGKEREFVAAITAASTTSYADSVKGRFTISRDNAKNTVYLQMNSLKPEDTAVYYCTAVLYDSYDYWGQGTQVTVSS

SEQ ID NO: 382 - protein, 119 aa, Artificial Sequence
  synthetic construct
QVQLVESGGGLVQPGGSLRLSCAASGEDYSYNAAHWFRQAPGKEREWVAAINSGGGTYYADSVKGRFTISRDNAKNTVYLQMNSLKPEDTAVYYCAYLGALDRDGYDAWGQGTQVTVSS

SEQ ID NO: 383 - protein, 119 aa, Artificial Sequence
  synthetic construct
QVQLVESGGGLVQPGGSLRLSCAASGSISSINSMHWYRQAPGKEREWVAAVSSYGSDYYADSVKGRFTISRDNAKNTVYLQMNSLKPEDTAVYYCAARTVTGFTYGDYWGQGTQVTVSS

SEQ ID NO: 384 - protein, 121 aa, Artificial Sequence
  synthetic construct
QVQLVESGGGLVQPGGSLRLSCAASGPQFSSNAVSWFRQAPGKEREWVAGITSGGSTDYADSVKGRFTISRDNAKNTVYLQMNSLKPEDTAVYYCTAPSVSDADRRTRDYWGQGTQVTVSS

SEQ ID NO: 385 - protein, 121 aa, Artificial Sequence
  synthetic construct
QVQLVESGGGLVQPGGSLRLSCAASGSQFSHNLMSWFRQAPGKQREFVAAITSFGSTGYADSVKGRFTISRDNAKNTVYLQMNSLKPEDTAVYYCAALSSEDTSHYPRNYWGQGTQVTVSS

SEQ ID NO: 386 - protein, 119 aa, Artificial Sequence
  synthetic construct
QVQLVESGGGLVQPGGSLRLSCAASGYILSENLTAWFRQAPGKAREVVATVTSGDYTNYDDSVKGRFTISRDNAKNTVYLQMNSLKPEDTAVYYCAAGTSDWEVKWSHWGQGTQVTVSS

SEQ ID NO: 387 - protein, 121 aa, Artificial Sequence
  synthetic construct
QVQLVESGGGLVQPGGSLRLSCAASGIPASYLNMHWFRQAPGKQREWVAGAGGTNSNYYQDSVKGRFTISRDNAKNTVYLQMNSLKPEDTAVYYCAALDGAESYDYVYPVWGQGTQVTVSS

SEQ ID NO: 388 - protein, 121 aa, Artificial Sequence
  synthetic construct
QVQLVESGGGLVQPGGSLRLSCAASGSTGSDYAMHWFRQAPGKDREWVAGISSYGSDSYGDSVKGRFTISRDNAKNTVYLQMNSLKPEDTAVYYCAALQASAGAGYYYDYWGQGTQVTVSS

SEQ ID NO: 389 - protein, 24 aa, Artificial Sequence
  synthetic construct
TVILEYAHRLSQDILCDALQQWAC

SEQ ID NO: 390 - protein, 24 aa, Artificial Sequence
  synthetic construct
TVILEYAHRLSQDILSDALQQWAS

SEQ ID NO: 391 - protein, 8 aa, Artificial Sequence
  synthetic construct
GQITAENY

SEQ ID NO: 392 - protein, 7 aa, Artificial Sequence
  synthetic construct
ITTGGST

SEQ ID NO: 393 - protein, 10 aa, Artificial Sequence
  synthetic construct
NADRSYFPDY

SEQ ID NO: 394 - protein, 8 aa, Artificial Sequence
  synthetic construct
GSIREINA

SEQ ID NO: 395 - protein, 7 aa, Artificial Sequence
  synthetic construct
IDSGSLD

SEQ ID NO: 396 - protein, 10 aa, Artificial Sequence
  synthetic construct
AASRSGDYRY

SEQ ID NO: 397 - protein, 8 aa, Artificial Sequence
  synthetic construct
GSILQINA

SEQ ID NO: 398 - protein, 7 aa, Artificial Sequence
  synthetic construct
ITSGGST

SEQ ID NO: 399 - protein, 13 aa, Artificial Sequence
  synthetic construct
AATSTRGGLDSDY

SEQ ID NO: 400 - protein, 8 aa, Artificial Sequence
  synthetic construct
GRQFGAWR

SEQ ID NO: 401 - protein, 7 aa, Artificial Sequence
  synthetic construct
ITSGTSG

SEQ ID NO: 402 - protein, 12 aa, Artificial Sequence
  synthetic construct
AATSHTTYHAEY

SEQ ID NO: 403 - protein, 8 aa, Artificial Sequence
  synthetic construct
GTEYDISA

SEQ ID NO: 404 - protein, 7 aa, Artificial Sequence
  synthetic construct
VTSDNST

SEQ ID NO: 405 - protein, 11 aa, Artificial Sequence
  synthetic construct
TATVKTSQYRY

SEQ ID NO: 406 - protein, 8 aa, Artificial Sequence
  synthetic construct
GSSININY

SEQ ID NO: 407 - protein, 7 aa, Artificial Sequence
  synthetic construct
ITSGGGT

SEQ ID NO: 408 - protein, 11 aa, Artificial Sequence
  synthetic construct
AASEAWRRFDS

SEQ ID NO: 409 - protein, 8 aa, Artificial Sequence
  synthetic construct
GTWGNIQY

SEQ ID NO: 410 - protein, 7 aa, Artificial Sequence
  synthetic construct
ITPNNGT

SEQ ID NO: 411 - protein, 15 aa, Artificial Sequence
  synthetic construct
TAVLWYTTKEGPYDY

SEQ ID NO: 412 - protein, 8 aa, Artificial Sequence
  synthetic construct
GSITSINW

SEQ ID NO: 413 - protein, 7 aa, Artificial Sequence
  synthetic construct
ISQTGRG

SEQ ID NO: 414 - protein, 12 aa, Artificial Sequence
  synthetic construct
AAASRTGTPYNY

SEQ ID NO: 415 - protein, 8 aa, Artificial Sequence
  synthetic construct
GSSESWNA

SEQ ID NO: 416 - protein, 7 aa, Artificial Sequence
  synthetic construct
ITSGTST

SEQ ID NO: 417 - protein, 11 aa, Artificial Sequence
  synthetic construct
NGYEHTTGYDY

SEQ ID NO: 418 - protein, 8 aa, Artificial Sequence
  synthetic construct
GSYASIAS

SEQ ID NO: 419 - protein, 7 aa, Artificial Sequence
  synthetic construct
LTSGGSR

SEQ ID NO: 420 - protein, 13 aa, Artificial Sequence
  synthetic construct
TALILHYAGMYDY

SEQ ID NO: 421 - protein, 8 aa, Artificial Sequence
  synthetic construct
GYADEINM

SEQ ID NO: 422 - protein, 7 aa, Artificial Sequence
  synthetic construct
IASGGST

SEQ ID NO: 423 - protein, 16 aa, Artificial Sequence
  synthetic construct
AAIPRPSLSYAHDYAY

SEQ ID NO: 424 - protein, 8 aa, Artificial Sequence
  synthetic construct
GGVTRGNA

SEQ ID NO: 425 - protein, 7 aa, Artificial Sequence
  synthetic construct
IDNGGSH

SEQ ID NO: 426 - protein, 17 aa, Artificial Sequence
  synthetic construct
AVFPDHDSEQASIDRVY

SEQ ID NO: 427 - protein, 8 aa, Artificial Sequence
  synthetic construct
GTYASYET

SEQ ID NO: 428 - protein, 7 aa, Artificial Sequence
  synthetic construct
ITGAGSS

SEQ ID NO: 429 - protein, 16 aa, Artificial Sequence
  synthetic construct
NADSIPPALRAANTGY

SEQ ID NO: 430 - protein, 8 aa, Artificial Sequence
  synthetic construct
GRILAANI

SEQ ID NO: 431 - protein, 7 aa, Artificial Sequence
  synthetic construct
ITTGGSN

SEQ ID NO: 432 - protein, 14 aa, Artificial Sequence
  synthetic construct
NALGLGSASGSYES

SEQ ID NO: 433 - protein, 8 aa, Artificial Sequence
  synthetic construct
GSIYKYSD

SEQ ID NO: 434 - protein, 7 aa, Artificial Sequence
  synthetic construct
LTGAGTT

SEQ ID NO: 435 - protein, 14 aa, Artificial Sequence
  synthetic construct
AAGQGFKATRTFDS

SEQ ID NO: 436 - protein, 8 aa, Artificial Sequence
  synthetic construct
GSGASIGA

SEQ ID NO: 437 - protein, 7 aa, Artificial Sequence
  synthetic construct
ITSAGNT

SEQ ID NO: 438 - protein, 17 aa, Artificial Sequence
  synthetic construct
NALAGANYESQWEEYNY

SEQ ID NO: 439 - protein, 8 aa, Artificial Sequence
  synthetic construct
GHIFSINN

SEQ ID NO: 440 - protein, 7 aa, Artificial Sequence
  synthetic construct
IHSGGGT

SEQ ID NO: 441 - protein, 18 aa, Artificial Sequence
  synthetic construct
NAEVSTGAGSRGRVSDVY

SEQ ID NO: 442 - protein, 8 aa, Artificial Sequence
  synthetic construct
GSAMMINR

SEQ ID NO: 443 - protein, 7 aa, Artificial Sequence
  synthetic construct
ITWQGST

SEQ ID NO: 444 - protein, 16 aa, Artificial Sequence
  synthetic construct
NALSADGAYVRRYKDY

SEQ ID NO: 445 - protein, 8 aa, Artificial Sequence
  synthetic construct
GHQLSTNA

SEQ ID NO: 446 - protein, 7 aa, Artificial Sequence
  synthetic construct
IGSGSSS

SEQ ID NO: 447 - protein, 15 aa, Artificial Sequence
  synthetic construct
TALGGDELDGMTYDY

SEQ ID NO: 448 - protein, 8 aa, Artificial Sequence
  synthetic construct
GETFSSNA

SEQ ID NO: 449 - protein, 7 aa, Artificial Sequence
  synthetic construct
ITSGGND

SEQ ID NO: 450 - protein, 15 aa, Artificial Sequence
  synthetic construct
NAPATEDTSHYPRNY

SEQ ID NO: 451 - protein, 8 aa, Artificial Sequence
  synthetic construct
GAELSYTA

SEQ ID NO: 452 - protein, 7 aa, Artificial Sequence
  synthetic construct
INSGGSS

SEQ ID NO: 453 - protein, 10 aa, Artificial Sequence
  synthetic construct
TALLDSTYDY

SEQ ID NO: 454 - protein, 8 aa, Artificial Sequence
  synthetic construct
GSAFATNG

SEQ ID NO: 455 - protein, 7 aa, Artificial Sequence
  synthetic construct
ITSNGSI

SEQ ID NO: 456 - protein, 15 aa, Artificial Sequence
  synthetic construct
TALLMSYGGGEEYDY

SEQ ID NO: 457 - protein, 8 aa, Artificial Sequence
  synthetic construct
GQLDRSNA

SEQ ID NO: 458 - protein, 7 aa, Artificial Sequence
  synthetic construct
ITSGAST

SEQ ID NO: 459 - protein, 14 aa, Artificial Sequence
  synthetic construct
AADRYRAIQRTQYS

SEQ ID NO: 460 - protein, 8 aa, Artificial Sequence
  synthetic construct
GDDFIENA

SEQ ID NO: 461 - protein, 7 aa, Artificial Sequence
  synthetic construct
VTLGGHS

SEQ ID NO: 462 - protein, 19 aa, Artificial Sequence
  synthetic construct
NYPVGIPAVAIDGRRSYGY

SEQ ID NO: 463 - protein, 8 aa, Artificial Sequence
  synthetic construct
GAFFSYGA

SEQ ID NO: 464 - protein, 7 aa, Artificial Sequence
  synthetic construct
ITSGGST

SEQ ID NO: 465 - protein, 15 aa, Artificial Sequence
  synthetic construct
NAGVVWLPSNAAYNL

SEQ ID NO: 466 - protein, 8 aa, Artificial Sequence
  synthetic construct
GDIFSINA

SEQ ID NO: 467 - protein, 7 aa, Artificial Sequence
  synthetic construct
ITSGGST

SEQ ID NO: 468 - protein, 13 aa, Artificial Sequence
  synthetic construct
AAQQARGGLDSDY

SEQ ID NO: 469 - protein, 8 aa, Artificial Sequence
  synthetic construct
GSAMMINR

SEQ ID NO: 470 - protein, 7 aa, Artificial Sequence
  synthetic construct
ITWQGST

SEQ ID NO: 471 - protein, 12 aa, Artificial Sequence
  synthetic construct
NAVAPVHENENY

SEQ ID NO: 472 - protein, 8 aa, Artificial Sequence
  synthetic construct
GNDWYINS

SEQ ID NO: 473 - protein, 7 aa, Artificial Sequence
  synthetic construct
VSRGGSN

SEQ ID NO: 474 - protein, 13 aa, Artificial Sequence
  synthetic construct
AALLETSSGDYTY

SEQ ID NO: 475 - protein, 8 aa, Artificial Sequence
  synthetic construct
GYDFDILR

SEQ ID NO: 476 - protein, 7 aa, Artificial Sequence
  synthetic construct
ITSAESV

SEQ ID NO: 477 - protein, 13 aa, Artificial Sequence
  synthetic construct
ATYDYTDDYGYAS

SEQ ID NO: 478 - protein, 8 aa, Artificial Sequence
  synthetic construct
GSISSIEA

SEQ ID NO: 479 - protein, 7 aa, Artificial Sequence
  synthetic construct
MASGGDT

SEQ ID NO: 480 - protein, 15 aa, Artificial Sequence
  synthetic construct
AAQTYRPDTWGRYTF

SEQ ID NO: 481 - protein, 8 aa, Artificial Sequence
  synthetic construct
GYHASILA

SEQ ID NO: 482 - protein, 7 aa, Artificial Sequence
  synthetic construct
MTWSDMT

SEQ ID NO: 483 - protein, 13 aa, Artificial Sequence
  synthetic construct
AATYVTRDRPWTH

SEQ ID NO: 484 - protein, 8 aa, Artificial Sequence
  synthetic construct
GGISVANV

SEQ ID NO: 485 - protein, 7 aa, Artificial Sequence
  synthetic construct
LTSEGSR

SEQ ID NO: 486 - protein, 13 aa, Artificial Sequence
  synthetic construct
AAEPTGNTHEFEY

SEQ ID NO: 487 - protein, 8 aa, Artificial Sequence
  synthetic construct
GSPFKTQT

SEQ ID NO: 488 - protein, 7 aa, Artificial Sequence
  synthetic construct
ITSGSGT

SEQ ID NO: 489 - protein, 10 aa, Artificial Sequence
  synthetic construct
AAANDGYGVD

SEQ ID NO: 490 - protein, 8 aa, Artificial Sequence
  synthetic construct
GSIGQIVL

SEQ ID NO: 491 - protein, 7 aa, Artificial Sequence
  synthetic construct
ITSPSST

SEQ ID NO: 492 - protein, 20 aa, Artificial Sequence
  synthetic construct
NTELEGKTQGFTGKTVWYDY

SEQ ID NO: 493 - protein, 8 aa, Artificial Sequence
  synthetic construct
GLDVSINF

SEQ ID NO: 494 - protein, 7 aa, Artificial Sequence
  synthetic construct
ITWQGST

SEQ ID NO: 495 - protein, 12 aa, Artificial Sequence
  synthetic construct
ATVWAGGTPNDY

SEQ ID NO: 496 - protein, 8 aa, Artificial Sequence
  synthetic construct
GDIFSKNA

SEQ ID NO: 497 - protein, 7 aa, Artificial Sequence
  synthetic construct
IYSDGSS

SEQ ID NO: 498 - protein, 11 aa, Artificial Sequence
  synthetic construct
AALDYSARYRT

SEQ ID NO: 499 - protein, 8 aa, Artificial Sequence
  synthetic construct
GSGFSNNA

SEQ ID NO: 500 - protein, 7 aa, Artificial Sequence
  synthetic construct
ITSGSVS

SEQ ID NO: 501 - protein, 17 aa, Artificial Sequence
  synthetic construct
AAVTGSSTGNAGQGYLT

SEQ ID NO: 502 - protein, 8 aa, Artificial Sequence
  synthetic construct
GSIFSLNW

SEQ ID NO: 503 - protein, 7 aa, Artificial Sequence
  synthetic construct
INSGGHA

SEQ ID NO: 504 - protein, 12 aa, Artificial Sequence
  synthetic construct
AAGPYGAYNFNS

SEQ ID NO: 505 - protein, 7 aa, Artificial Sequence
  synthetic construct
YIFSATV

SEQ ID NO: 506 - protein, 7 aa, Artificial Sequence
  synthetic construct
INSTGSS

SEQ ID NO: 507 - protein, 13 aa, Artificial Sequence
  synthetic construct
AADGADVVYGNRY

SEQ ID NO: 508 - protein, 7 aa, Artificial Sequence
  synthetic construct
SFFSINA

SEQ ID NO: 509 - protein, 7 aa, Artificial Sequence
  synthetic construct
ISVGDSR

SEQ ID NO: 510 - protein, 14 aa, Artificial Sequence
  synthetic construct
AAQIYITTYELYDY

SEQ ID NO: 511 - protein, 7 aa, Artificial Sequence
  synthetic construct
TIPSINY

SEQ ID NO: 512 - protein, 7 aa, Artificial Sequence
  synthetic construct
LTSGDNR

SEQ ID NO: 513 - protein, 15 aa, Artificial Sequence
  synthetic construct
NAKTVGSSYRDSYNY

SEQ ID NO: 514 - protein, 7 aa, Artificial Sequence
  synthetic construct
SYFKINS

SEQ ID NO: 515 - protein, 7 aa, Artificial Sequence
  synthetic construct
IHSGDGT

SEQ ID NO: 516 - protein, 14 aa, Artificial Sequence
  synthetic construct
AADQAWVRAGTYDY

SEQ ID NO: 517 - protein, 8 aa, Artificial Sequence
  synthetic construct
GQTNSILA

SEQ ID NO: 518 - protein, 7 aa, Artificial Sequence
  synthetic construct
ITSGSNS

SEQ ID NO: 519 - protein, 16 aa, Artificial Sequence
  synthetic construct
AARNGKLSPKANQRLY

SEQ ID NO: 520 - protein, 8 aa, Artificial Sequence
  synthetic construct
GSATSINR

SEQ ID NO: 521 - protein, 7 aa, Artificial Sequence
  synthetic construct
IDSGGYT

SEQ ID NO: 522 - protein, 15 aa, Artificial Sequence
  synthetic construct
NAVPVTTDKTPMYQY

SEQ ID NO: 523 - protein, 8 aa, Artificial Sequence
  synthetic construct
GSIESINA

SEQ ID NO: 524 - protein, 7 aa, Artificial Sequence
  synthetic construct
ITENDVD

SEQ ID NO: 525 - protein, 14 aa, Artificial Sequence
  synthetic construct
ASLSSGYQYVGFRY

SEQ ID NO: 526 - protein, 8 aa, Artificial Sequence
  synthetic construct
GSISSYNA

SEQ ID NO: 527 - protein, 7 aa, Artificial Sequence
  synthetic construct
LTSSGSD

SEQ ID NO: 528 - protein, 13 aa, Artificial Sequence
  synthetic construct
NAFSYHTYSRYDF

SEQ ID NO: 529 - protein, 8 aa, Artificial Sequence
  synthetic construct
GSIWSIAF

SEQ ID NO: 530 - protein, 7 aa, Artificial Sequence
  synthetic construct
ITTGNDE

SEQ ID NO: 531 - protein, 16 aa, Artificial Sequence
  synthetic construct
AANTYERTPDGRTFSY

SEQ ID NO: 532 - protein, 8 aa, Artificial Sequence
  synthetic construct
GSSTSINI

SEQ ID NO: 533 - protein, 7 aa, Artificial Sequence
  synthetic construct
ITGGSNG

SEQ ID NO: 534 - protein, 14 aa, Artificial Sequence
  synthetic construct
AAVATSYTGVEYEY

SEQ ID NO: 535 - protein, 8 aa, Artificial Sequence
  synthetic construct
GSRDSIVA

SEQ ID NO: 536 - protein, 7 aa, Artificial Sequence
  synthetic construct
ITSRWVT

SEQ ID NO: 537 - protein, 13 aa, Artificial Sequence
  synthetic construct
NAPVYYDTEWYDI

SEQ ID NO: 538 - protein, 8 aa, Artificial Sequence
  synthetic construct
GRILAANI

SEQ ID NO: 539 - protein, 7 aa, Artificial Sequence
  synthetic construct
ITSGGGS

SEQ ID NO: 540 - protein, 19 aa, Artificial Sequence
  synthetic construct
NALGLGQSSLYRSADPYDS

SEQ ID NO: 541 - protein, 8 aa, Artificial Sequence
  synthetic construct
GSDFTINA

SEQ ID NO: 542 - protein, 7 aa, Artificial Sequence
  synthetic construct
ITSTSST

SEQ ID NO: 543 - protein, 18 aa, Artificial Sequence
  synthetic construct
AAFAVRDADTAGWTARDY

SEQ ID NO: 544 - protein, 8 aa, Artificial Sequence
  synthetic construct
GSSFSINW

SEQ ID NO: 545 - protein, 7 aa, Artificial Sequence
  synthetic construct
INSTGSD

SEQ ID NO: 546 - protein, 20 aa, Artificial Sequence
  synthetic construct
AAAITQPMPQGKGAPPGFYY

SEQ ID NO: 547 - protein, 8 aa, Artificial Sequence
  synthetic construct
GSIYVINT

SEQ ID NO: 548 - protein, 7 aa, Artificial Sequence
  synthetic construct
IAKGGSG

SEQ ID NO: 549 - protein, 15 aa, Artificial Sequence
  synthetic construct
AADRTYGYEYSTYQY

SEQ ID NO: 550 - protein, 8 aa, Artificial Sequence
  synthetic construct
GQISSNNG

SEQ ID NO: 551 - protein, 7 aa, Artificial Sequence
  synthetic construct
ATTGGTT

SEQ ID NO: 552 - protein, 13 aa, Artificial Sequence
  synthetic construct
TALLMSRAGEFDY

SEQ ID NO: 553 - protein, 8 aa, Artificial Sequence
  synthetic construct
GSQLSINY

SEQ ID NO: 554 - protein, 7 aa, Artificial Sequence
  synthetic construct
ITITGSS

SEQ ID NO: 555 - protein, 13 aa, Artificial Sequence
  synthetic construct
NTVDRTDNYGYDY

SEQ ID NO: 556 - protein, 8 aa, Artificial Sequence
  synthetic construct
GSIFGIND

SEQ ID NO: 557 - protein, 7 aa, Artificial Sequence
  synthetic construct
ITNGLSA

SEQ ID NO: 558 - protein, 14 aa, Artificial Sequence
  synthetic construct
AAQVYITTYELYDY

SEQ ID NO: 559 - protein, 8 aa, Artificial Sequence
  synthetic construct
GRILAANI

SEQ ID NO: 560 - protein, 7 aa, Artificial Sequence
  synthetic construct
ITSGGST

SEQ ID NO: 561 - protein, 13 aa, Artificial Sequence
  synthetic construct
AAKPLGSPDDFDY

SEQ ID NO: 562 - protein, 8 aa, Artificial Sequence
  synthetic construct
GSIPYVQA

SEQ ID NO: 563 - protein, 7 aa, Artificial Sequence
  synthetic construct
IGWGGGT

SEQ ID NO: 564 - protein, 13 aa, Artificial Sequence
  synthetic construct
AAKFRENFSFYDY

SEQ ID NO: 565 - protein, 8 aa, Artificial Sequence
  synthetic construct
GFEFSTSA

SEQ ID NO: 566 - protein, 7 aa, Artificial Sequence
  synthetic construct
ITSGGSD

SEQ ID NO: 567 - protein, 13 aa, Artificial Sequence
  synthetic construct
AAYVGTDNYGYDY

SEQ ID NO: 568 - protein, 8 aa, Artificial Sequence
  synthetic construct
GTIKSINA

SEQ ID NO: 569 - protein, 7 aa, Artificial Sequence
  synthetic construct
IGSNGAK

SEQ ID NO: 570 - protein, 14 aa, Artificial Sequence
  synthetic construct
TALITVAESDDYDY

SEQ ID NO: 571 - protein, 8 aa, Artificial Sequence
  synthetic construct
GSRDSINT

SEQ ID NO: 572 - protein, 7 aa, Artificial Sequence
  synthetic construct
LDNSTTG

SEQ ID NO: 573 - protein, 15 aa, Artificial Sequence
  synthetic construct
AAVTGTPGGLGFYDY

SEQ ID NO: 574 - protein, 8 aa, Artificial Sequence
  synthetic construct
GSYASIAS

SEQ ID NO: 575 - protein, 7 aa, Artificial Sequence
  synthetic construct
VTSGGST

SEQ ID NO: 576 - protein, 17 aa, Artificial Sequence
  synthetic construct
NALAGANYESQWEEYNY

SEQ ID NO: 577 - protein, 8 aa, Artificial Sequence
  synthetic construct
GGEFSDNG

SEQ ID NO: 578 - protein, 7 aa, Artificial Sequence
  synthetic construct
ASSDGST

SEQ ID NO: 579 - protein, 13 aa, Artificial Sequence
  synthetic construct
TALLMSSAGEYDV

SEQ ID NO: 580 - protein, 8 aa, Artificial Sequence
  synthetic construct
GFSESINA

SEQ ID NO: 581 - protein, 7 aa, Artificial Sequence
  synthetic construct
LDGGGSD

SEQ ID NO: 582 - protein, 20 aa, Artificial Sequence
  synthetic construct
AAQLTSSFRGYDFTPSNYDY

SEQ ID NO: 583 - protein, 8 aa, Artificial Sequence
  synthetic construct
GSSDSWNA

SEQ ID NO: 584 - protein, 7 aa, Artificial Sequence
  synthetic construct
ISSAGSG

SEQ ID NO: 585 - protein, 11 aa, Artificial Sequence
  synthetic construct
NGYEHTTGYDY

SEQ ID NO: 586 - protein, 8 aa, Artificial Sequence
  synthetic construct
GSAFSLNY

SEQ ID NO: 587 - protein, 7 aa, Artificial Sequence
  synthetic construct
IGSGGSS

SEQ ID NO: 588 - protein, 13 aa, Artificial Sequence
  synthetic construct
AAQPNGSPDDFDY

SEQ ID NO: 589 - protein, 8 aa, Artificial Sequence
  synthetic construct
GSTLSNNA

SEQ ID NO: 590 - protein, 7 aa, Artificial Sequence
  synthetic construct
IGSSDST

SEQ ID NO: 591 - protein, 13 aa, Artificial Sequence
  synthetic construct
TALLDSGPGEYNY

SEQ ID NO: 592 - protein, 8 aa, Artificial Sequence
  synthetic construct
GSIQSYEA

SEQ ID NO: 593 - protein, 7 aa, Artificial Sequence
  synthetic construct
ITWGDST

SEQ ID NO: 594 - protein, 10 aa, Artificial Sequence
  synthetic construct
AADVRQGYSS

SEQ ID NO: 595 - protein, 8 aa, Artificial Sequence
  synthetic construct
GSIKNTNA

SEQ ID NO: 596 - protein, 7 aa, Artificial Sequence
  synthetic construct
ITSGGNS

SEQ ID NO: 597 - protein, 15 aa, Artificial Sequence
  synthetic construct
AAFGYRYDQDYTYDY

SEQ ID NO: 598 - protein, 8 aa, Artificial Sequence
  synthetic construct
GGIYSPYS

SEQ ID NO: 599 - protein, 7 aa, Artificial Sequence
  synthetic construct
IVGGGGT

SEQ ID NO: 600 - protein, 12 aa, Artificial Sequence
  synthetic construct
NANYQEYDEYDY

SEQ ID NO: 601 - protein, 8 aa, Artificial Sequence
  synthetic construct
GAAFSKNA

SEQ ID NO: 602 - protein, 7 aa, Artificial Sequence
  synthetic construct
ITSGSGT

SEQ ID NO: 603 - protein, 11 aa, Artificial Sequence
  synthetic construct
AALAYSGGYDY

SEQ ID NO: 604 - protein, 8 aa, Artificial Sequence
  synthetic construct
GSIASINA

SEQ ID NO: 605 - protein, 7 aa, Artificial Sequence
  synthetic construct
ITSGGST

SEQ ID NO: 606 - protein, 10 aa, Artificial Sequence
  synthetic construct
AAQPGQSYAV

SEQ ID NO: 607 - protein, 8 aa, Artificial Sequence
  synthetic construct
GSIISRVK

SEQ ID NO: 608 - protein, 7 aa, Artificial Sequence
  synthetic construct
IDSGTAS

SEQ ID NO: 609 - protein, 10 aa, Artificial Sequence
  synthetic construct
AAENRGYGVD

SEQ ID NO: 610 - protein, 8 aa, Artificial Sequence
  synthetic construct
GSIENIER

SEQ ID NO: 611 - protein, 7 aa, Artificial Sequence
  synthetic construct
IDSGGTT

SEQ ID NO: 612 - protein, 14 aa, Artificial Sequence
  synthetic construct
AAVWLTNAESTQEY

SEQ ID NO: 613 - protein, 8 aa, Artificial Sequence
  synthetic construct
GDIYSQSA

SEQ ID NO: 614 - protein, 7 aa, Artificial Sequence
  synthetic construct
ITTTGSR

SEQ ID NO: 615 - protein, 16 aa, Artificial Sequence
  synthetic construct
AAFAVRSSSVSDAYDY

SEQ ID NO: 616 - protein, 8 aa, Artificial Sequence
  synthetic construct
GYIFEGGP

SEQ ID NO: 617 - protein, 7 aa, Artificial Sequence
  synthetic construct
ITTSGET

SEQ ID NO: 618 - protein, 12 aa, Artificial Sequence
  synthetic construct
NATDRDETGYDY

SEQ ID NO: 619 - protein, 8 aa, Artificial Sequence
  synthetic construct
GSYFSKNA

SEQ ID NO: 620 - protein, 7 aa, Artificial Sequence
  synthetic construct
LTNSGSG

SEQ ID NO: 621 - protein, 14 aa, Artificial Sequence
  synthetic construct
TANISESTGQGYDY

SEQ ID NO: 622 - protein, 8 aa, Artificial Sequence
  synthetic construct
GFEFTYRA

SEQ ID NO: 623 - protein, 7 aa, Artificial Sequence
  synthetic construct
VVSGGST

SEQ ID NO: 624 - protein, 13 aa, Artificial Sequence
  synthetic construct
NAGPQAEEDTYDY

SEQ ID NO: 625 - protein, 8 aa, Artificial Sequence
  synthetic construct
GSSFRILA

SEQ ID NO: 626 - protein, 7 aa, Artificial Sequence
  synthetic construct
ITSNGNT

SEQ ID NO: 627 - protein, 15 aa, Artificial Sequence
  synthetic construct
NADTLDLDDGATYNY

SEQ ID NO: 628 - protein, 8 aa, Artificial Sequence
  synthetic construct
GLRFAMTE

SEQ ID NO: 629 - protein, 7 aa, Artificial Sequence
  synthetic construct
ITEGGSG

SEQ ID NO: 630 - protein, 19 aa, Artificial Sequence
  synthetic construct
AALHYTEGANTESATTFDY

SEQ ID NO: 631 - protein, 8 aa, Artificial Sequence
  synthetic construct
GSSFEGNA

SEQ ID NO: 632 - protein, 7 aa, Artificial Sequence
  synthetic construct
ITTSTSY

SEQ ID NO: 633 - protein, 15 aa, Artificial Sequence
  synthetic construct
AARLKDRGPQSEYDY

SEQ ID NO: 634 - protein, 8 aa, Artificial Sequence
  synthetic construct
GENFSGNL

SEQ ID NO: 635 - protein, 7 aa, Artificial Sequence
  synthetic construct
VTSTGSG

SEQ ID NO: 636 - protein, 13 aa, Artificial Sequence
  synthetic construct
AAKHQKGEIPYYD

SEQ ID NO: 637 - protein, 8 aa, Artificial Sequence
  synthetic construct
GADFSSNA

SEQ ID NO: 638 - protein, 7 aa, Artificial Sequence
  synthetic construct
IDSDKSG

SEQ ID NO: 639 - protein, 13 aa, Artificial Sequence
  synthetic construct
NAYSGTDNYGYDY

SEQ ID NO: 640 - protein, 8 aa, Artificial Sequence
  synthetic construct
GSMYSRAV

SEQ ID NO: 641 - protein, 7 aa, Artificial Sequence
  synthetic construct
IGRTGDS

SEQ ID NO: 642 - protein, 13 aa, Artificial Sequence
  synthetic construct
AADNLDVVYGNRY

SEQ ID NO: 643 - protein, 8 aa, Artificial Sequence
  synthetic construct
GYYFQIVA

SEQ ID NO: 644 - protein, 7 aa, Artificial Sequence
  synthetic construct
ITSTGST

SEQ ID NO: 645 - protein, 13 aa, Artificial Sequence
  synthetic construct
NAVSTDAQGAAEY

SEQ ID NO: 646 - protein, 8 aa, Artificial Sequence
  synthetic construct
GIDDSIGA

SEQ ID NO: 647 - protein, 7 aa, Artificial Sequence
  synthetic construct
LSGTGTT

SEQ ID NO: 648 - protein, 10 aa, Artificial Sequence
  synthetic construct
AAAFPAGGDY

SEQ ID NO: 649 - protein, 8 aa, Artificial Sequence
  synthetic construct
GNYFEINK

SEQ ID NO: 650 - protein, 7 aa, Artificial Sequence
  synthetic construct
IGSGGST

SEQ ID NO: 651 - protein, 18 aa, Artificial Sequence
  synthetic construct
NALIYLADRRDGYYSYDY

SEQ ID NO: 652 - protein, 8 aa, Artificial Sequence
  synthetic construct
GLQTSDNA

SEQ ID NO: 653 - protein, 7 aa, Artificial Sequence
  synthetic construct
LTSGTSR

SEQ ID NO: 654 - protein, 16 aa, Artificial Sequence
  synthetic construct
AAFPDVGDASSIGFVY

SEQ ID NO: 655 - protein, 8 aa, Artificial Sequence
  synthetic construct
GSISEINL

SEQ ID NO: 656 - protein, 7 aa, Artificial Sequence
  synthetic construct
MTSGGST

SEQ ID NO: 657 - protein, 13 aa, Artificial Sequence
  synthetic construct
AADEFTKYGHGRY

SEQ ID NO: 658 - protein, 8 aa, Artificial Sequence
  synthetic construct
GGEKSISA

SEQ ID NO: 659 - protein, 7 aa, Artificial Sequence
  synthetic construct
ITAASTT

SEQ ID NO: 660 - protein, 10 aa, Artificial Sequence
  synthetic construct
TAVLYDSYDY

SEQ ID NO: 661 - protein, 8 aa, Artificial Sequence
  synthetic construct
GEDYSYNA

SEQ ID NO: 662 - protein, 7 aa, Artificial Sequence
  synthetic construct
INSGGGT

SEQ ID NO: 663 - protein, 13 aa, Artificial Sequence
  synthetic construct
AYLGALDRDGYDA

SEQ ID NO: 664 - protein, 8 aa, Artificial Sequence
  synthetic construct
GSISSINS

SEQ ID NO: 665 - protein, 7 aa, Artificial Sequence
  synthetic construct
VSSYGSD

SEQ ID NO: 666 - protein, 13 aa, Artificial Sequence
  synthetic construct
AARTVTGFTYGDY

SEQ ID NO: 667 - protein, 8 aa, Artificial Sequence
  synthetic construct
GPQFSSNA

SEQ ID NO: 668 - protein, 7 aa, Artificial Sequence
  synthetic construct
ITSGGST

SEQ ID NO: 669 - protein, 15 aa, Artificial Sequence
  synthetic construct
TAPSVSDADRRTRDY

SEQ ID NO: 670 - protein, 8 aa, Artificial Sequence
  synthetic construct
GSQFSHNL

SEQ ID NO: 671 - protein, 7 aa, Artificial Sequence
  synthetic construct
ITSFGST

SEQ ID NO: 672 - protein, 15 aa, Artificial Sequence
  synthetic construct
AALSSEDTSHYPRNY

SEQ ID NO: 673 - protein, 8 aa, Artificial Sequence
  synthetic construct
GYILSENL

SEQ ID NO: 674 - protein, 7 aa, Artificial Sequence
  synthetic construct
VTSGDYT

SEQ ID NO: 675 - protein, 13 aa, Artificial Sequence
  synthetic construct
AAGTSDWEVKWSH

SEQ ID NO: 676 - protein, 8 aa, Artificial Sequence
  synthetic construct
GIPASYLN

SEQ ID NO: 677 - protein, 7 aa, Artificial Sequence
  synthetic construct
AGGTNSN

SEQ ID NO: 678 - protein, 15 aa, Artificial Sequence
  synthetic construct
AALDGAESYDYVYPV

SEQ ID NO: 679 - protein, 8 aa, Artificial Sequence
  synthetic construct
GSTGSDYA

SEQ ID NO: 680 - protein, 7 aa, Artificial Sequence
  synthetic construct
ISSYGSD

SEQ ID NO: 681 - protein, 15 aa, Artificial Sequence
  synthetic construct
AALQASAGAGYYYDY